(19) United States
(12) Patent Application Publication
(10) Pub. No.: US 2024/0417387 A1
(43) Pub. Date: Dec. 19, 2024

(54) SULFONYLPIPERAZINYL COMPOUNDS FOR TREATMENT OF BACTERIAL INFECTIONS

(71) Applicant: Hoffmann-La Roche Inc., Little Falls, NJ (US)

(72) Inventors: Kurt Amrein, Basel (CH); Fabian Dey, Zurich (CH); Xiao Ding, Shanghai (CN); Xinyi Huang, Shanghai (CN); Christian Lerner, Bottmingen (CH); Houguang Shi, Shanghai (CN); Xuefei Tan, Shanghai (CN); Jun Wu, Shanghai (CN); Jiamin Zheng, Shanghai (CN); Mingwei Zhou, Shanghai (CN)

(73) Assignee: Hoffmann-La Roche Inc., Little Falls, NJ (US)

(21) Appl. No.: 18/700,259

(22) PCT Filed: Dec. 20, 2021
(86) PCT No.: PCT/EP2021/086690

§ 371 Date: Apr. 10, 2024

(30) Foreign Application Priority Data
Oct. 12, 2021 (WO) PCT/CN2021/123259

(51) Int. Cl. C07D 401/14; A61K 31/4545; A61K 31/496; A61K 31/497; A61K 31/506; A61K 31/5377; A61P 31/04; C07D 413/14; C07D 487/10

(52) U.S. Cl. CPC C07D 401/14; A61K 31/4545; A61K 31/496; A61K 31/497; A61K 31/506; A61K 31/5377; A61P 31/04; C07D 413/14; C07D 487/10

(57) ABSTRACT

The present invention relates to compounds of formula (I), wherein R¹ to R³, A, Q¹, Q², W and X are as described herein, and their pharmaceutically acceptable salt thereof, and compositions including the compounds and methods of using the compounds.

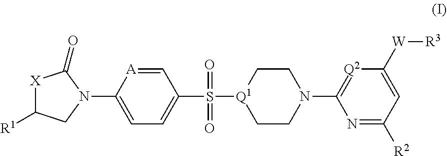

The present invention relates to organic compounds useful for the treatment and/or prevention of bacterial infections in a mammal. Specifically these molecules can inhibit the LPS synthesis pathway, in particular to inhibit LpxH, and are useful for treating bacterial infections.

### BACKGROUND OF THE INVENTION

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients. In hospital settings, an increasing number of strains of Staphylococcus aureus, Streptococcus pneumoniae, Enterococcus spp., Enterobacteriaceae such as Klebsiella pneumonia, Acinetobacter baumannii and Pseudomonas aeruginosa, major sources of infections, are becoming multi-drug resistant and therefore difficult to treat. This is particularly the case for Gram-negative organisms where the situation is getting worrisome since no novel agents with a differentiated mechanism of action have been approved for decades. Therefore, there is an important medical need for new antibacterial compounds addressing Gram-negative resistant bacteria, in particular third generation cephalosporins- and carbapenem-resistant Enterobacteriaceae and multi-drug-resistant Pseudomonas aeruginosa and Acinetobacter baumannii. One way to tackle the problem of cross-resistance to established classes of antibiotics is to inhibit an essential protein or function not targeted by current antibiotics.

Gram-negative bacteria are unique in that their outer membrane contains Lipopolysaccharide (LPS), which is crucial for maintaining membrane integrity, and is essential for bacterial viability (reviewed in Ann. Rev. Biochem 76: 295-329, 2007). The major lipid component of LPS is Lipid A, and inhibition of Lipid A biosynthesis is lethal to bacteria. Lipid A is synthesized on the cytoplasmic surface of the bacterial inner membrane via a pathway that consists of nine different enzymes. These enzymes are highly conserved in most Gram-negative bacteria. LpxH, a calcineurin-like phosphatase (CLP), catalyzes the hydrolysis of UDP-2,3-diacyl-glucosamine (UDP-DAGn) to yield Lipid X and UMP (22, 24, 25). LpxH has no mammalian homologue, making it a good target for the development of novel antibiotics targeting Gram-negative bacteria.

### SUMMARY OF THE INVENTION

The present invention relates to novel compounds of formula (I),

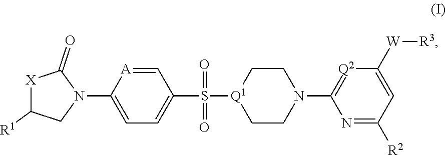

wherein
R¹ is amino, hydroxyC₁₋₆alkyl, C₁₋₆alkylamino, aminoC₁₋₆alkylamino or piperazinyl;
R² is halogen or C₁₋₆alkyl;
R³ is —Y—C(O)—R⁴; wherein
   Y is C₃₋₁₀cycloalkyl, phenyl, pyrazinyl or pyridinyl;
   R⁴ is ((aminoC₁₋₆alkyl)₂amino)C₁₋₆alkylamino, ((aminoC₁₋₆alkyl)pyrrolidiniumyl)C₁₋₆alkylamino, ((aminoC₁₋₆alkylcarbonyl)amino)C₁₋₆alkylamino, ((C₁₋₆alkyl)₂amino)C₁₋₆alkylamino, ((C₁₋₆alkyl)₃ammonio)C₁₋₆alkylamino, (aminoC₁₋₆alkoxy)C₁₋₆alkylamino, (aminoC₁₋₆alkyl(C₁₋₆alkyl)₂ammonio)C₁₋₆alkylamino, (C₁₋₆alkyl(aminoC₁₋₆alkyl)₂ammonio)C₁₋₆alkylamino, ((aminoC₁₋₆alkylcarbonyl)amino)C₃₋₁₀cycloalkylamino, ((aminoC₁₋₆alkyl)₂pyrrolidinyl)C₃₋₁₀cycloalkylamino, (aminoC₁₋₆ alkyl)₂amino, (aminoC₁₋₆alkyl)azetidinyl, (aminoC₁₋₆alkyl)C₃₋₁₀cycloalkylamino, (aminoC₁₋₆alkyl)piperazinyl, (aminoC₁₋₆alkylamino)C₁₋₆alkylamino, (aminoC₁₋₆alkylcarbonyl)aminocarbamoyl, (aminopyrrolidinyl)C₃₋₁₀cycloalkylamino, (azetidinylC₁₋₆alkylamino)C₁₋₆alkylamino, (C₁₋₆alkylamino)C₁₋₆alkylamino, (C₁₋₆alkylpyrrolidiniumyl)C₁₋₆alkylamino, (diaminopiperidinyl)C₁₋₆alkylamino, (diaminopiperidinyl)C₃₋₁₀cycloalkylamino, (piperidinylcarbonylamino)carbamoyl, 3-azabicyclo[3.1.0]hexanylamino, amino, amino(C₁₋₆alkoxy)C₁₋₆alkylamino, amino(C₁₋₆alkyl)piperidinyl, (aminoC₁₋₆alkyl)₂pyrrolidinyl, amino(hydroxy)C₁₋₆alkylamino, aminoC₁₋₆alkylamino, aminoC₃₋₁₀cycloalkylamino, aminocarbonylC₁₋₆alkylamino, aminopyrrolidinylamino, amino(C₁₋₆alkyl)pyrrolidinylamino, azetidinylC₁₋₆alkylamino, C₁₋₆alkoxy, C₁₋₆alkyl(aminoC₁₋₆alkyl)amino, C₁₋₆alkylpyrrolidinylamino, C₁₋₆alkyl(aminoC₁₋₆alkyl)piperaziniumyl, C₁₋₆alkyl-2,6-diazaspiro[3.3]heptanyl, C₁₋₆alkylamino, diaminoC₁₋₆alkylamino, diaminopiperidinyl, hydroxyC₁₋₆alkylamino, hydroxypyrrolidinylamino, morpholinylC₁₋₆alkylamino or pyrrolidinylC₁₋₆alkylamino;
X is CH₂ or O;
A is CH or N;
Q¹ is CH or N;
Q² is CH or N;
W is haloC₁₋₆alkyl;
or a pharmaceutically acceptable salt thereof.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and propyl.

The term “C₁₋₆alkoxy” denotes C₁₋₆alkyl-O—.

The term “amino”, alone or in combination, signifies the primary amino group, the secondary amino group, or the tertiary amino group.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, trifluoroethyl, fluoromethyl, difluoromethyl, difluoroethyl or trifluoromethyl.

The term “C₃₋₁₀cycloalkyl” denotes saturated carbon monocyclic, bicyclic or tricyclic ring containing from 3 to 10 carbon atoms, particularly from 3 to 8 carbon atoms. C₃₋₁₀cycloalkyl includes, for example, cyclopropyl, cyclobutyl, cyclopentyl, cyclohexyl, cycloheptyl, bicyclo[1.1.1]pentanyl, bicyclo[2.2.2]octanyl, bicyclo[3.1.0]hexanyl, norbornanyl, adamantanyl and the like. Particular “C₃₋₁₀cycloalkyl” groups are bicyclo[1.1.1]pentanyl, bicyclo[2.2.2]octanyl, bicyclo[3.1.0]hexanyl, cyclohexanyl, cyclopentanyl and norbornanyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Inhibitor of UDP-2,3-Diacylglucosamine Hydrolase (LpxH)

The present invention relates to (i) a compound of formula (I),

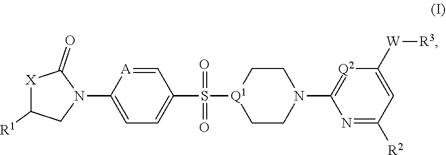

wherein
R¹ is amino, hydroxyC₁₋₆alkyl, C₁₋₆alkylamino, aminoC₁₋₆alkylamino or piperazinyl;
R² is halogen or C₁₋₆alkyl;
R³ is —Y—C(O)—R⁴; wherein
   Y is C₃₋₁₀cycloalkyl, phenyl, pyrazinyl or pyridinyl;
   R⁴ is ((aminoC₁₋₆alkyl)₂amino)C₁₋₆alkylamino, ((aminoC₁₋₆alkyl)pyrrolidiniumyl)C₁₋₆alkylamino, ((aminoC₁₋₆alkylcarbonyl)amino)C₁₋₆alkylamino, ((C₁₋₆alkyl)₂amino)C₁₋₆alkylamino, ((C₁₋₆alkyl)₃ammonio)C₁₋₆alkylamino, (aminoC₁₋₆alkoxy)C₁₋₆alkylamino, (aminoC₁₋₆alkyl(C₁₋₆alkyl)₂ammonio)C₁₋₆alkylamino, (C₁₋₆alkyl(aminoC₁₋₆alkyl)₂ammonio)C₁₋₆alkylamino, ((aminoC₁₋₆alkylcarbonyl)amino)C₃₋₁₀ cycloalkylamino, ((aminoC₁₋₆alkyl)₂pyrrolidinyl)C₃₋₁₀cycloalkylamino, (aminoC₁₋₆ alkyl)₂amino, (aminoC₁₋₆alkyl)azetidinyl, (aminoC₁₋₆alkyl)C₃₋₁₀cycloalkylamino, (aminoC₁₋₆alkyl)piperazinyl, (aminoC₁₋₆alkylamino)C₁₋₆alkylamino, (aminoC₁₋₆alkylcarbonyl)aminocarbamoyl, (aminopyrrolidinyl)C₃₋₁₀cycloalkylamino, (azetidinylC₁₋₆alkylamino)C₁₋₆alkylamino, (C₁₋₆alkylamino)C₁₋₆alkylamino, (C₁₋₆alkylpyrrolidiniumyl)C₁₋₆alkylamino, (diaminopiperidinyl)C₁₋₆alkylamino, (diaminopiperidinyl)C₃₋₁₀cycloalkylamino, (piperidinylcarbonylamino)carbamoyl, 3-azabicyclo[3.1.0]hexanylamino, amino, amino(C₁₋₆alkoxy)C₁₋₆alkylamino, amino(C₁₋₆alkyl)piperidinyl, (aminoC₁₋₆alkyl)₂pyrrolidinyl, amino(hydroxy)C₁₋₆alkylamino, aminoC₁₋₆alkylamino, aminoC₃₋₁₀cycloalkylamino, aminocarbonylC₁₋₆alkylamino, aminopyrrolidinylamino, amino(C₁₋₆alkyl)pyrrolidinylamino, azetidinylC₁₋₆alkylamino, C₁₋₆alkoxy, C₁₋₆alkyl(aminoC₁₋₆alkyl)amino, C₁₋₆alkylpyrrolidinylamino, C₁₋₆ alkyl(aminoC₁₋₆alkyl)piperaziniumyl, C₁₋₆alkyl-2,6-diazaspiro[3.3]heptanyl, C₁₋₆ alkylamino, diaminoC₁₋₆alkylamino, diaminopiperidinyl, hydroxyC₁₋₆alkylamino, hydroxypyrrolidinylamino, morpholinylC₁₋₆alkylamino or pyrrolidinylC₁₋₆alkylamino;
X is CH₂ or O;
A is CH or N;
Q¹ is CH or N;
Q² is CH or N;
W is haloC₁₋₆alkyl;
or a pharmaceutically acceptable salt thereof.

The present invention relates to (i′) a compound of formula (I),

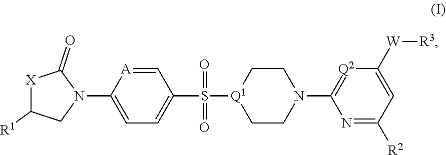

wherein
R¹ is amino, hydroxyC₁₋₆alkyl, aminoC₁₋₆alkylamino or piperazinyl;
R² is halogen or C₁₋₆alkyl;
R³ is —Y—C(O)—R⁴; wherein
   Y is C₃₋₁₀cycloalkyl, phenyl, pyrazinyl or pyridinyl;
   R⁴ is ((aminoC₁₋₆alkyl)₂amino)C₁₋₆alkylamino, ((aminoC₁₋₆alkyl)pyrrolidiniumyl)C₁₋₆alkylamino, ((aminoC₁₋₆alkylcarbonyl)amino)C₁₋₆alkylamino, ((C₁₋₆alkyl)₂amino)C₁₋₆alkylamino, ((C₁₋₆alkyl)₃ammonio)C₁₋₆alkylamino, (aminoC₁₋₆alkoxy)C₁₋₆alkylamino, (aminoC₁₋₆alkyl(C₁₋₆alkyl)₂ammonio)C₁₋₆alkylamino, (aminoC₁₋₆alkyl)₂amino, (aminoC₁₋₆alkyl)azetidinyl, (aminoC₁₋₆alkyl)C₃₋₁₀cycloalkylamino, (aminoC₁₋₆alkyl)piperazinyl, (aminoC₁₋₆alkylamino)C₁₋₆alkylamino, (aminoC₁₋₆alkylcarbonyl)aminocarbamoyl, (azetidinylC₁₋₆alkylamino)C₁₋₆alkylamino, (C₁₋₆alkylamino)C₁₋₆alkylamino, (C₁₋₆alkylpyrrolidiniumyl)C₁₋₆alkylamino, (diaminopiperidinyl)C₁₋₆alkylamino, (piperidinylcarbonylamino)carbamoyl, 3-azabicyclo[3.1.0]hexanylamino, amino, amino(C₁₋₆alkoxy)C₁₋₆alkylamino, amino(C₁₋₆alkyl)piperidinyl, amino(hydroxy)C₁₋₆alkylamino, aminoC₁₋₆alkylamino, aminoC₃₋₁₀cycloalkylamino, aminocarbonylC₁₋₆alkylamino, aminopyrrolidinylamino, azetidinylC₁₋₆alkylamino, C₁₋₆alkoxy, C₁₋₆alkyl(aminoC₁₋₆alkyl)amino, C₁₋₆alkyl(aminoC₁₋₆alkyl)piperaziniumyl, C₁₋₆alkyl-2,6-diazaspiro[3.3]heptanyl, C₁₋₆ alkylamino, diaminoC₁₋₆alkylamino, diaminopiperidinyl, hydroxyC₁₋₆alkylamino, hydroxypyrrolidinylamino, morpholinylC₁₋₆alkylamino or pyrrolidinylC₁₋₆alkylamino;
X is CH₂ or O;
A is CH or N;
Q¹ is CH or N;
Q² is CH or N;
W is haloC₁₋₆alkyl;
or a pharmaceutically acceptable salt thereof.

A further embodiment of present invention is (ii′) a compound of formula (I) according to (i), or a pharmaceutically acceptable salt thereof, wherein R¹ is amino or hydroxyC₁₋₆alkyl.

A further embodiment of present invention is (ii) a compound of formula (I) according to (i), or a pharmaceutically acceptable salt thereof, wherein R¹ is amino, C₁₋₆alkylamino or hydroxyC₁₋₆alkyl.

A further embodiment of present invention is (iii′) a compound of formula (I) according to (i) or (ii), or a pharmaceutically acceptable salt thereof, wherein R¹ is amino or hydroxymethyl.

A further embodiment of present invention is (iii) a compound of formula (I) according to (i) or (ii), or a pharmaceutically acceptable salt thereof, wherein R¹ is amino, methylamino or hydroxymethyl.

A further embodiment of present invention is (iv) a compound of formula (I), according to any one of (i) to (iii), or a pharmaceutically acceptable salt thereof, wherein R² is halogen.

A further embodiment of present invention is (v) a compound of formula (I) according to any one of (i) to (iv), wherein R² is chloro.

A further embodiment of present invention is (vi) a compound of formula (I), or a pharmaceutically acceptable salt thereof, according to any one of (i) to (v), wherein R³ is —Y—C(O)—R⁴; wherein Y is C₃₋₁₀cycloalkyl, phenyl or pyrazinyl.

A further embodiment of present invention is (vii′) a compound of formula (I), or a pharmaceutically acceptable salt thereof, according to any one of (i) to (vi), wherein R³ is —Y—C(O)—R⁴; wherein Y is bicyclo[2.2.2]octanyl, bicyclo[3.1.0]hexanyl, cyclohexanyl, phenyl or pyrazinyl.

A further embodiment of present invention is (vii) a compound of formula (I), or a pharmaceutically acceptable salt thereof, according to any one of (i) to (vi), wherein R³ is —Y—C(O)—R⁴; wherein Y is bicyclo[2.2.2]octanyl, bicyclo[3.1.0]hexanyl, cyclohexanyl, norbornanyl, phenyl or pyrazinyl.

A further embodiment of present invention is (viii′) a compound of formula (I), or a pharmaceutically acceptable salt thereof, according to any one of (i) to (vii), wherein R³ is —Y—C(O)—R⁴; wherein R⁴ is ((aminoC₁₋₆alkyl)₂amino)C₁₋₆alkylamino, ((aminoC₁₋₆ alkyl)pyrrolidiniumyl)C₁₋₆alkylamino, (aminoC₁₋₆alkyl(C₁₋₆alkyl)₂ammonio)C₁₋₆alkylamino, (aminoC₁₋₆alkyl)₂amino, (diaminopiperidinyl)C₁₋₆alkylamino, amino, amino(hydroxy)C₁₋₆alkylamino or aminoC₁₋₆alkylamino.

A further embodiment of present invention is (viii) a compound of formula (I), or a pharmaceutically acceptable salt thereof, according to any one of (i) to (vii), wherein R³ is —Y—C(O)—R⁴; wherein R⁴ is amino(C₁₋₆alkyl)pyrrolidinylamino, ((aminoC₁₋₆alkyl)₂amino)C₁₋₆alkylamino, ((aminoC₁₋₆alkyl)₂pyrrolidinyl)C₃₋₁₀cycloalkylamino, ((aminoC₁₋₆ alkyl)pyrrolidiniumyl)C₁₋₆alkylamino, (aminoC₁₋₆alkyl(C₁₋₆alkyl)₂ammonio)C₁₋₆alkylamino, (aminoC₁₋₆alkyl)₂amino, (aminoC₁₋₆alkyl)₂pyrrolidinyl, (aminoC₁₋₆alkyl)C₃₋₁₀cycloalkylamino, (C₁₋₆alkyl(aminoC₁₋₆alkyl)₂ammonio)C₁₋₆alkylamino, (aminopyrrolidinyl)C₃₋₁₀cycloalkylamino, (C₁₋₆alkyl(aminoC₁₋₆alkyl)₂ammonio)C₁₋₆alkylamino, (diaminopiperidinyl)C₁₋₆alkylamino, (diaminopiperidinyl)C₃₋₁₀cycloalkylamino, amino, amino(hydroxy)C₁₋₆alkylamino, aminoC₁₋₆alkylamino, aminoC₃₋₁₀cycloalkylamino, C₁₋₆alkylpyrrolidinylamino, diaminoC₁₋₆alkylamino or diaminopiperidinyl.

A further embodiment of present invention is (ix′) a compound of formula (I), or a pharmaceutically acceptable salt thereof, according to any one of (i) to (viii), wherein R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (3-aminopropyl)₂amino, (3,5-diamino-1-piperidinyl)propylamino, amino, 3-amino-2-hydroxy-propylamino, 3-aminopropylamino, 3-aminopropylamino or 2-aminoethylamino.

A further embodiment of present invention is (ix) a compound of formula (I), or a pharmaceutically acceptable salt thereof, according to any one of (i) to (viii), wherein R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (2-aminoethyl)₂amino, (3,5-diamino-1-piperidinyl)propylamino, (3-aminocyclopentyl)amino, (3-aminopropyl)₂amino, (4-methylpyrrolidin-3-yl)amino, [3-(3,5-diamino-1-piperidinyl)cyclopentyl]amino, [3-[3,4-bis(aminomethyl)pyrrolidin-1-yl]cyclopentyl]amino, [3-[3-aminopyrrolidin-1-yl]cyclopentyl]amino, 1-(aminomethyl)cyclopropylamino, 2-(2-aminoethylamino)ethylamino, 2,3-diaminopropylamino, 2-aminoethylamino, 3,4-bis(aminomethyl)pyrrolidin-1-yl, 3,5-diaminopiperidinyl, 3-[3-aminopropyl(dimethyl)ammonio]propylamino, 3-[bis(2-aminoethyl)-methyl-ammonio]propylamino, 3-amino-2-hydroxy-propylamino, 3-aminopropylamino, 3-hydroxypropylamino, 4-amino-2-methyl-pyrrolidin-1-yl, amino or aminopropylamino.

A further embodiment of present invention is (x) a compound of formula (I), or a pharmaceutically acceptable salt thereof, according to any one of (i) to (ix), wherein A is CH.

A further embodiment of present invention is (xi) a compound of formula (I), or a pharmaceutically acceptable salt thereof, according to any one of (i) to (x), wherein Q¹ is N.

A further embodiment of present invention is (xii) a compound of formula (I), or a pharmaceutically acceptable salt thereof, according to any one of (i) to (xi), wherein Q² is CH.

A further embodiment of present invention is (xiii) a compound of formula (I), or a pharmaceutically acceptable salt thereof, according to any one of (i) to (xii), wherein W is CF₂.

A further embodiment of present invention is (xiv′) a compound of formula (I), according to any one of (i) to (xiii), wherein
R¹ is amino or hydroxyC₁₋₆alkyl;
R² is halogen;
R³ is —Y—C(O)—R⁴; wherein
   Y is C₃₋₁₀cycloalkyl, phenyl or pyrazinyl;
   R⁴ is ((aminoC₁₋₆alkyl)₂amino)C₁₋₆alkylamino, ((aminoC₁₋₆alkyl)pyrrolidiniumyl)C₁₋₆alkylamino, (aminoC₁₋₆alkyl(C₁₋₆alkyl)₂ammonio)C₁₋₆alkylamino, (aminoC₁₋₆alkyl)₂amino, (diaminopiperidinyl)C₁₋₆alkylamino, amino, amino(hydroxy)C₁₋₆alkylamino or aminoC₁₋₆alkylamino;
X is CH₂ or O;
A is CH;
Q¹ is N;
Q² is CH;
W is haloC₁₋₆alkyl;
   or a pharmaceutically acceptable salt thereof.

A further embodiment of present invention is (xiv) a compound of formula (I), according to any one of (i) to (xiii), wherein
R¹ is amino or hydroxyC₁₋₆alkyl;
R² is halogen;
R³ is —Y—C(O)—R⁴; wherein
   Y is C₃₋₁₀cycloalkyl, phenyl or pyrazinyl;
   R⁴ is amino(C₁₋₆alkyl)pyrrolidinylamino, ((aminoC₁₋₆alkyl)₂amino)C₁₋₆alkylamino, ((aminoC₁₋₆alkyl)₂pyrrolidinyl)C₃₋₁₀cycloalkylamino, ((aminoC₁₋₆ alkyl)pyrrolidiniumyl)C₁₋₆alkylamino, (aminoC₁₋₆alkyl(C₁₋₆alkyl)₂ammonio)C₁₋₆alkylamino, (aminoC₁₋₆alkyl)₂amino, (aminoC₁₋₆alkyl)₂pyrrolidinyl, (aminoC₁₋₆ alkyl)C₃₋₁₀cycloalkylamino, (C₁₋₆alkyl(aminoC₁₋₆alkyl)₂ammonio)C₁₋₆alkylamino, (aminopyrrolidinyl)C₃₋₁₀cycloalkylamino, (C₁₋₆alkyl(aminoC₁₋₆alkyl)₂ammonio)C₁₋₆alkylamino, (diaminopiperidinyl)C₁₋₆alkylamino, (diaminopiperidinyl)C₃₋₁₀ cycloalkylamino, amino, amino(hydroxy)C₁₋₆alkylamino, aminoC₁₋₆alkylamino, aminoC₃₋₁₀cycloalkylamino, C₁₋₆alkylpyrrolidinylamino, diaminoC₁₋₆alkylamino or diaminopiperidinyl;
X is CH₂ or O;
A is CH;
Q¹ is N;
Q² is CH;
W is haloC₁₋₆alkyl;
   or a pharmaceutically acceptable salt thereof.

A further embodiment of present invention is (xv′) a compound of formula (I), according to any one of (i) to (xiv), wherein
R¹ is amino or hydroxymethyl;
R² is chloro;
R³ is —Y—C(O)—R⁴; wherein
   Y is bicyclo[2.2.2]octanyl, bicyclo[3.1.0]hexanyl, cyclohexanyl, phenyl or pyrazinyl;
   R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (3-aminopropyl)₂amino, (3,5-diamino-1-piperidinyl)propylamino, amino, 3-amino-2-hydroxy-propylamino, 3-aminopropylamino, 3-aminopropylamino or 2-aminoethylamino;
X is CH₂ or O;
A is CH;
Q¹ is N;
Q² is CH;
W is CF₂;
or a pharmaceutically acceptable salt thereof.

A further embodiment of present invention is (xv) a compound of formula (I), according to any one of (i) to (xiv), wherein
R¹ is amino or hydroxymethyl;
R² is chloro;
R³ is —Y—C(O)—R⁴; wherein
   Y is bicyclo[2.2.2]octanyl, bicyclo[3.1.0]hexanyl, cyclohexanyl, phenyl or pyrazinyl;
   R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (2-aminoethyl)₂amino, (3,5-diamino-1-piperidinyl)propylamino, (3-aminocyclopentyl)amino, (3-aminopropyl)2amino, (4-methylpyrrolidin-3-yl)amino, [3-(3,5-diamino-1-piperidinyl)cyclopentyl]amino, [3-[3,4-bis(aminomethyl)pyrrolidin-1-yl]cyclopentyl]amino, [3-[3-aminopyrrolidin-1-yl]cyclopentyl]amino, 1-(aminomethyl)cyclopropylamino, 2-(2-aminoethylamino)ethylamino, 2,3-diaminopropylamino, 2-aminoethylamino, 3,4-bis(aminomethyl)pyrrolidin-1-yl, 3,5-diaminopiperidinyl, 3-[3-aminopropyl(dimethyl)ammonio]propylamino, 3-[bis(2-aminoethyl)-methyl-ammonio]propylamino, 3-amino-2-hydroxy-propylamino, 3-aminopropylamino, 3-hydroxypropylamino, 4-amino-2-methyl-pyrrolidin-1-yl, amino or aminopropylamino;
X is CH₂ or O;
A is CH;
Q¹ is N;
Q² is CH;
W is CF₂;
or a pharmaceutically acceptable salt thereof.

Another embodiment of present invention is a compound of formula (I) selected from the following:
Trans-methyl 4-[[2-[4-[4-(4-amino-2-oxo-pyrrolidin-1-yl)phenyl]sulfonylpiperazin-1-yl]-6-chloro-4-pyridyl]-difluoro-methyl]cyclohexanecarboxylate;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(azetidin-3-ylmethyl)cyclohexanecarboxamide;
Trans-4-[[2-[4-[4-(4-amino-2-oxo-pyrrolidin-1-yl)phenyl]sulfonylpiperazin-1-yl]-6-chloro-4-pyridyl]-difluoro-methyl]-N-(3-hydroxypropyl)cyclohexanecarboxamide;
Trans-(4R)-4-amino-1-[4-[4-[4-[[4-(4-amino-4-methyl-piperidine-1-carbonyl)cyclohexyl]-difluoro-methyl]-6-chloro-2-pyridyl]piperazin-1-yl]sulfonylphenyl]pyrrolidin-2-one;
Trans-(4R)-4-amino-1-[4-[4-[6-chloro-4-[difluoro-[4-(6-methyl-2,6-diazaspiro[3.3]heptane-2-carbonyl)cyclohexyl]methyl]-2-pyridyl]piperazin-1-yl]sulfonylphenyl]pyrrolidin-2-one;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(4-hydroxypyrrolidin-3-yl)cyclohexanecarboxamide;
Trans-N-(3-aminocyclobutyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-(2-amino-1-methyl-ethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2-pyrrolidin-1-ylethyl)cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[(1S,5R)-3-azabicyclo[3.1.0]hexan-6-yl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[cis-4-aminopyrrolidin-3-yl]cyclohexanecarboxamide;
Trans-N-[1-(aminomethyl)cyclopropyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-(4R)-4-amino-1-[4-[4-[4-[[4-[4-(2-aminoethyl)piperazine-1-carbonyl]cyclohexyl]-difluoro-methyl]-6-chloro-2-pyridyl]piperazin-1-yl]sulfonylphenyl]pyrrolidin-2-one;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
4-[[2-[4-[4-(4-amino-2-oxo-pyrrolidin-1-yl)phenyl]sulfonylpiperazin-1-yl]-6-chloro-4-pyridyl]-difluoro-methyl]-N-(3-hydroxypropyl)benzamide;
4-[[2-[4-[4-(4-amino-2-oxo-pyrrolidin-1-yl)phenyl]sulfonylpiperazin-1-yl]-6-chloro-4-pyridyl]-difluoro-methyl]-N-(3-aminopropyl)benzamide;
N-(2-aminoethyl)-4-[[2-[4-[4-(4-amino-2-oxo-pyrrolidin-1-yl)phenyl]sulfonylpiperazin-1-yl]-6-chloro-4-pyridyl]-difluoro-methyl]benzamide;
N-(3-amino-2-hydroxy-propyl)-4-[[2-[4-[4-(4-amino-2-oxo-pyrrolidin-1-yl)phenyl]sulfonylpiperazin-1-yl]-6-chloro-4-pyridyl]-difluoro-methyl]benzamide;
N-(3-amino-2-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]benzamide;
4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]benzamide;
N-(3-aminopropyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
N-(3-amino-2-hydroxy-propyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]pyrazine-2-carboxamide;
N-(2-aminoethyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2-morpholinoethyl)pyrazine-2-carboxamide;
N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]pyridine-3-carboxamide;
6-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]pyridine-3-carboxamide;
N-[2-(2-aminoethylamino)ethyl]-6-[[2-chloro-6-[4-[4-[(4R)-4-amino-2-oxo-pyrrolidin-1-yl]phenyl]sulfonylpiperazin-1-yl]-4-pyridyl]-difluoro-methyl]pyridine-3-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(methylamino)propyl]pyridine-2-carboxamide;
Trans-N-(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-methyl-cyclohexanecarboxamide;
Cis-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Cis-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(3-hydroxypropyl)cyclohexanecarboxamide;
N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[2.2.2]octane-1-carboxamide;
N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]norbornane-1-carboxamide;
N-(3-aminopropyl)-3-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclopentanecarboxamide;
Trans-N-(3-amino-2-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
3-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]benzoyl]amino]propyl-trimethyl-ammonium;
4-[[2-Chloro-6-[4-[4-[(4R)-4-amino-2-oxo-pyrrolidin-1-yl]phenyl]sulfonylpiperazin-1-yl]-4-pyridyl]-difluoro-methyl]-N-[3-(1-methylpyrrolidin-1-ium-1-yl)propyl]benzamide;
5-[[2-Chloro-6-[4-[4-[(4R)-4-amino-2-oxo-pyrrolidin-1-yl]phenyl]sulfonylpiperazin-1-yl]-4-pyridyl]-difluoro-methyl]-N-[3-(2-hydroxyethylamino)propyl]pyridine-2-carboxamide;
N-(3-aminopropyl)-3-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[1.1.1]pentane-1-carboxamide;
Trans-N-(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-[2-(2-aminoethoxy)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N,N-bis(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[2.2.2]octane-1-carboxamide;
N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]norbornane-1-carboxamide;
Trans-N-[3-[bis(2-aminoethyl)amino]propyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[6-chloro-4-[[4-cis-(3,5-diaminopiperidine-1-carbonyl)cyclohexyl]-difluoro-methyl]-2-pyridyl]piperazin-1-yl]sulfonylphenyl]pyrrolidin-2-one;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-[cis-3,5-diamino-1-piperidyl]propyl]cyclohexanecarboxamide;
Trans-N′-(2-aminoacetyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbohydrazide;
Trans-N′-[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]piperidine-4-carbohydrazide;
Trans-N-(2-amino-3-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
(2R,5S)-5-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(3-aminopropyl)bicyclo[2.2.2]octane-2-carboxamide;
Trans-N-(3-aminopropyl)-4-[difluoro-[6-methyl-2-[4-[4-[(4R)-4-amino-2-oxo-pyrrolidin-1-yl]phenyl]sulfonylpiperazin-1-yl]pyrimidin-4-yl]methyl]cyclohexanecarboxamide;
N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[3.1.0]hexane-3-carboxamide;
Trans-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[4-[[4-[3-(aminomethyl)azetidine-1-carbonyl]cyclohexyl]-difluoro-methyl]-6-chloro-2-pyridyl]piperazin-1-yl]sulfonylphenyl]pyrrolidin-2-one;
Trans-N-(3-aminopropyl)-4-[[2-chloro-6-[4-[4-[(5S)-5-(hydroxymethyl)-2-oxo-oxazolidin-3-yl]phenyl]sulfonylpiperazin-1-yl]-4-pyridyl]-difluoro-methyl]cyclohexanecarboxamide;
Trans-N-(3-aminopropyl)-4-[[2-chloro-6-[4-[4-[(5R)-5-(hydroxymethyl)-2-oxo-oxazolidin-3-yl]phenyl]sulfonylpiperazin-1-yl]-4-pyridyl]-difluoro-methyl]cyclohexanecarboxamide;
Trans-N-(3-aminopropyl)-4-[[2-chloro-6-[4-[4-(2-oxo-4-piperazin-1-yl-pyrrolidin-1-yl)phenyl]sulfonylpiperazin-1-yl]-4-pyridyl]-difluoro-methyl]cyclohexanecarboxamide;
Trans-N-(3-aminopropyl)-4-[[2-chloro-6-[4-[[6-[(4R)-4-amino-2-oxo-pyrrolidin-1-yl]-3-pyridyl]sulfonyl]piperazin-1-yl]-4-pyridyl]-difluoro-methyl]cyclohexanecarboxamide;
Trans-N-(3-aminopropyl)-4-[[2-chloro-6-[4-[4-[(4R)-4-amino-2-oxo-pyrrolidin-1-yl]phenyl]sulfonyl-1-piperidyl]-4-pyridyl]-difluoro-methyl]cyclohexanecarboxamide;
Cis-N-(2-amino-2-oxo-ethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
(4R)-4-amino-1-[4-[4-[4-[[4-[(2S,4S)-4-amino-2-methyl-pyrrolidine-1-carbonyl]cyclohexyl]-difluoro-methyl]-6-chloro-2-pyridyl]piperazin-1-yl]sulfonylphenyl]pyrrolidin-2-one;
Trans-bis(2-aminoethyl)-[3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl]-methyl-ammonium;
Trans-N-[(1R,3S)-3-aminocyclopentyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-(4R)-4-amino-1-[4-[4-[4-[[4-[cis-3,4-bis(aminomethyl)pyrrolidine-1-carbonyl]cyclohexyl]-difluoro-methyl]-6-chloro-2-pyridyl]piperazin-1-yl]sulfonylphenyl]pyrrolidin-2-one;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(3R,4S)-4-methylpyrrolidin-3-yl]cyclohexanecarboxamide;
Trans-N-(3-aminopropyl)-4-[[2-chloro-6-[4-[4-[(4R)-4-(methylamino)-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[4-[[4-[4-(2-aminoethyl)-4-methyl-piperazin-4-ium-1-carbonyl]cyclohexyl]-difluoro-methyl]-6-chloro-2-pyridyl]piperazin-1-yl]sulfonylphenyl]pyrrolidin-2-one;
Trans-N-[2-(2-aminoethylamino)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-2-aminoethyl-[2-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]amino]ethyl]-dimethyl-ammonium;
Trans-N-[2-[1-(2-aminoethyl)pyrrolidin-1-ium-1-yl]ethyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-N-[2-[(2-aminoacetyl)amino]ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-(3-amino-2-methoxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2,3-diaminopropyl)cyclohexanecarboxamide;
4-[[2-[4-[4-[4-(3-Aminopropylamino)-2-oxo-pyrrolidin-1-yl]phenyl]sulfonylpiperazin-1-yl]-6-chloro-4-pyridyl]-difluoro-methyl]-N-methyl-benzamide;
Trans-N-[(1R,3S)-3-[(2-aminoacetyl)amino]cyclopentyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[cis-3,4-bis(aminomethyl)pyrrolidin-1-yl]cyclopentyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[(1S)-3-[cis-3,5-diamino-1-piperidyl]cyclopentyl]cyclohexanecarboxamide;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[(3R)-3-aminopyrrolidin-1-yl]cyclopentyl]cyclohexanecarboxamide; and
Trans-3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl-(3-aminopropyl)-dimethyl-ammonium;
   or a pharmaceutically acceptable salt thereof.

A further embodiment of present invention is (xviii) a compound of formula (I), or a pharmaceutically acceptable salt thereof, according to any one of (i) to (xvi), for use as therapeutically active substance.

A further embodiment of present invention is (xix) a pharmaceutical composition comprising a compound according to any one of (i) to (xvi), and a therapeutically inert carrier.

A further embodiment of present invention is (xx) the use of a compound according to any one of (i) to (xvi), for the inhibition of LpxH.

A further embodiment of present invention is (xxi) the use of a compound according to any one of (i) to (xvi), for the treatment or prophylaxis of bacterial infection, particularly the bacteria is gram-negative bacteria.

A further embodiment of present invention is (xxii) the use of a compound according to any one of (i) to (xvi), for the preparation of a medicament for the treatment or prophylaxis of bacterial infection, particularly the bacteria is gram-negative bacteria.

A further embodiment of present invention is (xxiii) the use of a compound according to (xxi) or (xxii), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxiv) the use of a compound according to (xxiii) wherein the gram-negative bacteria is Enterobacteriaceae, wherein Enterobacteriaceae is Klebsiella pneumoniae or Escherichia coli.

A further embodiment of present invention is (xxv) a compound according to any one of (i) to (xvi), for the treatment or prophylaxis of bacterial infection, particularly the bacteria is gram-negative bacteria.

A further embodiment of present invention is (xxvi) a compound according to (xxv), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxvii) a compound according to (xxvi), wherein the gram-negative bacteria is Enterobacteriaceae, wherein Enterobacteriaceae is Klebsiella pneumoniae or Escherichia coli.

A further embodiment of present invention is (xxviii) a method for the treatment or prophylaxis of bacterial infection, particularly the bacteria is gram-negative bacteria, which method comprises administering a therapeutically effective amount of a compound according to any one of (i) to (xvi).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments.

In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of Lipid A biosynthesis by targeting LpxH enzyme. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day, with the typical initial range of compound used being 0.3 to 15 mg/kg/day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or a stereoisomer or pharmaceutically acceptable salt thereof. In a further embodiment includes a pharmaceutical composition comprising a compound of Formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment and/or prevention of bacterial infections.

The following composition A and B illustrate typical compositions of the present invention, but serve merely as representative thereof.

### Composition A

A compound of the present invention can be used in a manner known per se as the active ingredient for the production of tablets of the following composition:

### Per Tablet

Active ingredient 200 mg
Microcrystalline cellulose 155 mg
Corn starch 25 mg
Talc 25 mg
Hydroxypropylmethylcellulose 20 mg
   425 mg

### Composition B

A compound of the present invention can be used in a manner known per se as the active ingredient for the production of capsules of the following composition:

### Per Capsule

Active ingredient 100.0 mg
Corn starch 20.0 mg
Lactose 95.0 mg
Talc 4.5 mg
Magnesium stearate 0.5 mg
   220.0 mg

### Indications and Methods of Treatment

The compounds of the invention are inhibitors of the LpxH enzyme, a key enzyme of the LPS synthesis pathway that is essential in most gram-negative bacteria. Accordingly, the compounds of the invention can prevent bacterial growth of susceptible organisms and are useful for: preventing or treating a bacterial infection, preferably a Gram-negative bacterial infection (all claimed) e.g. nosocomial pneumonia, urinary tract infections, systemic infections (bacteraemia and sepsis), skin and soft tissue infections, surgical infections, eye infections, intraabdominal infections, lung infections and diabetic foot infections caused by Gram-negative bacteria e.g. third generation cephalosporins- and carbapenem-resistant Enterobacteriaeceae (e.g. Klebsiella pneumoniae, Escherichia coli) and multi-drug-resistant Pseudomonas aeruginosa and Acinetobacter baumannii or Acinetobacter spp., e.g. Neisseria gonorrhoeae, Haemophilus influenzae, Helicobacter pylorus e.g. Bacteroides spp. e.g. Bacteroides fragilis, Bacteroides thetaiotaomicron, Bacteroides distasonis, Campylobacter jejuni, Campylobacter fetus or Campylobacter coil, Francisella tularensis and Providencia spp. e.g. Providencia stuartii, Providencia rettgeri or Providencia alcalifaciens and Pseudomonas spp.; and for cleaning purposes e.g. to remove pathogenic microbes and bacteria from surgical instruments, catheters and artificial implants or to make a room or an area aseptic.

The products of the invention can be administered, for example, parenterally e.g. by injection, or administered orally, perorally, such as in the form of tablets, coated tablets, dragees, hard and soft gelatin capsules, solutions, emulsions or suspensions, or rectally, such as in the form of suppositories. Pharmaceutical compositions containing these compounds can be prepared using conventional procedures familiar to those skilled in the art, such as by combining the ingredients into a dosage form together with suitable, non-toxic, inert, therapeutically compatible solid or liquid carrier materials and, if desired, the usual pharmaceutical adjuvants. It is contemplated that the compounds are ultimately embodied into compositions of suitable oral, parenteral or topical dosage forms. The compositions of this invention can contain, as optional ingredients, any of the various adjuvants, which are used ordinarily in the production of pharmaceutical preparations. Thus, for example, in formulating the present compositions into the desired oral dosage forms, one may use, as optional ingredients, fillers, such as co-precipitated aluminum hydroxide-calcium carbonate, di-calcium phosphate or lactose; disintegrating agents such as maize starch; and lubricating agents, such as talc, calcium stearate, and the like. It should be fully understood, however, that the optional ingredients herein named are given by way of example only and that the invention is not restricted to the use hereof. Other such adjuvants, which are well known in the art, can be employed in carrying out this invention. Suitable as such carrier materials are not only inorganic, but also organic carrier materials. Thus, for tablets, coated tablets, dragees and hard gelatin capsules there can be used, for example, lactose, maize starch or derivatives thereof, talc, stearic acid or its salts. Suitable carriers for soft gelatin capsules are, for example, vegetable oils, waxes, fats and semi-solid and liquid polyols (depending on the nature of the active substance; no carriers are, however, required in the case of soft gelatin capsules). Suitable carrier materials for the preparation of solutions and syrups are, for example, water, polyols, saccharose, invert sugar and glucose. Suitable carrier materials for suppositories are, for example, natural or hardened oils, waxes, fats and semi-liquid or liquid polyols. As pharmaceutical adjuvants there are contemplated the usual preservatives, solubilizers, stabilizers, wetting agents, emulsifiers, sweeteners, colorants, flavorings, salts for varying the osmotic pressure, buffers, coating agents and antioxidants.

Synthesis The compounds of the present invention can be prepared by any conventional means.

Suitable processes for synthesizing these compounds as well as their starting materials are provided in the schemes below and in the subsequent examples. All substituents, in particular, R¹ to R⁴, Y, A, Q¹, Q², W and X are defined as below unless otherwise indicated. Furthermore, and unless explicitly otherwise stated, all reactions, reaction conditions, abbreviations and symbols have the meanings well known to a person of ordinary skill in the art.

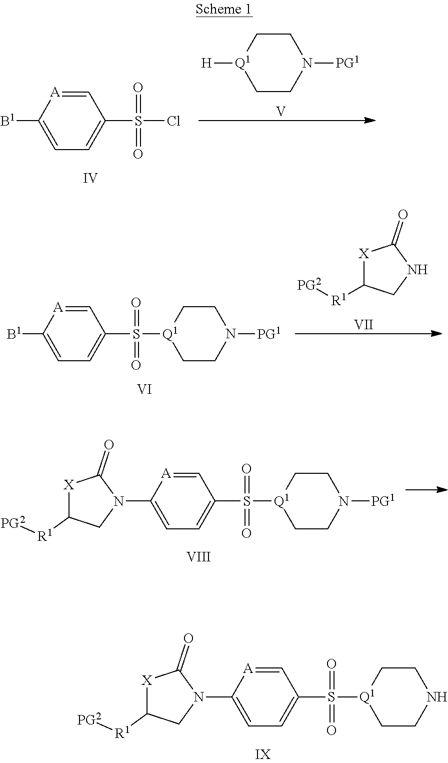

Wherein Q¹ is N; B¹ is halogen; PG¹ and PG² independently are protecting groups, such as tert-butoxycarbonyl and benzyloxycarbonyl group.

Compound of formula (VI) could be obtained from a sulfonating reaction between halogenated phenyl sulfonyl chlorides (IV) and compound of formula (V). The coupling reaction of compound of formula (VI) with a cyclic amide such as lactam or carbamate (VII), using catalysts, such as CuI, in the presence of a base, such as Cs₂CO₃, affords the compound of formula (VIII). Compound of formula (IX) can be obtained by the deprotection of compound of formula (VIII) with a suitable acid such as trifluoroacetic acid, or a reducing reagent such as palladium on carbon in the presence of H₂.

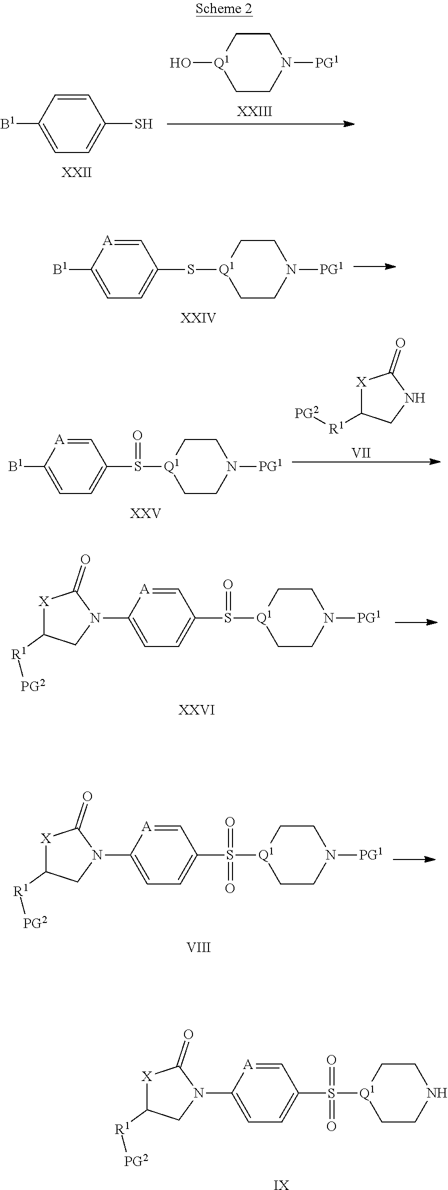

Wherein Q¹ is CH.

Alternatively compound of formula (IX) can also be prepared in the process illustrated in scheme 2. The reaction between compound of formula (XXIII) and compound of formula (XXII) will give compound of formula (XXIV) in the presence of suitable Mitsunobu reagent, such as N,N,N,N-tetramethylazodicarboxamide and tributylphosphine. Compound of formula (XXV) can be obtained from the oxidation of compound of formula (XXIV) using a suitable oxidizing reagent, such as m-CPBA. The coupling reaction of compound of formula (XXV) with a cyclic amide such as lactam or carbamate (VII), using catalysts such as CuI, in the presence of a base, such as Cs₂CO₃, affords sulfoxide (XXVI). The oxidation of sulfoxide (XXVI) with a suitable oxidizing reagent, such as m-CPBA, affords compound of formula (XXVII). Compound of formula (IX) can be obtained by the deprotection of compound of formula (VIII) with a suitable acid such as trifluoroacetic acid, or a reducing reagent such as palladium on carbon in the presence of H₂.

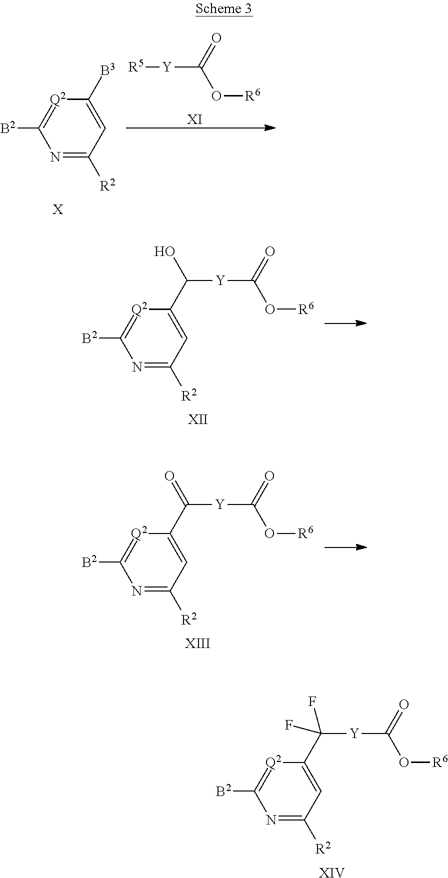

Wherein B³ is halogen; B² is halogen, or methylsulfonyl; R⁵ is aldehyde, acyl chloride, or ester; R⁶ is C₁₋₆alkyl.

A magnesium-halogen exchange of compound of formula (X) with a suitable Grignard reagent, such as isopropylmagnesium chloride lithium chloride complex, followed by addition of compound of formula (XI) affords the compound of formula (XII). Compound of formula (XIII) can be obtained from the oxidation of compound of formula (XII) in the presence of a suitable oxidant such as Dess-Martin reagent. Fluorination of compound of formula (XIII) in the presence of suitable reagent, such as DAST, can give the compound of formula (XIV).

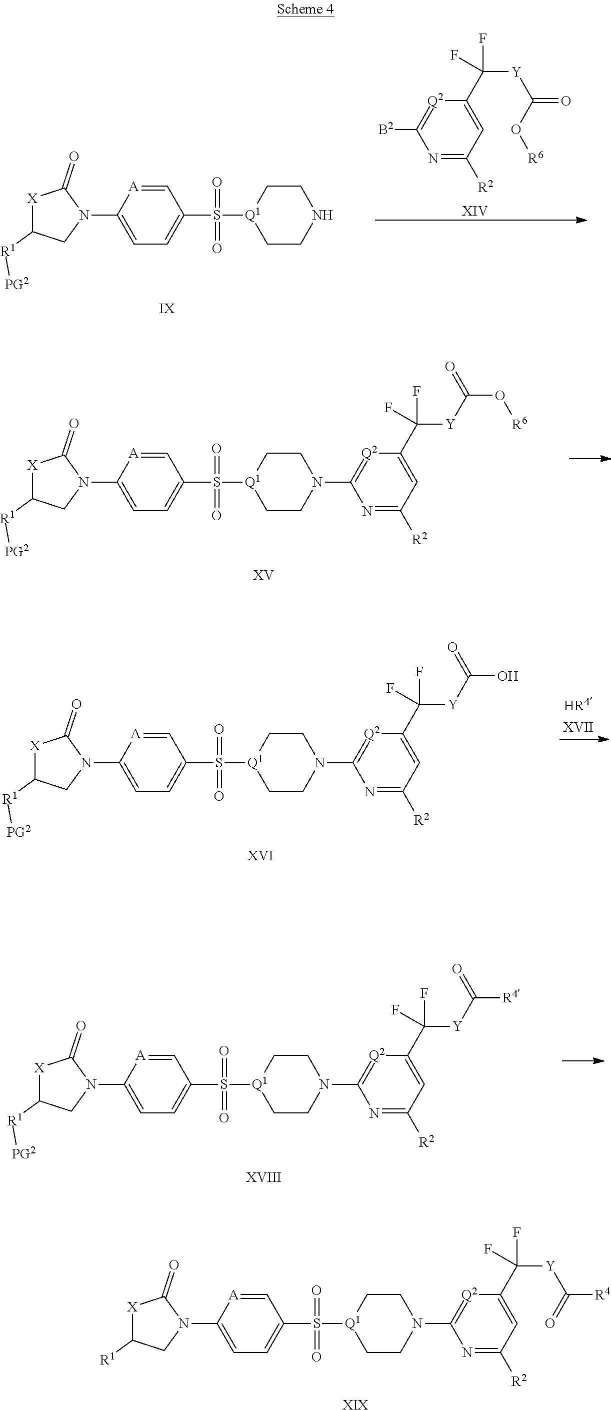

Wherein PG² is a protecting group, such as tert-butoxycarbonyl or benzyloxycarbonyl; R^{4′} is R⁴ or R⁴—PG³, wherein PG³ is a protecting group, such as tert-butoxycarbonyl or benzyloxycarbonyl.

The nucleophilic substitution between compound of formula (IX) and compound of formula (XIV) affords ester (XV). Hydrolysis of ester (XV) under suitable basic condition, such as LiOH, or suitable acid condition such as HCl, affords acid (XVI). The coupling of acid (XVI) with an amine or alcohol of compound of formula (XVII) using a suitable reagent such as HATU, gives the compound of formula (XVIII). The final compound of formula (XIX) could be obtained from deprotection of compound of formula (XVIII) using a suitable acid such as trifluoroacetic acid, or a reducing reagent such as palladium on carbon in the presence of H₂.

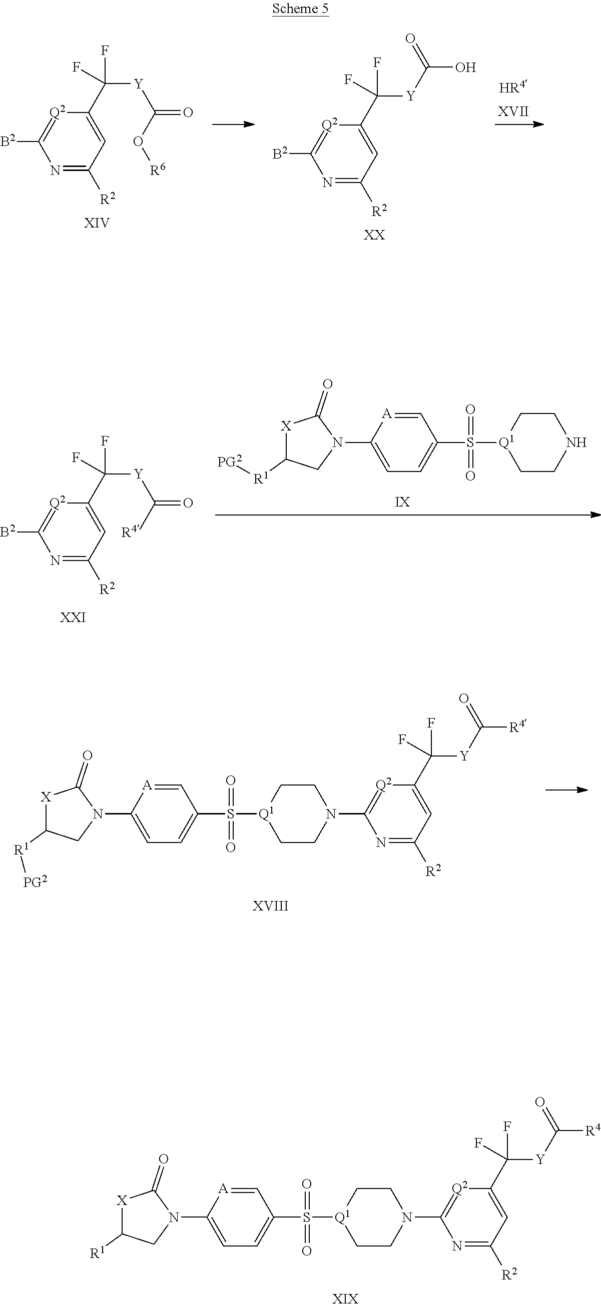

Alternatively, the compound of formula (XIX) can also be prepared according to the scheme 5. Hydrolysis of compound of formula (XIV) under suitable basic condition such as LiOH, or acid condition such as HCl, affords compound of formula (XX). The coupling of compound of formula (XX) with an amine or alcohol of compound of formula (XVII) using a suitable reagent such as HATU, gives the compound of formula (XXI). The nucleophilic substitution of compound of formula (XXI) with compound of formula (IX) in the presence of a suitable base, such as K₂CO₃, affords compound of formula (XVIII). The final compound of formula (XIX) could be obtained from the deprotection of compound of formula (XVIII) using a suitable acid such as trifluoroacetic acid, or a reducing reagent such as palladium on carbon in the presence of H₂.

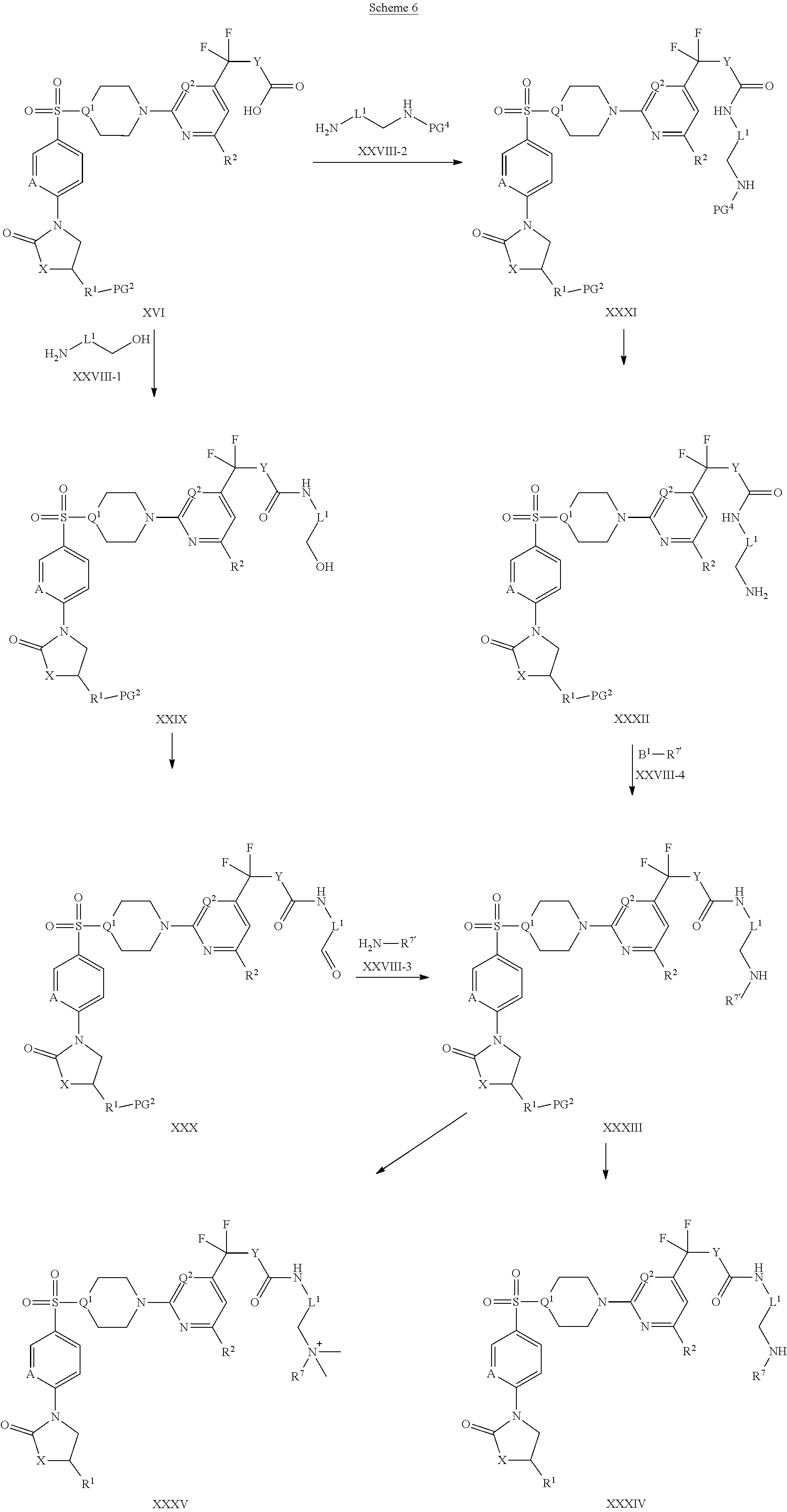

Wherein PG⁴ and PG⁵ are protecting groups, such as tert-butoxycarbonyl and benzyloxycarbonyl; L¹ is C₁₋₆alkyl or C₃₋₁₀cycloalkyl; R⁷ is C₁₋₆alkyl, aminoC₁₋₆alkyl or aminoC₃₋₁₀cycloalkyl; R^{7′} is R⁷—PG⁵.

Compound of formula (XVI) can react with compound of formula (XXVIII-1) using a suitable coupling reagent such as HATU to give compound of formula (XXIX). The oxidation of alcohol of the compound of formula (XXIX) with a suitable oxidant, such as Dess-Martin reagent, affords compound of formula (XXX). Compound of formula (XXXIII) can be obtained from the reductive amination of compound of formula (XXX) with compound of formula (XXVIII-3) in the presence of a suitable reducing reagent, such as sodium triacetoxyborohydride.

Compound of formula (XXXIII) can also be pursued from the alternative route in the scheme 6. Compound of formula (XXXI) can be prepared from the coupling of compound of formula (XVI) with amine (XXVIII-2) using a suitable coupling reagent such as HATU. The deprotection of compound of formula (XXXI) in the presence of a suitable acid, such as trifluoroacetic acid, gives the compound of formula (XXXII). Compound of formula (XXXIII) can be obtained from the substitution of compound of formula (XXXII) with compound of formula (XXVIII-4) under basic condition such as DIPEA, or through substitution reaction with coupling reagents such as HATU.

The final compound of formula (XXXIV) can be obtained from the deprotection of compound of formula (XXXIII) using a suitable acid, such as trifluoroacetic acid, or a reducing reagent such as palladium on carbon in the presence of H₂. Another final compound of formula (XXXV) can be obtained from the methylation of compound of formula (XXXIII) using a suitable reagent, such as MeI, followed by deprotection with a suitable acid, such as trifluoroacetic acid, or a reducing reagent such as palladium on carbon in the presence of H₂.

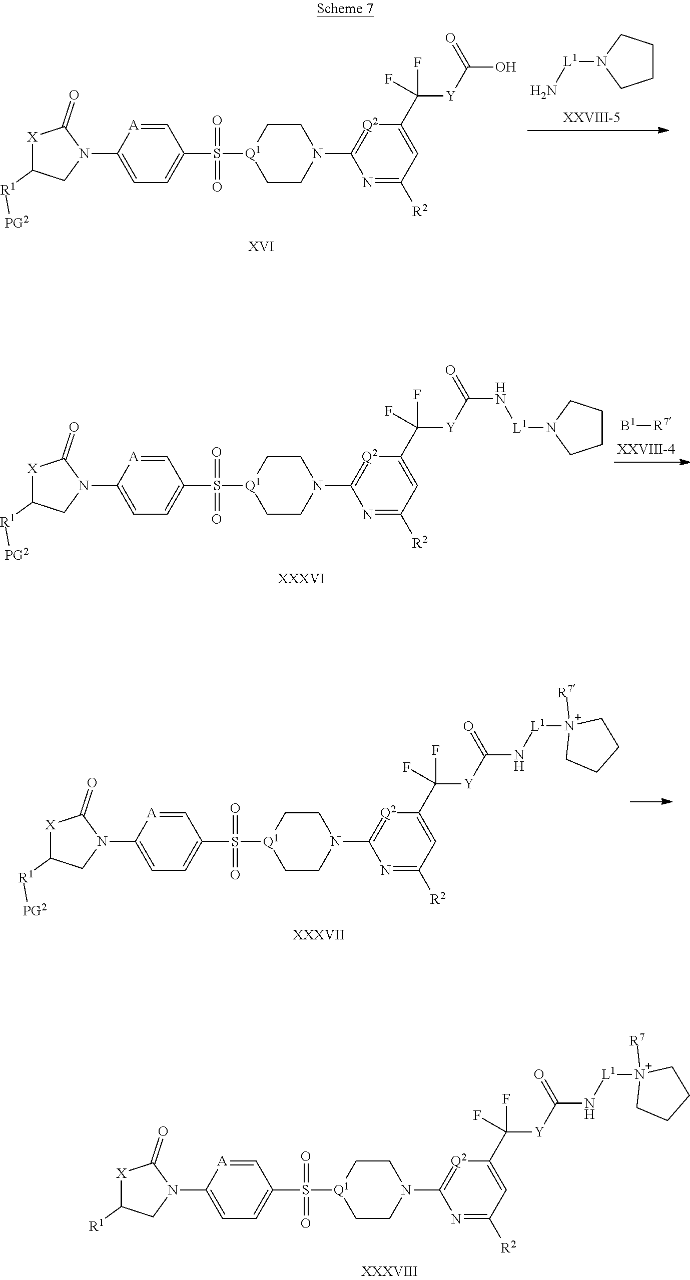

Wherein L¹ is C₁₋₆alkyl or C₃₋₁₀cycloalkyl; R⁷ is C₁₋₆alkyl, aminoC₁₋₆alkyl or aminoC₃₋₁₀cycloalkyl; R^{7′} is R⁷—PG⁵.

Compound of formula (XXXVI) can be obtained from the coupling of compound of formula (XVI) with compound of formula (XXVIII-5) using a suitable coupling reagent, such as HATU. The substitution of compound of formula (XXXVI) with compound of formula (XXVIII-4) gives the compound of formula (XXXVII) in the presence of a suitable base, such as DIPEA. The final compound of formula (XXXVIII) can be obtained from the deprotection of the compound of formula (XXXVII) with a suitable acid, such as trifluoroacetic acid, or a reducing reagent such as palladium on carbon in the presence of H₂.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising following step:
a) formation of compound of formula (XIX),

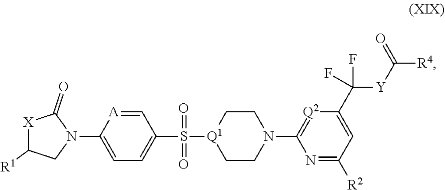

via deprotection of compound of formula (XVIII),

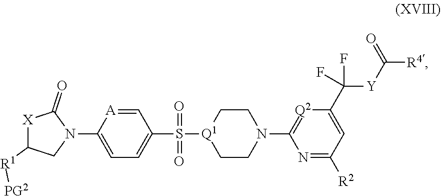

using a suitable acid, such as trifluoroacetic acid, or a reducing reagent, such as palladium on carbon in the presence of H₂;
b) formation of compound of formula (XXXV),

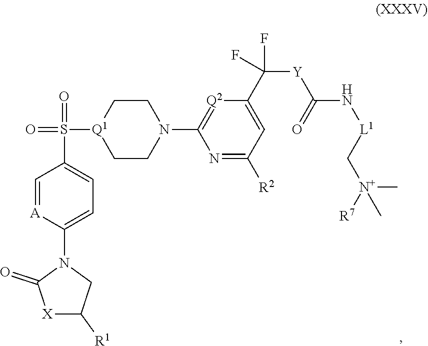

via methylation of compound of formula (XXXIII),

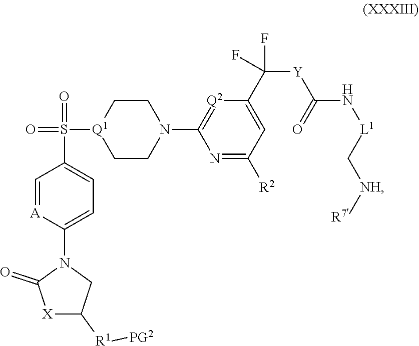

using a suitable reagent, such as MeI, followed by deprotection with a suitable acid, such as trifluoroacetic acid, or a reducing reagent such as palladium on carbon in the presence of H₂;
c) formation of compound of formula (XXXIV),

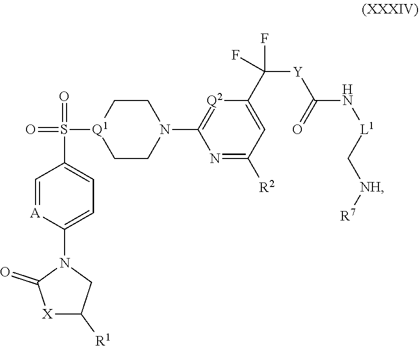

via deprotection of compound of formula (XXXIII),

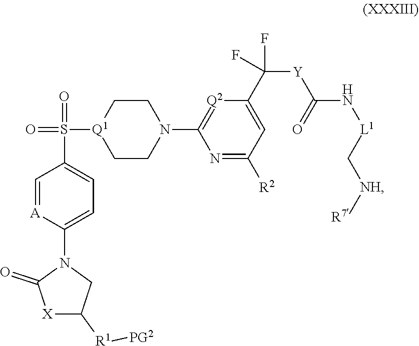

using a suitable acid, such as trifluoroacetic acid, or a reducing reagent such as palladium on carbon in the presence of H₂;
d) formation of compound of formula (XXXVIII),

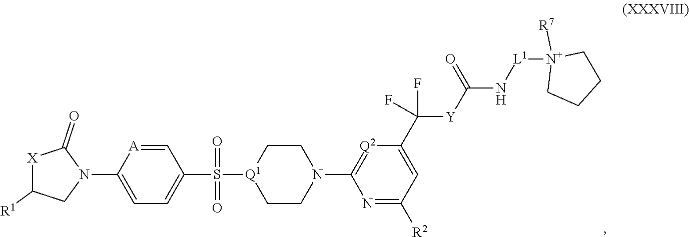

via deprotection of the compound of formula (XXXVII),

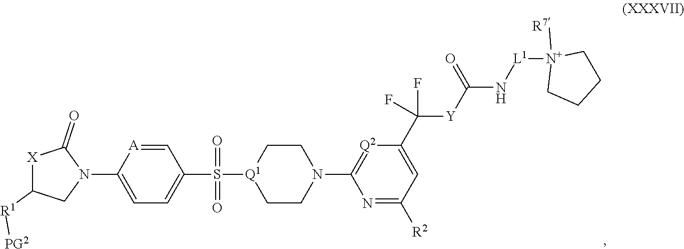

using a suitable acid, such as trifluoroacetic acid, or a reducing reagent such as palladium on carbon in the presence of H₂;
wherein R^{4′} is R⁴ or R⁴—PG³; L^{L} is C₁₋₆alkyl or C₃₋₁₀cycloalkyl; R⁷ is C₁₋₆alkyl, aminoC₁₋₆alkyl or aminoC₃₋₁₀cycloalkyl; R^{7′} is R⁷—PG⁵; PG², PG³ and PG⁵ are independently protecting groups, such as tert-butoxycarbonyl or benzyloxycarbonyl;

A compound of formula (I) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
MeCN: acetonitrile
aq.: aqueous
DAST: diethylaminosulfur trifluoride
DBU: 1,8-diazabicyclo[5.4.0]undec-7-ene
DMAP: 4-dimethylaminopyridine
DMEDA: 1,2-dimethylethylenediamine
DMP: Dess-Martin periodinane
FA: formic acid
IC₅₀: the molar concentration of an inhibitor, which produces 50% of the maximum possible response for that inhibitor.
FBS: fetal bovine serum
HPLC: high performance liquid chromatography
MS (ESI⁺): mass spectroscopy (electron spray ionization)
Ms: methylsulfonyl
MTBE: methyl tert-butyl ether
obsd.: observed
PE: petroleum ether
DCM: dichloromethane
TFA: trifluoroacetic acid
DIPEA: N,N-Diisopropylethylamine
TEA: triethylamine
HATU: 1-[bis(dimethylamino)methylene]-1H-1,2,3-triazolo[4,5-b]pyridinium 3-oxid hexafluorophosphate
δ: chemical shift
SFC: supercritical fluid chromatography
TLC: thin layer chromatography

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate 1: Tert-butyl N-[5-oxo-1-(4-piperazin-1-ylsulfonylphenyl)-pyrrolidin-3-yl]carbamate

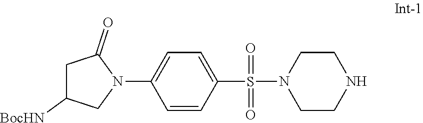 [SMILES: CC(C)(C)OC(=O)NC1CC(=O)N(c2ccc(S(=O)(=O)N3CCNCC3)cc2)C1]

### Step 1: Benzyl 4-(4-iodophenyl) sulfonylpiperazine-1-carboxylate

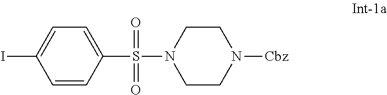 [SMILES: O=C(OCc1ccccc1)N1CCN(S(=O)(=O)c2ccc(I)cc2)CC1]

To a solution of 1-Cbz-piperazine (8.01 g, 36.4 mmol), TEA (6.45 mL, 46.3 mmol) in DCM (200 mL) was added 4-iodobenzenesulfonyl chloride (10.0 g, 33.06 mmol) at 0° C. and the mixture was stirred at room temperature for 2 hours. The mixture was concentrated in vacuo to give the residue, which was triturated in PE and washed by water to give the desired compound Int-1a (18.0 g, 37.0 mmol, 95.2% yield) as a white solid. MS obsd. (ESI⁺) [(M+H)⁺]: 487.0. ¹H NMR (400 MHz, CDCl₃) δ ppm 7.94 (br d, J=8.8 Hz, 2H), 7.89 (m, 2H), 7.43 (m, 1H), 7.32-7.33 (m, 4H), 5.01 (s, 2H), 3.12-3.14 (m, 8H).

### Step 2: Benzyl 4-((4-(4-((tert-butoxycarbonyl)amino)-2-oxopyrrolidin-1-yl)phenyl)sulfonyl) piperazine-1-carboxylate

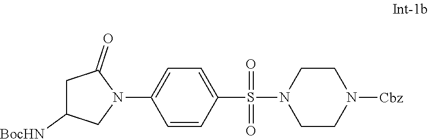 [SMILES: CC(C)(C)OC(=O)NC1CC(=O)N(c2ccc(S(=O)(=O)N3CCN(C(=O)OCc4ccccc4)CC3)cc2)C1]

To a mixture of compound Int-1a (18.0 g, 37.0 mmol), tert-butyl N-(5-oxopyrrolidin-3-yl)carbamate (8.89 g, 44.4 mmol) and K₂CO₃ (15.4 g, 111 mmol) in NMP (180 mL) was added CuI (7.05 g, 37.0 mmol) and N1,N2-dimethylcyclohexane-1,2-diamine (5.26 g, 37.0 mmol). The mixture was stirred at 90° C. for 2 hours under nitrogen. The mixture was diluted with EtOAc (200 mL) and then filtered through fritted glass funnel. The filtrate was washed with NH₃·H₂O (2% wt, 200 mL), aq. CaCl₂ solution (150 mL, 2 N) and brine (150 mL×2). The filtrate was collected and dried over anhydrous sodium sulfate, filtered and concentrated in vacuo to give yellow oil. The oil was purified by prep-HPLC to give the desired compound Int-1b (13.5 g, 24.2 mmol, 65.3% yield) as a gray solid. MS obsd. (ESI⁺) [(M+H)⁺]: 559.3.

### Step 3: Tert-butyl N-[5-oxo-1-(4-piperazin-1-ylsulfonylphenyl)pyrrolidin-3-yl]carbamate

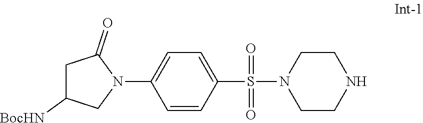 [SMILES: CC(C)(C)OC(=O)NC1CC(=O)N(c2ccc(S(=O)(=O)N3CCNCC3)cc2)C1]

To a solution of compound Int-1b (500 mg, 0.90 mmol) in methanol (10 mL) was added Pd/C (10% wt, 100.0 mg). The mixture was stirred under H₂ (15 psi) at room temperature for 2 hours. The mixture was diluted with MeOH (100 mL), filtered through a pad of Celite and concentrated in vacuo to give the desired compound Int-1 (300 mg, 0.710 mmol, 78.96% yield) as a grey solid. MS obsd. (ESI⁺) [(M+H)⁺]: 425.3.

### Intermediate 2: Tert-butyl N-[(3R)-5-oxo-1-(4-piperazin-1-ylsulfonylphenyl)pyrrolidin-3-yl]carbamate

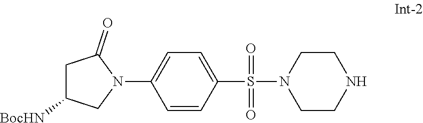 [SMILES: CC(C)(C)OC(=O)N[C@@H]1CC(=O)N(c2ccc(S(=O)(=O)N3CCNCC3)cc2)C1]

The title compound was prepared in analogy to the preparation of compound Int-1 by using tert-butyl N-[(3R)-5-oxopyrrolidin-3-yl]carbamate instead of tert-butyl N-(5-oxopyrrolidin-3-yl)carbamate. Compound Int-2 was obtained as a white solid. MS obsd. (ESI⁺) [(M+H)⁺]: 425.2.

### Intermediate 3A and 3B: (5S)-5-(hydroxymethyl)-3-(4-piperazin-1-ylsulfonylphenyl)oxazolidin-2-one and (5R)-5-(hydroxymethyl)-3-(4-piperazin-1-ylsulfonylphenyl)oxazolidin-2-one

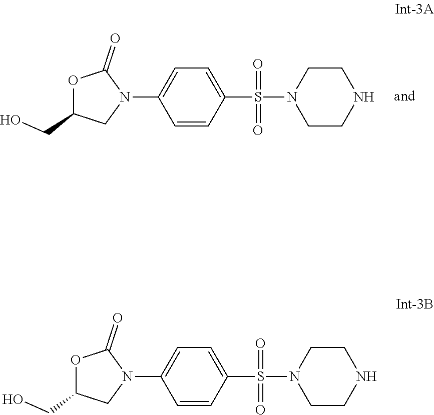 [SMILES: O=C1O[C@H](CO)CN1c1ccc(S(=O)(=O)N2CCNCC2)cc1 ; O=C1O[C@@H](CO)CN1c1ccc(S(=O)(=O)N2CCNCC2)cc1]

### Step 1: Tert-butyl 4-(4-iodophenyl)sulfonylpiperazine-1-carboxylate

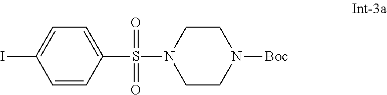 [SMILES: CC(C)(C)OC(=O)N1CCN(S(=O)(=O)c2ccc(I)cc2)CC1]

To a solution of 4-iodobenzenesulfonyl chloride (1.0 g, 3.31 mmol) and tert-butyl piperazine-1-carboxylate (739 mg, 3.97 mmol) in DCM (100 mL) was added TEA (668 mg, 6.61 mmol) dropwise at room temperature. The mixture was washed with aq. NaHCO₃ solution (100 mL), extracted with DCM (50 mL×3), dried over anhydrous sodium sulfate, filtered and concentrated in vacuo to give compound Int-3a (1.5 g, 3.32 mmol, 100% yield), which was used in the next step directly. MS obsd. (ESI⁺) [(M+H)⁺]: 452.98.

### Step 2: Tert-butyl 4-[4-[(5S)-5-(hydroxymethyl)-2-oxo-oxazolidin-3-yl]phenyl]sulfonylpiperazine-1-carboxylate (Int-5bA) and tert-butyl 4-[4-[(5R)-5-(hydroxymethyl)-2-oxo-oxazolidin-3-yl]phenyl]sulfonylpiperazine-1-carboxylate (Int-5bB)

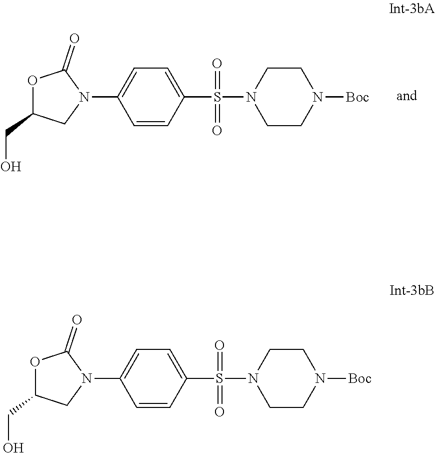 [SMILES: CC(C)(C)OC(=O)N1CCN(S(=O)(=O)c2ccc(N3C[C@@H](CO)OC3=O)cc2)CC1 ; CC(C)(C)OC(=O)N1CCN(S(=O)(=O)c2ccc(N3C[C@H](CO)OC3=O)cc2)CC1]

A degassed mixture of compound Int-3a (0.430 g, 951 μmol), 5-(hydroxymethyl)oxazolidin-2-one (111 mg, 951 μmol), N,N′-dimethylethane-1,2-diamine (29.3 mg, 333 μmol), CuI (63.4 mg, 333 μmol) and K₃PO₄ (404 mg, 1.9 mmol) in DMF (4 ml) was heated at 75° C. in a microwave reactor for 1 hour. After completion, the mixture was poured into ice water, extracted with DCM (25 mL×3), dried over anhydrous sodium sulfate, and concentrated in vacuo to give crude product, which was purified by silica gel chromatography (eluent with EtOAc:PE=0-80%) to afford the enantiomeric mixture of Int-3bA and Int-3bB (310 mg, 702 μmol, 73.9% yield). MS obsd. (ESI⁺) [(M+H)⁺]: 442.1. The resulting solid was then separated by SFC to give the two enantiomers Int-3bA (fast eluted) and Int-3bB (slow eluted). SFC condition: Column: IA (250 mm×30 mm, 5 um); 0.1% NH₃H₂O EtOH; begin B 50%, end B 50%; Flow Rate (ml/min): 50.

### Step 3: (5S)-5-(hydroxymethyl)-3-(4-piperazin-1-ylsulfonylphenyl)oxazolidin-2-one (Int-3A) and (5R)-5-(hydroxymethyl)-3-(4-piperazin-1-ylsulfonylphenyl)oxazolidin-2-one (Int-3B)

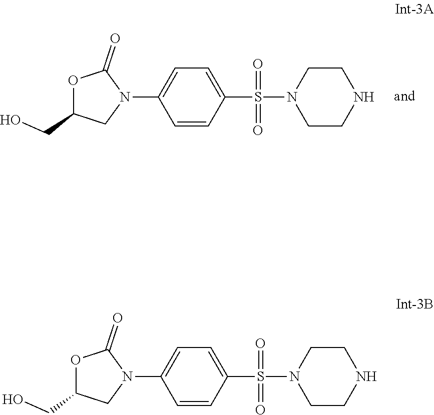 [SMILES: O=C1O[C@H](CO)CN1c1ccc(S(=O)(=O)N2CCNCC2)cc1 ; O=C1O[C@@H](CO)CN1c1ccc(S(=O)(=O)N2CCNCC2)cc1]

Compound Int-3bA (60 mg, 0.136 mmol) and TFA (0.5 mL, 6.49 mmol) were mixed in the DCM (5 mL). The reaction mixture was stirred at room temperature for 1 hour. After completion, the residue was concentrated in vacuo to give crude compound Int-3A (45 mg, 97%) as light yellow oil. MS obsd. (ESI⁺) [(M+H)⁺]: 342.1.

Int-3B was prepared in analogy to the procedure described for the preparation of compound Int-3A by using compound Int-3bB as the starting material instead of compound Int-3bA. MS obsd. (ESI⁺) [(M+H)⁺]: 342.1.

### Intermediate 4: Tert-butyl 4-[5-oxo-1-(4-piperazin-1-ylsulfonylphenyl)pyrrolidin-3-yl]piperazine-1-carboxylate

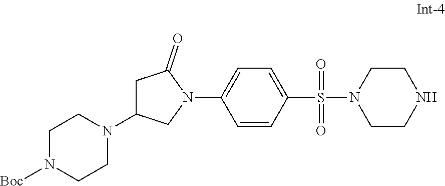 [SMILES: CC(C)(C)OC(=O)N1CCN(C2CC(=O)N(c3ccc(S(=O)(=O)N4CCNCC4)cc3)C2)CC1]

### Step 1: Benzyl 4-[4-(4-hydroxy-2-oxo-pyrrolidin-1-yl)phenyl]sulfonylpiperazine-1-carboxylate

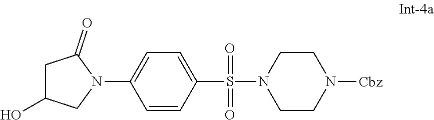 [SMILES: O=C(OCc1ccccc1)N1CCN(S(=O)(=O)c2ccc(N3CC(O)CC3=O)cc2)CC1]

To a solution of compound Int-3a (5.5 g, 11.31 mmol), 4-hydroxypyrrolidin-2-one (2.29 g, 22.62 mmol) and potassium carbonate (4.69 g, 33.93 mmol) in NMP (25 mL) was added copper iodide (1.51 g, 7.92 mmol) and trans-N,N-dimethylcyclohexane (1.13 g, 7.92 mmol) under nitrogen. The mixture was stirred at 90° C. After 2 hours, the mixture was diluted with EtOAc (600 mL) and filtered. The filtrate was washed with 2% aq. NH₃·H₂O solution (100 mL×2), aq. CaCl₂ (100 mL×3) and brine (100 mL×2). Then, the organic layer was dried over anhydrous sodium sulfate, filtered and concentrated in vacuo to give crude product, which was triturated in PE (100 mL) to give the compound Int-4a (4.5 g, 9.81 mmol, 86.78% yield) as a brown solid. MS obsd. (ESI⁺) [(M+H)⁺]: 459.9.

### Step 2: Benzyl 4-[4-(5-oxo-2H-pyrrol-1-yl)phenyl]sulfonylpiperazine-1-carboxylate

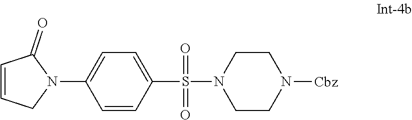 [SMILES: O=C(OCc1ccccc1)N1CCN(S(=O)(=O)c2ccc(N3CC=CC3=O)cc2)CC1]

To a solution of compound Int-4a (4.5 g, 9.79 mmol) and DIPEA (5.05 g, 39.17 mmol) in DCM (80 mL) was added methanesulfonyl chloride (1.58 mL, 20.35 mmol) slowly at 0° C. Then, the mixture was stirred at room temperature for 3 hours. After completion, the reaction mixture was poured into water (100 mL) at 0° C. and stirred at room temperature for 30 minutes. Then, the solution was extracted with DCM (200 mL×3) and washed with aq. NaOH solution (0.5 N, 100 mL×3) and brine (100 mL×3). The organic layer was dried over anhydrous sodium sulfate, filtered, and concentrated in vacuo to give crude product, which was triturated in PE/MTBE (100 mL, v/v=1:1) to give compound Int-4b (3.5 g, 7.95 mmol, 81.13% yield) as a grey solid. MS obsd. (ESI⁺) [(M+H)⁺]: 441.9.

### Step 3: Tert-butyl 4-[l-[4-(4-benzyloxycarbonylpiperazin-1-yl)sulfonylphenyl]-5-oxo-pyrrolidin-3-yl]piperazine-1-carboxylate

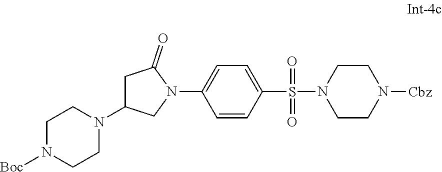 [SMILES: CC(C)(C)OC(=O)N1CCN(C2CC(=O)N(c3ccc(S(=O)(=O)N4CCN(C(=O)OCc5ccccc5)CC4)cc3)C2)CC1]

To a solution of compound Int-4b (2.0 g, 4.53 mmol) in NMP (0.5 mL) was added 1-BOC-piperazine (10.0 g, 53.69 mmol). The mixture was stirred at 90° C. for 2 hours. After completion, the mixture was diluted with water (100 mL), extracted with EtOAc (100 mL×2) and washed with brine (100 mL). The combined organic layer was dried over anhydrous sodium sulfate, and concentrated in vacuo to give the crude residue, which was purified by silica gel chromatography (eluent with EtOAc:PE=0-100%) to afford compound Int-4c (1.6 g, 2.55 mmol, 56.26% yield) as a yellow solid. MS obsd. (ESI⁺) [(M+H)⁺]: 628.3.

### Step 4: Tert-butyl 4-[5-oxo-1-(4-piperazin-1-ylsulfonylphenyl)pyrrolidin-3-yl]piperazine-1-carboxylate

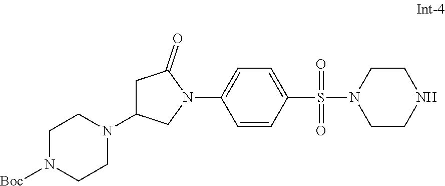 [SMILES: CC(C)(C)OC(=O)N1CCN(C2CC(=O)N(c3ccc(S(=O)(=O)N4CCNCC4)cc3)C2)CC1]

To a solution of compound Int-4c (1.5 g, 2.39 mmol) in THF (4 mL) was added Pd/C (800 mg, 2.39 mmol). The mixture was stirred at 25° C. for 2 hours under H₂ (15 psi). After completion, the mixture was filtered through a Celite pad, and the filtrate was concentrated in vacuo to give the crude product, which was purified by prep-HPLC to give compound Int-4 (550 mg, 1.11 mmol, 38.18% yield) as a white solid. MS obsd. (ESI⁺) [(M+H)⁺]: 494.3.

### Intermediate 5: Trans-4-[[2-[4-[4-[(4R)-4-(tert-butoxycarbonylamino)-2-oxo-pyrrolidin-1-yl]phenyl]sulfonylpiperazin-1-yl]-6-chloro-4-pyridyl]-difluoro-methyl]cyclohexanecarboxylic acid

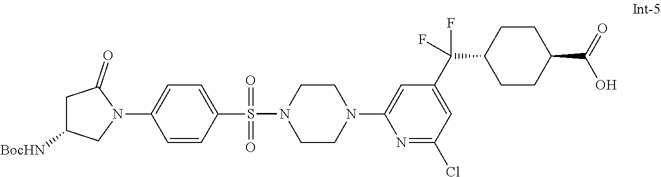 [SMILES: CC(C)(C)OC(=O)N[C@@H]1CC(=O)N(c2ccc(S(=O)(=O)N3CCN(c4cc(C(F)(F)[C@H]5CC[C@H](C(=O)O)CC5)cc(Cl)n4)CC3)cc2)C1]

### Step 1: Trans-methyl 4-(hydroxymethyl)cyclohexanecarboxylate

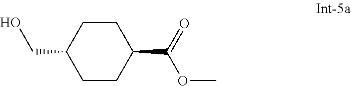

To a solution of trans-4-methoxycarbonylcyclohexanecarboxylic acid (50.0 g, 268.51 mmol) in THF (150 mL) was added BH₃·Me₂S (53.7 mL, 537.03 mmol) dropwise at 0˜10° C. for 1 hour under nitrogen. The mixture was stirred at 25° C. for 12 hours. After completion, the reaction was quenched by adding methanol (100 mL) dropwise at 0° C. The mixture was concentrated in vacuo at 40° C. to give crude residue, which was purified by silica gel chromatography (eluent with EtOAc:PE=0-50%) to give compound Int-5a (28 g, 162.58 mmol, 60.55% yield) as colorless oil.

### Step 2: Trans-methyl 4-formylcyclohexanecarboxylate

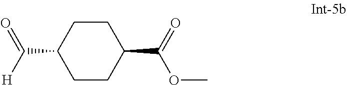

To a solution of oxalyl chloride (30.95 g, 243.87 mmol) in DCM (100 mL) was added dimethyl sulfoxide (25.4 g, 325.17 mmol) at −60° C. and stirred at −60° C. for 0.5 hour under nitrogen. Then the solution of compound Int-5a (28.0 g, 162.58 mmol) in DCM (100 mL) was added into the reaction at −60° C. and kept stirring for additional 1 hour. Then, DIPEA (84.05 g, 650.33 mmol) was added at −60° C. The reaction was allowed to warm to 0° C. and kept stirring for 2 hours. After completion, the reaction mixture was diluted with EtOAc (600 mL) and washed with aq. CaCl₂ (100 mL×3) and brine (50 mL×2). The organic layer was dried over anhydrous sodium sulfate, filtered and concentrated in vacuo to give crude residue, which was purified by silica gel chromatography (eluent with EtOAc:PE=3-50%) to give compound Int-5b (24 g, 141 mmol, 86.73% yield) as light yellow oil.

### Step 3: Trans-methyl 4-[(2,6-dichloro-4-pyridyl)-hydroxy-methyl]cyclohexanecarboxylate

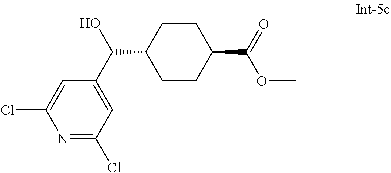 [SMILES: COC(=O)[C@H]1CC[C@H](C(O)c2cc(Cl)nc(Cl)c2)CC1]

To a solution of 2,6-dichloro-4-iodo-pyridine (42.48 g, 155.1 mmol) in THF (50.0 mL) was added isopropylmagnesium chloride lithium chloride complex in THF (130.16 mL, 169.2 mmol) at −40° C. under nitrogen. The mixture was stirred at 25° C. for 1 hour. The reaction mixture was then added into a solution of compound Int-5b (24.0 g, 141 mmol) in THF (100 mL) dropwise at −40° C. The mixture was stirred at 25° C. for 3 hours. After completion, the reaction mixture was poured into sat. aq. NH₄Cl solution (200 mL) and extracted with EtOAc (300 mL×3). The organic layer was washed with brine (100 mL×2), dried over anhydrous sodium sulfate, filtered and concentrated in vacuo to give crude residue, which was purified by silica gel chromatography (eluent with EtOAc:PE=0-30%) to give compound Int-5c (33 g, 103.71 mmol, 73.55% yield) as a white solid. MS obsd. (ESI⁺) [(M+Cl)⁺]: 317.8.

### Step 4: Trans-methyl 4-(2,6-dichloropyridine-4-carbonyl)cyclohexanecarboxylate

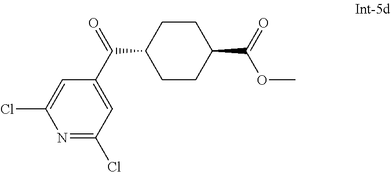 [SMILES: COC(=O)[C@H]1CC[C@H](C(=O)c2cc(Cl)nc(Cl)c2)CC1]

To a solution of compound Int-5c (33.0 g, 103.71 mmol) in THF (150 mL) was added DMP (48.39 g, 114.08 mmol) slowly at 0° C. under nitrogen. The reaction was stirred at 25° C. for 3 hours. After completion, the mixture was diluted with EtOAc (500 mL), then poured into aq. K₂CO₃ solution (200 mL) and kept stirring at room temperature for 0.5 hour. The organic layer was separated and washed with brine (100 mL×2), dried over anhydrous sodium sulfate, filtered and concentrated in vacuo to give crude compound Int-5d (30 g, 94.88 mmol, 91.49% yield) as a white solid, which was used in the next step directly. MS obsd. (ESI⁺) [(M+H)⁺]: 315.8.

### Step 5: Trans-methyl 4-[(2,6-dichloro-4-pyridyl)-difluoro-methyl]cyclohexanecarboxylate

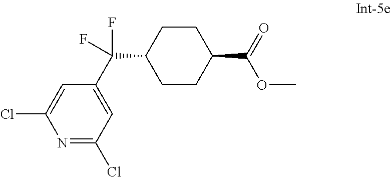 [SMILES: COC(=O)[C@H]1CC[C@H](C(F)(F)c2cc(Cl)nc(Cl)c2)CC1]

A solution of compound Int-5d (29.5 g, 93.3 mmol) in DCM (20 mL) was added DAST (135.35 g, 839.71 mmol) and stirred at 45° C. for 12 hours. After completion, the reaction mixture was poured into ice-water and extracted with EtOAc (200 mL×3). The combined organic layer was washed with aq. K₂CO₃ (100 mL×2) and brine (100 mL×2), dried over anhydrous sodium sulfate, filtered, and concentrated in vacuo to give a crude residue. The residue was recrystallized with MeOH (150 mL) to give compound Int-5e (25.6 g, 75.7 mmol, 81.13% yield) as a white solid. MS obsd. (ESI⁺) [(M+H)⁺]: 337.8.

### Step 6: Trans-methyl 4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarboxylate

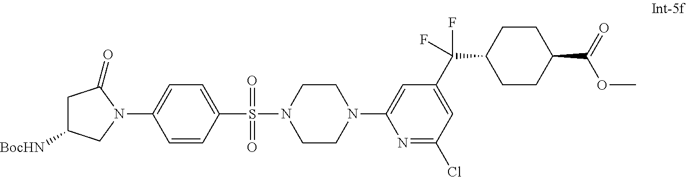 [SMILES: COC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound Int-5e (3.58 g, 10.6 mmol) and compound Int-2 (4.5 g, 10.6 mmol) in DMSO (10 mL) was added DIPEA (5.55 mL, 31.8 mmol). The mixture was stirred at 115° C. for 18 hours. After completion, the reaction mixture was concentrated in vacuo and purified by prep-HPLC to give compound Int-5f (7 g, 9.64 mmol, 90.93% yield) as a white solid. MS obsd. (ESI⁺) [(M+H)⁺]: 670.1.

### Step 7: Trans-4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarboxylic acid

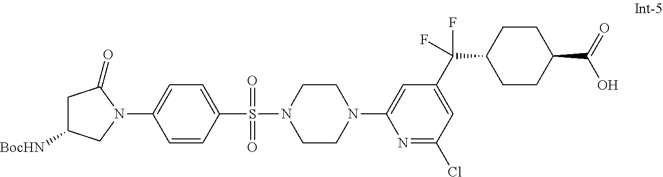 [SMILES: CC(C)(C)OC(=O)N[C@@H]1CC(=O)N(c2ccc(S(=O)(=O)N3CCN(c4cc(C(F)(F)[C@H]5CC[C@H](C(=O)O)CC5)cc(Cl)n4)CC3)cc2)C1]

To a solution of compound Int-5e (7.0 g, 9.64 mmol) in MeCN (100 mL) and water (10 mL) were added TEA (13.43 mL, 96.39 mmol), lithium bromide (16.74 g, 192.78 mmol) in one portion. The reaction was stirred at 40° C. for 12 hours. After completion, the reaction was diluted with 2-methyltetrahydrofuran (500 mL) and adjusted to pH 5 with 2N aq. HCl solution. The resulting organic layer was washed with 0.2 N aq. HCl solution (100.0 mL×2) and brine (100 mL×2), dried over anhydrous sodium sulfate, filtered and concentrated in vacuo to give crude product. The crude product was recrystallized with MeCN (100 mL) to give compound Int-5 (6.7 g, 9.41 mmol, 97.6% yield) as a white solid. MS obsd. (ESI⁺) [(M+H)⁺]: 712.1.

### Intermediate 6: Trans-4-[[2-chloro-6-[4-[4-(4-methyl-2-oxo-pyrrolidin-1-yl)phenyl]sulfonylpiperazin-1-yl]-4-pyridyl]-difluoro-methyl]cyclohexanecarboxylic acid

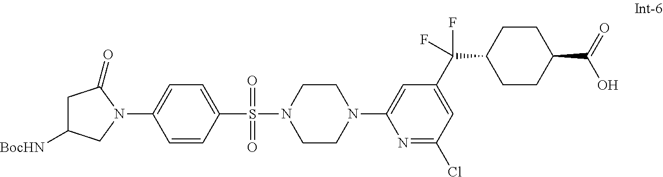 [SMILES: CC(C)(C)OC(=O)NC1CC(=O)N(c2ccc(S(=O)(=O)N3CCN(c4cc(C(F)(F)[C@H]5CC[C@H](C(=O)O)CC5)cc(Cl)n4)CC3)cc2)C1]

Compound Int-6 was prepared in analogy to the procedure described for the preparation of compound Int-5 by using compound Int-2 as the coupling reagent instead of compound Int-1 in Step 6. MS obsd. (ESI⁺) [(M+H)⁺]: 656.1.

### Intermediate 7: Cis-4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarboxylic acid

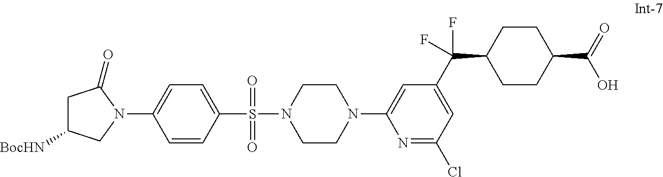 [SMILES: CC(C)(C)OC(=O)N[C@@H]1CC(=O)N(c2ccc(S(=O)(=O)N3CCN(c4cc(C(F)(F)[C@H]5CC[C@@H](C(=O)O)CC5)cc(Cl)n4)CC3)cc2)C1]

Compound Int-7 was prepared in analogy to the procedure described for the preparation of compound Int-5 by using cis-4-methoxycarbonylcyclohexanecarboxylic acid instead of trans-4-methoxycarbonylcyclohexanecarboxylic acid in Step 1. MS obsd. (ESI⁺) [(M+H)⁺]: 712.2.

### Intermediate 8: 3-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclopentanecarboxylic acid

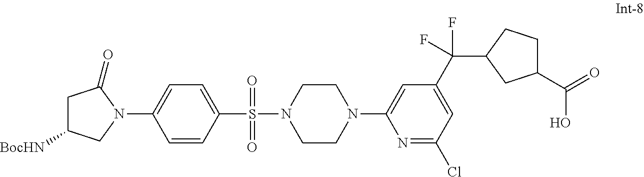 [SMILES: CC(C)(C)OC(=O)N[C@@H]1CC(=O)N(c2ccc(S(=O)(=O)N3CCN(c4cc(C(F)(F)C5CCC(C(=O)O)C5)cc(Cl)n4)CC3)cc2)C1]

The title compound was prepared in analogy to the preparation of compound Int-5 by using 3-methoxycarbonylcyclopentanecarboxylic acid instead of trans-4-methoxycarbonylcyclohexanecarboxylic acid in Step 1. Compound Int-8 (20.0 mg) was obtained as light yellow oil. MS obsd. (ESI⁺) [(M+Cl)⁺]: 698.1.

### Intermediate 9: 4-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]norbornane-1-carboxylic acid

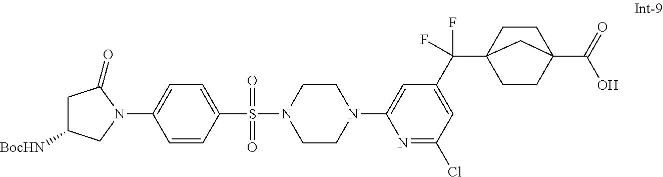 [SMILES: CC(C)(C)OC(=O)N[C@@H]1CC(=O)N(c2ccc(S(=O)(=O)N3CCN(c4cc(C(F)(F)C56CCC(C(=O)O)(CC5)C6)cc(Cl)n4)CC3)cc2)C1]

The title compound was prepared in analogy to the preparation of compound Int-5 by using 4-(methoxycarbonyl)bicyclo[2.2.1]heptane-1-carboxylic acid instead of trans-4-methoxycarbonylcyclohexanecarboxylic acid in Step 1. Compound Int-9 (150.0 mg) was obtained as a white solid. MS obsd. (ESI⁺) [(M+H)⁺]: 724.4.

### Intermediate 10: 4-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]bicyclo[2.2.2]octane-1-carboxylic acid

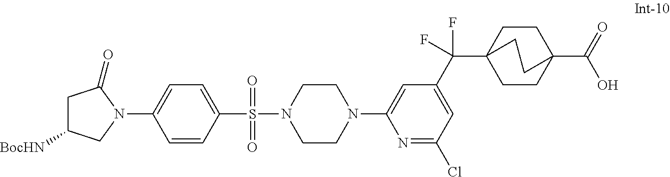 [SMILES: CC(C)(C)OC(=O)N[C@@H]1CC(=O)N(c2ccc(S(=O)(=O)N3CCN(c4cc(C(F)(F)C56CCC(C(=O)O)(CC5)CC6)cc(Cl)n4)CC3)cc2)C1]

The title compound was prepared in analogy to the preparation of compound Int-5 by using 4-(methoxycarbonyl)bicyclo[2.2.2]octane-1-carboxylic acid instead of trans-4-methoxycarbonylcyclohexanecarboxylic acid in Step 1. Compound Int-10 (250.0 mg) was obtained as a light yellow solid. MS obsd. (ESI⁺) [(M+H)⁺]: 738.4.

### Intermediate 11: 3-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]bicyclo[1.1.1]pentane-1-carboxylic acid

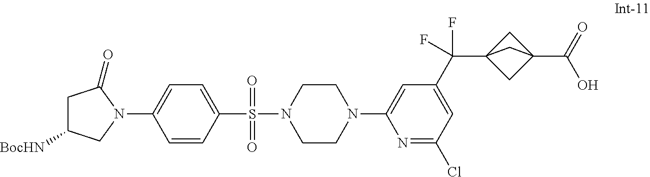 [SMILES: CC(C)(C)OC(=O)N[C@@H]1CC(=O)N(c2ccc(S(=O)(=O)N3CCN(c4cc(C(F)(F)C56CC(C(=O)O)(C5)C6)cc(Cl)n4)CC3)cc2)C1]

The title compound was prepared in analogy to the preparation of compound Int-5 by using 3-(methoxycarbonyl)bicyclo[1.1.1]pentane-1-carboxylic acid instead of trans-4-methoxycarbonylcyclohexanecarboxylic acid in Step 1. Compound Int-11 (150.0 mg) was obtained as a light yellow oil. MS obsd. (ESI⁺) [(M+H)⁺]: 696.0.

### Intermediate 12: 4-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]benzoic acid

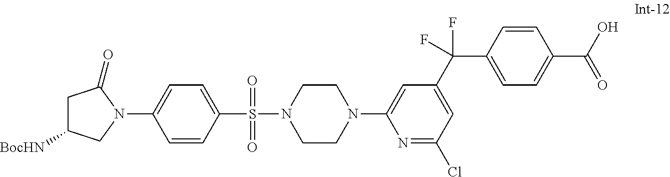 [SMILES: CC(C)(C)OC(=O)N[C@@H]1CC(=O)N(c2ccc(S(=O)(=O)N3CCN(c4cc(C(F)(F)c5ccc(C(=O)O)cc5)cc(Cl)n4)CC3)cc2)C1]

### Step 1: Methyl 4-[(2,6-dichloro-4-pyridyl)-hydroxy-methyl]benzoate

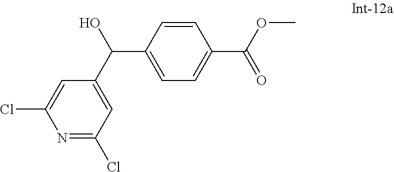 [SMILES: COC(=O)c1ccc(C(O)c2cc(Cl)nc(Cl)c2)cc1]

To a mixture of 2,6-dichloro-4-iodo-pyridine (36.71 g, 134.02 mmol) in THF (180 mL) was added isopropylmagnesium chloride lithium chloride complex solution (1.3 M in THF, 121.83 mL) under nitrogen at −40° C. The mixture was stirred at 25° C. for 2 hours. Then to the mixture was added methyl 4-formylbenzoate (20 g, 121.83 mmol) in THF (200 mL) at −40° C. The mixture was stirred at 25° C. for 2 hours. After completion, the mixture was added to sat. aq NH₄Cl solution (1 L) in ice-water bath and extracted with EtOAc (500 mL×2). The combined organic layer was washed with brine (500 mL×2), dried over anhydrous sodium sulfate, filtered and concentrated in vacuo to give crude compound Int-12a (30 g, 96.11 mmol, 78.88% yield) as a yellow solid. MS obsd. (ESI⁺) [(M+H)⁺]: 312.1.

### Step 2: Methyl 4-(2,6-dichloropyridine-4-carbonyl)benzoate

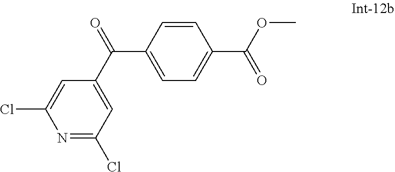 [SMILES: COC(=O)c1ccc(C(=O)c2cc(Cl)nc(Cl)c2)cc1]

To a solution of compound Int-12a (25 g, 80.09 mmol) in THF (250 mL) was added DMP (37.37 g, 88.10 mmol) at 0° C. The mixture was stirred at 0° C. for 1 hour. After completion, the reaction mixture was filtered, and the filtrate was diluted with water (200 mL) and extracted with EtOAc (200 mL×2). The combined organic layer was washed with brine (300 mL), dried over anhydrous sodium sulfate, filtered and concentrated in vacuo to give a residue. The residue was suspended in PE (50 mL) and the resulting precipitate was collected by filtration. Compound Int-12b (20.3 g, 65.46 mmol, 81.73% yield) was obtained as a white solid. ¹H NMR (400 MHz, CHLOROFORM-d) δ ppm 8.21 (d, J=8.44 Hz, 2H) 7.86 (d, J=8.44 Hz, 2H) 7.54 (s, 2H) 3.99 (s, 3H).

### Step 3: Methyl 4-[(2,6-dichloro-4-pyridyl)-difluoro-methyl]benzoate

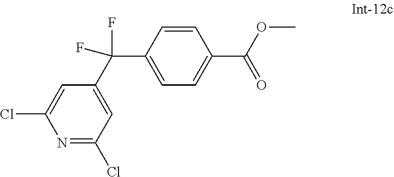 [SMILES: COC(=O)c1ccc(C(F)(F)c2cc(Cl)nc(Cl)c2)cc1]

To a mixture of compound Int-12b (20.3 g, 65.46 mmol) in DCM (150 mL) was added DAST (84.41 g, 523.65 mmol) and stirred at 40° C. for 12 hours. After completion, the mixture was added into aq NaHCO₃ solution (500 mL) in ice-water bath and extracted with DCM (200 mL×2). The combined organic layer was washed with brine (200 mL×2), dried over anhydrous sodium sulfate, filtered and concentrated in vacuo to give crude residue, which was purified by silica gel chromatography (EtOAc:PE=2-5%) to give compound Int-12c (20.2 g, 60.82 mmol, 92.92% yield) as a white solid. MS obsd. (ESI⁺) [(M+H)⁺]: 332.0.

### Step 4: Methyl 4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]benzoate

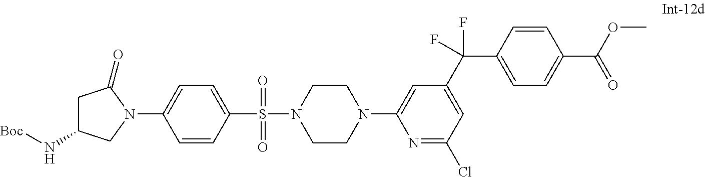 [SMILES: COC(=O)c1ccc(C(F)(F)c2cc(Cl)nc(N3CCN(S(=O)(=O)c4ccc(N5C[C@H](NC(=O)OC(C)(C)C)CC5=O)cc4)CC3)c2)cc1]

To a solution of compound Int-2 (100.0 mg, 0.240 mmol) and compound Int-12c (78.24 mg, 0.240 mmol) in DMSO (1 mL) was added DIPEA (0.2 mL, 0.710 mmol). The mixture was stirred at 110° C. for 12 hours. After completion, the mixture was diluted with EtOAc (50 mL) and water (30 mL). The organic phase was washed with brine (50 mL), dried over anhydrous sodium sulfate, concentrated in vacuo to give the crude residue compound Int-12d (120 mg, 0.170 mmol, 70.73% yield) as a yellow solid, which was used directly in the next step. MS obsd. (ESI⁺) [(M+H)⁺]: 720.3.

### Step 5: 4-[[2-Chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]benzoic acid

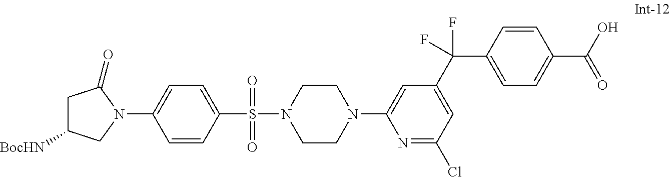 [SMILES: CC(C)(C)OC(=O)N[C@@H]1CC(=O)N(c2ccc(S(=O)(=O)N3CCN(c4cc(C(F)(F)c5ccc(C(=O)O)cc5)cc(Cl)n4)CC3)cc2)C1]

To a solution of compound Int-12d (116.74 mg, 0.160 mmol) in MeCN (5 mL) and water (1 mL) was added LiBr (69.71 mg, 0.810 mmol) and TEA (100.5 mg, 0.810 mmol). The mixture was stirred at 60° C. for 12 hours. After completion, the mixture was diluted with EtOAc (50 mL) and water (30 mL). The organic phase was washed with aq. HCl (1 M, 50 mL) and brine (30 mL), dried over anhydrous sodium sulfate, filtered concentrated in vacuo to give a residue compound Int-12 (105 mg, 0.150 mmol, 91.73% yield) as a yellow solid, which was used directly in the next step. MS obsd. (ESI⁺) [(M+H)⁺]: 706.4.

### Intermediate 13: 4-[[2-[4-[4-[4-(Tert-butoxycarbonylamino)-2-oxo-pyrrolidin-1-yl]phenyl]sulfonylpiperazin-1-yl]-6-chloro-4-pyridyl]-difluoro-methyl]benzoic acid

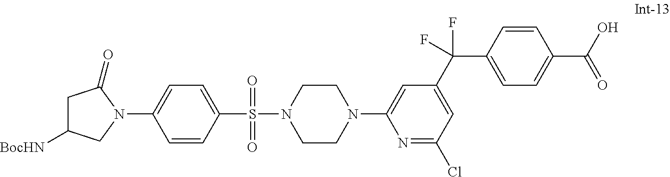 [SMILES: CC(C)(C)OC(=O)NC1CC(=O)N(c2ccc(S(=O)(=O)N3CCN(c4cc(C(F)(F)c5ccc(C(=O)O)cc5)cc(Cl)n4)CC3)cc2)C1]

The title compound was prepared in analogy to the preparation of compound Int-12 by using compound Int-1 instead of compound Int-2 in Step 4. Compound Int-13 (280.0 mg) was obtained as a white solid. MS obsd. (ESI⁺) [(M+H)⁺]: 706.0.

### Intermediate 14: 5-[[2-Chloro-6-[4-[4-[(4R)-4-methyl-2-oxo-pyrrolidin-1-yl]phenyl]sulfonylpiperazin-1-yl]-4-pyridyl]-difluoro-methyl]pyridine-2-carboxylic acid

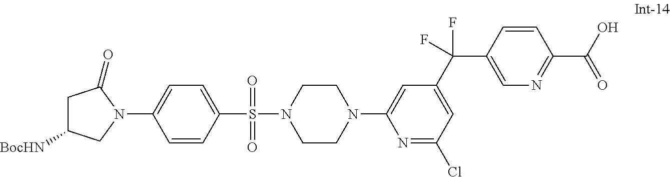 [SMILES: CC(C)(C)OC(=O)N[C@@H]1CC(=O)N(c2ccc(S(=O)(=O)N3CCN(c4cc(C(F)(F)c5ccc(C(=O)O)nc5)cc(Cl)n4)CC3)cc2)C1]

The title compound was prepared in analogy to the preparation of compound Int-12 by using methyl 5-formylpyridine-2-carboxylate instead of methyl 4-formylbenzoate in Step 1. Compound Int-14 (105 mg, 0.150 mmol, 91.73% yield) was obtained as a yellow solid. MS obsd. (ESI⁺) [(M+H)⁺]: 707.4.

### Intermediate 15: 6-[[2-Chloro-6-[4-[4-[(4R)-4-methyl-2-oxo-pyrrolidin-1-yl]phenyl]sulfonylpiperazin-1-yl]-4-pyridyl]-difluoro-methyl]pyridine-3-carboxylic acid

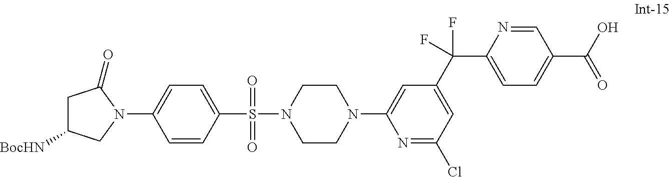 [SMILES: CC(C)(C)OC(=O)N[C@@H]1CC(=O)N(c2ccc(S(=O)(=O)N3CCN(c4cc(C(F)(F)c5ccc(C(=O)O)cn5)cc(Cl)n4)CC3)cc2)C1]

The title compound was prepared in analogy to the preparation of compound Int-12 by using methyl 6-formylpyridine-3-carboxylate instead of methyl 4-formylbenzoate in Step 1. Compound Int-15 (886 mg, 1.25 mmol, 80% yield) was obtained as a yellow solid. MS obsd. (ESI⁺) [(M+H)⁺]: 707.4.

### Intermediate 16: 5-[[2-Chloro-6-[4-[4-[(4R)-4-methyl-2-oxo-pyrrolidin-1-yl]phenyl]sulfonylpiperazin-1-yl]-4-pyridyl]-difluoro-methyl]pyrazine-2-carboxylic acid

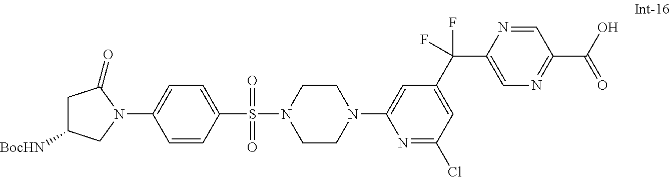 [SMILES: CC(C)(C)OC(=O)N[C@@H]1CC(=O)N(c2ccc(S(=O)(=O)N3CCN(c4cc(C(F)(F)c5cnc(C(=O)O)cn5)cc(Cl)n4)CC3)cc2)C1]

### Step 1: Pyrazine-2,5-dicarbonyl chloride

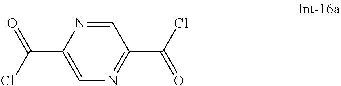 [SMILES: O=C(Cl)c1cnc(C(=O)Cl)cn1]

To a solution of pyrazine-2,5-dicarboxylic acid (25.0 g, 148.71 mmol) in thionyl chloride (106.15 g, 892.27 mmol) was added DMF (0.5 mL, 148.71 mmol). The reaction was stirred at 60° C. for 3 hours. After completion, the mixture was concentrated in vacuo and give crude Compound Int-16a (27.8 g, 135.61 mmol, 91.19% yield) as a brown solid, which was used in the next step directly. MS obsd. (ESI⁺) [(M+H)⁺]: 197.2 (quenched with MeOH).

### Step 2: Dimethyl pyrazine-2,5-dicarboxylate

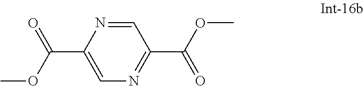

To a solution of methanol (100 mL, 2469 mmol) and TEA (53.03 mL, 380.49 mmol) in THF (100 mL) was added compound Int-16a (26.0 g, 126.83 mmol) in THF (150 mL) dropwise at 0° C. Then the reaction was stirred at 20° C. for 3 hours. After completion, the mixture was diluted with EtOAc (600 mL) and water (50 mL). The organic layer was washed with brine (100 mL×2), dried over anhydrous sodium sulfate, filtered and concentrated in vacuo to give crude product, which was recrystallized with MTBE (200 mL) to give Compound Int-16b (23.5 g, 119.8 mmol, 94.46% yield) as a light yellow solid. MS obsd. (ESI⁺) [(M+H)⁺]: 197.2 ([M+H]⁺).

### Step 3: Methyl 5-(2,6-dichloropyridine-4-carbonyl)pyrazine-2-carboxylate

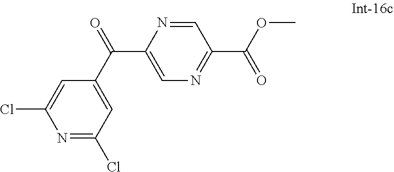 [SMILES: COC(=O)c1cnc(C(=O)c2cc(Cl)nc(Cl)c2)cn1]

To a solution of 2,6-dichloro-4-iodopyridine (20.0 g, 73.02 mmol) in THF (60 mL) was added isopropylmagnesium chloride lithium chloride complex (59 mL, 76.67 mmol) under nitrogen. The reaction mixture was stirred at 20° C. for 1 hour. Then the reaction mixture was added into the solution of compound Int-16b (23.5 g, 119.8 mmol) in THF (360 mL) at 70° C. dropwise for 1 hour. After addition, the reaction was quenched with sat. aq. NH₄Cl solution (100 mL) and extracted with EtOAc (300 mL×3). The combined organic layer was washed with brine (100 mL×3), dried over anhydrous sodium sulfate, filtered and the concentrated in vacuo to give a crude residue, which was purified by silica gel chromatography (eluent with EtOAc:PE=5-50%) to give compound Int-16c (10.8 g, 34.6 mmol, 47.39% yield) as a light yellow solid. MS obsd. (ESI⁺) [(M+H)⁺]: 312.1.

### Step 4: Methyl 5-[(2,6-dichloro-4-pyridyl)-difluoro-methyl]pyrazine-2-carboxylate

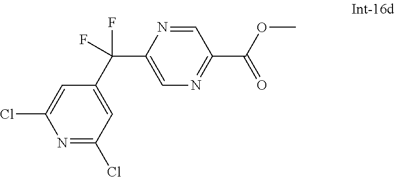 [SMILES: COC(=O)c1cnc(C(F)(F)c2cc(Cl)nc(Cl)c2)cn1]

To a solution of compound Int-16c (10.8 g, 34.6 mmol) in DCM (40 mL) was added DAST (55.78 g, 346.03 mmol) in one portion. The reaction was stirred at 50° C. for 32 hours. After completion, the reaction was poured into ice water and then adjusted pH to 7-8 with sat. aq. NaHCO₃ solution. The aqueous phase was extracted with EtOAc (300 mL×3). The combined organic layer was washed with brine (100 mL×3), dried over anhydrous sodium sulfate, filtered and concentrated in vacuo to give crude product, which was purified by silica gel chromatography (eluent with EtOAc:PE=5 to 25%) to give Int-16d (10.2 g, 30.53 mmol, 86.46% yield) as a white solid. MS obsd. (ESI⁺) [(M+H)⁺]: 334.1. ¹H NMR (400 MHz, DMSO-d₆) δ ppm: 9.38-9.43 (m, 1H), 9.30-9.35 (m, 1H), 7.95-8.01 (m, 2H), 3.98-4.02 (m, 3H).

### Step 5: Methyl 5-[[2-chloro-6-[4-[4-[(4R)-4-methyl-2-oxo-pyrrolidin-1-yl]phenyl]sulfonylpiperazin-1-yl]-4-pyridyl]-difluoro-methyl]pyrazine-2-carboxylate

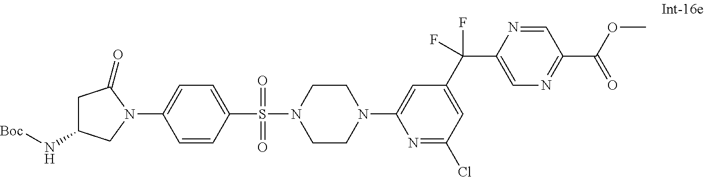 [SMILES: COC(=O)c1cnc(C(F)(F)c2cc(Cl)nc(N3CCN(S(=O)(=O)c4ccc(N5C[C@H](NC(=O)OC(C)(C)C)CC5=O)cc4)CC3)c2)cn1]

A mixture of compound Int-2 (639 mg, 1.51 mmol), compound Int-16d (504 mg, 1.51 mmol), DIPEA (584 mg, 4.52 mmol) in DMSO (3 mL) was stirred at 120° C. for 12 hours. After completion, the mixture was poured into water, extracted with EtOAc (200 mL×3). The combined organic layer was dried over sodium sulfate, filtered and concentrated in vacuo. The crude was purified by silica gel chromatography (eluent with EtOAc:PE=10-25%) to give compound Int-16e (762 mg, 70% yield) as a white solid. MS obsd. (ESI⁺) [(M+H)⁺]: 722.2.

### Step 6: 5-[[2-Chloro-6-[4-[4-[(4R)-4-methyl-2-oxo-pyrrolidin-1-yl]phenyl]sulfonylpiperazin-1-yl]-4-pyridyl]-difluoro-methyl]pyrazine-2-carboxylic acid

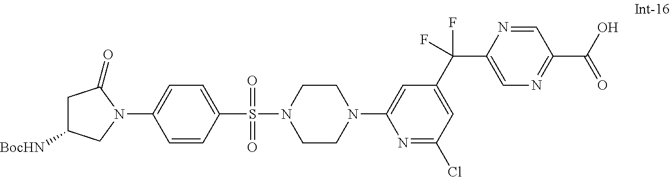 [SMILES: CC(C)(C)OC(=O)N[C@@H]1CC(=O)N(c2ccc(S(=O)(=O)N3CCN(c4cc(C(F)(F)c5cnc(C(=O)O)cn5)cc(Cl)n4)CC3)cc2)C1]

A mixture of compound Int-16e (150 mg, 208 μmol), lithium hydroxide hydrate (85.2 mg, 2.08 mmol) in MeOH (3 mL) and water (0.5 mL) was heated at 60° C. for 1 hour. After removal of the solvent, the resulting residue was treated with 5 N aq. HCl solution and adjusted to pH=5. The mixture was extracted with EtOAc (100 mL), and the organic layer was dried over anhydrous sodium sulfate, filtered, and concentrated in vacuo to afford compound Int-16 (132.4 mg, 0.187 mmol, 90% yield) as a yellow solid. MS obsd. (ESI⁺) [(M+H)⁺]: 708.2.

### Intermediate 17: Tert-butyl N-[1-(3-aminopropyl)-5-(tert-butoxycarbonylamino)-3-piperidyl]carbamate

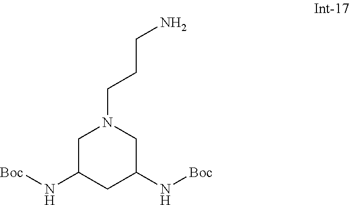 [SMILES: CC(C)(C)OC(=O)NC1CC(NC(=O)OC(C)(C)C)CN(CCCN)C1]

### Step 1: Tert-butyl N-[5-(tert-butoxycarbonylamino)-3-pyridyl]carbamate

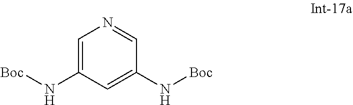 [SMILES: CC(C)(C)OC(=O)Nc1cncc(NC(=O)OC(C)(C)C)c1]

To a solution of pyridine-3,5-diamine (200 mg, 1.74 mmol) in 1,4-dioxane (10 mL) was added Boc₂O (1.9 g, 8.71 mmol) and DMAP (638.10 mg, 5.22 mmol) at 25° C. The mixture was stirred at 110° C. for 12 hours. After completion, the reaction mixture was poured into water (50 mL) and extracted by EtOAc (50 mL×3). The organic layer was washed by brine (50 ml×2), dried over anhydrous sodium sulfate and concentrated in vacuo to give a residue. To the resulting residue was added MeOH (20 mL) and K₂CO₃, and the mixture was stirred at 25° C. for 1 hour. Then, the mixture was filtered and concentrated in vacuo to give the crude compound, which was purified by silica gel chromatography (eluent with EtOAc:PE=0.5-50%) to give compound Int-17a (500 mg, yield: 92.8%) as a yellow solid. MS obsd. (ESI⁺) [(M+H)⁺]: 309.9.

### Step 2: Tert-butyl N-[5-(tert-butoxycarbonylamino)-3-piperidyl]carbamate

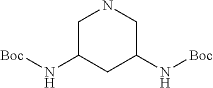 [SMILES: CC(C)(C)OC(=O)NC1CNCC(NC(=O)OC(C)(C)C)C1]

To a solution of compound Int-17a (6.2 g, 20.04 mmol) in THF (1 mL) and acetic acid (4.0 mL, 0.320 mmol) was added PtO₂ (100.0 mg, 0.320 mmol). The mixture was stirred at 60° C. for 12 hours under H₂. Then, the mixture was filtered through a Celite pad, and the filtrate was diluted with water (100 mL) and extracted with EtOAc (100 mL×2). The organic layer was washed with brine (100 mL), dried over anhydrous sodium sulfate and concentrated in vacuo to give compound Int-17b (1.6 g, 5.07 mmol, 25.31% yield). MS obsd. (ESI⁺) [(M+H)⁺]: 316.2.

### Step 3: Tert-butyl N-[1-[3-(benzyloxycarbonylamino)propyl]-5-(tert-butoxycarbonylamino)-3-piperidyl]carbamate

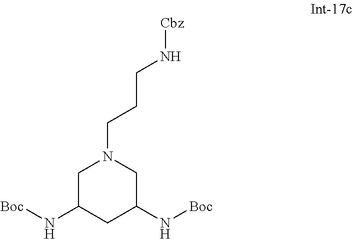 [SMILES: CC(C)(C)OC(=O)NC1CC(NC(=O)OC(C)(C)C)CN(CCCNC(=O)OCc2ccccc2)C1]

To a solution of benzyl N-(3-oxopropyl)carbamate (105 mg, 0.510 mmol) and compound Int-17b (160.0 mg, 0.510 mmol) in methanol (3 mL) was stirred for 1 hour. Then sodium cyanoborohydride (95.63 mg, 1.52 mmol) was added and the reaction was stirred at 20° C. for 2 hours. After completion, the reaction was added with aq. LiOH solution (1 M, 20 mL) and diluted with EtOAc (20 mL). The organic layer was separated and the aqueous phase was extracted with EtOAc (10 mL×2). The combined organic layer was washed with brine (50 mL), dried over anhydrous sodium sulfate, filtered, and concentrated in vacuo to afford compound Int-17c (77 mg, 0.150 mmol, 29.96% yield) as a white solid. MS obsd. (ESI⁺) [(M+H)⁺]: 507.4.

### Step 4: Tert-butyl N-[1-(3-aminopropyl)-5-(tert-butoxycarbonylamino)-3-piperidyl]carbamate

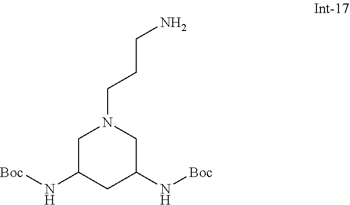 [SMILES: CC(C)(C)OC(=O)NC1CC(NC(=O)OC(C)(C)C)CN(CCCN)C1]

To a solution of compound Int-17c (67.0 mg, 0.130 mmol) in methanol (1 mL) were added Pd/C (280.36 mg, 2.64 mmol) and formic acid (3043.35 mg, 66.12 mmol) in one portion. The reaction was stirred at 25° C. for 1 hour. After completion, the mixture was filtered and the filtrate was concentrated in vacuo to give the resulting residue, which was re-suspended in the sat. aq. Na₂CO₃ solution (20 mL) and extracted with EtOAc (20 mL×3). The combined organic layer was washed with brine (50 mL), dried over anhydrous sodium sulfate, filtered, and concentrated in vacuo to afford compound Int-17 (49 mg, 0.130 mmol, 99.47% yield) as a white solid. MS obsd. (ESI⁺) [(M+H)⁺]: 373.3.

### Intermediate 18: Tert-butyl N-[2-[3-aminopropyl-[2-(tert-butoxycarbonyl-amino)ethyl]amino]ethyl]carbamate

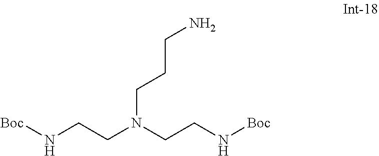 [SMILES: CC(C)(C)OC(=O)NCCN(CCCN)CCNC(=O)OC(C)(C)C]

### Step 1: Tert-butyl N-[2-[3-(benzyloxycarbonylamino)propyl-[2-(tert-butoxycarbonylamino)ethyl]amino]ethyl]carbamate

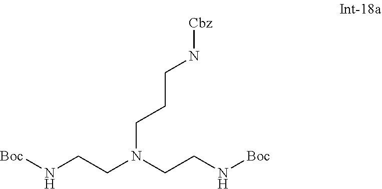 [SMILES: CC(C)(C)OC(=O)NCCN(CCCNC(=O)OCc1ccccc1)CCNC(=O)OC(C)(C)C]

To a solution of tert-butyl N-[2-[2-(tert-butoxycarbonylamino)ethylamino]ethyl]carbamate (500.0 mg, 1.65 mmol) in THF (5 mL) and MeOH (5 mL) was added benzyl N-(3-oxopropyl)carbamate (512.27 mg, 2.47 mmol) and NaBH₃CN (1035.6 mg, 16.48 mmol). After stirring at 45° C. for 3 hours, the reaction was concentrated in vacuo. The resulting residue was taken up in EtOAc (20 ml) and washed with water (30 mL) and brine (20 mL). The combined organic layer was dried over anhydrous sodium sulfate and concentrated in vacuo to give the crude compound, which was purified by prep-HPLC to give compound Int-18a (350 mg, 0.710 mmol, 42.94% yield) as colorless oil. MS obsd. (ESI⁺) [(M+H)⁺]: 495.0.

### Step 2: Tert-butyl N-[2-[3-aminopropyl-[2-(tert-butoxycarbonylamino)ethyl]amino]ethyl]carbamate

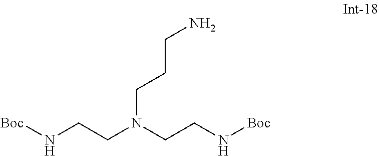 [SMILES: CC(C)(C)OC(=O)NCCN(CCCN)CCNC(=O)OC(C)(C)C]

To a solution of compound Int-18a (350 mg, 0.710 mmol) in MeOH (2 mL) was added Pd/C (1000 mg, 0.710 mmol) under nitrogen, then degassed by H₂ (1.0 L×3) and stirred at 20° C. for 2 hours. After completion, the reaction mixture was filtered through a Celite pad. The filtrate was then washed with MeOH (20 mL) and concentrated in vacuo to give compound Int-18 (200 mg, 0.550 mmol, 78.4% yield) as colorless oil. MS obsd. (ESI⁺) [(M+H)⁺]: 361.1.

### Intermediate 19: Tert-butyl 6-[(2,6-dichloro-4-pyridyl)-difluoro-methyl]bicyclo[3.1.0]-hexane-3-carboxylate

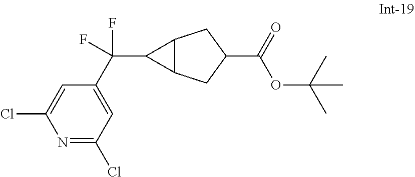 [SMILES: CC(C)(C)OC(=O)C1CC2C(C1)C2C(F)(F)c1cc(Cl)nc(Cl)c1]

### Step 1: Tert-butyl 6-formylbicyclo[3.1.0]hexane-3-carboxylate

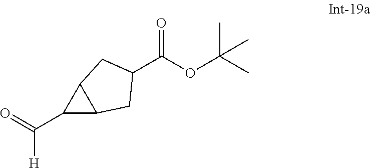

To a solution of tert-butyl 6-(hydroxymethyl)bicyclo[3.1.0]hexane-3-carboxylate (4.5 g, 21.2 mmol) and TEMPO (0.33 g, 2.12 mmol) in EtOAc (23.55 mL) was added trichloroisocyanuric acid (4.93 g, 21.2 mmol) at 0° C. under nitrogen. The mixture was stirred at 0° C. for 2 hours. After completion, the mixture was diluted with water (300 mL), extracted with EtOAc (500 mL×2) and washed with aq. HCl (1 N, 100 mL), aq. K₂CO₃ (300 ml) and brine (200 ml). The combined organic layer was dried by anhydrous sodium sulfate, filtered and concentrated in vacuo to give compound Int-19a (3.1 g, 14.74 mmol, 69.55% yield) as yellow oil.

### Step 2: Tert-butyl 6-[(2,6-dichloro-4-pyridyl)-hydroxy-methyl]bicyclo[3.1.0]-hexane-3-carboxylate

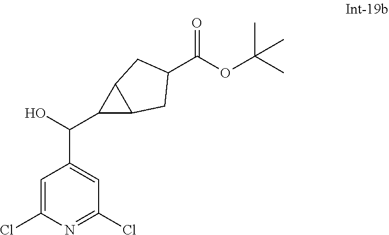 [SMILES: CC(C)(C)OC(=O)C1CC2C(C1)C2C(O)c1cc(Cl)nc(Cl)c1]

To a solution of 2,6-dichloro-4-iodopyridine (4.84 g, 17.69 mmol) in THF (10 mL) was added isopropylmagnesium chloride lithium chloride complex solution (1.3 M in THF, 17.01 mL, 22.11 mmol) at −40° C. The mixture was stirred at 25° C. for 1 hour. Then compound Int-19a (3.1 g, 14.74 mmol) in THF (10 mL) was added into the reaction at −40° C., and the mixture was stirred at 25° C. for 1 hour. After completion, the mixture was quenched with sat. aq. NH₄Cl solution (300 mL), extracted with EtOAc (500 mL×2) and washed with brine (300 mL). The combined organic layer was dried by anhydrous sodium sulfate, filtered, and concentrated in vacuo to give a crude residue, which was purified by silica gel column (eluent with EtOAc:PE=0-100%). Compound Int-19b (4.3 g, 12 mmol, 81.41% yield) was obtained as yellow oil. MS obsd. (ESI⁺) [(M+H)⁺]: 358.1.

### Step 3: Tert-butyl 6-(2,6-dichloropyridine-4-carbonyl)bicyclo[3.1.0]hexane-3-carboxylate

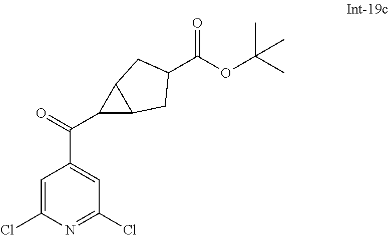 [SMILES: CC(C)(C)OC(=O)C1CC2C(C1)C2C(=O)c1cc(Cl)nc(Cl)c1]

To a solution of DMP (5.82 g, 13.73 mmol) in THF (30 mL) and was added compound Int-19b (4.1 g, 11.44 mmol). The reaction mixture was stirred at 25° C. for 1 hour. After completion, the mixture was diluted with EtOAc (300 mL) and adjusted to pH=9 with aq. K₂CO₃ solution. Then the resulting mixture was filtered through fritted glass funnel. The combined organic layer was washed with brine (200 mL), dried over anhydrous sodium sulfate, filtered and concentrated in vacuo to give compound Int-19c (3.9 g, 10.95 mmol, 95.66% yield) as yellow oil. MS obsd. (ESI⁺) [(M+H)⁺]: 356.1.

### Step 4: Tert-butyl 6-[(2,6-dichloro-4-pyridyl)-difluoro-methyl]bicyclo[3.1.0]-hexane-3-carboxylate

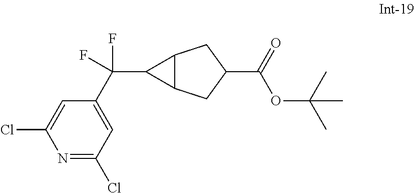 [SMILES: CC(C)(C)OC(=O)C1CC2C(C1)C2C(F)(F)c1cc(Cl)nc(Cl)c1]

To a solution of compound Int-19c (3.8 g, 10.67 mmol) in DCM (5 mL) was added DAST (30 mL, 10.67 mmol), and the mixture was stirred at 50° C. for 60 hours. After completion, the mixture was quenched with ice water and adjusted to pH=9 with 1 M aq. NaHCO₃ solution. The mixture was extracted with EtOAc (500 mL×2) and washed with brine (500 mL). The combined organic layer was dried over anhydrous sodium sulfate, filtered and concentrated in vacuo to give a crude residue, which was purified by silica gel column (eluent with EtOAc:PE=0-10%) and prep-HPLC to give compound Int-19 (200 mg, 0.260 mmol, 2.48% yield). MS obsd. (ESI⁺) [(M+H)⁺]: 378.3.

### Intermediate 20: Tert-butyl N-[(3R)-5-oxo-1-(5-piperazin-1-ylsulfonyl-2-pyridyl)pyrrolidin-3-yl]carbamate

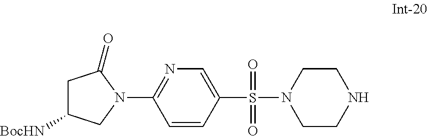 [SMILES: CC(C)(C)OC(=O)N[C@@H]1CC(=O)N(c2ccc(S(=O)(=O)N3CCNCC3)cn2)C1]

### Step 1: 6-Bromopyridine-3-sulfonyl chloride

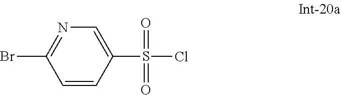 [SMILES: O=S(=O)(Cl)c1ccc(Br)nc1]

To a solution of 6-bromopyridin-3-amine (5.0 g, 28.9 mmol) in HCl (85 mL, 1020 mmol) was added NaNO₂ (1.79 g, 26.01 mmol) in water (25 mL) slowly at 0° C. The mixture was stirred at 0° C. for 2 hours. SO₂ was bubbled through a stirred solution of CuCl₂ (1976.76 mg, 11.56 mmol) in acetic acid (50 mL) at 0° C. for 0.5 hour. Then the diazotized reaction mixture prepared above was added. The resulting mixture was slowly warmed to room temperature and stirred for 30 minutes. After completion, the mixture was added EtOAc (200 mL) and water (200 mL). The organic phase was washed with aq. Na₂CO₃, dried over anhydrous sodium sulfate, concentrated in vacuo to give crude compound Int-20a (5 g, 23.58 mmol, 81.59% yield) as yellow oil, which was used directly in the next step.

### Step 2: Benzyl 4-[(6-chloro-3-pyridyl)sulfonyl]piperazine-1-carboxylate

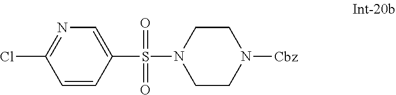 [SMILES: O=C(OCc1ccccc1)N1CCN(S(=O)(=O)c2ccc(Cl)nc2)CC1]

To a solution of compound Int-20a (4.13 g, 19.49 mmol) in DCM (1 mL) was added DIPEA (12.1 g, 97.47 mmol) and 1-Cbz-piperazine (4.29 g, 19.49 mmol). The mixture was stirred at 25° C. for 30 minutes. Then, the mixture was concentrated in vacuo to give a crude residue, which was triturated in MeOH to afford compound Int-20b (4.5 g, 11.37 mmol, 52.43% yield) as a white solid. MS obsd. (ESI⁺) [(M+H)]⁺: 395.8.

### Step 3: Benzyl 4-[(6-iodo-3-pyridyl)sulfonyl]piperazine-1-carboxylate

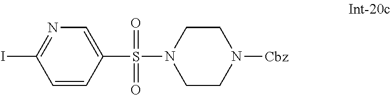 [SMILES: O=C(OCc1ccccc1)N1CCN(S(=O)(=O)c2ccc(I)nc2)CC1]

To a solution of compound Int-20c (4.0 g, 10.1 mmol) in acetone (100 mL) was added NaI (4.5 g, 30.31 mmol) and TFA (2.0 mL, 10.1 mmol). The mixture was stirred at 25° C. for 12 hours. After completion, the mixture was concentrated in vacuo to give a crude residue, which was triturated in MeOH to afford compound Int-20c (2.5 g, 5.13 mmol, 50.77% yield) as a white solid. MS obsd. (ESI⁺) [(M+H)]⁺: 487.7.

### Step 4: Benzyl 4-[[6-[(4R)-4-(tert-butoxycarbonylamino)-2-oxo-pyrrolidin-1-yl]-3-pyridyl]sulfonyl]piperazine-1-carboxylate

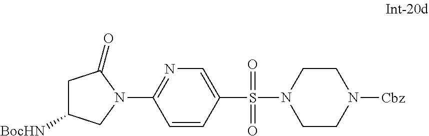 [SMILES: CC(C)(C)OC(=O)N[C@@H]1CC(=O)N(c2ccc(S(=O)(=O)N3CCN(C(=O)OCc4ccccc4)CC3)cn2)C1]

To a solution of tert-butyl N-[(3R)-5-oxopyrrolidin-3-yl]carbamate (123.27 mg, 0.620 mmol) and compound Int-20c (200.0 mg, 0.410 mmol) in DMF (1 mL) was added CuI (62.38 mg, 0.330 mmol), K₂CO₃ (169.91 mg, 1.23 mmol) and DMEDA (28.89 mg, 0.330 mmol). The mixture was stirred under nitrogen at 70° C. for 2 hours. After completion, the mixture was added EtOAc (50 mL) and filtered. The organic phase was added NH₃·H₂O (5%, 30 mL). The combined organic layer was washed with brine (50 mL), dried over anhydrous sodium sulfate, concentrated in vacuo to give a residue, which was purified by prep-HPLC to give compound Int-20d (160 mg, 0.290 mmol, 69.66% yield) as a white solid. MS obsd. (ESI⁺) [(M+H)]⁺: 560.0.

### Step 5: Tert-butyl N-[(3R)-5-oxo-1-(5-piperazin-1-ylsulfonyl-2-pyridyl)pyrrolidin-3-yl]carbamate

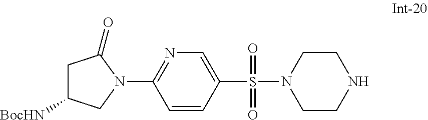 [SMILES: CC(C)(C)OC(=O)N[C@@H]1CC(=O)N(c2ccc(S(=O)(=O)N3CCNCC3)cn2)C1]

To a solution of compound Int-20d (160.0 mg, 0.290 mmol) in methanol (20 mL) was added Pd/C (80.0 mg, 0.060 mmol). The mixture was stirred under H₂ (15 psi) at 25° C. for 2 hours. After completion, the mixture was filtered through Celite. The filtrate was concentrated in vacuo to give the crude compound Int-20 (100 mg, 0.240 mmol, 82.2% yield) as a white solid, which was used directly in the next step. MS obsd. (ESI⁺) [(M+H)]⁺: 426.2.

### Intermediate 21: Tert-butyl N-[(3R)-5-oxo-1-[4-(4-piperidylsulfonyl)phenyl] pyrrolidin-3-yl]carbamate

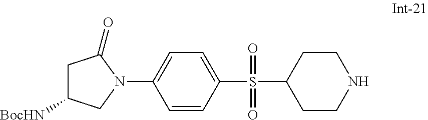 [SMILES: CC(C)(C)OC(=O)N[C@@H]1CC(=O)N(c2ccc(S(=O)(=O)C3CCNCC3)cc2)C1]

### Step 1: Benzyl 4-(4-iodophenyl)sulfanylpiperidine-1-carboxylate

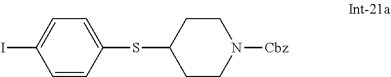 [SMILES: O=C(OCc1ccccc1)N1CCC(Sc2ccc(I)cc2)CC1]

To a solution of 4-bromobenzenethiol (5.0 g, 26.45 mmol), benzyl 4-hydroxypiperidine-1-carboxylate (4.4 mL, 29.09 mmol) and tributylphosphine (8.03 g, 39.67 mmol) in THF (150 mL) was added N,N,N,N-tetramethylazodicarboxamide (6.83 g, 39.67 mmol) at 0° C. The mixture was stirred at 45° C. for 2 hours. After completion, the reaction mixture was diluted with water (400 mL), extracted with EtOAc (400 mL×2) and washed with brine (500 mL). The combined organic layer was dried by anhydrous sodium sulfate, filtered and concentrated in vacuo to give a crude residue, which was purified by silica gel column (eluent with EtOAc:PE=0-10%) to give compound Int-21a (7.5 g, 18.46 mmol, 69.79% yield) as colorless oil. MS obsd. (ESI⁺) [(M+H)]⁺: 407.2.

### Step 2: Benzyl 4-(4-iodophenyl)sulfinylpiperidine-1-carboxylate

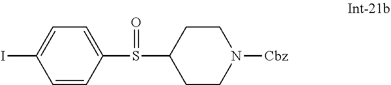 [SMILES: O=C(OCc1ccccc1)N1CCC(S(=O)c2ccc(I)cc2)CC1]

To a solution of compound Int-21a (7.3 g, 17.97 mmol) in THF (100 mL) was added m-CPBA (4.38 g, 21.56 mmol). The reaction was stirred at 25° C. for 2 hours. After completion, the reaction mixture was quenched with sat. aq. Na₂SO₃ solution and extracted with EtOAc (200 mL×2). The combined organic layer was washed with brine (50 mL), dried over anhydrous sodium sulfate, filtered and concentrated in vacuo to give a crude product, which was purified by silica gel column to give compound Int-21b (5.7 g, 13.5 mmol, 75.12% yield) as a white solid. MS obsd. (ESI⁺) [(M+H)]⁺: 424.1.

### Step 3: Benzyl 4-[4-[(4R)-4-(tert-butoxycarbonylamino)-2-oxo-pyrrolidin-1-yl]phenyl]sulfinylpiperidine-1-carboxylate

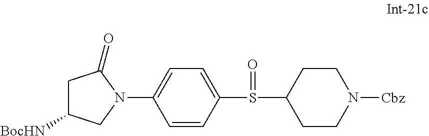 [SMILES: CC(C)(C)OC(=O)N[C@@H]1CC(=O)N(c2ccc(S(=O)C3CCN(C(=O)OCc4ccccc4)CC3)cc2)C1]

To a solution of compound Int-21c (237.04 mg, 1.18 mmol) in DMF (5 mL) was added tert-butyl N-[(3R)-5-oxopyrrolidin-3-yl]carbamate (355.57 mg, 1.78 mmol), CuI (157.74 mg, 0.830 mmol), DMEDA (72.93 mg, 0.830 mmol) and K₂CO₃ (490.13 mg, 3.55 mmol) at 30° C. The reaction mixture was stirred at 90° C. under nitrogen for 2 hours. After completion, the reaction mixture was filtered and the filtrate was dissolved in EtOAc (200 mL). The organic layer was washed with brine (50 mL×3), and concentrated in vacuo to give a crude residue. The residue was purified by prep-HPLC to afford compound Int-21c (450 mg, 0.830 mmol, 70.17% yield) as colorless oil. MS obsd. (ESI⁺) [(M+H)]⁺: 542.2.

### Step 4: Benzyl 4-[4-[(4R)-4-(tert-butoxycarbonylamino)-2-oxo-pyrrolidin-1-yl]phenyl]sulfonylpiperidine-1-carboxylate

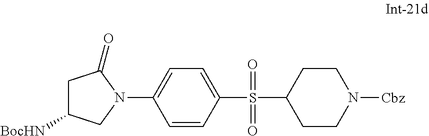 [SMILES: CC(C)(C)OC(=O)N[C@@H]1CC(=O)N(c2ccc(S(=O)(=O)C3CCN(C(=O)OCc4ccccc4)CC3)cc2)C1]

To a solution of compound Int-21c (220.0 mg, 0.410 mmol) in THF (5 mL) was added m-CPBA (164.92 mg, 0.810 mmol) at 30° C. The reaction mixture was stirred at 30° C. for 2 hours. After completion, the reaction mixture was quenched with sat. aq. Na₂SO₃ solution (20 mL), and extracted with EtOAc (100 mL). The organic layer was dried over anhydrous sodium sulfate, filtered and concentrated in vacuo to give a crude product, which was purified by prep-HPLC to give compound Int-21d (180 mg, 0.320 mmol, 79.47% yield) as a white solid. MS obsd. (ESI⁺) [(M+H)]⁺: 558.3.

### Step 5: Tert-butyl N-[(3R)-5-oxo-1-[4-(4-piperidylsulfonyl)phenyl] pyrrolidin-3-yl]carbamate

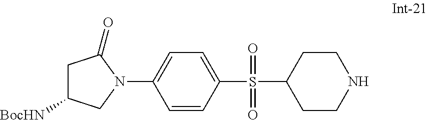 [SMILES: CC(C)(C)OC(=O)N[C@@H]1CC(=O)N(c2ccc(S(=O)(=O)C3CCNCC3)cc2)C1]

To a solution of compound Int-21d (180.0 mg, 0.320 mmol) in methanol (5 mL) was added formic acid (5 mL) and Pd/C (100 mg) at 30° C. under nitrogen. After stirring at 30° C. for 16 hours, the reaction mixture was filtered and the filtrate was concentrated in vacuo to give a crude residue, which was purified by prep-HPLC to give compound Int-21 (100 mg, 0.240 mmol, 73.15% yield) as a white solid. MS obsd. (ESI⁺) [(M+H)]⁺: 424.2.

### Intermediate 22: Tert-butyl N-[3-(tert-butoxycarbonylamino)propyl]-N-[5-oxo-1-(4-piperazin-1-ylsulfonylphenyl)pyrrolidin-3-yl]carbamate

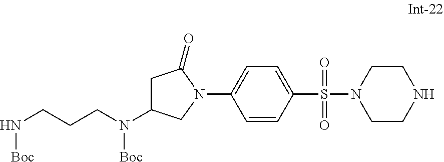 [SMILES: CC(C)(C)OC(=O)NCCCN(C(=O)OC(C)(C)C)C1CC(=O)N(c2ccc(S(=O)(=O)N3CCNCC3)cc2)C1]

### Step 1: Benzyl 4-[4-[4-(3-hydroxypropylamino)-2-oxo-pyrrolidin-1-yl]phenyl]sulfonylpiperazine-1-carboxylate

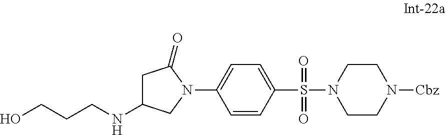 [SMILES: O=C(OCc1ccccc1)N1CCN(S(=O)(=O)c2ccc(N3CC(NCCCO)CC3=O)cc2)CC1]

To a solution of compound Int-4b (5.0 g, 11.33 mmol) in NMP (50 mL) was added 3-amino-1-propanol (5.2 mL, 67.95 mmol). The mixture was stirred at 90° C. for 2 hours. After completion, the mixture was diluted with water (150 mL) and extracted with EtOAc (80 mL×3). The combined organic layer was washed with brine (100 mL×3), dried over anhydrous sodium sulfate, filtered and concentrated in vacuo to give a crude residue compound Int-22a, which was used in the next step without further purification. MS obsd. (ESI⁺) [(M+H)⁺]: 517.2.

### Step 2: Benzyl 4-[4-[4-[tert-butoxycarbonyl(3-hydroxypropyl)amino]-2-oxo-pyrrolidin-1-yl]phenyl]sulfonylpiperazine-1-carboxylate

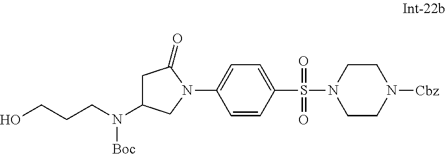 [SMILES: CC(C)(C)OC(=O)N(CCCO)C1CC(=O)N(c2ccc(S(=O)(=O)N3CCN(C(=O)OCc4ccccc4)CC3)cc2)C1]

To a solution of compound Int-22a (5.2 g, 10.07 mmol) in DCM (50 mL) was added DIPEA (7.01 mL, 40.26 mmol) and di-t-butyldicarbonate (4.63 mL, 20.13 mmol) in ice-water bath, then the reaction was stirred at 25° C. for 2 hours. After completion, the mixture was concentrated in vacuo and purified by silica gel chromatography (eluent with MeOH:DCM=0%-10%) to afford compound Int-22b (5.8 g, 9.4 mmol, 93.43% yield) as a light yellow solid. MS obsd. (ESI⁺) [(M+H)⁺]: 617.3.

### Step 3: Benzyl 4-[4-[4-[3-bromopropyl(tert-butoxycarbonyl)amino]-2-oxo-pyrrolidin-1-yl]phenyl]sulfonylpiperazine-1-carboxylate

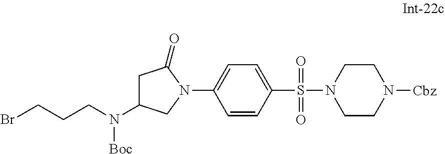 [SMILES: CC(C)(C)OC(=O)N(CCCBr)C1CC(=O)N(c2ccc(S(=O)(=O)N3CCN(C(=O)OCc4ccccc4)CC3)cc2)C1]

To a solution of compound Int-22b (5.8 g, 9.4 mmol) in DCM (60 mL) was added carbon tetrabromide (6.24 g, 18.81 mmol) at 0° C. under nitrogen, then triphenylphosphine (6.17 g, 23.51 mmol, 2.5 eq) was added into the solution. The mixture was stirred at 0° C. for 2 hours. After completion, the mixture was washed with brine (60 mL×3). The organic layer was concentrated in vacuo to give a crude, which was purified by reverse-phase chromatography to give compound Int-22c (5.4 g, 7.95 mmol, 84.49% yield) as a light yellow solid. MS obsd. (ESI⁺) [(M+H)⁺]: 679.2.

### Step 4: Benzyl 4-[4-[4-[3-aminopropyl(tert-butoxycarbonyl)amino]-2-oxo-pyrrolidin-1-yl]phenyl]sulfonylpiperazine-1-carboxylate

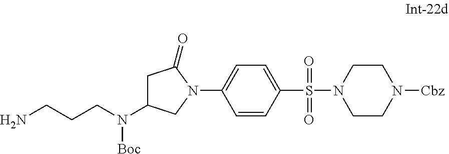 [SMILES: CC(C)(C)OC(=O)N(CCCN)C1CC(=O)N(c2ccc(S(=O)(=O)N3CCN(C(=O)OCc4ccccc4)CC3)cc2)C1]

To a solution of compound Int-22c (5.0 g, 7.36 mmol) in ethanol (50 mL) was added ammonium hydroxide (10.0 mL, 30%). The mixture was stirred at 50° C. for 4 hours. After completion, the mixture was concentrated in vacuo to afford compound Int-22d (3.2 g, 5.2 mmol, 64.99% yield) as a yellow solid. MS obsd. (ESI⁺) [(M+H)⁺]: 616.3.

### Step 5: Benzyl 4-[4-[4-[tert-butoxycarbonyl-[3-(tert-butoxycarbonylamino)-propyl]amino]-2-oxo-pyrrolidin-1-yl]phenyl]sulfonylpiperazine-1-carboxylate

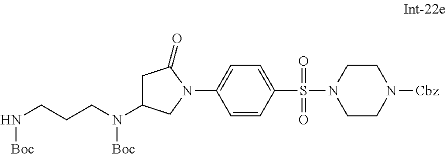 [SMILES: CC(C)(C)OC(=O)NCCCN(C(=O)OC(C)(C)C)C1CC(=O)N(c2ccc(S(=O)(=O)N3CCN(C(=O)OCc4ccccc4)CC3)cc2)C1]

To a solution of compound Int-22d (2.0 g, 3.25 mmol) in DCM (20 mL) was added DIPEA (1.7 mL, 9.74 mmol) and di-t-butyldicarbonate (1.49 mL, 6.5 mmol) in ice-water bath, then the reaction was stirred at 25° C. for 2 hours. After completion, the mixture was concentrated in vacuo and purified by silica gel chromatography (eluent with MeOH:DCM=0%-10%) to afford compound Int-22e (2.1 g, 2.93 mmol, 90.31% yield) as a white solid. MS obsd. (ESI⁺) [(M+Na)⁺]: 738.3.

### Step 6: Tert-butyl N-[3-(tert-butoxycarbonylamino)propyl]-N-[5-oxo-1-(4-piperazin-1-ylsulfonylphenyl)pyrrolidin-3-yl]carbamate

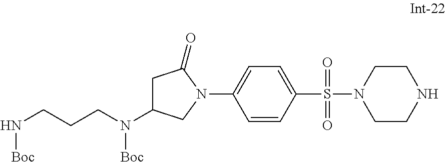 [SMILES: CC(C)(C)OC(=O)NCCCN(C(=O)OC(C)(C)C)C1CC(=O)N(c2ccc(S(=O)(=O)N3CCNCC3)cc2)C1]

To a solution of compound Int-22e (1.0 g, 1.4 mmol) in THF (30 mL) was added Pd/C (100 mg). The mixture was stirred at 50° C. under H₂ for 16 hours. After completion, the reaction mixture was filtered and the filtrate was concentrated in vacuo to give a crude product, which was purified by silica gel column (eluent with MeOH:DCM=0%-10%) to afford compound Int-22 (620 mg, 1.07 mmol, 72.48% yield) as a white solid. MS obsd. (ESI⁺) [(M+H)⁺]: 582.3. ¹H NMR (400 MHz, DMSO) δ 7.99 (d, J=8.7 Hz, 2H), 7.71 (d, J=8.8 Hz, 2H), 6.81 (s, 1H), 4.36 (s, 1H), 4.14 (t, J=9.2 Hz, 1H), 3.84 (dd, J=9.6, 5.3 Hz, 1H), 3.23 (d, J=5.7 Hz, 2H), 2.93-2.88 (m, 2H), 2.76-2.72 (m, 10H), 1.65-1.54 (m, 2H), 1.36 (d, J=11.1 Hz, 18H).

### Int-23: Trans-4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]sulfanyl]cyclohexanecarboxylic acid

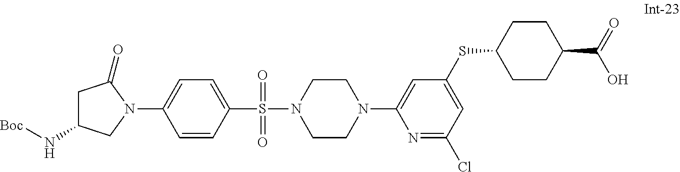 [SMILES: CC(C)(C)OC(=O)N[C@@H]1CC(=O)N(c2ccc(S(=O)(=O)N3CCN(c4cc(S[C@H]5CC[C@H](C(=O)O)CC5)cc(Cl)n4)CC3)cc2)C1]

### Step 1: 4-Benzylsulfanyl-2,6-dichloro-pyridine

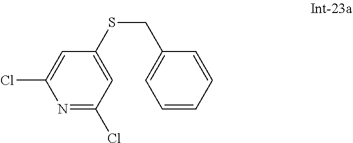 [SMILES: Clc1cc(SCc2ccccc2)cc(Cl)n1]

To a solution of benzyl mercaptan (4.58 g, 36.86 mmol), 4-bromo-2,6-dichloro-pyridine (8.2 g, 36.14 mmol) in DMF (50 mL) were added TEA (15.11 mL, 108.42 mmol), tris(dibenzylideneacetone)dipalladium(0) (1.32 g, 1.45 mmol) and 9,9-dimethyl-4,5-bis(diphenylphosphino)xanthene (2.09 g, 3.61 mmol) in one portion under nitrogen. The reaction was stirred at 80° C. for 2 hours. After completion, the reaction was diluted with water (100 ml) and extracted with EtOAc (200 mL×3). The combined organic layer was washed with sat. aq CaCl₂ (200 mL×3) and brine (200 mL×3). The organic layer was dried over anhydrous sodium sulfate and concentrated in vacuo to give a crude residue, which was purified by silica gel chromatography (eluent with EtOAc:PE=0-100%) to give compound Int-23a (8.8 g, 32.57 mmol, 90.12% yield) as a light yellow solid. MS obsd. (ESI⁺) [(M+H)⁺]: 269.8.

### Step 2: 2,6-Dichloropyridine-4-thiol

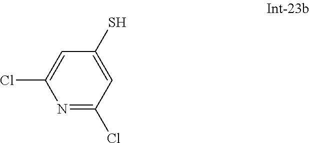

To a solution of compound Int-23a (4.0 g, 14.8 mmol) in DCM (50 mL) was added aluminum trichloride (9.87 g, 74.02 mmol). The reaction was stirred at 25° C. for 2 hours. After completion, the reaction mixture was poured into water (300 mL) in ice-water bath and extracted by EtOAc (300 mL×3). The combined organic layer was washed with brine (200 mL×2, dried over anhydrous sodium sulfate, filtered, and concentrated in vacuo to give a crude product, which was purified by prep-HPLC to afford compound Int-23b (1.5 g, 8.33 mmol, 56.27% yield) as a yellow solid. MS obsd. (ESI⁺) [(M+H)⁺]: 180.4.

### Step 3: Trans-methyl 4-[(2,6-dichloro-4-pyridyl)sulfanyl]cyclohexanecarboxylate

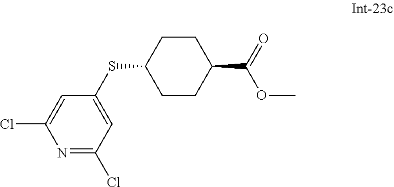 [SMILES: COC(=O)[C@H]1CC[C@H](Sc2cc(Cl)nc(Cl)c2)CC1]

To a solution of compound Int-23c (500.0 mg, 2.78 mmol), triphenylphosphine (3637.68 mg, 13.88 mmol) and methyl 4-hydroxycyclohexanecarboxylate (900.0 mg, 5.69 mmol) in THE (5 mL) was added diisopropyl azodicarboxylate (1683.61 mg, 8.33 mmol) dropwise at 0° C. The mixture was stirred at 70° C. for 16 hours. After completion, the mixture was diluted with water (100 mL) and extracted with EtOAc (200 mL×2). The combined organic layer was washed with brine (200 mL), dried over anhydrous sodium sulfate, concentrated in vacuo to give a crude residue, which was purified by prep-TLC (PE:EA=5:1, R_{f}=0.35) and prep-HPLC. The compound Int-23c (260 mg, 0.810 mmol, 29.33% yield) was obtained as colorless oil. MS obsd. (ESI⁺) [(M+H)⁺]: 320.2.

### Step 4: Trans-4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]sulfanyl]cyclohexanecarboxylic acid

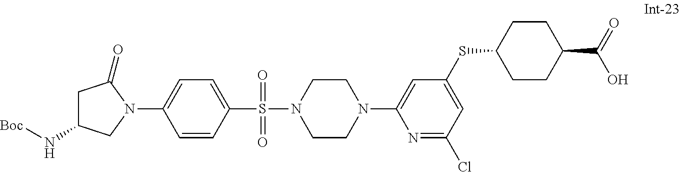 [SMILES: CC(C)(C)OC(=O)N[C@@H]1CC(=O)N(c2ccc(S(=O)(=O)N3CCN(c4cc(S[C@H]5CC[C@H](C(=O)O)CC5)cc(Cl)n4)CC3)cc2)C1]

The title compound was prepared in analogy to the preparation of compound Int-5 by replacing compound Int-23c instead of Int-5e in Step 6. Compound Int-23 (470 mg) was obtained as a light yellow solid. MS obsd. (ESI⁺) [(M+H)⁺]: 694.2.

### Int-24: Trans-4-[difluoro-[6-methyl-2-[4-[4-[(4R)-4-(tert-butoxycarbonylamino)-2-oxo-pyrrolidin-1-yl]phenyl]sulfonylpiperazin-1-yl]pyrimidin-4-yl]methyl]cyclohexanecarboxylic acid

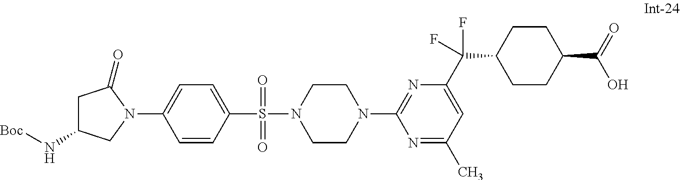 [SMILES: Cc1cc(C(F)(F)[C@H]2CC[C@H](C(=O)O)CC2)nc(N2CCN(S(=O)(=O)c3ccc(N4C[C@H](NC(=O)OC(C)(C)C)CC4=O)cc3)CC2)n1]

### Step 1: Chlorolithium; chloro-(2,2,6,6-tetramethyl-1-piperidyl)magnesium

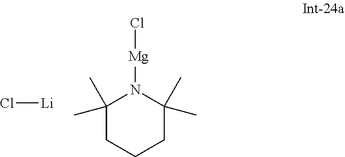

A solution of isopropylmagnesium chloride lithium chloride complex solution (1.3 M in THF, 50.0 mL, 65 mmol) was added 2,2,6,6-tetramethylpiperidine (12.07 mL, 71.5 mmol) dropwise at −40° C. The reaction was stirred at 25° C. under nitrogen for 1 day. The compound Int-24a (60 g, 247.52 mmol, 86.55% yield) in THF (60.0 mL) was obtained and used in the next step directly.

### Step 2: Trans-methyl 4-[(6-chloro-2-methylsulfanyl-pyrimidin-4-yl)-hydroxy-methyl]cyclohexanecarboxylate

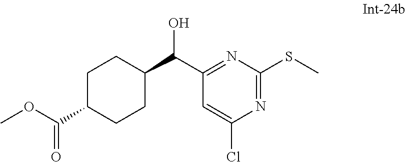

To a solution of 4-chloro-2-methylsulfanyl-pyrimidine (3.0 g, 18.68 mmol) in THF (8 mL) was added compound Int-24a (18.68 mL, 20.55 mmol) at −40° C. The mixture was stirred at 0° C. for 40 minutes and then added into the solution of trans-methyl 4-formylcyclohexanecarboxylate (3.5 g, 20.55 mmol) in THF (12 mL) at −40° C. The reaction mixture was stirred at 25° C. for 1 hour. After the starting material was consumed, the mixture was poured into water (200 mL) and extracted with EtOAc (200 mL×3). The combined organic layer was washed with brine (100 mL×2), dried over anhydrous sodium sulfate, filtered and concentrated in vacuo to give a crude residue, which was purified by silica gel chromatography on (eluent with EtOAc:PE=0-100%) to give compound Int-24b (1.8 g, 5.44 mmol, 29.13% yield) as light yellow oil. MS obsd. (ESI⁺) [(M+H)⁺]: 331.3.

### Step 3: Trans-methyl 4-(6-chloro-2-methylsulfanyl-pyrimidine-4-carbonyl)cyclohexanecarboxylate

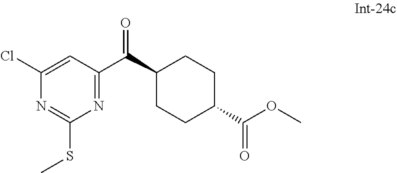 [SMILES: COC(=O)[C@H]1CC[C@H](C(=O)c2cc(Cl)nc(SC)n2)CC1]

To a solution of compound Int-24b (1.7 g, 5.14 mmol) in THF (10 mL) was added DMP (2.62 g, 6.17 mmol) at 0° C., and the mixture was stirred at 25° C. for 2 hours. After completion, the mixture was quenched with ice water and adjusted to pH=9 with aq. NaHCO₃ solution. The mixture was extracted with EtOAc (100 mL×2) and washed with brine (100 ml). The combined organic layer was dried over anhydrous sodium sulfate, filtered and concentrated in vacuo to give a crude residue, which was purified by silica gel column (eluent with EtOAc:PE=0-50%) to give compound Int-24c (1.6 g, 4.87 mmol, 94.7% yield) as a yellow solid. MS obsd. (ESI⁺) [(M+H)⁺]: 329.3.

### Step 4: Trans-methyl 4-[(6-chloro-2-methylsulfanyl-pyrimidin-4-yl)-difluoro-methyl]cyclohexanecarboxylate

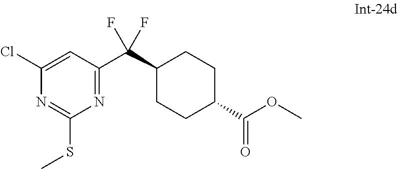 [SMILES: COC(=O)[C@H]1CC[C@H](C(F)(F)c2cc(Cl)nc(SC)n2)CC1]

To a solution of compound Int-24c (1.6 g, 4.87 mmol) in THF (10 mL) was added DAST (10.0 mL, 4.87 mmol). The mixture was stirred at 25° C. for 2 hours. After completion, the mixture was quenched with ice water, adjusted to pH=9 with aq. NaHCO₃ solution, and extracted with EtOAc (200 mL×2). The combined organic layer was washed with brine (100 mL), dried over anhydrous sodium sulfate, filtered and concentrated in vacuo to give a crude residue, which was purified by silica gel column (eluent with EtOAc:PE=0-20%) to give compound Int-24d (1.5 g, 4.28 mmol, 87.87% yield) as a yellow solid. MS obsd. (ESI⁺) [(M+H)⁺]: 351.3.

### Step 5: Trans-methyl 4-[difluoro-(6-methyl-2-methylsulfanyl-pyrimidin-4-yl)methyl]cyclohexanecarboxylate

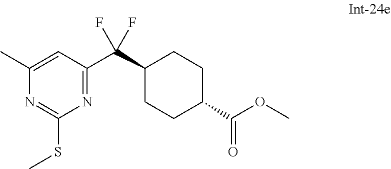

To a solution of compound Int-24d (900.0 mg, 2.57 mmol), chloro(2-dicyclohexylphosphino-2′,4′,6′-triisopropyl-1,1′-biphenyl)[2-(2′-amino-1,1′-biphenyl)]palladium(II) (201.85 mg, 0.260 mmol) and potassium phosphate (1.6 g, 7.7 mmol) in 1,4-dioxane (5 mL) and water (2 mL) was added trimethylboroxine (9.66 mL, 30.79 mmol). The mixture was stirred at 80° C. under nitrogen for 12 hours. After completion, the mixture was diluted with water (100 mL), and extracted with EtOAc (100 mL×2). The combined organic layer was washed with brine (100 mL), dried over anhydrous sodium sulfate, filtered and concentrated in vacuo to give a crude residue, which was purified by silica gel column (eluent with EtOAc:PE=10-30%) to afford compound Int-24e (420 mg, 1.27 mmol, 34.69% yield) as yellow oil. MS obsd. (ESI⁺) [(M+H)⁺]: 331.3.

### Step 6: Trans-methyl 4-[difluoro-(6-methyl-2-methylsulfonyl-pyrimidin-4-yl)methyl]cyclohexanecarboxylate

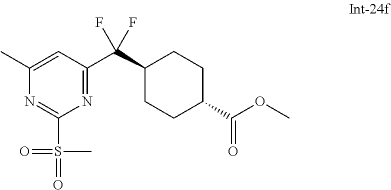

To a solution of compound Int-24e (400.0 mg, 0.850 mmol) in THF (5 mL) was added m-CPBA (516.18 mg, 2.54 mmol), and the mixture was stirred at 25° C. for 1 hour. After completion, the reaction mixture was quenched by sat. aq. Na₂SO₃ solution and extracted with EtOAc (100 mL×2). The combined organic layer was washed with brine (200 mL), dried over anhydrous sodium sulfate, filtered and concentrated in vacuo to give a residue, which was purified by silica gel column (eluent with EtOAc:PE=5-50%) to afford compound Int-24f (355 mg, 0.980 mmol, 87.85% yield) as yellow oil. MS obsd. (ESI⁺) [(M+H)⁺]: 363.2.

### Step 7: Trans-4-[difluoro-[6-methyl-2-[4-[4-[(4R)-4-(tert-butoxycarbonylamino)-2-oxo-pyrrolidin-1-yl]phenyl]sulfonylpiperazin-1-yl]pyrimidin-4-yl]methyl]cyclohexanecarboxylic acid

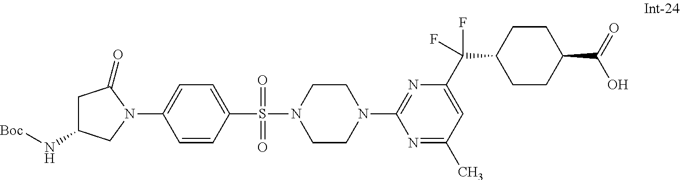 [SMILES: Cc1cc(C(F)(F)[C@H]2CC[C@H](C(=O)O)CC2)nc(N2CCN(S(=O)(=O)c3ccc(N4C[C@H](NC(=O)OC(C)(C)C)CC4=O)cc3)CC2)n1]

The title compound was prepared in analogy to the preparation of compound Int-5 by replacing compound Int-24f instead of compound Int-5e in Step 6. Compound Int-24 (260 mg) was obtained as a light yellow solid. MS obsd. (ESI⁺) [(M+H)⁺]: 693.4.

### Intermediate 25: Tert-butyl N-methyl-N-[(3R)-5-oxo-1-(4-piperazin-1-ylsulfonylphenyl)pyrrolidin-3-yl]carbamate

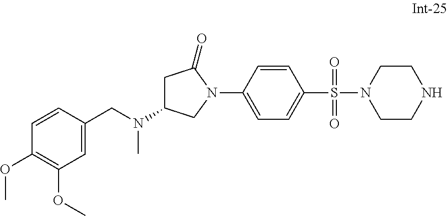 [SMILES: COc1ccc(CN(C)[C@@H]2CC(=O)N(c3ccc(S(=O)(=O)N4CCNCC4)cc3)C2)cc1OC]

### Step 1: Benzyl 4-[4-[(4R)-4-(tert-butoxycarbonylamino)-2-oxo-pyrrolidin-1-yl]phenyl]sulfonylpiperazine-1-carboxylate

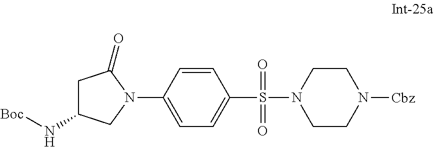 [SMILES: CC(C)(C)OC(=O)N[C@@H]1CC(=O)N(c2ccc(S(=O)(=O)N3CCN(C(=O)OCc4ccccc4)CC3)cc2)C1]

To a solution of Int-1a (412 mg, 2.06 mmol) in DMF (10 mL) was added benzyl 4-(4-iodophenyl)sulfonylpiperazine-1-carboxylate (1 g, 2.06 mmol), K₂CO₃ (853 mg, 6.17 mmol), CuI (195.81 mg, 1.03 mmol) and DMEDA (72.42 mg, 0.820 mmol). The suspension was stirred at 80° C. for 2 hours under N₂. After completion, the reaction was diluted with water (50 mL), extracted with EtOAc (50 mL×3). The combined organic layer was washed with brine (30 mL×2), dried over anhydrous sodium sulfate and concentrated. The crude product was purified by trituration in EtOAc (20 mL), which was filtered and the filtrate was concentrated to give Int-25a (660 mg, 1.18 mmol, 57.45% yield) as a white solid. MS obsd. (ESI⁺) [(M+H)⁺]: 559.1.

### Step 2: Benzyl 4-[4-[(4R)-4-amino-2-oxo-pyrrolidin-1-yl]phenyl]sulfonylpiperazine-1-carboxylate

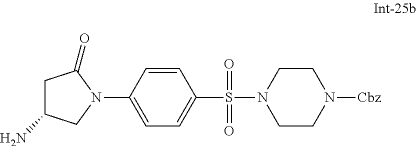 [SMILES: N[C@@H]1CC(=O)N(c2ccc(S(=O)(=O)N3CCN(C(=O)OCc4ccccc4)CC3)cc2)C1]

A mixture of Int-25a (660.0 mg, 1.18 mmol) and TFA (6.0 mL) in the DCM (6 mL) was stirred at room temperature for 2 hours. After the starting material was consumed, the mixture was concentrated in vacuo to afford Int-25b (660 mg, 1.15 mmol, 97.57% yield) as a light yellow gum. MS obsd. (ESI⁺) [(M+H)⁺]: 459.2.

### Step 3: Benzyl 4-[4-[(4R)-4-[(3,4-dimethoxyphenyl)methyl-methyl-amino]-2-oxo-pyrrolidin-1-yl]phenyl]sulfonylpiperazine-1-carboxylate

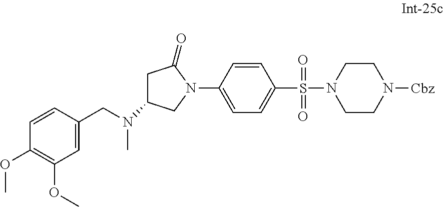 [SMILES: COc1ccc(CN(C)[C@@H]2CC(=O)N(c3ccc(S(=O)(=O)N4CCN(C(=O)OCc5ccccc5)CC4)cc3)C2)cc1OC]

To a solution of Int-25b (660.0 mg, 1.44 mmol) in THF (1 mL) was added Ti(i-PrO)₄ (4.1 g, 14.39 mmol) and 2,4-dimethoxybenzaldehyde (239.18 mg, 1.44 mmol) at 15° C. and then warmed to 25° C. The mixture was stirred at 25° C. for 2 hours. The mixture was cooled to 15° C. and then added methanol (1 mL) and NaBH₃CN (452.25 mg, 7.2 mmol). After stirring at 25° C. for 12 hours, paraformaldehyde (705.9 mg, 7.2 mmol, 5 eq) was added and the reaction was kept stirring at 25° C. for additional 12 hours. After completion, the mixture was diluted with DCM (50 mL) and water (50 mL). The aqueous phase was extracted with DCM (50 mL×2). The combined organic layer was washed with brine (50 mL), dried over anhydrous MgSO₄, filtered, and concentrated in vacuo to give the crude product, which was purified by silica gel chromatography (eluent with EtOAc:PE=0-50%) to afford Int-25c (210 mg, 0.340 mmol, 23.43% yield) as a white solid. MS obsd. (ESI⁺) [(M+H)⁺]: 623.3.

### Step 4: Tert-butyl N-methyl-N-[(3R)-5-oxo-1-(4-piperazin-1-ylsulfonylphenyl)pyrrolidin-3-yl]carbamate

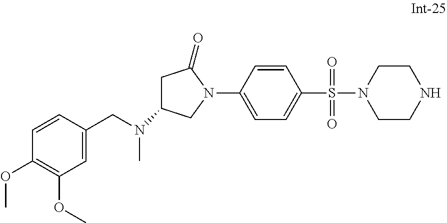 [SMILES: COc1ccc(CN(C)[C@@H]2CC(=O)N(c3ccc(S(=O)(=O)N4CCNCC4)cc3)C2)cc1OC]

To a solution of Int-25c (100.0 mg, 0.160 mmol) in the mixture of THF (0.1 mL) and IPA (1 mL) was added Pd/C (10.0 mg, 5%, wet) in one portion under N₂. The mixture was degassed under vacuum and purged with H₂ three times. The reaction mixture was stirred at 40° C. under H₂ balloon for 12 hours. After completion, the mixture was filtered and the filtration was concentrated in vacuo to afford Int-25 (75 mg, 0.150 mmol, 95.59% yield) as a white solid. MS obsd. (ESI⁺) [(M+H)⁺]: 489.3.

### Intermediate 26: 5-[[2-chloro-6-[4-[4-[(4R)-4-methyl-2-oxo-pyrrolidin-1-yl]phenyl]sulfonylpiperazin-1-yl]-4-pyridyl]-difluoro-methyl]bicyclo[2.2.2]octane-2-carboxylic acid

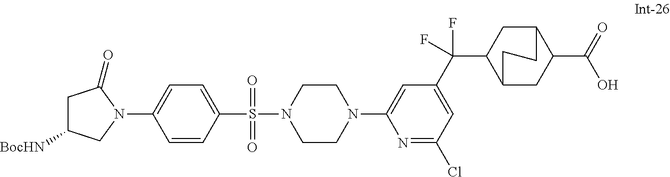 [SMILES: CC(C)(C)OC(=O)N[C@@H]1CC(=O)N(c2ccc(S(=O)(=O)N3CCN(c4cc(C(F)(F)C5CC6CCC5CC6C(=O)O)cc(Cl)n4)CC3)cc2)C1]

The title compound was prepared in analogy to the preparation of compound Int-5 by using 5-methoxycarbonylbicyclo[2.2.2]octane-2-carboxylic acid instead of trans-4-methoxycarbonylcyclohexanecarboxylic acid in Step 1. Compound Int-26 (450 mg) was obtained as a grey solid. MS obsd. (ESI⁺) [(M+H)⁺]: 738.3.

### Example 001: Trans-methyl 4-[[2-[4-[4-(4-amino-2-oxo-pyrrolidin-1-yl)phenyl]sulfonylpiperazin-1-yl]-6-chloro-4-pyridyl]-difluoro-methyl]cyclohexanecarboxylate

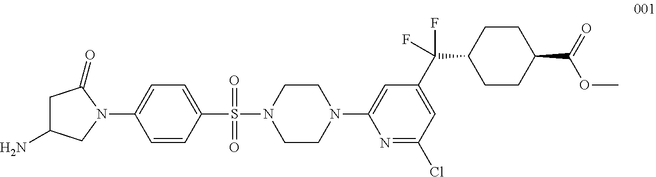 [SMILES: COC(=O)[C@H]1CC[C@H](C(F)(F)c2cc(Cl)nc(N3CCN(S(=O)(=O)c4ccc(N5CC(N)CC5=O)cc4)CC3)c2)CC1]

### Step 1:Trans-methyl 4-[[2-[4-[4-(4-amino-2-oxo-pyrrolidin-1-yl)phenyl]sulfonylpiperazin-1-yl]-6-chloro-4-pyridyl]-difluoro-methyl]cyclohexanecarboxylate

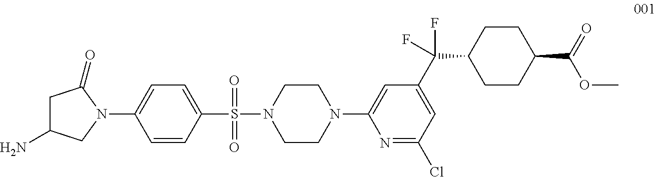 [SMILES: COC(=O)[C@H]1CC[C@H](C(F)(F)c2cc(Cl)nc(N3CCN(S(=O)(=O)c4ccc(N5CC(N)CC5=O)cc4)CC3)c2)CC1]

Compound Int-5e (250 mg, 344 μmol) was dissolved in the DCM (10 mL), followed by adding TFA (265 μL, 3.44 mmol). The reaction was stirred at room temperature for 1 hour. Then, the reaction mixture was concentrated in vacuo and purified by prep-HPLC to give product Example 001 (35 mg, 0.056 mmol, 15.4% yield) as a white solid. MS obsd. (ESI⁺) [(M+H)]⁺: 626.2.

Example 001: ¹H NMR (400 MHz, DMSO-d₆) δ ppm 7.91 (m, J=8.93 Hz, 2H), 7.75 (m, J=8.93 Hz, 2H), 6.76 (s, 1H), 6.69 (s, 1H), 4.00 (dd, J=9.90, 6.11 Hz, 1H), 3.60-3.70 (m, 6H), 3.45-3.54 (m, 1H), 3.32 (br s, 3H), 2.96 (br t, J=4.65 Hz, 4H), 2.78 (dd, J=16.87, 6.97 Hz, 1H), 2.52-2.53 (m, 1H), 2.11-2.38 (m, 3H), 1.91 (br d, J=10.64 Hz, 2H), 1.66 (br d, J=10.64 Hz, 2H), 1.11-1.35 (m, 4H). ¹⁹F NMR (376 MHz, DMSO-d₆) δ ppm −103.88 (br d, J=14.98 Hz, 1F).

### Example 002: Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(azetidin-3-ylmethyl)cyclohexanecarboxamide

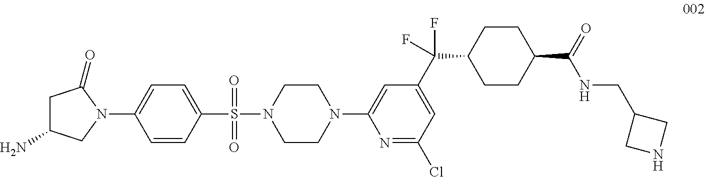 [SMILES: N[C@@H]1CC(=O)N(c2ccc(S(=O)(=O)N3CCN(c4cc(C(F)(F)[C@H]5CC[C@H](C(=O)NCC6CNC6)CC5)cc(Cl)n4)CC3)cc2)C1]

### Step 1: Trans-4-[[2-chloro-6-[4-[4-[(4R)-4-methyl-2-oxo-pyrrolidin-1-yl]phenyl]sulfonylpiperazin-1-yl]-4-pyridyl]-difluoro-methyl]-N-(cyclobutylmethyl)cyclohexanecarboxamide

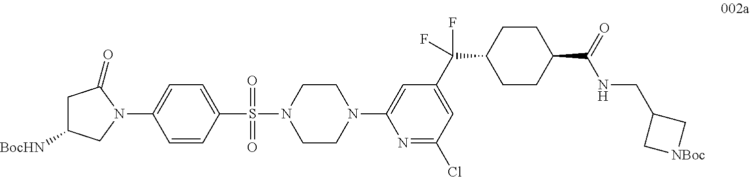 [SMILES: CC(C)(C)OC(=O)N[C@@H]1CC(=O)N(c2ccc(S(=O)(=O)N3CCN(c4cc(C(F)(F)[C@H]5CC[C@H](C(=O)NCC6CN(C(=O)OC(C)(C)C)C6)CC5)cc(Cl)n4)CC3)cc2)C1]

A mixture of azetidin-3-ylmethanamine (24.52 mg, 0.132 mmol), compound Int-5 (75 mg, 0.105 mmol), HATU (57 mg, 0.15 mmol) and DIPEA (55.12 μL, 0.316 mmol) in DCM (15 mL) was stirred at room temperature overnight. After completion, the reaction mixture was diluted with EtOAc (50 mL×3), washed with brine (50 mL). Then, the combined organic layer was dried over anhydrous sodium sulfate, filtered, concentrated in vacuo to give crude compound 002a (80 mg, 86.28% yield) as a light yellow solid, which was used directly in the next step. MS obsd. (ESI⁺) [(M+H)]⁺: 880.5.

### Step 2: Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(azetidin-3-ylmethyl)cyclohexanecarboxamide

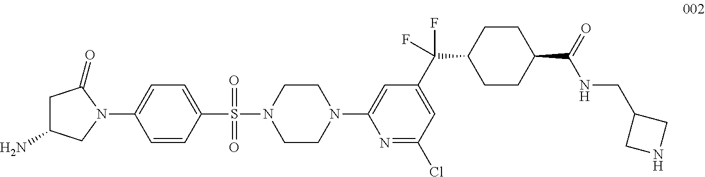 [SMILES: N[C@@H]1CC(=O)N(c2ccc(S(=O)(=O)N3CCN(c4cc(C(F)(F)[C@H]5CC[C@H](C(=O)NCC6CNC6)CC5)cc(Cl)n4)CC3)cc2)C1]

Compound 002a (80 mg, 91 μmol) was dissolved in the DCM (5 mL), followed by adding TFA (265 μL, 3.44 mmol). The reaction was stirred at room temperature for 1 hour and went completed. Then, the reaction was concentrated in vacuo and purified by prep-HPLC to give product Example 002 (19 mg, 30.7% yield) as white solid. MS obsd. (ESI⁺) [(M+H)]⁺: 680.2.

Example 002: ¹H NMR (400 MHz, METHANOL-d₄) δ ppm 7.95 (m, J=8.93 Hz, 2H), 7.86 (m, J=8.93 Hz, 2H), 6.67 (d, J=9.41 Hz, 2H), 5.27-5.46 (m, 1H), 4.95-5.16 (m, 2H), 4.42 (dd, J=11.43, 7.03 Hz, 1H), 4.14-4.27 (m, 1H), 4.02-4.11 (m, 2H), 3.84-4.00 (m, 3H), 3.64-3.77 (m, 4H), 3.36-3.44 (m, 2H), 3.21 (dd, J=18.16, 8.25 Hz, 1H), 3.03-3.13 (m, 5H), 2.71 (dd, J=18.03, 3.00 Hz, 1H), 2.50 (br s, 1H), 2.00-2.07 (m, 2H), 1.57 (br d, J=5.75 Hz, 6H), 1.33 (br d, J=18.10 Hz, 1H). ¹⁹F NMR (376 MHz, METHANOL-d₄) δ ppm −111.94-−101.94 (m, 1F).

The following Examples 003 to 052 were prepared in analogy to the procedure described for the preparation of Example 002, replacing compound Int-5 with “LINKER” in Step 1, and azetidin-3-ylmethanamine with “TAIL” in Step 1, by the reagents indicated in Table 1.

TABLE 1
Compound synthesis and characterization

| | Compound Names and | LINKER and | |
|---|---|---|---|
| Ex. | Structures | TAIL | NMR and (ESI⁺) |
| 003 | Trans-4-[[2-[4-[4-(4-amino-2- | LINKER:Int-6 | ¹H NMR (400 MHz, |
| | oxo-pyrrolidin-1- | TAIL: 3- | METHANOL-d₄) δ |
| | yl)phenyl]sulfonylpiperazin-1- | aminopropan-1- | ppm 7.92 (m, 2H), |
| | yl]-6-chloro-4-pyridyl]-difluoro- | ol | 7.83 (m, 2H), 6.66 (d, |
| | methyl]-N-(3- | | J = 7.09 Hz, 2H), 4.38 |
| | hydroxypropyl)cyclohexanecar- | | (dd, J = 11.37, 6.97 Hz, |
| | boxamide | | 1H), 4.12-4.22 (m, |
| | 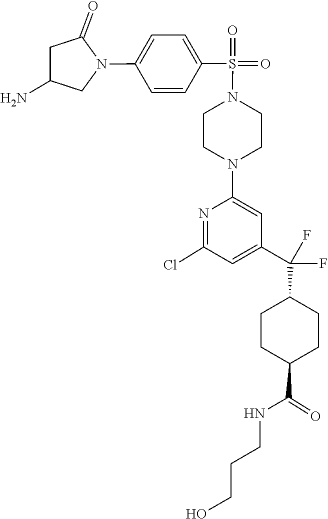 [SMILES: NC1CC(=O)N(c2ccc(S(=O)(=O)N3CCN(c4cc(C(F)(F)[C@H]5CC[C@H](C(=O)NCCCO)CC5)cc(Cl)n4)CC3)cc2)C1] | | 1H), 3.86-3.99 (m, 1H), 3.64-3.76 (m, 4H), 3.56 (t, J = 6.30 Hz, 2H), 3.32-3.36 (m, 1H), 3.12-3.27 (m, 3H), 3.06 (t, J = 4.89 Hz, 4H), 2.66 (dd, J = 18.10, 3.06 Hz, 1H), 1.93-2.14 (m, 2H), 1.64-1.87 (m, 6H), 1.34-1.51 (m, 2H), 1.16-1.31 (m, 2H). ¹⁹F NMR (376 MHz, DMSO-d₆) δ ppm −104.05 (br d, J = 13.62 Hz, 2F). MS obsd. (ESI⁺) [(M + H)⁺]: 669.3. |
| 004 | Trans-(4R)-4-amino-1-[4-[4-[4- | LINKER: Int-5 | ¹H NMR (400 MHz, |
| | [[4-(4-amino-4-methyl- | TAIL: tert-butyl | DMSO-d₆) δ ppm 7.85 |
| | piperidine-1- | N-(4-methyl-4- | (s, 1H), 7.83 (s, 1H), |
| | carbonyl)cyclohexyl]-difluoro- | piperidyl) | 7.64-7.70 (m, 2H), |
| | methyl]-6-chloro-2- | carbamate | 6.69 (s, 1H), 6.61 (s, |
| | pyridyl]piperazin-1- | | 1H), 3.91 (br dd, |
| | yl]sulfonylphenyl]pyrrolidin-2- | | J = 9.84, 6.05 Hz, 1H), |
| | one | | 3.47-3.67 (m, 6H), |
| | 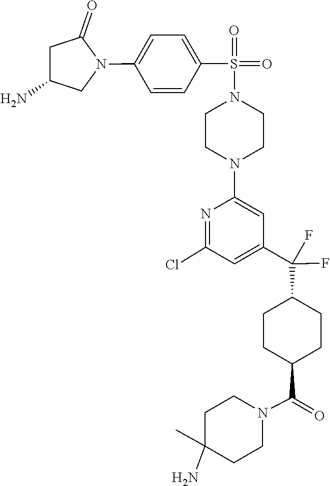 [SMILES: CC1(N)CCN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)CC1] | | 3.38-3.43 (m, 2H), 2.85-2.93 (m, 4H), 2.64-2.75 (m, 3H), 2.23-2.29 (m, 1H), 2.17 (br dd, J = 16.75, 4.03 Hz, 1H), 2.08 (br s, 1H), 1.67 (br d, J = 9.41 Hz, 2H), 1.51 (br s, 2H), 1.31-1.40 (m, 4H), 1.28 (br s, 2H), 1.21 (br s, 2H), 0.98 (s, 3H). ¹⁹F NMR (376 MHz, DMSO-d₆) δ ppm −102.64 (br d, J = 13.62 Hz, 2F). MS obsd. (ESI⁺) [(M + H)⁺]: 708.5. |
| 005 | Trans-(4R)-4-amino-1-[4-[4-[6- | LINKER: Int-5 | ¹H NMR (400 MHz, |
| | chloro-4-[difluoro-[4-(6-methyl- | TAIL: 2-methyl- | DMSO-d₆) δ ppm 7.91 |
| | 2,6-diazaspiro[3.3]heptane-2- | 2,6- | (d, J = 8.93 Hz, 2H), |
| | carbonyl)cyclohexyl]methyl]-2- | diazaspiro[3.3] | 7.74 (d, J = 9.05 Hz, |
| | pyridyl]piperazin-1- | heptane | 2H), 6.74 (s, 1H), 6.67 |
| | yl]sulfonylphenyl]pyrrolidin-2- | | (s, 1H), 4.14 (s, 2H), |
| | one | | 3.99 (dd, J = 9.78, |
| | 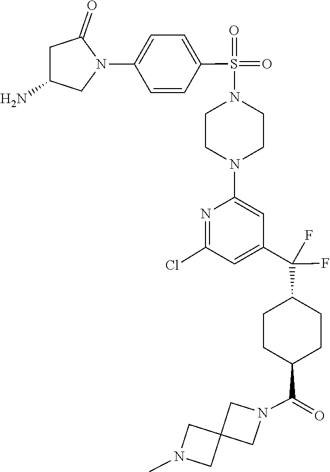 [SMILES: CN1CC2(C1)CN(C(=O)[C@H]1CC[C@H](C(F)(F)c3cc(Cl)nc(N4CCN(S(=O)(=O)c5ccc(N6C[C@H](N)CC6=O)cc5)CC4)c3)CC1)C2] | | 6.11 Hz, 1H), 3.83 (s, 2H), 3.57-3.72 (m, 4H), 3.46-3.51 (m, 2H), 3.14-3.22 (m, 6H), 2.89-3.03 (m, 4H), 2.76 (dd, J = 16.87, 6.97 Hz, 1H), 2.43-2.48 (m, 1H), 2.18-2.39 (m, 1H), 2.08-2.18 (m, 4H), 1.75 (br d, J = 11.00 Hz, 2H), 1.44-1.58 (m, 2H), 1.33-1.44 (m, 4H). ¹⁹F NMR (376 MHz, DMSO-d₆) δ ppm- −102.89 (s, 1F), −102.93 (s, 1F). MS obsd. (ESI⁺) [(M + H)⁺]: 706.4. |
| 006 | Trans-4-[2-chloro-6-[4-[4- | LINKER: Int-5 | ¹H NMR (400 MHz, |
| | [(4R)-4-amino-2-oxo-pyrrolidin- | TAIL: tert-butyl | METHANOL-d₄) δ |
| | 1-yl]phenyl]sulfonylpiperazin-1- | 3-amino-4- | ppm 7.82 (m, |
| | yl]-4-pyridyl]-difluoro-methyl]- | hydroxy- | J = 8.93 Hz, 2H), 7.71 |
| | N-(4-hydroxypyrrolidin-3- | pyrrolidine-1- | (m, J = 8.93 Hz, 2H), |
| | yl)cyclohexanecarboxamide | carboxylate | 6.56 (d, J = 9.29 Hz, |
| | 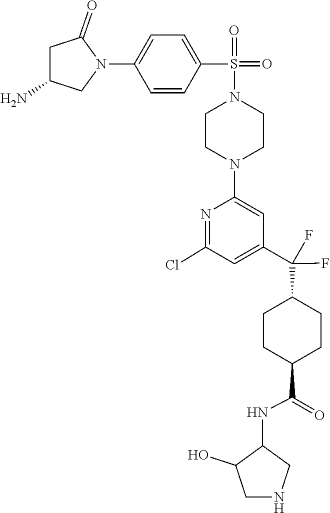 [SMILES: N[C@@H]1CC(=O)N(c2ccc(S(=O)(=O)N3CCN(c4cc(C(F)(F)[C@H]5CC[C@H](C(=O)NC6CNCC6O)CC5)cc(Cl)n4)CC3)cc2)C1] | | 2H), 4.08-4.24 (m, 3H), 4.05 (br d, J = 6.24 Hz, 1H), 3.73- 3.91 (m, 2H), 3.50- 3.68 (m, 7H), 3.28 (br dd, J = 12.35, 4.03 Hz, 2H), 3.05-3.18 (m, 2H), 2.88-3.03 (m, 5H), 2.40 (dd, J = 17.61, 3.79 Hz, 1H), 1.87-2.12 (m, 2H), 1.65-1.79 (m, 4H), 1.26-1.41 (m, 2H), 1.08-1.25 (m, 2H). ¹⁹F NMR (376 MHz, METHANOL-d₄) δ ppm −107.12 (s, 1F), −107.15 (s, 1F). MS obsd. (ESI⁺) [(M + H)⁺]: 696.5. |
| 007 | Trans-N-(3-aminocyclobutyl)- | LINKER: Int-5 | ¹H NMR (400 MHz, |
| | 4-[[2-chloro-6-[4-[4-[(4R)-4- | TAIL: tert-butyl | METHANOL-d₄) δ |
| | amino-2-oxo-pyrrolidin-1- | N-(3- | ppm 8.41 (br s, 1H), |
| | yl]phenyl]sulfonylpiperazin-1- | aminocyclobutyl) | 7.82 (d, J = 8.93 Hz, |
| | yl]-4-pyridyl]-difluoro- | carbamate | 2H), 7.71 (d, |
| | methyl]cyclohexanecarboxamide | | J = 8.93 Hz, 2H), 6.56 |
| | 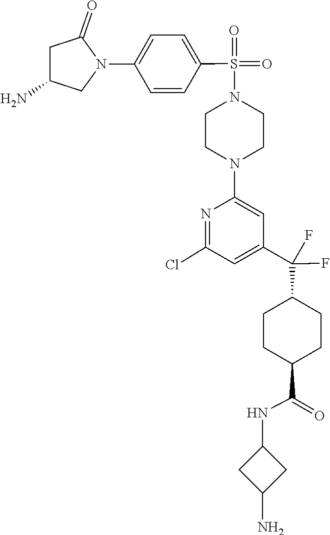 [SMILES: NC1CC(NC(=O)[C@H]2CC[C@H](C(F)(F)c3cc(Cl)nc(N4CCN(S(=O)(=O)c5ccc(N6C[C@H](N)CC6=O)cc5)CC4)c3)CC2)C1] | | (d, J = 9.29 Hz, 2H), 4.23-4.42 (m, 1H), 4.15 (dd, J = 10.58, 6.66 Hz, 1H), 3.81-4.03 (m, 2H), 3.63-3.77 (m, 2H), 3.55-3.62 (m, 4H), 3.31-3.45 (m, 1H), 2.89-3.01 (m, 5H), 2.61 (m, 1H), 2.25-2.48 (m, 4H), 1.91-2.06 (m, 3H), 1.71 (m, 4H), 1.25- 1.38 (m, 2H), 1.07- 1.22 (m, 2H). ¹⁹F NMR (376 MHz, METHANOL-d₄) δ ppm -107.09 (br t, J = 6.81 Hz, 1F), −107.13 (s, 1F). MS obsd. (ESI⁺) [(M + H)⁺]: 680.5. |
| 008 | Trans-N-(2-amino-1-methyl- | LINKER: Int-5 | ¹H NMR (400 MHz, |
| | ethyl)-4-[[2-chloro-6-[4-[4- | TAIL: tert-butyl | METHANOL-d₄) δ |
| | [(4R)-4-amino-2-oxo-pyrrolidin- | N-(2- | ppm 8.40 (br s, 1H), |
| | 1-yl]phenyl]sulfonylpiperazin-1- | aminopropyl) | 7.83 (s, 1H), 7.81 (s, |
| | yl]-4-pyridyl]-difluoro- | carbamate | 1H), 7.69-7.73 (m, |
| | methyl]cyclohexanecarboxamide | | 2H), 6.57 (d, |
| | 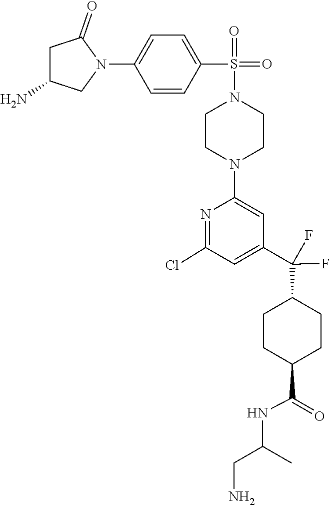 [SMILES: CC(CN)NC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | J = 9.41 Hz, 2H), 4.48 (br s, 2H), 4.13 (dd, J = 10.45, 6.54 Hz, 1H), 3.93-4.01 (m, 1H), 3.77-3.83 (m, 1H), 3.56-3.65 (m, 5H), 3.24-3.25 (m, 1H), 2.86-3.00 (m, 6H), 2.75-2.83 (m, 1H), 2.39 (dd, J = 17.55, 3.97 Hz, 1H), 1.94-2.09 (m, 2H), 1.79 (br s, 2H), 1.70 (br d, J = 12.72 Hz, 2H), 1.26- 1.38 (m, 2H), 1.08- 1.24 (m, 6H). ¹⁹F NMR (376 MHz, METHANOL-d₄) δ ppm −107.11 (s, 1F), −107.15 (s, 1F). MS obsd. (ESI⁺) [(M + H)⁺]: 668.6. |
| 009 | Trans-4-[[2-chloro-6-[4-[4- | LINKER: Int-5 | ¹H NMR (400 MHz, |
| | [(4R)-4-amino-2-oxo-pyrrolidin- | TAIL: 2- | METHANOL-d₄) δ |
| | 1-yl]phenyl]sulfonylpiperazin-1- | pyrrolidin-1- | ppm 7.84 (s, 1H), 7.82 |
| | yl]-4-pyridyl]-difluoro-methyl]- | ylethanamine | (s, 1H), 7.74 (d, |
| | N-(2-pyrrolidin-1- | | J = 8.93 Hz, 2H), 6.57 |
| | ylethyl)cyclohexanecarboxamide | | (d, J = 7.70 Hz, 2H), |
| | 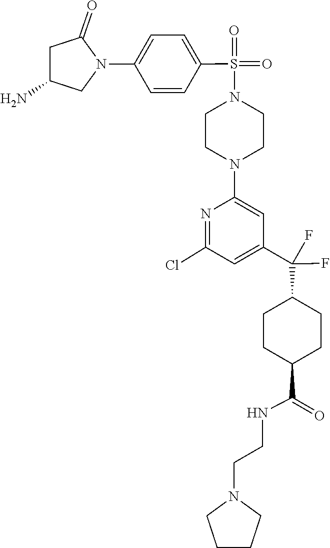 [SMILES: N[C@@H]1CC(=O)N(c2ccc(S(=O)(=O)N3CCN(c4cc(C(F)(F)[C@H]5CC[C@H](C(=O)NCCN6CCCC6)CC5)cc(Cl)n4)CC3)cc2)C1] | | 4.30 (dd, J = 11.43, 7.03 Hz, 1H), 4.05-4.13 (m, 1H), 3.85 (dd, J = 11.43, 2.38 Hz, 1H), 3.55-3.64 (m, 5H), 3.30-3.52 (m, 3H), 3.24-3.25 (m, 1H), 3.17-3.20 (m, 1H), 2.92-3.13 (m, 7H), 2.59 (dd, J = 18.03, 3.00 Hz, 1H), 1.90-2.14 (m, 6H), 1.80 (br d, J = 10.64 Hz, 2H), 1.70 (br d, J = 12.96 Hz, 2H), 1.28-1.38 (m, 2H), 1.09-1.24 (m, 3H). ¹⁹F NMR (376 MHz, METHANOL-d₄) δ ppm −107.09 (s, 1F), −107.13 (s, 1F). MS obsd. (ESI⁺) [(M + H)⁺]: 708.4. |
| 010 | Trans-4-[2-chloro-6-[4-[4- | LINKER: Int-5 | ¹H NMR (400 MHz, |
| | [(4R)-4-amino-2-oxo-pyrrolidin- | TAIL: tert-butyl | METHANOL-d₄) δ |
| | 1-yl]phenyl]sulfonylpiperazin-1- | 6-amino-3- | ppm 7.91 (d, |
| | yl]-4-pyridyl]-difluoro-methyl]- | azabicyclo[3.1.0] | J = 7.90 Hz, 1H), 7.78- |
| | N-[3-azabicyclo[3.1.0]hexan-6- | hexane-3- | 7.81 (m, 1H), 6.65 (d, |
| | yl]cyclohexanecarboxamide | carboxylate | J = 9.66 Hz, 1H), 5.34 |
| | 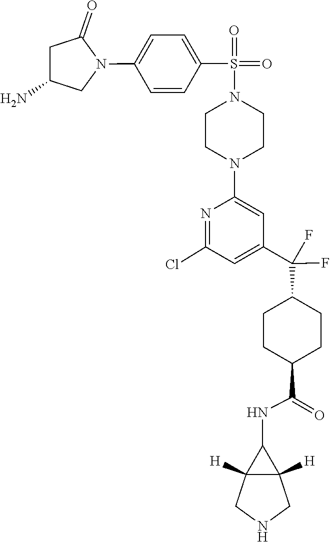 [SMILES: N[C@@H]1CC(=O)N(c2ccc(S(=O)(=O)N3CCN(c4cc(C(F)(F)[C@H]5CC[C@H](C(=O)NC6[C@H]7CNC[C@@H]67)CC5)cc(Cl)n4)CC3)cc2)C1] | | (br t, J = 4.65 Hz, 1H), 4.58 (br s, 3H), 3.61- 3.80 (m, 3H), 3.04- 3.16 (m, 5H), 2.77- 3.03 (m, 2H), 2.36- 2.45 (m, 1H), 2.14- 2.24 (m, 1H), 1.96- 2.11 (m, 2H), 1.78 (br t, J = 11.55 Hz, 2H), 1.54-1.62 (m, 2H), 1.37-1.48 (m, 1H), 1.15-1.37 (m, 8H), 0.80-0.99 (m, 1H), 0.00 (s, 3H). ¹⁹F NMR (376 MHz, METHANOL-d₄) δ ppm −107.08 (s, 1F), −107.12 (s, 1F). MS obsd. (ESI⁺) [(M + H)⁺]: 692.4. |
| 011 | Trans-4-[[2-chloro-6-[4-[4- | LINKER: Int-5 | ¹H NMR (400 MHz, |
| | [(4R)-4-amino-2-oxo-pyrrolidin- | TAIL: tert- | METHANOL-d₄) δ |
| | 1-yl]phenyl]sulfonylpiperazin-1- | butyl-cis-3,4- | ppm 8.52 (br s, 1H), |
| | yl]-4-pyridyl]-difluoro-methyl]- | diaminopyrrolidine- | 7.91 (d, J = 8.25 Hz, |
| | N-[cis-4-aminopyrrolidin-3- | 1-carboxylate | 2H), 7.80 (d, |
| | yl]cyclohexanecarboxamide | | J = 8.24 Hz, 2H), 6.65 |
| | 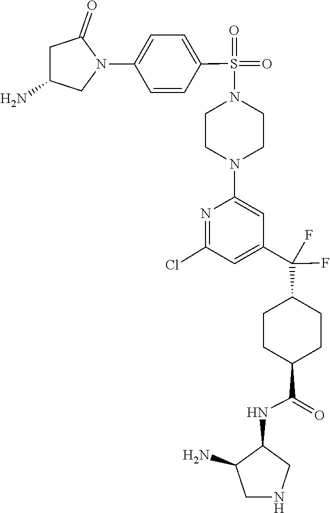 [SMILES: N[C@@H]1CC(=O)N(c2ccc(S(=O)(=O)N3CCN(c4cc(C(F)(F)[C@H]5CC[C@H](C(=O)N[C@H]6CNC[C@H]6N)CC5)cc(Cl)n4)CC3)cc2)C1] | | (d, J = 9.66 Hz, 2H), 5.34 (m, 1H), 4.74 (br s, 1H), 4.63 (br s, 1H), 4.28-4.34 (m, 1H), 4.20 (dd, J = 10.45, 6.54 Hz, 1H), 3.87 (tt, J = 7.00, 3.70 Hz, 1H), 3.65-3.73 (m, 6H), 3.35-3.49 (m, 2H), 2.94-3.29 (m, 8H), 2.56 (br s, 1H), 2.47 (dd, J = 17.36, 3.91 Hz, 1H), 1.97-2.09 (m, 3H), 1.48-1.68 (m, 6H), 1.25-1.35 (m, 1H). ¹⁹F NMR (376 MHz, METHANOL-d₄) δ ppm −105.35 (s, 2F). MS obsd. (ESI⁺) [(M + H)⁺]: 695.3. |
| 012 | Trans-N-[1- | LINKER: Int-5 | ¹H NMR (400 MHz, |
| | (aminomethyl)cyclopropyl]-4- | TAIL: tert-butyl | METHANOL-d₄) δ |
| | [[2-[4-[4-[(4R)-4-amino-2-oxo- | N-[(1- | ppm 8.52 (br s, 1H), |
| | pyrrolidin-1- | aminocyclopropyl) | 7.92-7.93 (m, 1H), |
| | yl]phenyl]sulfonylpiperazin-1- | methyl] | 7.90 (s, 1H), 7.78-7.83 |
| | yl]-6-chloro-4-pyridyl]-difluoro- | carbamate | (m, 2H), 6.66 (d, |
| | methyl]cyclohexanecarboxamide | | J = 9.05 Hz, 2H), 4.23 |
| | 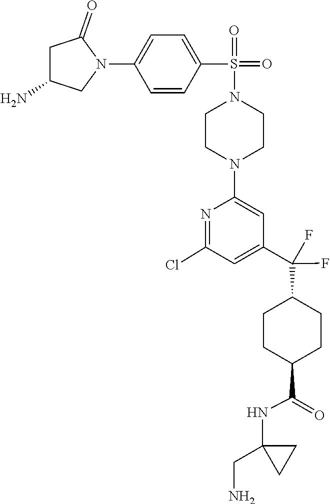 [SMILES: NCC1(NC(=O)[C@H]2CC[C@H](C(F)(F)c3cc(Cl)nc(N4CCN(S(=O)(=O)c5ccc(N6C[C@H](N)CC6=O)cc5)CC4)c3)CC2)CC1] | | (dd, J = 10.58, 6.54 Hz, 1H), 3.93 (m, 1H), 3.62-3.79 (m, 5H), 2.98-3.10 (m, 7H), 2.51 (dd, J = 17.61, 3.79 Hz, 1H), 1.96-2.13 (m, 2H), 1.73-1.89 (m, 4H), 1.28-1.47 (m, 2H), 1.15-1.27 (m, 2H), 0.94-0.99 (m, 2H), 0.84-0.90 (m, 2H). ¹⁹F NMR (376 MHz, METHANOL-d₄) δ ppm −107.06 (s, 1F), −107.10 (s, 1F). MS obsd. (ESI⁺) [(M + H)⁺]: 680.3 |
| 013 | Trans-(4R)-4-amino-1-[4-[4-[4- | LINKER: Int-5 | ¹H NMR (400 MHz, |
| | [[4-[4-(2-aminoethyl)piperazine- | TAIL: tert-butyl | METHANOL-d₄) δ |
| | 1-carbonyl]cyclohexyl]-difluoro- | N-(2-piperazin- | ppm 7.92 (m, |
| | methyl]-6-chloro-2- | 1- | J = 9.05 Hz, 2H), 7.82 |
| | pyridyl]piperazin-1- | ylethyl)carbamate | (m, J = 9.05 Hz, 2H), |
| | yl]sulfonylphenyl]pyrrolidin-2- | | 6.67 (d, J = 7.34 Hz, |
| | one | | 2H), 4.34 (dd, |
| | 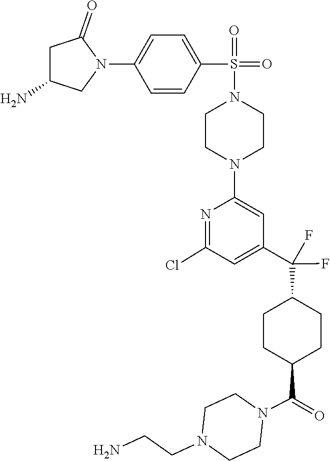 [SMILES: NCCN1CCN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)CC1] | | J = 11.13, 6.85 Hz, 1H), 3.94-4.17 (m, 3H), 3.89 (br dd, J = 11.07, 2.63 Hz, 2H), 3.79 (br d, J = 15.16 Hz, 1H), 3.65-3.73 (m, 4H), 3.41-3.62 (m, 6H), 3.22-3.29 (m, 4H), 3.01-3.16 (m, 5H), 2.57-2.69 (m, 2H), 1.99-2.15 (m, 1H), 1.81 (m, 4H), 1.45 (m, 2H), 1.25-1.39 (m, 2H). ¹⁹F NMR (376 MHz, METHANOL-d₄) δ ppm −106.86 (s, 1F), −106.89 (s, 1F). MS obsd. (ESI⁺) [(M + H)⁺]: 723.4. |
| 014 | Trans-4-[2-[4-[4-[(4R)-4- | LINKER: Int-5 | ¹H NMR (400 MHz, |
| | amino-2-oxo-pyrrolidin-1- | TAIL: | DMSO-d₆) δ ppm 7.92 |
| | yl]phenyl]sulfonylpiperazin-1- | ammonium | (m, J = 8.78 Hz, 2H), |
| | yl]-6-chloro-4-pyridyl]-difluoro- | chloride | 7.75 (m, J = 8.78 Hz, |
| | methyl]cyclohexanecarboxamide | | 2H), 6.78 (s, 1H), |
| | 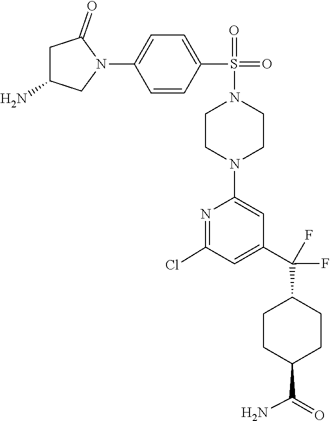 [SMILES: NC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 6.66-6.73 (m, 1H), 3.99-4.05 (m, 1H), 3.70 (br s, 1H), 3.65 (br s, 4H), 3.53 (br d, J = 9.29 Hz, 1H), 2.96 (br s, 4H), 2.80 (br dd, J = 17.19, 6.90 Hz, 1H), 2.24-2.34 (m, 2H), 2.07-2.22 (m, 1H), 1.95-2.04 (m, 2H), 1.76 (br d, J = 11.54 Hz, 2H), 1.65 (br d, J = 11.54 Hz, 2H), 1.20- 1.37 (m, 4H), 1.01- 1.19 (m, 2H). ¹⁹F NMR (376 MHz, METHANOL-d₄) δ ppm −107.23 (s, 1F), −107.27 (s, 1F). MS obsd. (ESI⁺) [(M + H)⁺]: 610.8 |
| 015 | 4-[[2-[4-[4-(4-amino-2-oxo- | LINKER: Int- | ¹H NMR (400 MHz, |
| | pyrrolidin-1- | 13 | METHANOL-d₄) δ |
| | yl)phenyl]sulfonylpiperazin-1- | TAIL: 3- | ppm 7.75-7.83 (m, |
| | yl]-6-chloro-4-pyridyl]-difluoro- | aminopropan-1- | 4H), 7.67-7.71 (m, |
| | methyl]-N-(3- | ol | 2H), 7.51 (d, |
| | hydroxypropyl)benzamide | | J = 8.56 Hz, 2H), 6.70 |
| | 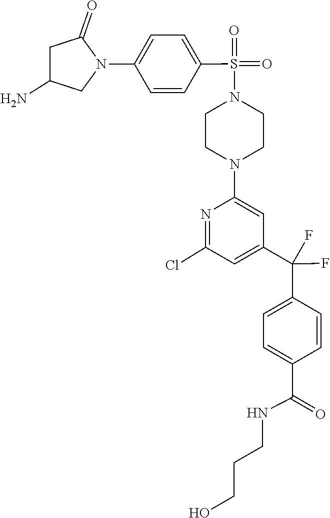 [SMILES: NC1CC(=O)N(c2ccc(S(=O)(=O)N3CCN(c4cc(C(F)(F)c5ccc(C(=O)NCCCO)cc5)cc(Cl)n4)CC3)cc2)C1] | | (s, 1H), 6.59 (s, 1H), 5.07 (br s, 1H), 4.08 (dd, J = 10.27, 6.48 Hz, 1H), 3.67-3.80 (m, 1H), 3.51-3.64 (m, 7H), 3.32-3.43 (m, 3H), 2.92-2.98 (m, 4H), 2.85 (dd, J = 17.36, 7.46 Hz, 1H), 2.34 (dd, J = 17.36, 4.03 Hz, 1H), 1.73 (m, J = 6.60 Hz, 2H). ¹⁹F NMR (376 MHz, METHANOL-d₄) δ ppm −95.14 (s, 2F). MS obsd. (ESI⁺) [(M + H)⁺]: 663.3. |
| 016 | 4-[[2-[4-[4-(4-amino-2-oxo- | LINKER: Int- | ¹H NMR (400 MHz, |
| | pyrrolidin-1- | 13 | METHANOL-d₄) δ |
| | yl)phenyl]sulfonylpiperazin-1- | TAIL: propane- | ppm 8.55 (s, 1H), 7.90 |
| | yl]-6-chloro-4-pyridyl]-difluoro- | 1,3-diamine | (d, J = 8.80 Hz, 4H), |
| | methyl]-N-(3- | | 7.76-7.81 (m, 2H), |
| | aminopropyl)benzamide | | 7.63 (d, J = 8.44 Hz, |
| | 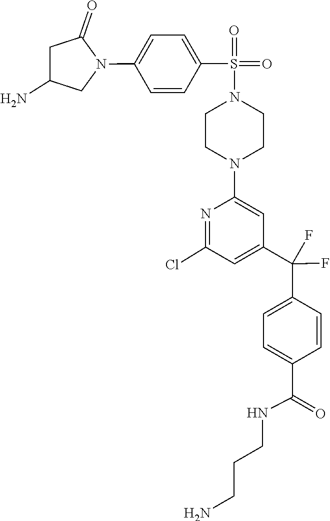 [SMILES: NCCCNC(=O)c1ccc(C(F)(F)c2cc(Cl)nc(N3CCN(S(=O)(=O)c4ccc(N5CC(N)CC5=O)cc4)CC3)c2)cc1] | | 2H), 6.81 (s, 1H), 6.68 (s, 1H), 4.14 (dd, J = 10.15, 6.36 Hz, 1H), 3.72-3.84 (m, 1H), 3.61-3.72 (m, 5H), 3.45-3.52 (m, 3H), 3.02-3.09 (m, 4H), 2.88-2.98 (m, 3H), 2.41 (dd, J = 17.18, 4.22 Hz, 1H), 1.92 (m, 2H). ¹⁹F NMR (376 MHz, METHANOL-d₄) δ ppm −95.26 (s, 2F). MS obsd. (ESI⁺) [(M + H)⁺]: 662.3. |
| 017 | N-(2-aminoethyl)-4-[[2-[4-[4-(4- | LINKER: Int- | ¹H NMR (400 MHz, |
| | amino-2-oxo-pyrrolidin-1- | 13 | METHANOL-d₄) δ |
| | yl)phenyl]sulfonylpiperazin-1- | TAIL: ethane- | ppm 7.88-7.93 (m, |
| | yl]-6-chloro-4-pyridyl]-difluoro- | 1,2-diamine | 4H), 7.77-7.81 (m, |
| | methyl]benzamide | | 2H), 7.62 (d, |
| | 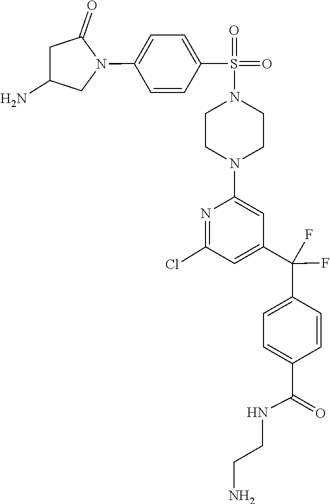 [SMILES: NCCNC(=O)c1ccc(C(F)(F)c2cc(Cl)nc(N3CCN(S(=O)(=O)c4ccc(N5CC(N)CC5=O)cc4)CC3)c2)cc1] | | J = 8.56 Hz, 2H), 6.81 (s, 1H), 6.68 (s, 1H), 4.14 (dd, J = 10.09, 6.42 Hz, 1H), 3.75-3.80 (m, 1H), 3.60-3.71 (m, 5H), 3.50 (t, J = 6.30 Hz, 2H), 3.03-3.09 (m, 4H), 2.88-2.95 (m, 3H), 1.28 (d, J = 1.34 Hz, 2H). ¹⁹F NMR (376 MHz, METHANOL-d₄) δ ppm −95.21 (s, 2F). MS obsd. (ESI⁺) [(M + H)⁺]: 648.1. |
| 018 | N-(3-amino-2-hydroxy-propyl)- | LINKER: Int- | ¹H NMR (400 MHz, |
| | 4-[[2-[4-[4-(4-amino-2-oxo- | 13 | METHANOL-d₄) δ |
| | pyrrolidin-1- | TAIL: 1,3- | ppm 7.88-7.92 (m, |
| | yl)phenyl]sulfonylpiperazin-1- | diaminopropan- | 4H), 7.77-7.80 (m, |
| | yl]-6-chloro-4-pyridyl]-difluoro- | 2-ol | 2H), 7.63 (d, |
| | methyl]benzamide | | J = 8.19 Hz, 2H), 6.81 |
| | 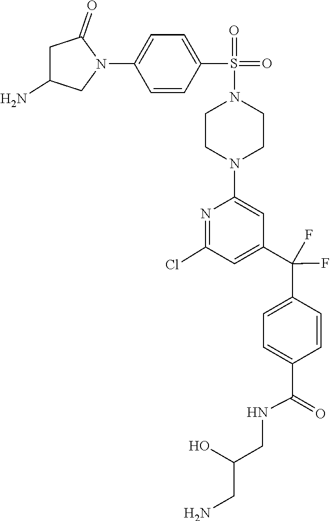 [SMILES: NCC(O)CNC(=O)c1ccc(C(F)(F)c2cc(Cl)nc(N3CCN(S(=O)(=O)c4ccc(N5CC(N)CC5=O)cc4)CC3)c2)cc1] | | (s, 1H), 6.68 (s, 1H), 4.58 (br s, 1H), 4.14 (d, J = 3.91 Hz, 1H), 3.60-3.74 (m, 7H), 3.36-3.54 (m, 6H), 3.03-3.09 (m, 5H), 2.86-2.94 (m, 2H), 2.42 (s, 1H). ¹⁹F NMR (376 MHz, METHANOL-d₄) δ ppm −95.09 (s, 1F), −95.24 (s, 1F). MS obsd. (ESI⁺) [(M + H)⁺]: 678.2 |
| 019 | N-(3-amino-2-hydroxy-propyl)- | LINKER: Int- | ¹H NMR (400 MHz, |
| | 4-[[2-chloro-6-[4-[4-[(4R)-4- | 12 | METHANOL-d₄) δ |
| | amino-2-oxo-pyrrolidin-1- | TAIL: 1,3- | ppm 7.89-7.94 (m, |
| | yl]phenyl]sulfonylpiperazin-1- | diaminopropan- | 4H), 7.77-7.81 (m, |
| | yl]-4-pyridyl]-difluoro- | 2-o1 | 2H), 7.64 (d, |
| | methyl]benzamide | | J = 8.56 Hz, 2H), 6.81 |
| | 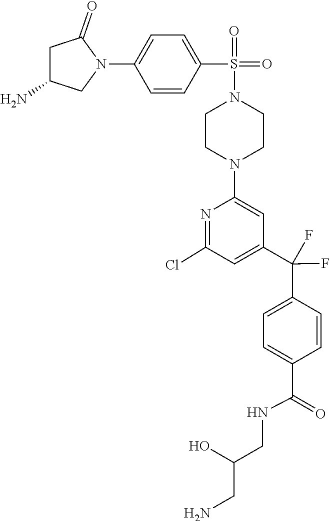 [SMILES: NCC(O)CNC(=O)c1ccc(C(F)(F)c2cc(Cl)nc(N3CCN(S(=O)(=O)c4ccc(N5C[C@H](N)CC5=O)cc4)CC3)c2)cc1] | | (s, 1H), 6.67 (s, 1H), 4.58 (br s, 1H), 4.18 (dd, J = 10.21, 6.42 Hz, 1H), 3.96-4.02 (m, 1H), 3.84 (m, 1H), 3.63-3.73 (m, 5H), 3.33-3.54 (m, 3H), 3.01-3.13 (m, 5H), 2.82-2.98 (m, 2H), 2.44 (dd, J = 17.36, 4.03 Hz, 1H). ¹⁹F NMR (376 MHz, METHANOL-d₄) δ ppm −95.30 (s, 2F). MS obsd. (ESI⁺) [(M + H)⁺]: 678.3. |
| 020 | 4-[[2-chloro-6-[4-[4-[(4R)-4- | LINKER: Int- | ¹H NMR (400 MHz, |
| | amino-2-oxo-pyrrolidin-1- | 12 | METHANOL-d₄) δ |
| | yl]phenyl]sulfonylpiperazin-1- | TAIL: N1,N1- | ppm 7.92 (d, |
| | yl]-4-pyridyl]-difluoro-methyl]- | dimethylpropane- | J = 8.93 Hz, 4H), 7.81- |
| | N-[3- | 1,3-diamine | 7.85 (m, 2H), 7.64 (d, |
| | (dimethylamino)propyl]benzamide | | J = 8.56 Hz, 2H), 6.83 |
| | 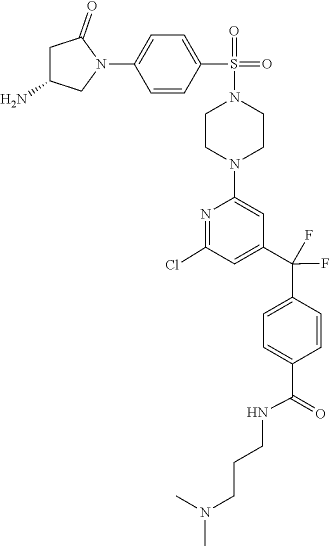 [SMILES: CN(C)CCCNC(=O)c1ccc(C(F)(F)c2cc(Cl)nc(N3CCN(S(=O)(=O)c4ccc(N5C[C@H](N)CC5=O)cc4)CC3)c2)cc1] | | (s, 1H), 6.67 (s, 1H), 4.40 (dd, J = 11.49, 6.97 Hz, 1H), 4.16-4.22 (m, 1H), 3.95 (dd, J = 11.31, 2.51 Hz, 1H), 3.64-3.73 (m, 4H), 3.49 (t, J = 6.60 Hz, 2H), 3.14-3.23 (m, 3H), 3.05-3.10 (m, 4H), 2.91 (s, 6H), 2.64-2.74 (m, 1H), 1.99-2.07 (m, 2H). ¹⁹F NMR (376 MHz, METHANOL-d₄) δ ppm −95.25 (s, 2F). MS obsd. (ESI⁺) [(M + H)⁺]: 690.3. |
| 021 | N-(3-aminopropyl)-5-[[2-chloro- | LINKER: Int- | ¹H NMR (400 MHz, |
| | 6-[4-[4-[(4R)-4-amino-2-oxo- | 16 | METHANOL-d₄) δ |
| | pyrrolidin-1- | TAIL: tert-butyl | ppm 9.31 (m, 1H), |
| | yl]phenyl]sulfonylpiperazin-1- | (3- | 9.24 (s, 1H), 9.10 (d, |
| | yl]-4-pyridyl]-difluoro- | aminopropyl) | J = 1.47 Hz, 1H), 7.90- |
| | methyl]pyrazine-2-carboxamide | carbamate | 7.95 (m, 2H), 7.80- |
| | 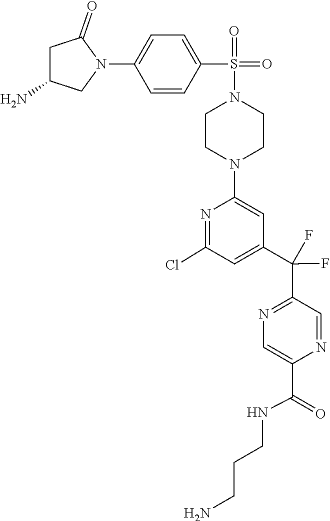 [SMILES: NCCCNC(=O)c1cnc(C(F)(F)c2cc(Cl)nc(N3CCN(S(=O)(=O)c4ccc(N5C[C@H](N)CC5=O)cc4)CC3)c2)cn1] | | 7.84 (m, 2H), 6.90 (s, 1H), 6.80 (s, 1H), 4.39 (dd, J = 11.37, 6.97 Hz, 1H), 4.16-4.22 (m, 1H), 3.95 (dd, J = 11.37, 2.57 Hz, 1H), 3.64-3.75 (m, 4H), 3.33-3.58 (m, 2H), 3.19 (dd, J = 18.10, 8.31 Hz, 1H), 2.97-3.10 (m, 6H), 2.69 (dd, J = 18.10, 2.93 Hz, 1H), 1.93-2.03 (m, 2H). ¹⁹F NMR (376 MHz, METHANOL-d₄) δ ppm −100.07 (s, 2F). MS obsd. (ESI⁺) [(M + H)⁺]: 664.3. |
| 022 | N-(3-amino-2-hydroxy-propyl)- | LINKER: Int- | ¹H NMR (400 MHz, |
| | 5-[[2-chloro-6-[4-[4-[(4R)-4- | 16 | METHANOL-d₄) δ |
| | amino-2-oxo-pyrrolidin-1- | TAIL: 1,3- | ppm 9.23 (s, 1H), 9.10 |
| | yl]phenyl]sulfonylpiperazin-1- | diaminopropan- | (d, J = 1.35 Hz, 1H), |
| | yl]-4-pyridyl]-difluoro- | 2-ol | 7.87-7.93 (m, 2H), |
| | methyl]pyrazine-2- | | 7.76-7.81 (m, 2H), |
| | carboxamide | | 6.90 (s, 1H), 6.80 (s, |
| | 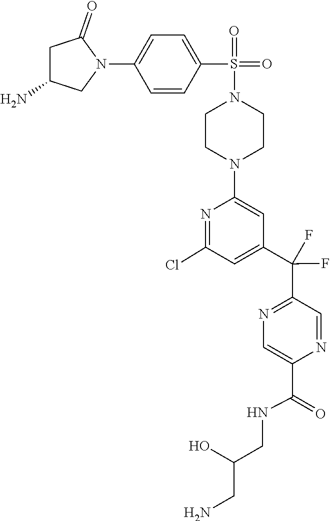 [SMILES: NCC(O)CNC(=O)c1cnc(C(F)(F)c2cc(Cl)nc(N3CCN(S(=O)(=O)c4ccc(N5C[C@H](N)CC5=O)cc4)CC3)c2)cn1] | | 1H), 4.46-4.73 (m, 1H), 4.21 (dd, J = 10.45, 6.54 Hz, 1H), 4.02 (m, 1H), 3.89 (m, 1H), 3.65-3.74 (m, 5H), 3.47-3.59 (m, 2H), 2.95-3.13 (m, 6H), 2.87 (dd, J = 12.96, 8.80 Hz, 1H), 2.48 (dd, J = 17.48, 3.91 Hz, 1H). ¹⁹F NMR (376 MHz, METHANOL-d₄) δ ppm −100.21 (s, 2F). MS obsd. (ESI⁺) [(M + H)⁺]: 680.3. |
| 023 | 5-[[2-chloro-6-[4-[4-[(4R)-4- | LINKER: Int- | ¹H NMR (400 MHz, |
| | amino-2-oxo-pyrrolidin-1- | 16 | METHANOL-d₄) δ |
| | yl]phenyl]sulfonylpiperazin-1- | TAIL: NI,NI- | ppm 9.21-9.24 (m, |
| | yl]-4-pyridyl]-difluoro-methyl]- | dimethylpropane- | 1H), 9.10 (d, |
| | N-[3- | 1,3-diamine | J = 1.35 Hz, 1H), 7.90 |
| | (dimethylamino)propyl]pyrazine- | | (d, J = 8.93 Hz, 2H), |
| | 2-carboxamide | | 7.77 (d, J = 8.93 Hz, |
| | 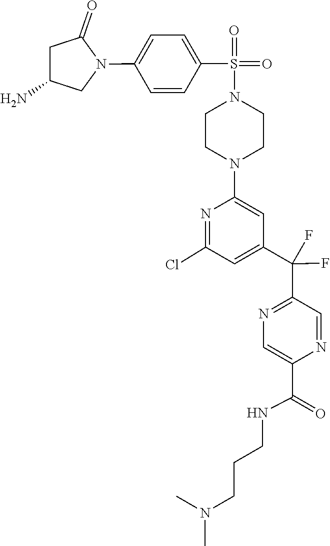 [SMILES: CN(C)CCCNC(=O)c1cnc(C(F)(F)c2cc(Cl)nc(N3CCN(S(=O)(=O)c4ccc(N5C[C@H](N)CC5=O)cc4)CC3)c2)cn1] | | 2H), 6.89 (s, 1H), 6.81 (s, 1H), 4.25 (dd, J = 10.58, 6.66 Hz, 1H), 3.95 (tt, J = 7.06, 3.45 Hz, 1H), 3.77 (dd, J = 10.58, 3.12 Hz, 1H), 3.64-3.71 (m, 4H), 3.48-3.57 (m, 2H), 2.97-3.10 (m, 7H), 2.81-2.89 (m, 1H), 2.79 (s, 6H), 2.53 (dd, J = 17.55, 3.73 Hz, 1H), 1.95-2.08 (m, 2H). ¹⁹F NMR (376 MHz, METHANOL-d₄) δ ppm −100.14 (s, 2F). MS obsd. (ESI⁺) [(M + H)⁺]: 692.4. |
| 024 | N-(2-aminoethyl)-5-[[2-chloro- | LINKER: Int- | ¹H NMR (400 MHz, |
| | 6-[4-[4-[(4R)-4-amino-2-oxo- | 16 | METHANOL-d₄) δ |
| | pyrrolidin-1- | TAIL: ethane- | ppm 9.31 (m, 1H), |
| | yl]phenyl]sulfonylpiperazin-1- | 1,2-diamine | 9.24 (s, 1H), 9.10 (d, |
| | yl]-4-pyridyl]-difluoro- | | J = 1.47 Hz, 1H), 7.90- |
| | methyl]pyrazine-2-carboxamide | | 7.95 (m, 2H), 7.80- |
| | 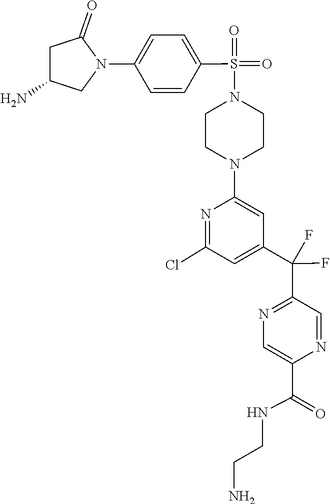 [SMILES: NCCNC(=O)c1cnc(C(F)(F)c2cc(Cl)nc(N3CCN(S(=O)(=O)c4ccc(N5C[C@H](N)CC5=O)cc4)CC3)c2)cn1] | | 7.84 (m, 2H), 6.90 (s, 1H), 6.80 (s, 1H), 4.39 (dd, J = 11.37, 6.97 Hz, 1H), 4.16-4.22 (m, 1H), 3.95 (dd, J = 11.37, 2.57 Hz, 1H), 3.64-3.75 (m, 4H), 3.33-3.58 (m, 2H), 3.19 (dd, J = 18.10, 8.31 Hz, 1H), 2.97-3.10 (m, 6H), 2.69 (dd, J = 18.10, 2.93 Hz, 1H), 1.93-2.03 (m, 2H). ¹⁹F NMR (376 MHz, METHANOL-d₄) δ ppm −100.07 (s, 2F). MS obsd. (ESI⁺) [(M + H)⁺]: 650.3. |
| 025 | 5-[[2-chloro-6-[4-[4-[(4R)-4- | LINKER: Int- | ¹H NMR (400 MHz, |
| | amino-2-oxo-pyrrolidin-1- | 16 | METHANOL-d₄) δ |
| | yl]phenyl]sulfonylpiperazin-1- | TAIL: 2- | ppm 9.21 (s, 1H), 9.10 |
| | yl]-4-pyridyl]-difluoro-methyl]- | morpholinoethan- | (d, J = 1.35 Hz, 1H), |
| | N-(2-morpholinoethyl)pyrazine- | 1-amine | 7.91 (m, J = 8.93 Hz, |
| | 2-carboxamide | | 2H), 7.80 (m, |
| | 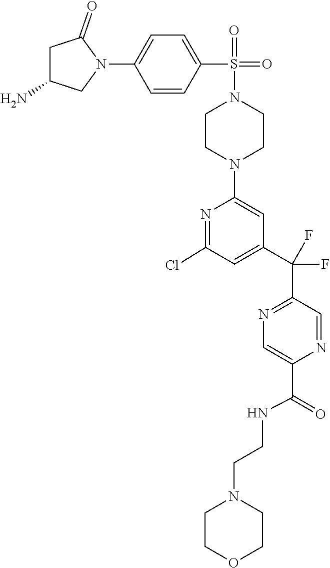 [SMILES: N[C@@H]1CC(=O)N(c2ccc(S(=O)(=O)N3CCN(c4cc(C(F)(F)c5cnc(C(=O)NCCN6CCOCC6)cn5)cc(Cl)n4)CC3)cc2)C1] | | J = 9.05 Hz, 2H), 6.88 (s, 1H), 6.81 (s, 1H), 4.35 (dd, J = 11.13, 6.85 Hz, 1H), 4.07-4.16 (m, 1H), 3.89 (dd, J = 11.13, 2.57 Hz, 1H), 3.65-3.75 (m, 8H), 3.55-3.64 (m, 2H), 3.09-3.19 (m, 1H), 2.99-3.08 (m, 4H), 2.57-2.73 (m, 7H). 19F NMR (376 MHz, METHANOL-d₄) δ ppm −100.09 (s, 2F). MS obsd. (ESI⁺) [(M + H)⁺]: 720.3. |
| 026 | N-(3-aminopropyl)-6-[[2-chloro- | LINKER: Int- | ¹H NMR (400 MHz, |
| | 6-[4-[4-[(4R)-4-amino-2-oxo- | 15 | METHANOL-d₄) δ |
| | pyrrolidin-1- | TAIL: 3- | ppm 9.01 (d, |
| | yl]phenyl]sulfonylpiperazin-1- | aminopropylamine | J = 1.59 Hz, 1H), 8.39 |
| | yl]-4-pyridyl]-difluoro- | | (dd, J = 8.19, 2.20 Hz, |
| | methyl]pyridine-3-carboxamide | | 1H), 7.91-7.98 (m, |
| | 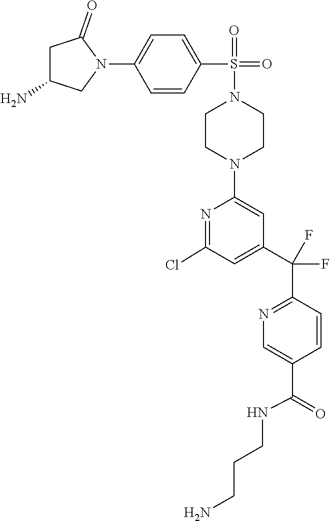 [SMILES: NCCCNC(=O)c1ccc(C(F)(F)c2cc(Cl)nc(N3CCN(S(=O)(=O)c4ccc(N5C[C@H](N)CC5=O)cc4)CC3)c2)nc1] | | 3H), 7.81-7.86 (m, 2H), 6.90 (s, 1H), 6.76 (s, 1H), 4.42 (dd, J = 11.43, 7.03 Hz, 1H), 4.19-4.25 (m, 1H), 3.99 (dd, J = 11.37, 2.57 Hz, 1H), 3.65-3.74 (m, 4H), 3.53 (t, J = 6.66 Hz, 2H), 3.33 (dt, J = 3.30, 1.65 Hz, 3H), 3.21 (dd, J = 18.16, 8.25 Hz, 1H), 3.01-3.11 (m, 6H), 2.73 (dd, J = 18.10, 3.06 Hz, 1H), 1.95-2.06 (m, 2H). ¹⁹F NMR (376 MHz, METHANOL-d₄) δ ppm −99.84 (s, 2F). MS obsd. (ESI⁺) [(M + H)⁺]: 663.3. |
| 027 | 6-[[2-chloro-6-[4-[4-[(4R)-4- | LINKER: Int- | ¹H NMR (400 MHz, |
| | amino-2-oxo-pyrrolidin-1- | 15 | METHANOL-d₄) δ |
| | yl]phenyl]sulfonylpiperazin-1- | TAIL: NI,NI- | ppm 8.84 (s, 1H), |
| | yl]-4-pyridyl]-difluoro-methyl]- | dimethylpropane- | 8.14-8.22 (m, 2H), |
| | N-[3- | 1,3-diamine | 7.90-7.96 (m, 2H), |
| | (dimethylamino)propyl]pyridine- | | 7.80-7.86 (m, 2H), |
| | 3-carboxamide | | 6.88 (s, 1H), 6.76 (s, |
| | 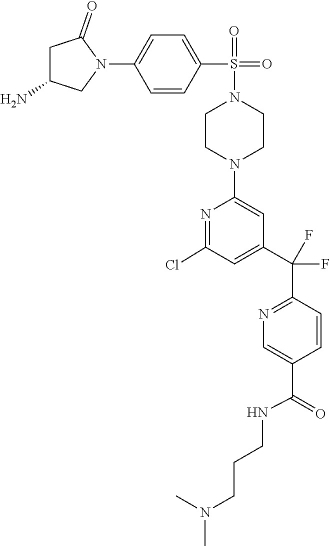 [SMILES: CN(C)CCCNC(=O)c1ccc(C(F)(F)c2cc(Cl)nc(N3CCN(S(=O)(=O)c4ccc(N5C[C@H](N)CC5=O)cc4)CC3)c2)nc1] | | J = 11.43, 7.03 Hz, 1H), 4.18-4.26 (m, 1H), 3.96-4.04 (m, 1H), 3.67-3.76 (m, 4H), 3.55 (t, J = 6.54 Hz, 2H), 3.15-3.26 (m, 3H), 3.05-3.12 (m, 4H), 2.92 (s, 6H), 2.69-2.78 (m, 1H), 2.02-2.11 (m, 2H). ¹⁹F NMR (376 MHz, METHANOL-d₄) δ ppm −95.38 (s, 1F), −95.40 (s, 1F). MS obsd. (ESI⁺) [(M + H)⁺]: 691.2. |
| 028 | N-[2-(2- | LINKER: Int- | ¹H NMR (400 MHz, |
| | aminoethylamino)ethyl]-6-[[2- | 15 | METHANOL-d₄) δ |
| | chloro-6-[4-[4-[(4R)-4-amino-2- | TAIL: bis(2- | ppm 9.04 (d, |
| | oxo-pyrrolidin-1- | aminoethyl)amine | J = 1.71 Hz, 1H), 8.42 |
| | yl]phenyl]sulfonylpiperazin-1- | | (dd, J = 8.31, 2.20 Hz, |
| | yl]-4-pyridyl]-difluoro- | | 1H), 7.91-7.98 (m, |
| | methyl]pyridine-3- | | 3H), 7.80-7.89 (m, |
| | carboxamide | | 2H), 6.89 (s, 1H), 6.76 |
| | 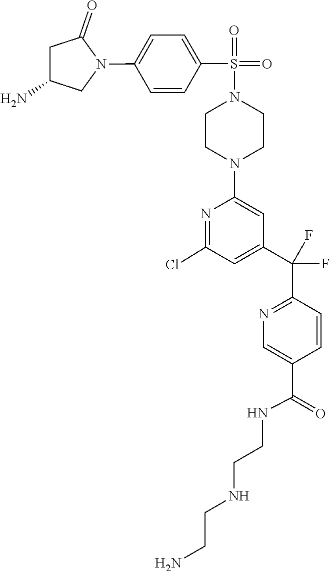 [SMILES: NCCNCCNC(=O)c1ccc(C(F)(F)c2cc(Cl)nc(N3CCN(S(=O)(=O)c4ccc(N5C[C@H](N)CC5=O)cc4)CC3)c2)nc1] | | (s, 1H), 4.42 (dd, J = 11.43, 7.03 Hz, 1H), 4.15-4.28 (m, 1H), 4.08 (br d, J = 18.95 Hz, 1H), 3.98 (dd, J = 11.43, 2.51 Hz, 1H), 3.88 (br s, 1H), 3.78 (t, J = 5.50 Hz, 2H), 3.67- 3.74 (m, 5H), 3.57- 3.66 (m, 1H), 3.34- 3.51 (m, 7H), 3.22 (dd, J = 18.10, 8.31 Hz, 1H), 3.06-3.12 (m, 4H), 2.98-3.04 (m, 1H), 2.72 (dd, J = 18.10, 3.06 Hz, 1H). ¹⁹F NMR (376 MHz, METHANOL-d₄) δ ppm −99.99 (s, 2F). MS obsd. (ESI⁺) [(M + H)⁺]: 692.2. |
| 029 | 5-[[2-chloro-6-[4-[4-[(4R)-4- | LINKER: Int- | ¹H NMR (400 MHz, |
| | amino-2-oxo-pyrrolidin-1- | 14 | METHANOL-d₄) δ |
| | yl]phenyl]sulfonylpiperazin-1- | TAIL: N1- | ppm 8.82 (s, 1H), |
| | yl]-4-pyridyl]-difluoro-methyl]- | methylpropane- | 8.10-8.19 (m, 2H), |
| | N-[3- | 1,3-diamine | 7.88-7.94 (m, 2H), |
| | (methylamino)propyl]pyridine- | | 7.79-7.84 (m, 2H), |
| | 2-carboxamide | | 6.86 (s, 1H), 6.74 (s, |
| | 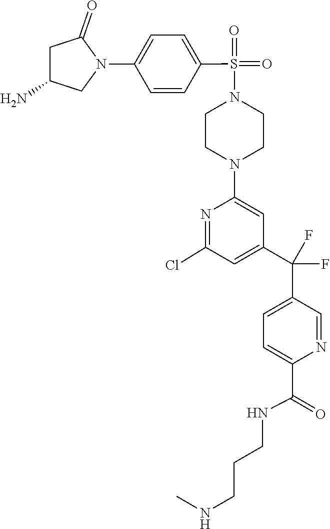 [SMILES: CNCCCNC(=O)c1ccc(C(F)(F)c2cc(Cl)nc(N3CCN(S(=O)(=O)c4ccc(N5C[C@H](N)CC5=O)cc4)CC3)c2)cn1] | | 1H), 4.40 (dd, J = 11.37, 6.97 Hz, 1H), 4.20 (br t, J = 7.52 Hz, 1H), 3.92-4.03 (m, 1H), 3.64-3.74 (m, 4H), 3.53 (t, J = 6.54 Hz, 2H), 3.19 (dd, J = 18.10, 8.31 Hz, 1H), 2.99-3.09 (m, 6H), 2.67-2.75 (m, 3H), 1.94-2.05 (m, 2H). ¹⁹F NMR (376 MHz, METHANOL-d₄) δ ppm −95.35 (s, 2F). MS obsd. (ESI⁺) [(M + H)⁺]: 677.4. |
| 030 | Trans-N-(2-aminoethyl)-4-[[2- | LINKER: Int-5 | ¹H NMR (400 MHz, |
| | chloro-6-[4-[4-[(4R)-4-amino-2- | TAIL: tert-butyl | DMSO-d₆) δ ppm 7.91 |
| | oxo-pyrrolidin-1- | 2- | (br d, J = 9.05 Hz, 4H), |
| | yl]phenyl]sulfonylpiperazin-1- | (methylamino) | 7.79 (d, J = 8.93 Hz, |
| | yl]-4-pyridyl]-difluoro-methyl]- | ethylcarbamate | 2H), 6.78 (s, 1H), |
| | N-methyl- | | 6.68-6.71 (m, 1H), |
| | cyclohexanecarboxamide | | 4.26 (dd, J = 11.19, |
| | 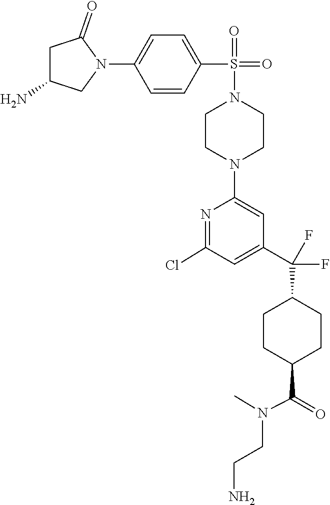 [SMILES: CN(CCN)C(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 6.79 Hz, 1H), 4.02-4.11 (m, 1H), 3.89 (dd, J = 10.76, 1.71 Hz, 1H), 3.66 (br s, 5H), 3.06 (dd, J = 17.85, 8.19 Hz, 1H), 3.00 (s, 2H), 2.96 (br d, J = 4.89 Hz, 4H), 2.84-2.90 (m, 2H), 2.78 (s, 1H), 2.64-2.69 (m, 1H), 2.62 (d, J = 2.81 Hz, 1H), 2.33 (dt, J = 3.64, 1.67 Hz, 1H), 2.13-2.28 (m, 1H), 1.61-1.80 (m, 4H), 1.13-1.38 (m, 4H). ¹⁹F NMR (376 MHz, DMSO-d₆) δ ppm −103.55 (br s, 2F). MS obsd. (ESI⁺) [(M + H)⁺]: 668.2. |
| 031 | Cis-N-(3-aminopropyl)-4-[2- | LINKER: Int-7 | ¹H NMR (400 MHz, |
| | chloro-6-[4-[4-[(4R)-4-amino-2- | TAIL: Tert- | DMSO-d₆) δ ppm |
| | oxo-pyrrolidin-1- | butyl N-(3- | 8.59-8.75 (m, 2H), |
| | yl]phenyl]sulfonylpiperazin-1- | aminopropyl) | 7.88-8.09 (m, 6H), |
| | yl]-4-pyridyl]-difluoro- | carbamate | 7.80 (d, J = 8.93 Hz, |
| | methyl]cyclohexanecarboxamide | | 2H), 6.78 (s, 1H), 6.69 |
| | 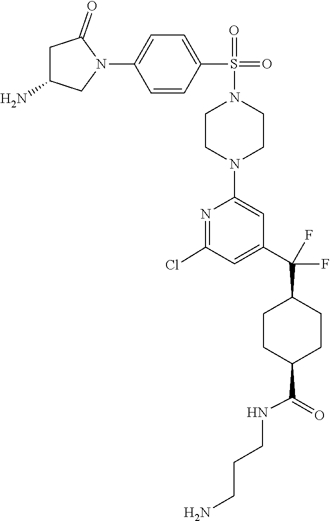 [SMILES: NCCCNC(=O)[C@H]1CC[C@@H](C(F)(F)c2cc(Cl)nc(N3CCN(S(=O)(=O)c4ccc(N5C[C@H](N)CC5=O)cc4)CC3)c2)CC1] | | (s, 1H), 4.27 (dd, J = 11.13, 6.97 Hz, 1H), 4.06 (br s, 1H), 3.89- 3.98 (m, 1H), 3.63- 3.69 (m, 4H), 3.01- 3.17 (m, 3H), 2.97 (br s, 4H), 2.61-2.81 (m, 3H), 2.37-2.42 (m, 1H), 2.16 (br s, 1H), 1.95 (br d, J = 9.05 Hz, 2H), 1.61-1.78 (m, 2H), 1.42 (br s, 5H). ¹⁹F NMR (376 MHz, DMSO-d₆) δ ppm −103.33 (br s, 2F). MS obsd. (ESI⁺) [(M+H)⁺]: 668.2. |
| 032 | Cis-4-[2-chloro-6-[4-[4-[(4R)- | LINKER: Int-7 | ¹H NMR (400 MHz, |
| | 4-amino-2-oxo-pyrrolidin-1- | TAIL: 3- | DMSO-d₆) δ ppm 8.33 |
| | yl]phenyl]sulfonylpiperazin-1- | aminopropan-1- | (br d, J = 2.32 Hz, 3H), |
| | yl]-4-pyridyl]-difluoro-methyl]- | ol | 7.92 (d, J = 8.93 Hz, |
| | N-(3-hydroxypropyl) | | 2H), 7.79 (d, J = |
| | cyclohexanecarboxamide | | 9.05 Hz, 2H), 7.65 (t, J = |
| | 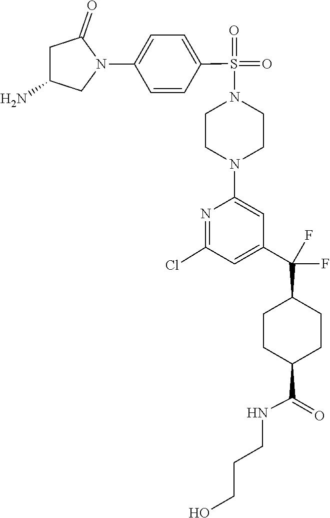 [SMILES: N[C@@H]1CC(=O)N(c2ccc(S(=O)(=O)N3CCN(c4cc(C(F)(F)[C@H]5CC[C@@H](C(=O)NCCCO)CC5)cc(Cl)n4)CC3)cc2)C1] | | 5.56 Hz, 1H), 6.76 (s, 1H) 6.68 (s, 1H) 4.26 (dd, J = 11.19, 7.03 Hz, 1H), 4.01-4.10 (m, 1H), 3.81-3.88 (m, 1H), 3.64 (br d, J = 4.89 Hz, 4H), 3.38 (br s, 2H), 3.03-3.11 (m, 3H), 2.97 (br d, J = 4.40 Hz, 4H), 2.60 (dd, J = 17.91, 2.63 Hz, 1H), 2.33-2.38 (m, 1H), 2.14 (br s, 1H), 1.92 (br d, J = 10.15 Hz, 2H), 1.33- 1.54 (m, 8H). ¹⁹F NMR (376 MHz, DMSO-d₆) δ ppm −103.09 (br s, 2F). MS obsd. (ESI⁺) [(M + H)⁺]: 669.2. |
| 033 | N-(3-aminopropyl)-4-[[2-chloro- | LINKER: Int- | ¹H NMR (400 MHz, |
| | 6-[4-[4-[(4R)-4-amino-2-oxo- | 10 | DMSO-d₆) δ ppm |
| | pyrrolidin-1- | TAIL: Tert- | 8.56-8.65 (m, 3H) |
| | yl]phenyl]sulfonylpiperazin-1- | butyl N-(3- | 7.94-8.01 (m, 3H) |
| | yl]-4-pyridyl]-difluoro- | aminopropyl) | 7.89-7.94 (m, 2H) |
| | methyl]bicyclo[2.2.2]octane-1- | carbamate | 7.77-7.82 (m, 2H) |
| | carboxamide | | 7.69-7.75 (m, 1H) |
| | 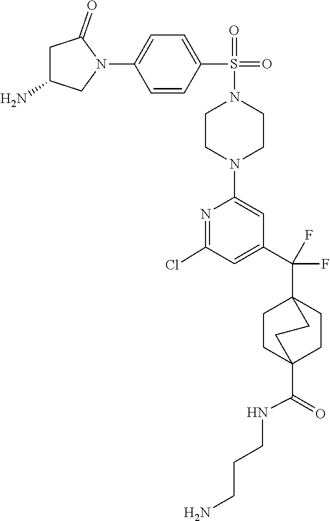 [SMILES: NCCCNC(=O)C12CCC(C(F)(F)c3cc(Cl)nc(N4CCN(S(=O)(=O)c5ccc(N6C[C@H](N)CC6=O)cc5)CC4)c3)(CC1)CC2] | | 6.62-6.65 (m, 1H) 6.58-6.61 (m, 1H) 4.23-4.31 (m, 1H) 4.01-4.13 (m, 1H) 3.87-3.95 (m, 1H) 3.61-3.70 (m, 4H) 3.02-3.13 (m, 3H) 2.93-3.01 (m, 4H) 2.64-2.74 (m, 3H) 1.59-1.71 (m, 8H) 1.48-1.55 (m, 6H). ¹⁹F NMR (376 MHz, DMSO-d₆) δ ppm −117.70- −95.37 (m, 2F). MS obsd. (ESI⁺) [(M + H)⁺]: 694.4. |
| 034 | N-(3-aminopropyl)-4-[[2-chloro- | LINKER: Int-9 | ¹H NMR (400 MHz, |
| | 6-[4-[4-[(4R)-4-amino-2-oxo- | TAIL: Tert- | DMSO-d₆) δ ppm 8.69 |
| | pyrrolidin-1- | butyl N-(3- | (br d, J = 4.03 Hz, 2H), |
| | yl]phenyl]sulfonylpiperazin-1- | aminopropyl) | 8.03 (br s, 2H), 7.82- |
| | yl]-4-pyridyl]-difluoro- | carbamate | 7.94 (m, 2H), 7.78 (d, |
| | methyl]norbornane-1- | | J = 8.93 Hz, 2H), 6.68 |
| | carboxamide | | (s, 1H), 6.62 (s, 1H), |
| | 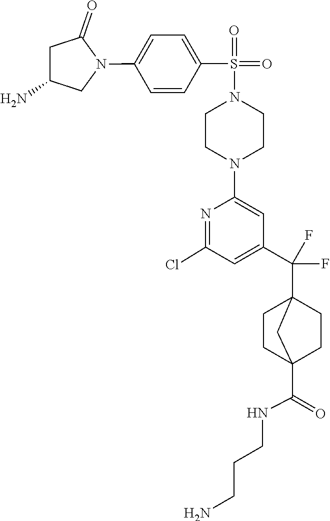 [SMILES: NCCCNC(=O)C12CCC(C(F)(F)c3cc(Cl)nc(N4CCN(S(=O)(=O)c5ccc(N6C[C@H](N)CC6=O)cc5)CC4)c3)(CC1)C2] | | 4.26 (dd, J = 11.07, 6.91 Hz, 1H), 4.06 (br s, 1H), 3.92 (dd, J = 10.94, 1.90 Hz, 1H), 3.60-3.63 (m, 4H), 3.02-3.13 (m, 3H), 2.96 (br s, 4H), 2.63- 2.76 (m, 3H), 1.55- 1.79 (m, 10H), 1.28 (br t, J = 8.01 Hz, 2H). ¹⁹F NMR (376 MHz, DMSO-d₆) δ ppm −101.53 (br s, 2F). MS obsd. (ESI⁺) [(M + H)⁺]: 680.3. |
| 035 | N-(3-aminopropyl)-3-[[2-chloro- | LINKER: Int-8 | ¹HNMR (400 MHz, |
| | 6-[4-[4-[(4R)-4-amino-2-oxo- | TAIL: Tert- | DEUTERIUM |
| | pyrrolidin-1- | butyl N-(3- | OXIDE) δ ppm 7.71- |
| | yl]phenyl]sulfonylpiperazin-1- | aminopropyl) | 7.81 (m, 4H), 6.77 (br |
| | yl]-4-pyridyl]-difluoro- | carbamate | s, 1H), 6.68 (s, 1H), |
| | methyl]cyclopentanecarboxamide | | 4.33-4.42 (m, 1H), |
| | 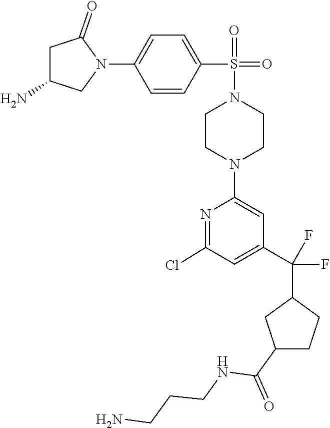 [SMILES: NCCCNC(=O)C1CCC(C(F)(F)c2cc(Cl)nc(N3CCN(S(=O)(=O)c4ccc(N5C[C@H](N)CC5=O)cc4)CC3)c2)C1] | | 4.15-4.24 (m, 1H), 3.94 (br d, J = 11.74 Hz, 1H), 3.46-3.58 (m, 4H), 3.11-3.26 (m, 3H), 2.96-3.05 (m, 4H), 2.88-2.95 (m, 2H), 2.59-2.76 (m, 3H), 1.74-1.91 (m, 4H), 1.56-1.72 (m, 4H). ¹⁹F NMR (376 MHz, DEUTERIUM OXIDE) δ ppm −103.85 (br s, 2F). MS obsd. (ESI⁺) [(M + H)⁺]: 654.3. |
| 036 | Trans-N-(3-amino-2-hydroxy- | LINKER: Int-7 | ¹H NMR (400 MHz, |
| | propyl)-4-[[2-chloro-6-[4-[4- | TAIL: Tert- | DMSO-d₆) δ ppm |
| | [(4R)-4-amino-2-oxo-pyrrolidin- | butyl (3-amino- | 8.77-8.43 (m, 2H), |
| | 1-yl]phenyl]sulfonylpiperazin-1- | 2- | 8.18-7.86 (m, 5H), |
| | yl]-4-pyridyl]-difluoro- | hydroxypropyl) | 7.79 (d, J = 8.8 Hz, |
| | methyl]cyclohexanecarboxamide | carbamate | 2H), 6.79 (s, 1H), 6.71 |
| | 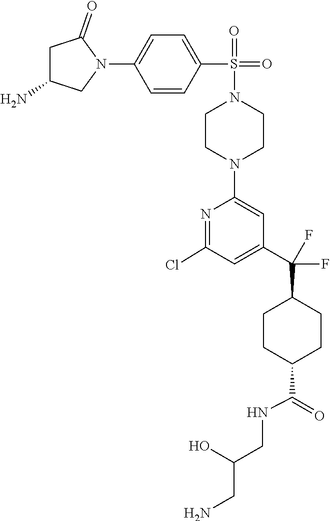 [SMILES: NCC(O)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | (s, 1H), 5.57 (d, J = 5.0 Hz, 1H), 4.26 (br dd, J = 6.8, 11.0 Hz, 1H), 4.12-4.00 (m, 1H), 3.90 (br d, J = 10.9 Hz, 1H), 3.77-3.59 (m, 5H), 3.17-2.90 (m, 7H), 2.81 (br d, J = 10.5 Hz, 1H), 2.72-2.52 (m, 4H), 2.22-2.01 (m, 2H), 1.81-1.56 (m, 4H), 1.40-1.03 (m, 4H). ¹⁹F NMR (376 MHz, DMSO-d₆) δ ppm: −104.03 (br s, 2F). MS obsd. (ESI⁺) [(M + H)⁺]: 684.3. |
| 037 | 3-[4-[2-chloro-6-[4-[4-[(4R)-4- | LINKER: Int- | ¹H NMR (400 MHz, |
| | amino-2-oxo-pyrrolidin-1- | 12 | METHANOL-d₄) δ |
| | yl]phenyl]sulfonylpiperazin-1- | TAIL: 3- | ppm 8.50 (s, 1H), |
| | yl]-4-pyridyl]-difluoro- | Aminopropyl | 7.88-7.95 (m, 4H), |
| | methyl]benzoyl]amino]propyl- | (trimethyl) | 7.79 (d, J = 8.08 Hz, |
| | trimethyl-ammonium | ammonium; iodide | 2H), 7.64 (d, |
| | 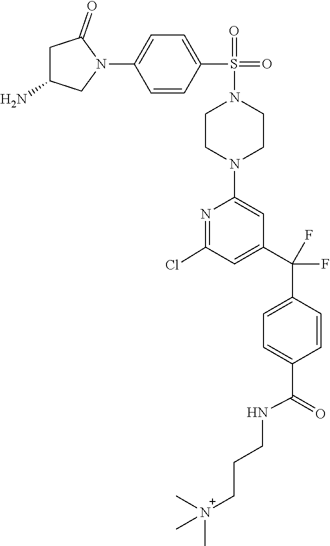 [SMILES: C[N+](C)(C)CCCNC(=O)c1ccc(C(F)(F)c2cc(Cl)nc(N3CCN(S(=O)(=O)c4ccc(N5C[C@H](N)CC5=O)cc4)CC3)c2)cc1] | | J = 8.44 Hz, 2H), 6.82 (s, 1H), 6.67 (s, 1H), 4.26 (dd, J = 10.70, 6.66 Hz, 1H), 3.91-4.01 (m, 1H), 3.80 (dd, J = 10.76, 3.06 Hz, 1H), 3.62-3.72 (m, 4H), 3.39-3.53 (m, 4H), 2.96-3.23 (m, 15H), 2.55 (dd, J = 17.61, 3.67 Hz, 1H), 2.06-2.18 (m, 2H). ¹⁹F NMR (376 MHz, METHANOL-d₄) δ ppm −95.29 (s, 2F). MS obsd. (ESI⁺) [(M + H)⁺]: 704.3. |
| 038 | 4-[[2-Chloro-6-[4-[4-[(4R)-4- | LINKER: Int- | ¹H NMR (400 MHz, |
| | amino-2-oxo-pyrrolidin-1- | 12 | METHANOL-d₄) δ |
| | yl]phenyl]sulfonylpiperazin-1- | TAIL: 3-(1- | ppm 8.53 (br s, 1H), |
| | yl]-4-pyridyl]-difluoro-methyl]- | Methylpyrrolidin- | 8.02 (br s, 1H), 7.88- |
| | N-[3-(1-methylpyrrolidin-1-ium- | 1-ium-1- | 7.95 (m, 3H), 7.75- |
| | 1-yl)propyl]benzamide | yl)propan-1- | 7.85 (m, 2H), 7.64 (d, |
| | 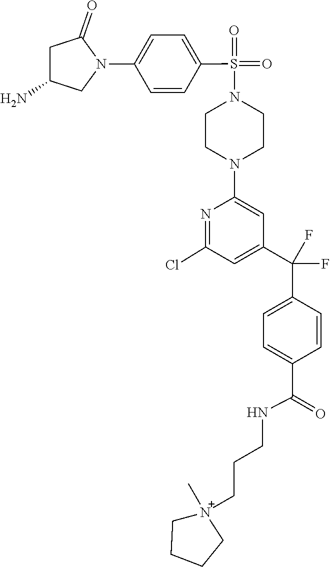 [SMILES: C[N+]1(CCCNC(=O)c2ccc(C(F)(F)c3cc(Cl)nc(N4CCN(S(=O)(=O)c5ccc(N6C[C@H](N)CC6=O)cc5)CC4)c3)cc2)CCCC1] | amine; iodide | J = 8.44 Hz, 2H), 6.82 (s, 1H), 6.67 (s, 1H), 4.39-4.75 (m, 6H), 4.22 (dd, J = 10.45, 6.54 Hz, 1H), 3.81-3.98 (m, 1H), 3.63-3.75 (m, 4H), 3.39-3.60 (m, 6H), 2.96-3.10 (m, 6H), 2.49 (dd, J = 17.42, 3.85 Hz, 1H), 2.22 (br s, 3H), 2.06- 2.20 (m, 2H). ¹⁹F NMR (376 MHz, METHANOL-d₄) δ ppm −95.33 (s, 2F). MS obsd. (ESI⁺) [(M + H)⁺]: 730.5. |
| 039 | 5-[[2-Chloro-6-[4-[4-[(4R)-4- | LINKER: Int- | ¹H NMR (400 MHz, |
| | amino-2-oxo-pyrrolidin-1- | 14 | METHANOL-d₄) δ |
| | yl]phenyl]sulfonylpiperazin-1- | TAIL: 2-(3- | ppm 8.82 (s, 1H), 8.19 |
| | yl]-4-pyridyl]-difluoro-methyl]- | Aminopropyl- | (d, J = 8.31 Hz, 1H), |
| | N-[3-(2- | amino)ethanol | 8.13 (dd, J = 8.38, |
| | hydroxyethylamino)propyl]pyridine- | | 2.14 Hz, 1H), 7.93 (m, |
| | 2-carboxamide | | J = 9.05 Hz, 2H), 7.83 |
| | 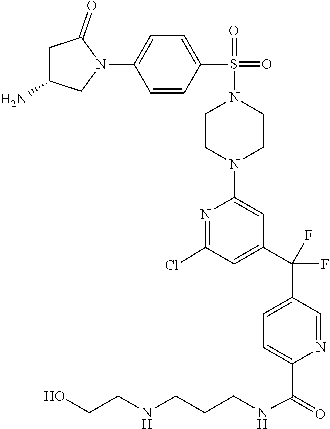 [SMILES: N[C@@H]1CC(=O)N(c2ccc(S(=O)(=O)N3CCN(c4cc(C(F)(F)c5ccc(C(=O)NCCCNCCO)nc5)cc(Cl)n4)CC3)cc2)C1] | | (m, J = 8.93 Hz, 2H), 6.87 (s, 1H), 6.74 (s, 1H), 4.89-4.95 (m, 2H), 4.40 (dd, J = 11.37, 6.97 Hz, 1H), 4.16-4.21 (m, 1H), 3.94 (dd, J = 11.37, 2.45 Hz, 1H), 3.77-3.82 (m, 2H), 3.67-3.74 (m, 4H), 3.54 (t, J = 6.48 Hz, 2H), 3.04-3.28 (m, 10H), 2.68 (dd, J = 18.03, 3.00 Hz, 1H), 1.98-2.06 (m, 2H). ¹⁹F NMR (376 MHz, METHANOL-d₄) δ ppm −95.37 (s, 2F). MS obsd. (ESI⁺) [(M + H)⁺]: 707.2. |
| 040 | N-(3-aminopropyl)-3-[2-chloro- | LINKER: Int- | ¹H NMR (400 MHz, |
| | 6-[4-[4-[(4R)-4-amino-2-oxo- | 11 | DMSO-d₆) δ ppm 8.03 |
| | pyrrolidin-1- | TAIL: Tert- | (m, 2H), 7.91 (d, |
| | yl]phenyl]sulfonylpiperazin-1- | butyl (3-amino- | J = 8.93 Hz, 3H), 7.79 |
| | yl]-4-pyridyl]-difluoro- | 2- | (d, J = 8.93 Hz, 2H), |
| | methyl]bicyclo[1.1.1]pentane-1- | hydroxypropyl) | 6.68 (s, 1H), 6.60 (s, |
| | carboxamide | carbamate | 1H), 4.26 (dd, J = 11.13, |
| | 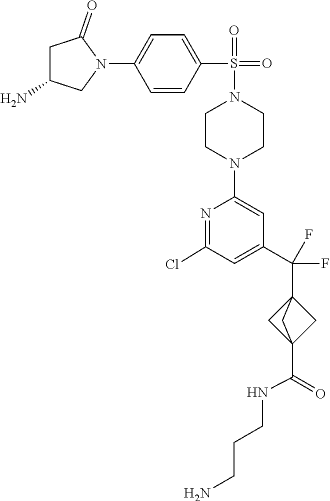 [SMILES: NCCCNC(=O)C12CC(C(F)(F)c3cc(Cl)nc(N4CCN(S(=O)(=O)c5ccc(N6C[C@H](N)CC6=O)cc5)CC4)c3)(C1)C2] | | 6.85 Hz, 1H), 4.03-4.10 (m, 1H), 3.83-3.91 (m, 1H), 3.66 (br d, J = 4.28 Hz, 4H), 3.02- 3.11 (m, 4H), 2.97 (br d, J = 4.28 Hz, 4H), 2.72 (br t, J = 7.46 Hz, 2H), 2.58-2.68 (m, 2H), 2.29-2.36 (m, 1H), 1.65 (m, 2H) 1.12-1.29 (m, 1H). ¹⁹F NMR (376 MHz, DMSO-d₆) δ ppm −103.59 (br s, 2F). MS obsd. (ESI⁺) [(M + H)⁺]: 652.2. |
| 041 | Trans-N-(2-aminoethyl)-4-[[2- | LINKER: Int-5 | ¹H NMR (400 MHz, |
| | chloro-6-[4-[4-[(4R)-4-amino-2- | TAIL: Tert- | DMSO-d₆) δ ppm |
| | oxo-pyrrolidin-1- | butyl (3-amino- | 8.05-8.52 (m, 4H) 7.91 |
| | yl]phenyl]sulfonylpiperazin-1- | 2- | (br d, J = 8.80 Hz, 2H) |
| | yl]-4-pyridyl]-difluoro- | hydroxypropyl) | 7.79 (br d, J = 8.80 Hz, |
| | methyl]cyclohexanecarboxamide | carbamate | 2H), 6.78 (s, 1H) 6.70 |
| | 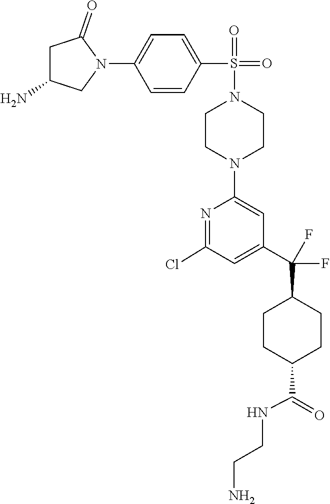 [SMILES: NCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | (s, 1H) 4.26 (br dd, J = 11.07, 6.91 Hz, 1H) 4.01-4.10 (m, 1H) 3.89 (br d, J = 10.03 Hz, 1H) 3.65 (br s, 4H) 3.23- 3.29 (m, 3H) 3.06 (br dd, J = 17.85, 8.19 Hz, 1H) 2.96 (br s, 4H) 2.81 (br t, J = 6.30 Hz, 2H) 2.64 (br dd, J = 17.85, 2.45 Hz, 1H) 2.17 (m, 1H) 1.99-2.08 (m, 1H) 1.79 (br d, J = 11.13 Hz, 2H) 1.60- 1.69 (m, 2H) 1.25-1.38 (m, 2H) 1.05-1.18 (m, 2H). ¹⁹F NMR (376 MHz, DMSO-d₆) δ ppm −104.05 (br s, 2F). MS obsd. (ESI⁺) [(M + H)⁺]: 654.2. |
| 042 | Trans-N-[2-(2- | LINKER: Int-5 | ¹H NMR (400 MHz, |
| | aminoethoxy)ethyl]-4-[[2- | TAIL: 2,2′- | DMSO-d₆) δ ppm |
| | chloro-6-[4-[4-[(4R)-4-amino-2- | Oxydiethanamine | 8.70-8.58 (m, 3H), |
| | oxo-pyrrolidin-1- | | 8.18-8.07 (m, 3H), |
| | yl]phenyl]sulfonylpiperazin-1- | | 8.05-8.00 (m, 1H), |
| | yl]-4-pyridyl]-difluoro- | | 7.94-7.87 (m, 2H), |
| | methyl]cyclohexanecarboxamide | | 7.79 (s, 2H), 6.77 (s, |
| | 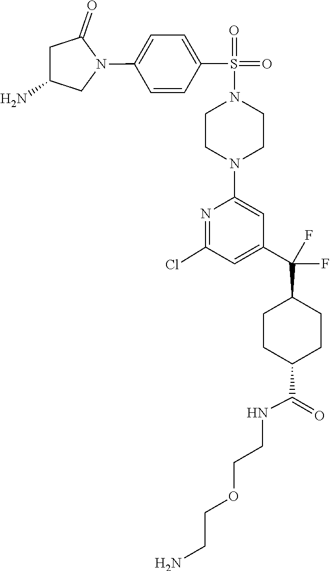 [SMILES: NCCOCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 1H), 6.69 (s, 1H), 4.32-4.20 (m, 1H), 4.13-4.01 (m, 1H), 3.95-3.86 (m, 1H), 3.64 (br s, 4H), 3.41 (br s, 2H), 3.24-3.17 (m, 2H), 3.10-3.02 (m, 1H), 2.96 (br s, 6H), 2.71-2.62 (m, 1H), 2.23-2.09 (m, 2H), 1.78-1.59 (m, 4H), 1.41-1.26 (m, 2H), 1.17-1.03 (m, 2H). ¹⁹F NMR (376 MHz, DMSO-d₆) δ ppm −104.09 (br s, 2F). MS obsd. (ESI⁺) [(M + H)⁺]: 698.2. |
| 043 | Trans-N,N-bis(2-aminoethyl)-4- | LINKER: Int-5 | ¹H NMR (400 MHz, |
| | [2-chloro-6-[4-[4-[(4R)-4- | TAIL: bis(2- | DMSO-d₆) δ ppm 8.43 |
| | amino-2-oxo-pyrrolidin-1- | tert- | (br d, J = 4.04 Hz, 2H), |
| | yl]phenyl]sulfonylpiperazin-1- | butyloxycarbonyl- | 8.15 (br s, 2H), 7.91 |
| | yl]-4-pyridyl]-difluoro- | aminoethyl)amine | (br d, J = 8.93 Hz, 5H), |
| | methyl]cyclohexanecarboxamide | | 7.79 (d, J = 8.93 Hz, |
| | 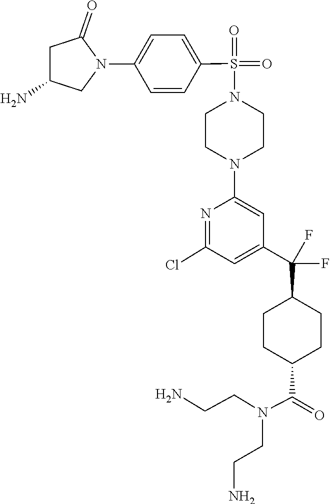 [SMILES: NCCN(CCN)C(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 2H), 6.77 (s, 1H), 6.70 (s, 1H), 4.22-4.30 (m, 1H), 4.07 (br dd, J = 7.76, 2.87 Hz, 2H), 3.82-3.90 (m, 2H), 3.65 (br s, 4H), 3.54- 3.60 (m, 4H), 3.07 (dd, J = 17.79, 8.13 Hz, 1H), 2.92-3.00 (m, 4H), 2.85-2.92 (m, 2H), 2.58-2.70 (m, 2H), 1.73-1.81 (m, 2H), 1.61-1.69 (m, 2H), 1.21-1.36 (m, 5H). ¹⁹F NMR (376 MHz, DMSO-d₆) δ ppm −103.49 (br s, 2F). MS obsd. (ESI⁺) [(M + H)⁺]: 697.1. |
| 044 | Trans-N,N-bis(3-aminopropyl)- | LINKER: Int-5 | ¹H NMR (400 MHz, |
| | 4-[[2-chloro-6-[4-[4-[(4R)-4- | TAIL: Tert- | DMSO-d₆) δ ppm 8.69 |
| | amino-2-oxo-pyrrolidin-1- | butyl N-[3-[3- | (s, 2H), 8.21 (s, 2H), |
| | yl]phenyl]sulfonylpiperazin-1- | (tert- | 8.05 (s, 3H), 7.90 (d, |
| | yl]-4-pyridyl]-difluoro- | butoxycarbonyl- | J = 8.93 Hz, 2H), 7.77 |
| | methyl]cyclohexanecarboxamide | amino)propyl- | (s, 2H), 6.77 (s, 1H), |
| | 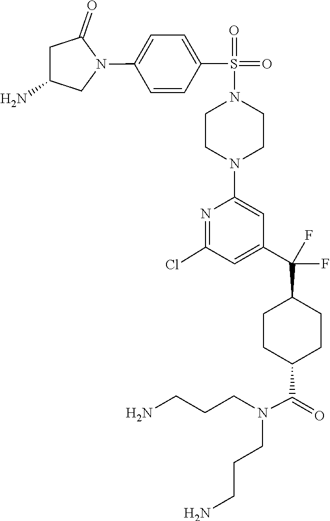 [SMILES: NCCCN(CCCN)C(=O)[C@H]1CC[C@H](C(F)(F)c2cc(Cl)nc(N3CCN(S(=O)(=O)c4ccc(N5C[C@H](N)CC5=O)cc4)CC3)c2)CC1] | amino]propyl] carbamate | 6.69 (s, 1H), 4.26 (dd, J = 11.07, 6.91 Hz, 1H), 4.06 (s, 1H), 3.92 (d, J = 10.64 Hz, 1H), 3.65 (s, 4H), 3.38 (s, 2H), 3.29 (t, J = 6.60 Hz, 2H), 2.90-3.15 (m, 5H), 2.81 (d, J = 6.11 Hz, 2H), 2.62-2.73 (m, 3H), 2.54 (s, 1H) 2.10- 2.30 (m, 1H), 1.72- 1.87 (m, 4H), 1.65 (t, J = 9.11 Hz, 4H). ¹⁹F NMR (376 MHz, DMSO-d₆) δ ppm −108.93-−99.31 (m, 2F). MS obsd. (ESI⁺) [(M + H)⁺]: 725.5. |
| 045 | N,N-bis(3-aminopropyl)-4-[[2- | LINKER: Int- | ¹H NMR (400 MHz, |
| | chloro-6-[4-[4-[(4R)-4-amino-2- | 10 | DMSO-d₆) δ ppm 8.62 |
| | oxo-pyrrolidin-1- | TAIL: Tert- | (s, 3H), 8.07 (s, 6H), |
| | yl]phenyl]sulfonylpiperazin-1- | butyl N-[3-[3- | 7.92 (d, J = 8.68 Hz, |
| | yl]-4-pyridyl]-difluoro- | (tert- | 2H), 7.80 (d, J = 8.56 |
| | methyl]bicyclo[2.2.2]octane-1- | butoxycarbonyl- | Hz, 2H), 6.64 (s, 1H), |
| | carboxamide | amino)propyl- | 6.60 (s, 1H), 4.27 (t, |
| | 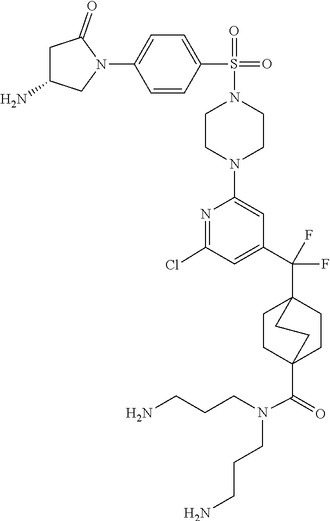 [SMILES: NCCCN(CCCN)C(=O)C12CCC(C(F)(F)c3cc(Cl)nc(N4CCN(S(=O)(=O)c5ccc(N6C[C@H](N)CC6=O)cc5)CC4)c3)(CC1)CC2] | amino]propyl] carbamate | J = 7.64 Hz, 1H), 4.07 (s, 1H), 3.91 (d, J = 10.39 Hz, 1H), 3.65 (s, 4H), 3.03-3.13 (m, 2H), 2.97 (s, 4H), 2.63-2.84 (m, 7H), 1.74 (s, 10H), 1.55 (s, 6H). ¹⁹F NMR (376 MHz, DMSO-d₆) δ ppm −106.67-−105.20 (m, 2F). MS obsd. (ESI⁺) [(M + H)⁺]: 751.5. |
| 046 | N,N-bis(3-aminopropyl)-4-[2- | LINKER: Int-9 | ¹H NMR (400 MHz, |
| | chloro-6-[4-[4-[(4R)-4-amino-2- | TAIL: Tert- | DMSO-d₆) δ ppm 8.69 |
| | oxo-pyrrolidin-1- | butyl N-[3-[3- | (br d, J = 4.03 Hz, 3H), |
| | yl]phenyl]sulfonylpiperazin-1- | (tert- | 8.03 (br s, 3H), 7.82- |
| | yl]-4-pyridyl]-difluoro- | butoxycarbonyl- | 7.94 (m, 3H), 7.78 (d, |
| | methyl]norbornane-1- | amino)propyl- | J = 8.93 Hz, 2H), 6.68 |
| | carboxamide | amino]propyl] | (s, 1H), 6.62 (s, 1H), |
| | 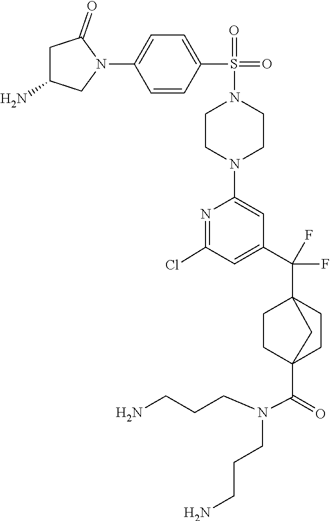 [SMILES: NCCCN(CCCN)C(=O)C12CCC(C(F)(F)c3cc(Cl)nc(N4CCN(S(=O)(=O)c5ccc(N6C[C@H](N)CC6=O)cc5)CC4)c3)(CC1)C2] | carbamate | 4.26 (dd, J = 11.07, 6.91 Hz, 1H), 4.06 (br s, 1H), 3.92 (dd, J = 10.94, 1.90 Hz, 1H), 3.60-3.63 (m, 4H), 3.02-3.13 (m, 3H), 2.96 (br s, 4H), 2.63- 2.76 (m, 3H), 1.55- 1.79 (m, 10H), 1.28 (br t, J = 8.01 Hz, 2H). ¹⁹F NMR (376 MHz, DMSO-d₆) δ ppm −101.53 (br s, 2F). MS obsd. (ESI⁺) [(M + H)⁺]: 737.1. |
| 047 | Trans-N-[3-[bis(2- | LINKER: Int-5 | ¹H NMR (400 MHz, |
| | aminoethyl)amino]propyl]-4-[2- | TAIL: Int-18 | DMSO-d₆) δ ppm |
| | chloro-6-[4-[4-[(4R)-4-amino-2- | | 8.44-8.55 (m, 3H), |
| | oxo-pyrrolidin-1- | | 7.88-7.99 (m, 3H), |
| | yl]phenyl]sulfonylpiperazin-1- | | 7.76-7.84 (m, 2H), |
| | yl]-4-pyridyl]-difluoro- | | 6.75-6.81 (m, 1H), |
| | methyl]cyclohexanecarboxamide | | 6.69-6.72 (m, 1H), |
| | 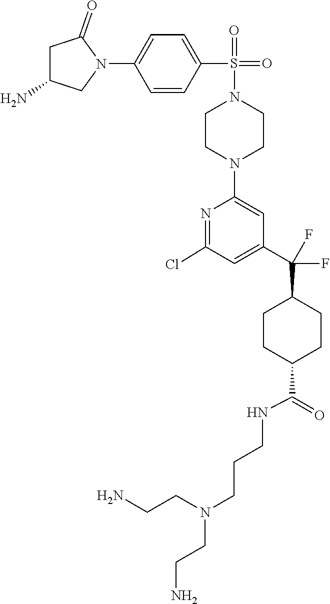 [SMILES: NCCN(CCN)CCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 4.21-4.31 (m, 1H), 4.02-4.12 (m, 1H), 3.85-3.92 (m, 1H), 3.65 (br s, 5H), 3.03- 3.12 (m, 5H), 2.93- 3.01 (m, 5H), 2.66 (br s, 1H), 1.99-2.25 (m, 3H), 1.71-1.80 (m, 3H), 1.60-1.69 (m, 3H), 1.25-1.40 (m, 2H), 1.02-1.19 (m, 3H). ¹⁹F NMR (376 MHz, DMSO-d₆) δ ppm −104.07 (br s, 2F). MS obsd. (ESI⁺) [(M + H)⁺]: 754.1. |
| 048 | Trans-(4R)-4-amino-1-[4-[4-[6- | LINKER: Int-5 | ¹H NMR (400 MHz, |
| | chloro-4-[[4-cis-(3,5- | TAIL: Int-17a | DMSO-d₆) δ ppm |
| | diaminopiperidine-1- | | 8.42-8.74 (m, 9H), |
| | carbonyl)cyclohexyl]-difluoro- | | 7.90 (d, J = 8.93 Hz, |
| | methyl]-2-pyridyl]piperazin-1- | | 2H), 7.79 (d, J = 8.93 Hz, |
| | yl]sulfonylphenyl]pyrrolidin-2- | | 2H), 6.77 (s, 1H), |
| | one | | 6.69 (s, 1H), 4.65 (d, |
| | 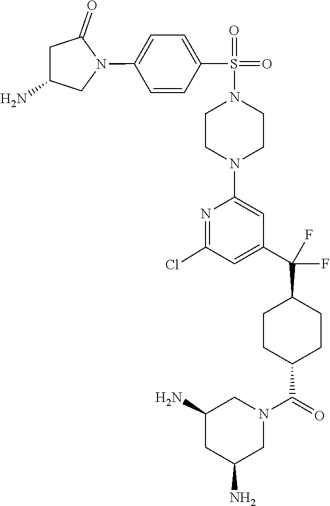 [SMILES: N[C@@H]1CC(=O)N(c2ccc(S(=O)(=O)N3CCN(c4cc(C(F)(F)[C@H]5CC[C@H](C(=O)N6C[C@H](N)C[C@H](N)C6)CC5)cc(Cl)n4)CC3)cc2)C1] | | J = 10.88 Hz, 1H), 4.21- 4.36 (m, 2H), 4.06 (s, 1H), 3.90 (d, J = 10.03 Hz, 1H), 3.65 (s, 5H), 2.90-3.23 (m, 8H), 2.66 (d, J = 17.85 Hz, 1H), 2.46 (br s, 2H), 2.20 ( t, J = 12.72 Hz, 1H), 1.60-1.85 (m, 5H), 1.11-1.40 (m, 4H). ¹⁹F NMR (376 MHz, DMSO-d₆) δ ppm −104.07-103.35 (m, 2F). MS obsd. (ESI⁺) [(M + H)⁺]: 709.2. |
| 049 | Trans-4-[[2-chloro-6-[4-[4- | LINKER: Int-5 | ¹H NMR (400 MHz, |
| | [(4R)-4-amino-2-oxo-pyrrolidin- | TAIL: Int-17 | DMSO-d₆) δ ppm 8.43 |
| | 1-yl]phenyl]sulfonylpiperazin-1- | | (br d, J = 4.04 Hz, 3H), |
| | yl]-4-pyridyl]-difluoro-methyl]- | | 8.15 (br s, 3H), 7.91 |
| | N-[3-[cis-3,5-diamino-1- | | (br d, J = 8.93 Hz, 5H), |
| | piperidyl]propyl]cyclohexane- | | 7.79 (d, J = 8.93 Hz, |
| | carboxamide | | 2H), 6.77 (s, 1H), 6.70 |
| | 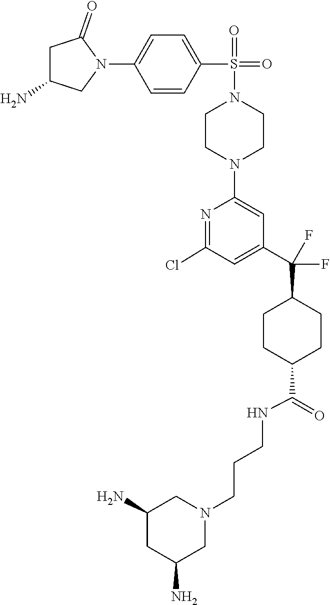 [SMILES: N[C@@H]1C[C@H](N)CN(CCCNC(=O)[C@H]2CC[C@H](C(F)(F)c3cc(Cl)nc(N4CCN(S(=O)(=O)c5ccc(N6C[C@H](N)CC6=O)cc5)CC4)c3)CC2)C1] | | (s, 1H), 4.22-4.30 (m, 1H), 4.07 (br dd, J = 7.76, 2.87 Hz, 2H), 3.82-3.90 (m, 2H), 3.65 (br s, 5H), 3.54- 3.60 (m, 4H), 3.07 (dd, J = 17.79, 8.13 Hz, 1H), 2.92-3.00 (m, 6H), 2.85-2.92 (m, 2H), 2.58-2.70 (m, 2H), 1.73-1.81 (m, 2H), 1.61-1.69 (m, 2H), 1.21-1.36 (m, 5H). ¹⁹F NMR (376 MHz, DMSO-d₆) δ ppm −104.071 (br s, 2 F). MS obsd. (ESI⁺) [(M + H)⁺]: 766.1. |
| 050 | Trans-N′-(2-aminoacetyl)-4-[[2- | LINKER: Int-5 | ¹H NMR (400 MHz, |
| | chloro-6-[4-[4-[(4R)-4-amino-2- | TAIL: Tert- | DMSO-d₆) δ ppm |
| | oxo-pyrrolidin-1- | butyl 2- | 10.24 (br s, 1H), 9.95 |
| | yl]phenyl]sulfonylpiperazin-1- | hydrazinyl-2- | (s, 1H), 8.07-8.34 (m, |
| | yl]-4-pyridyl]-difluoro- | oxoethylcarbamate | 4H), 7.91 (m, J = 8.80 |
| | methyl]cyclohexanecarbohydrazide | | Hz, 2H), 7.79 (m, |
| | 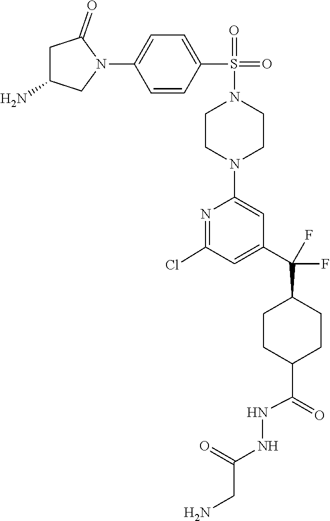 | | J = 8.80 Hz, 2H), 6.79 (s, 1H), 6.72 (s, 1H), 4.26 (br dd, J = 11.19, 6.91 Hz, 1H), 4.02- 4.15 (m, 1H), 3.82 (br d, J = 10.76 Hz, 1H), 3.56-3.70 (m, 5H), 3.14-3.20 (m, 2H), 3.07 (dd, J = 17.91, 8.25 Hz, 1H), 2.97 (br s, 3H), 2.53-2.61 (m, 1H), 2.18 (br d, J = 11.37 Hz, 2H), 1.77 (br d, J = 11.62 Hz, 2H), 1.64-1.72 (m, 2H), 1.37 (br d, J = 12.47 Hz, 2H), 1.14 (br d, J = 12.10 Hz, 2H). ¹⁹F NMR (376 MHz, DMSO-d₆) δ ppm −104.11 (br s, 2F). MS obsd. (ESI⁺) [(M + H)⁺]: 683.1. |
| 051 | Trans-N′-[4-[[2-chloro-6-[4-[4- | LINKER: Int-5 | ¹H NMR (400 MHz, |
| | [(4R)-4-amino-2-oxo-pyrrolidin- | TAIL: Tert- | DMSO-d₆) δ ppm |
| | 1-yl]phenyl]sulfonylpiperazin-1- | butyl 4- | 9.56-9.89 (m, 1H), |
| | yl]-4-pyridyl]-difluoro- | (hydrazine | 7.87-7.97 (m, 2H), |
| | methyl]cyclohexanecarbonyl] | carbonyl) | 7.71-7.86 (m, 1H), |
| | piperidine-4-carbohydrazide | piperidine-1- | 6.74-6.83 (m, 1H), |
| | 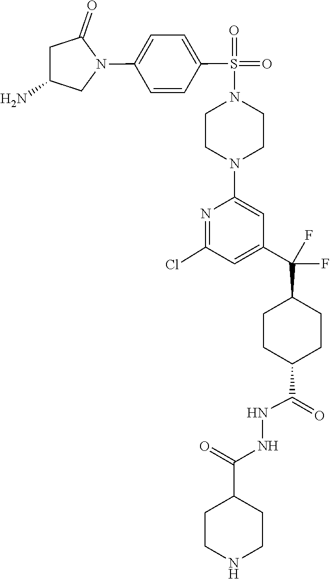 [SMILES: N[C@@H]1CC(=O)N(c2ccc(S(=O)(=O)N3CCN(c4cc(C(F)(F)[C@H]5CC[C@H](C(=O)NNC(=O)C6CCNCC6)CC5)cc(Cl)n4)CC3)cc2)C1] | carboxylate | 6.72 (s, 1H), 4.20-4.33 (m, 1H), 3.99-4.16 (m, 5H), 3.78-3.89 (m, 1H), 3.62-3.72 (m, 4H), 3.22-3.28 (m, 1H), 3.02-3.11 (m, 1H), 2.93-3.02 (m, 3H), 2.82-2.93 (m, 1H), 2.55-2.72 (m, 1H), 2.05-2.30 (m, 3H), 1.56-1.89 (m, 7H), 0.99-1.45 (m, 4H). ¹⁹F NMR (376 MHz, DMSO-d₆) δ ppm −104.09 (br s, 2F). MS obsd. (ESI⁺) [(M + H)⁺]: 737.1. |
| 052 | Trans-N-(2-amino-3-hydroxy- | LINKER: Int-5 | ¹H NMR (400 MHz, |
| | propyl)-4-[[2-chloro-6-[4-[4- | TAIL: N-Boc- | DMSO-d₆) δ ppm |
| | [(4R)-4-amino-2-oxo-pyrrolidin- | 1,3- | 8.14-8.21 (m, 2H), |
| | 1-yl]phenyl]sulfonylpiperazin-1- | diaminopropane | 7.87-7.97 (m, 2H), |
| | yl]-4-pyridyl]-difluoro- | | 7.72-7.84 (m, 2H), |
| | methyl]cyclohexanecarboxamide | | 6.75-6.85 (m, 1H), |
| | 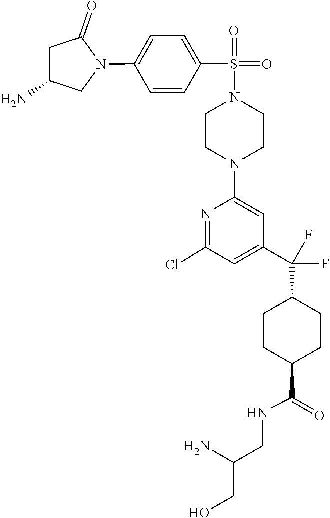 [SMILES: NC(CO)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 6.62-6.74 (m, 1H), 5.20-5.36 (m, 1H), 4.20-4.34 (m, 1H), 4.02-4.12 (m, 1H), 3.83-3.94 (m, 1H), 3.60-3.69 (m, 4H), 3.51-3.58 (m, 1H), 3.43-3.51 (m, 2H), 3.20-3.29 (m, 2H), 3.02-3.17 (m, 2H), 2.90-3.02 (m, 4H), 2.59-2.71 (m, 1H), 1.98-2.30 (m, 2H), 1.73-1.89 (m, 2H), 1.58-1.73 (m, 2H), 1.22-1.45 (m, 2H), 0.98-1.23 (m, 3H). ¹⁹F NMR (376 MHz, DMSO- ppm −104.68-103.32 (m, 2F). MS obsd. (ESI⁺) [(M + H)⁺]: 684.2. |
| 053 | (2R,5S)-5-[2-[4-[4-[(4R)-4- | LINKER: Int- | ¹H NMR (400 MHz, |
| | amino-2-oxo-pyrrolidin-1- | 26 | DMSO-d₆) δ ppm |
| | yl]phenyl]sulfonylpiperazin-1- | TAIL: N-Boc- | 1.21-1.34 (m, 2H), |
| | yl]-6-chloro-4-pyridyl]-difluoro- | 1,3- | 1.45-1.59 (m, 5H), |
| | methyl]-N-(3- | diaminopropane | 1.67 (dt, J = 14.09, |
| | aminopropyl)bicyclo[2.2.2] | | 6.83 Hz, 3H), 1.79-1.93 |
| | octane-2-carboxamide | | (m, 2H), 2.38-2.47 (m, |
| | 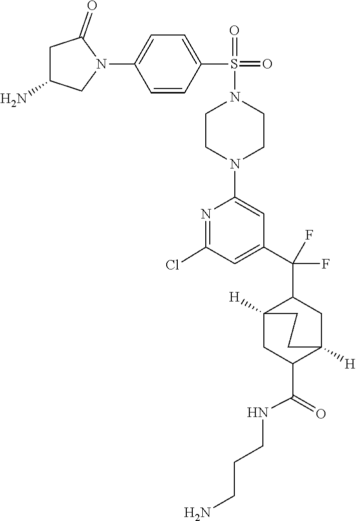 [SMILES: NCCCNC(=O)C1C[C@@H]2CC[C@H]1CC2C(F)(F)c1cc(Cl)nc(N2CCN(S(=O)(=O)c3ccc(N4C[C@H](N)CC4=O)cc3)CC2)c1] | | 2H), 2.63-2.76 (m, 3H), 2.96 (br s, 4H), 3.01-3.16 (m, 3H), 3.66 (br s, 4H), 3.91 (br d, J = 11.25 Hz, 1H), 4.06 (br s, 1H), 4.26 (dd, J = 11.19, 6.91 Hz, 1H), 6.75 (s, 1H), 6.83 (s, 1H), 7.79 (d, J = 8.93 Hz, 2H), 7.91 (d, J = 8.93 Hz, 2H), 8.01 (br d, J = 5.87 Hz, 4H), 8.64 (br s, 3H). ¹⁹F NMR (376 MHz, DMSO-d₆) δ ppm −105.60-98.08 (m, 2F). MS obsd. (ESI⁺) [(M + H), ⁺]: 694.2. |
| 054 | Trans-N-(3-aminopropyl)-4- | LINKER: Int- | ¹H NMR (400 MHz, |
| | [difluoro-[6-methyl-2-[4-[4- | 24 | DMSO-d₆) δ ppm 9.18 |
| | [(4R)-4-amino-2-oxo-pyrrolidin- | TAIL: N-Boc- | (d, J = 4.64 Hz, 2H), |
| | 1-yl]phenyl]sulfonylpiperazin-1- | 1,3- | 8.94-9.11 (m, 2H), |
| | yl]pyrimidin-4- | diaminopropane | 8.43-8.74 (m, 1H), |
| | yl]methyl]cyclohexanecarboxamide | | 7.82 (br t, J = 9.72 Hz, |
| | 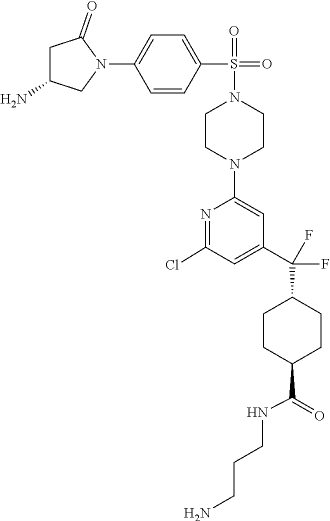 [SMILES: NCCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 2H), 7.54 (dd, J = 8.91, 5.40 Hz, 2H), 6.87 (d, J = 6.65 Hz, 1H), 6.82 (d, J = 4.27 Hz, 1H), 4.50 (br d, J = 12.80 Hz, 1H), 4.33 (br d, J = 12.30 Hz, 1H), 3.82- 4.05 (m, 2H), 3.46- 3.57 (m, 2H), 2.77- 3.05 (m, 7H), 2.67- 2.76 (m, 1H), 2.22- 2.37 (m, 1H), 1.81- 2.14 (m, 4H), 0.40- 0.81 (m, 2H). ¹⁹F NMR (376 MHz, DMSO-d₆) δ ppm - 108.99 (br s, 2F). MS obsd. (ESI⁺) [(M + H), ⁺]: 649.5. |

### Example 055: N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[3.1.0]hexane-3-carboxamide

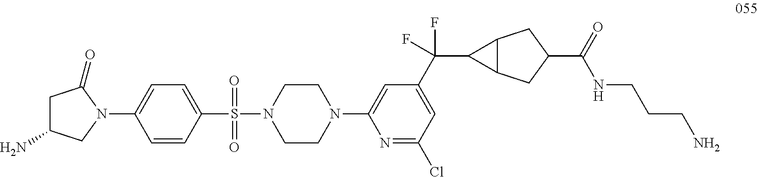 [SMILES: NCCCNC(=O)C1CC2C(C1)C2C(F)(F)c1cc(Cl)nc(N2CCN(S(=O)(=O)c3ccc(N4C[C@H](N)CC4=O)cc3)CC2)c1]

### Step 1: 6-[(2,6-Dichloro-4-pyridyl)-difluoro-methyl]bicyclo[3.1.0]hexane-3-carboxylic acid

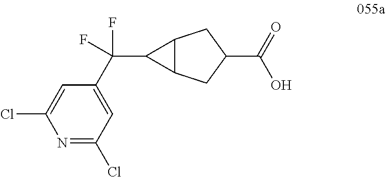 [SMILES: O=C(O)C1CC2C(C1)C2C(F)(F)c1cc(Cl)nc(Cl)c1]

To a solution of compound Int-19 (200.0 mg, 0.530 mmol) in DCM (2 mL) was added TFA (2.0 mL, 25.96 mmol), and the mixture was stirred at 25° C. for 2 hours. After completion, the mixture was concentrated in vacuo to give a crude residue, which was purified by prep-HPLC. The compound 055a (120 mg, 0.370 mmol, 70.45% yield) was obtained as yellow oil. MS obsd. (ESI⁺) [(M+H)⁺]: 322.2.

### Step 2: Tert-butyl N-[3-[[6-[(2,6-dichloro-4-pyridyl)-difluoro-methyl]bicyclo[3.1.0]hexane-3-carbonyl]amino]propyl]carbamate

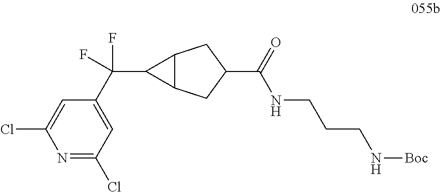 [SMILES: CC(C)(C)OC(=O)NCCCNC(=O)C1CC2C(C1)C2C(F)(F)c1cc(Cl)nc(Cl)c1]

To a solution of compound 055a (120.0 mg, 0.370 mmol), N-Boc-1,3-diaminopropane (194.72 mg, 1.12 mmol) and DIPEA (0.19 mL, 1.12 mmol) in DMF (0.5 mL) was added HATU (212.47 mg, 0.560 mmol). The mixture was stirred at 25° C. for 2 hours. After completion, the mixture was diluted with water (100 mL), extracted with EtOAc (100 mL×2) and washed with brine (200 mL). The combined organic layer was dried over anhydrous sodium sulfate, filtered and concentrated in vacuo to give a crude residue, which was purified by prep-HPLC to afford compound 055b (120 mg, 0.250 mmol, 67.34% yield) as a yellow solid. MS obsd. (ESI⁺) [(M+H)⁺]: 421.9.

### Step 3: Tert-butyl N-[3-[[6-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]bicyclo[3.1.0]hexane-3-carbonyl]amino]propyl]carbamate

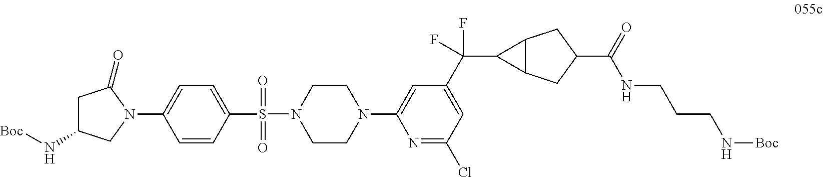 [SMILES: CC(C)(C)OC(=O)NCCCNC(=O)C1CC2C(C1)C2C(F)(F)c1cc(Cl)nc(N2CCN(S(=O)(=O)c3ccc(N4C[C@H](NC(=O)OC(C)(C)C)CC4=O)cc3)CC2)c1]

To a solution of compound 055b (120 mg, 0.250 mmol) and compound Int-5 (117.14 mg, 0.280 mmol) in DMSO (2 mL) was added DIPEA (1.0 mL, 5.74 mmol). The mixture was stirred at 100° C. for 12 hours. After completion, the mixture was diluted with water (100 mL), extracted with EtOAc (100 mL×2) and washed with brine (100 mL). The combined organic layer was dried over anhydrous sodium sulfate, filtered and concentrated in vacuo to give a residue, which was purified by prep-TLC (eluent with EtOAc:PE=33%) and prep-HPLC. Compound 054c (40 mg, 0.050 mmol, 18.4% yield) was obtained as a yellow solid. MS obsd. (ESI⁺) [(M+H)⁺]: 866.3.

### Step 4: N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[3.1.0]hexane-3-carboxamide

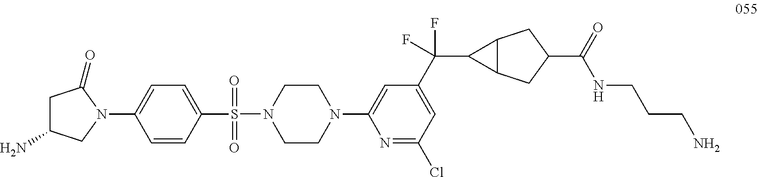 [SMILES: NCCCNC(=O)C1CC2C(C1)C2C(F)(F)c1cc(Cl)nc(N2CCN(S(=O)(=O)c3ccc(N4C[C@H](N)CC4=O)cc3)CC2)c1]

To a solution of compound 055c (40.0 mg, 0.050 mmol) in DCM (2 mL) was added TFA (2.0 mL, 25.96 mmol). The mixture was stirred at 25° C. for 1 hour. After completion, the mixture was concentrated in vacuo to give a crude residue, which was purified by prep-HPLC to give Example 055 (10.6 mg, 0.020 mmol, 29.24% yield) as a white solid. MS obsd. (ESI⁺) [(M+H)⁺]: 666.5.

Example 055: ¹H NMR (400 MHz, DMSO-d₆) δ ppm 8.61 (br s, 3H), 8.02-8.27 (m, 1H), 7.94-8.02 (m, 3H), 7.91 (d, J=8.93 Hz, 2H), 7.76-7.81 (m, 2H), 6.65-6.88 (m, 2H), 4.26 (br dd, J=11.07, 6.91 Hz, 1H), 4.05 (br s, 1H), 3.90 (br d, J=11.00 Hz, 1H), 3.66 (br s, 4H), 3.01-3.16 (m, 3H), 2.96 (br s, 4H), 2.58-2.79 (m, 3H), 1.94-2.39 (m, 2H), 1.28-2.45 (m, 3H). ¹⁹F NMR (376 MHz, DMSO-d₆) δ ppm −96.02 (br s, 2F).

### Example 056: Trans-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

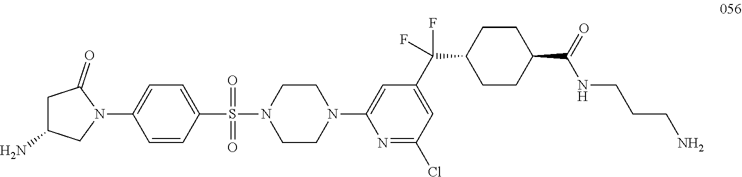 [SMILES: NCCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-4-[(2,6-dichloro-4-pyridyl)-difluoro-methyl]cyclohexanecarboxylic acid

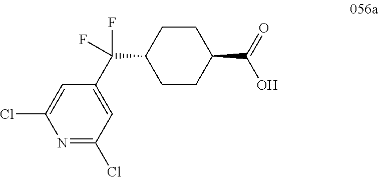 [SMILES: O=C(O)[C@H]1CC[C@H](C(F)(F)c2cc(Cl)nc(Cl)c2)CC1]

To a solution of compound Int-5e (2.0 g, 5.91 mmol) and lithium bromide (5.1 g, 60 mmol) in MeCN (10 mL) and water (3 mL) was added TEA (3.0 mL, 21.52 mmol). The mixture was stirred at 50° C. for 12 hours. After completion, the mixture was adjusted to pH=5 with 1 M HCl solution, extracted with EtOAc (300 mL×2) and washed with brine (300 mL). The combined organic layer was dried over anhydrous sodium sulfate, filtered and concentrated in vacuo to give compound 056a (1.8 g, 5.55 mmol, 93.9% yield) as a white solid. MS obsd. (ESI⁺) [(M+H)⁺]: 323.8.

### Step 2: Trans-N-butyl-4-[(2,6-dichloro-4-pyridyl)-difluoro-methyl]cyclohexanecarboxamide

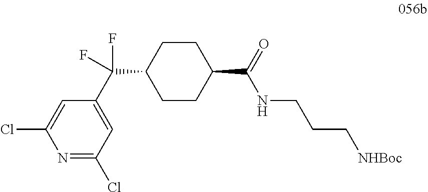 [SMILES: CC(C)(C)OC(=O)NCCCNC(=O)[C@H]1CC[C@H](C(F)(F)c2cc(Cl)nc(Cl)c2)CC1]

To a solution of compound 056a (150 mg, 463 μmol), HATU (264 mg, 694 μmol,) and DIPEA (242 μL, 1.39 mmol) in DMF (5 mL) was added tert-butyl N-(3-aminopropyl)carbamate (96.8 mg, 555 μmol). The mixture was stirred at room temperature for 2 hours. After completion, the reaction mixture was diluted with EtOAc (10 mL) and washed with brine (50 mL). The organic layer was dried over anhydrous sodium sulfate and concentrated in vacuo to give crude product, which was purified by silica gel chromatography (eluent with EtOAc:PE=0 to 40%) to give compound 056b (180 mg, 375 μmol, 81% yield) as yellow oil. MS obsd. (ESI⁺) [(M+H)⁺]: 480.6.

### Step 3: Trans-tert-butyl N-[3-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]propyl]carbamate

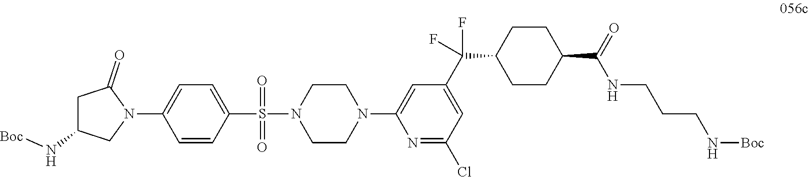 [SMILES: CC(C)(C)OC(=O)NCCCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

A solution of compound Int-5 (100 mg, 236 μmol), compound 056b (147 mg, 306 μmol) and DIPEA (152 mg, 1.18 mmol) in DMSO (2 mL) was stirred at 100° C. overnight. After completion, the mixture was diluted with EtOAc (10 mL) and washed with brine (10 mL). The organic layer was dried over anhydrous sodium sulfate and concentrated in vacuo to give the desired product compound 056c (205 mg, 236 μmol, 100% yield) as yellow oil, which was used in the next step directly. MS obsd. (ESI⁺) [(M+H)⁺]: 868.8.

### Step 4: Trans-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

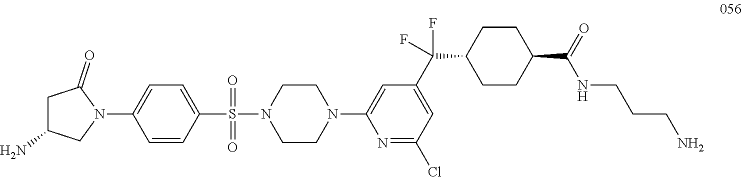 [SMILES: NCCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

A solution of compound 056c (205 mg, 236 μmol) and TFA (364 μL, 4.72 mmol) was stirred at room temperature for 3 hours. The mixture was then concentrated in vacuo to give the crude product, which was purified by prep-HPLC to give the desired product Example 056 (89 mg, 124 μmol, 52.5% yield) as a yellow solid. MS obsd. (ESI⁺) [(M+H)]⁺: 668.2.

Example 056: ¹H NMR (400 MHz, METHANOL-d₄) ppm δ 7.84 (br d, J=8.8 Hz, 2H), 7.72 (d, J=8.7 Hz, 2H), 6.58 (s, 1H), 6.55 (s, 1H), 4.32 (br dd, J=11.2, 6.9 Hz, 1H), 4.12 (br s, 1H), 3.98-3.84 (m, 1H), 3.59 (br s, 4H), 3.19-2.98 (m, 4H), 2.96 (br s, 4H), 2.91-2.78 (m, 2H), 2.64 (dd, J=18.1, 2.8 Hz, 1H), 2.10-1.89 (m, 3H), 1.82-1.65 (m, 7H), 1.41-1.26 (m, 2H), 1.24-1.04 (m, 3H). ¹⁹F NMR (376 MHz, METHANOL-d₄) δ ppm −106.88 (s, 1F), −106.92 (s, 1F).

The following Examples 057 to 062 were prepared in analogy to the procedure described for the preparation of Example 056, replacing compound Int-5 with “LINKER” in Step 3, and replacing tert-butyl N-(3-aminopropyl)carbamate with “TAIL” in Step 2 by the reagents indicated in Table 2.

TABLE 2
Compound synthesis and characterization

| | Compound Names and | LINKER and | |
|---|---|---|---|
| Ex. | Structures | TAIL | NMR and (ESI⁺) |
| 057 | Trans-(4R)-4-amino-1-[4-[4- | LINKER: Int- | ¹H NMR (400 MHz, |
| | [4-[4-[3- | 5 | DMSO-d₆) δ ppm 7.91 |
| | (aminomethyl)azetidine-1- | TAIL: tert- | (m, J = 9.05 Hz, 2H), |
| | carbonyl]cyclohexyl]-difluoro- | butyl N- | 7.71-7.77 (m, 2H), |
| | methyl]-6-chloro-2- | (azetidin-3- | 6.76 (s, 1H), 6.69 (s, |
| | pyridyl]piperazin-1- | ylmethyl) | 1H), 4.04-4.14 (m, |
| | yl]sulfonylphenyl]pyrrolidin-2- | carbamate | 1H), 3.98 (dd, J = 9.84, |
| | one | | 6.05 Hz, 1H), 3.70-3.82 |
| | | | (m, 2H), 3.60-3.69 (m, |
| | 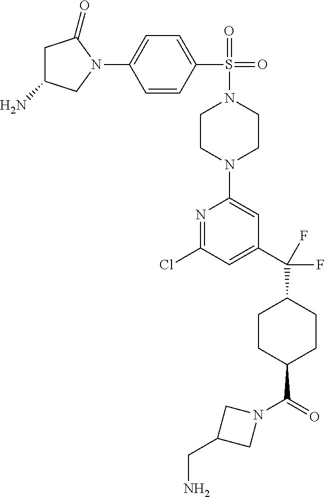 [SMILES: NCC1CN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)C1] | | 4H), 3.43-3.51 (m, 2H), 3.12 (br d, J = 7.09 Hz, 2H), 2.88- 3.03 (m, 4H), 2.64- 2.79 (m, 3H), 2.30- 2.36 (m, 1H), 2.02- 2.29 (m, 3H), 1.66 (br d, J = 9.78Hz, 4H), 1.20-1.35 (m, 2H), 1.05-1.20 (m, 2H). ¹⁹F NMR (376 MHz, DMSO-d₆) δ ppm −103.24 (br dd, J = 44.3, 14.3 Hz, 1F), −103.81 (br dd, J = 50.4, 13.6 Hz, 1F). MS obsd. (ESI+) [(M + H)⁺]: 680.2 |
| 058 | Trans-N-(3-aminopropyl)-4- | LINKER: Int- | ¹H NMR (400 MHz, |
| | [[2-chloro-6-[4-[4-[(5S)-5- | 3A | METHANOL-d₄) δ |
| | (hydroxymethyl)-2-oxo- | TAIL: tert- | ppm 7.83-7.88 (m, |
| | oxazolidin-3- | butyl N-(3- | 2H), 7.78-7.81 (m, |
| | yl]phenyl]sulfonylpiperazin-1- | aminopropyl) | 2H), 6.66 (d, |
| | y1]-4-pyridyl]-difluoro- | carbamate | J = 8.19 Hz, 2H), 4.58 |
| | methyl]cyclohexanecarboxamide | | (s, 3H), 4.18 (t, |
| | | | J = 9.05 Hz, 1H), 3.98 |
| | 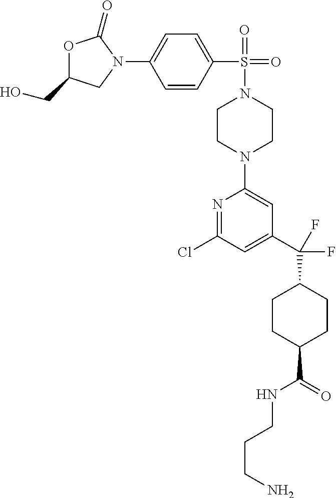 [SMILES: NCCCNC(=O)[C@H]1CC[C@H](C(F)(F)c2cc(Cl)nc(N3CCN(S(=O)(=O)c4ccc(N5C[C@@H](CO)OC5=O)cc4)CC3)c2)CC1] | | (dd, J = 8.93, 6.24 Hz, 1H), 3.86 (dd, J = 12.59, 3.18 Hz, 1H), 3.63-3.76 (m, 5H), 3.34-3.35 (m, 2H), 3.23-3.27 (m, 2H), 3.04-3.13 (m, 4H), 2.88 (t, J = 7.21 Hz, 2H), 1.99-2.15 (m, 2H), 1.74-1.87 (m, 6H), 1.44 (q, J = 12.63 Hz, 2H), 1.16-1.28 (m, 2H), 0.00 (s, 1H). ¹⁹F NMR (376 MHz, METHANOL-d₄) δ ppm −107.07 (s, 1F), −107.11 (s, 1F). MS obsd. (ESI+) [(M + H)⁺]: 685.5. |
| 059 | Trans-N-(3-aminopropyl)-4- | LINKER: Int- | ¹H NMR (500 MHz, |
| | [[2-chloro-6-[4-[4-[(5R)-5- | 3B | METHANOL-d₄) δ |
| | (hydroxymethyl)-2-oxo- | TAIL: tert- | ppm 7.85 (m, |
| | oxazolidin-3- | butyl N-(3- | J = 9.00 Hz, 2H), 7.80 |
| | yl]phenyl]sulfonylpiperazin-1- | aminopropyl) | (m, J = 9.00 Hz, 2H), |
| | yl]-4-pyridyl]-difluoro- | carbamate | 6.67 (s, 1H), 6.64 (s, |
| | methyl]cyclohexanecarboxamide | | 1H), 4.73-4.81 (m, |
| | | | 2H), 4.58 (br s, 1H), |
| | 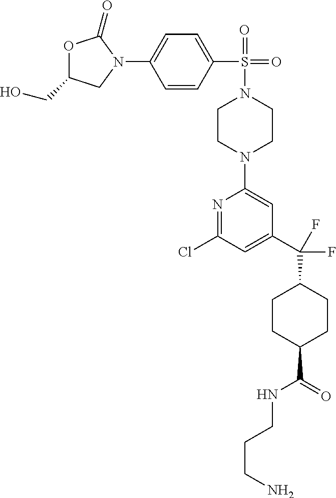 [SMILES: NCCCNC(=O)[C@H]1CC[C@H](C(F)(F)c2cc(Cl)nc(N3CCN(S(=O)(=O)c4ccc(N5C[C@H](CO)OC5=O)cc4)CC3)c2)CC1] | | 4.18 (t, J = 9.00 Hz, 1H), 3.98 (dd, J = 8.93, 6.33 Hz, 1H), 3.86 (dd, J = 12.59, 3.13 Hz, 1H), 3.66-3.73 (m, 4H), 3.22-3.29 (m, 2H), 3.01-3.12 (m, 4H), 2.90 (t, J = 7.25 Hz, 2H), 1.99-2.20 (m, 3H), 1.74-1.89 (m, 5H), 1.37-1.51 (m, 2H), 1.33 (br s, 1H), 1.19- 1.31 (m, 4H). ¹⁹F NMR (471 MHz, METHANOL-d₄) δ ppm −123.59-−94.82 (m, 2F). MS obsd. (ESI+) [(M + H)⁺]: 685.1. |
| 060 | Trans-N-(3-aminopropyl)-4- | LINKER: Int- | ¹H NMR (400 MHz, |
| | [[2-chloro-6-[4-[4-(2-oxo-4- | 4 | METHANOL-d₄) δ |
| | piperazin-1-yl-pyrrolidin-1- | TAIL: tert- | ppm 8.44 (s, 1H), 7.83 |
| | yl)phenyl]sulfonylpiperazin-1- | butyl N-(3- | (s, 1H), 7.81 (s, 1H), |
| | y1]-4-pyridyl]-difluoro- | aminopropyl) | 7.70 (d, J = 8.93Hz, |
| | methyl]cyclohexanecarboxamide | carbamate | 2H), 6.57 (d, |
| | | | J = 8.44 Hz, 2H), 4.01 |
| | 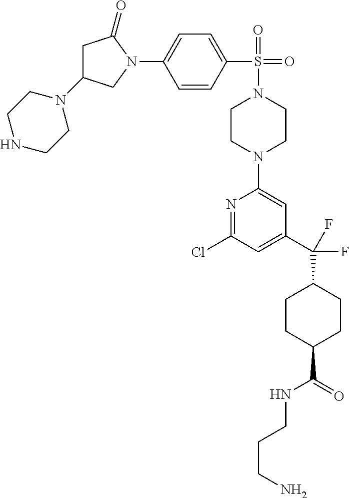 [SMILES: NCCCNC(=O)[C@H]1CC[C@H](C(F)(F)c2cc(Cl)nc(N3CCN(S(=O)(=O)c4ccc(N5CC(N6CCNCC6)CC5=O)cc4)CC3)c2)CC1] | | (dd, J = 9.84, 7.52 Hz, 1H), 3.75 (dd, J = 9.90, 6.48 Hz, 1H), 3.55-3.62 (m, 4H), 3.24-3.33 (m, 2H), 3.12-3.19 (m, 2H), 2.93-3.09 (m, 8H), 2.67-2.87 (m, 4H), 2.52-2.65 (m, 5H), 1.83-2.10 (m, 3H), 1.64-1.80 (m, 6H), 1.24-1.45 (m, 2H), 1.05-1.24 (m, 2H). ¹⁹F NMR (376 MHz, METHANOL-d₄) δ ppm −107.03 (s, 1F), −107.07 (s, 1F). MS obsd. (ESI+) [(M + H)⁺]: 737.5. |
| 061 | Trans-N-(3-aminopropyl)-4- | LINKER: Int- | ¹H NMR (400 MHz, |
| | [[2-chloro-6-[4-[[6-[(4R)-4- | 20 | DMSO-d₆) δ ppm 8.76 |
| | amino-2-oxo-pyrrolidin-1-yl]- | TAIL: tert- | (d, J = 2.08 Hz, 1H), |
| | 3-pyridyl]sulfonyl]piperazin-1- | butyl N-(3- | 8.53 (d, J = 9.05 Hz, |
| | yl]-4-pyridyl]-difluoro- | aminopropyl) | 1H), 8.41 (s, 2H), 8.23 |
| | methyl]cyclohexanecarboxamide | carbamate | (dd, J = 9.05, 2.45 Hz, |
| | | | 1H), 7.97 (t, J = 5.87 Hz, |
| | 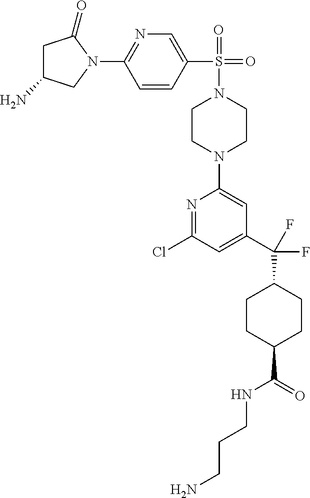 [SMILES: NCCCNC(=O)[C@H]1CC[C@H](C(F)(F)c2cc(Cl)nc(N3CCN(S(=O)(=O)c4ccc(N5C[C@H](N)CC5=O)nc4)CC3)c2)CC1] | | 1H), 7.86 ( s, 2H), 6.78-6.86 (m, 1H), 6.70-6.76 (m, 1H), 4.23-4.32 (m, 1H), 4.13-4.21 (m, 1H), 4.01-4.10 (m, 1H), 3.67 (s, 4H), 3.17 (dd, J = 18.03, 8.13 Hz, 1H), 3.05 ( s, 6H), 2.67- 2.75 (m, 3H), 2.14- 2.26 (m, 1H), 1.99- 2.09 (m, 1H), 1.75 ( d, J = 14.06 Hz, 2H), 1.61- 1.69 (m, 4H), 1.27- 1.40 (m, 2H), 1.06- 1.18 (m, 2H). ¹⁹F NMR (376 MHz, DMSO-d₆) δ ppm −104.88-−103.36 (m, 2 F). MS obsd. (ESI+) [(M + H)]⁺: 669.5. |
| 062 | Trans-N-(3-aminopropyl)-4- | LINKER: Int- | ¹H NMR (400 MHz, |
| | [[2-chloro-6-[4-[4-[(4R)-4- | 21 | DMSO-d₆) δ ppm |
| | amino-2-oxo-pyrrolidin-1- | TAIL: tert- | 7.87-8.01 (m, 6H), |
| | yl]phenyl]sulfonyl-1- | butyl N-(3- | 6.78-6.86 (m, 1H), |
| | piperidyl]-4-pyridyl]-difluoro- | aminopropyl) | 6.67-6.73 (m, 1H), |
| | methyl]cyclohexanecarboxamide | carbamate | 4.35-4.47 (m, 2H), |
| | | | 4.23-4.35 (m, 1H), |
| | 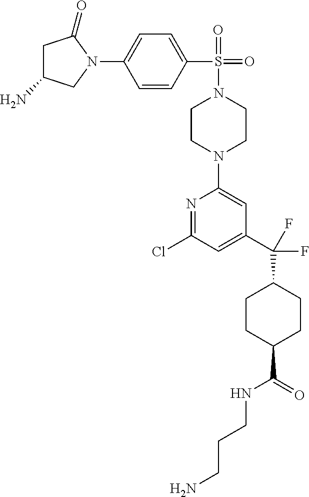 [SMILES: NCCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 4.05-4.17 (m, 1H), 3.81-3.96 (m, 1H), 3.51-3.69 (m, 1H), 3.01-3.21 (m, 4H), 2.84-2.99 (m, 2H), 2.69-2.79 (m, 2H), 2.13-2.31 (m, 2H), 1.97-2.12 (m, 1H), 1.83-1.95 (m, 2H), 1.55-1.82 (m, 7H), 1.24-1.55 (m, 5H), 1.01-1.22 (m, 3H). ¹⁹F NMR (376 MHz, DMSO-d₆) δ ppm −104.034 (br s, 2F). MS obsd. (ESI+) [(M + H)]⁺: 667.2. |
| 063 | Cis-N-(2-amino-2-oxo-ethyl)- | LINKER: Int- | ¹H NMR (400 MHz, |
| | 4-[[2-chloro-6-[4-[4-[(4R)-4- | 5 | DMSO-d₆) δ ppm 1.46 |
| | amino-2-oxo-pyrrolidin-1- | TAIL: N- | (br s, 4H) 1.91-2.00 |
| | yl]phenyl]sulfonylpiperazin-1- | ethyl-N- | (m, 4H) 2.23-2.28 (m, |
| | y1]-4-pyridyl]-difluoro- | isopropylpropan- | 1H) 2.46 (br s, 1H) |
| | methyl]cyclohexanecarboxamide | 2-amine | 2.78 (dd, J =16.87, |
| | | | 7.09 Hz, 1H) 2.96 (br t, |
| | 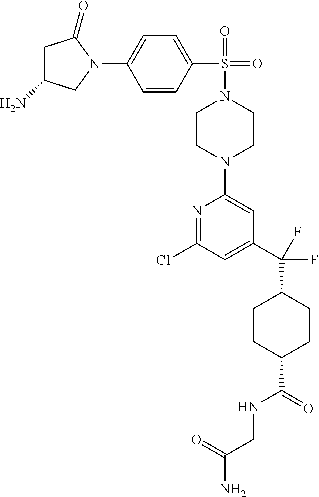 [SMILES: NC(=O)CNC(=O)[C@H]1CC[C@@H](C(F)(F)c2cc(Cl)nc(N3CCN(S(=O)(=O)c4ccc(N5C[C@H](N)CC5=O)cc4)CC3)c2)CC1] | | J = 4.77 Hz, 4H) 3.50 (br dd, J = 9.78, 2.93 Hz, 1H) 3.62-3.70 (m, 4H) 4.00 (dd, J = 9.78, 6.36 Hz, 1H) 6.65-6.68 (m, 1H) 6.75 (s, 1H) 6.96 (br s, 2H) 7.21 (br s, 2H) 7.75 (d, J = 8.80 Hz, 2H) 7.89- 7.96 (m, 2H). ¹⁹F NMR (376 MHz, DMSO-d₆) δ ppm −103.66-−103.25 (m, 1F), −103.09-−102.71 (m, 1F). MS obsd. (ESI+) [(M + H)⁺]: 668.2 |
| 064 | (4R)-4-amino-1-[4-[4-[4-[[4- | LINKER: Int- | ¹H NMR (400 MHz, |
| | [(2S,4S)-4-amino-2-methyl- | 5 | DMSO-d₆) δ ppm |
| | pyrrolidine-1- | TAIL: tert- | 8.70-8.61 (m, 3H), |
| | carbonyl]cyclohexyl]-difluoro- | butyl (2S,4S)- | 8.50-8.40 (m, 3H), |
| | methyl]-6-chloro-2- | 4-amino-2- | 7.93-7.88 (m, 2H), |
| | pyridyl]piperazin-1- | methyl- | 7.81-7.76 (m, 2H), |
| | yl]sulfonylphenyl]pyrrolidin-2-one | pyrrolidine-1- | 6.80-6.76 (m, 1H), |
| | | carboxylate | 6.71-6.68 (m, 1H), |
| | 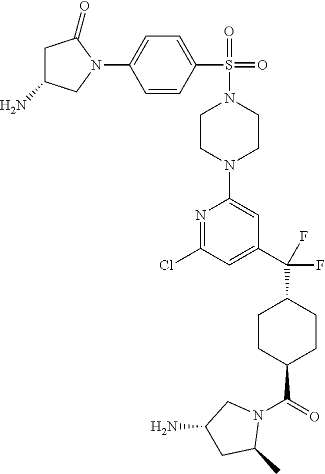 [SMILES: C[C@H]1C[C@H](N)CN1C(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 4.27-4.23 (m, 1H), 4.13-4.01 (m, 2H), 3.95-3.89 (m, 1H), 3.83-3.80 (m, 1H), 3.67-3.63 (m, 4H), 3.53-3.44 (m, 1H), 3.10-3.02 (m, 1H), 2.96 (br s, 4H), 2.70- 2.64 (m, 1H), 2.28- 2.16 (m, 2H), 2.14- 1.93 (m, 1H), 1.89- 1.73 (m, 2H), 1.71- 1.59 (m, 3H), 1.21 (br s, 3H), 1.20-1.05 (m, 5H). ¹⁹F NMR (376 MHz, DMSO-d₆) δ ppm −103.9-−103.6 (m, 2F). MS obsd. (ESI+) [(M + H)⁺]: 694.3. |
| 065 | Trans-bis(2-aminoethyl)-[3- | LINKER: Int- | ¹H NMR (400 MHz, |
| | [[4-[[2-[4-[4-[(4R)-4-amino-2- | 5 | DMSO-d₆) δ ppm- |
| | oxo-pyrrolidin-1- | TAIL: - | 0.14-0.01 (m, 1H), |
| | yl]phenyl]sulfonylpiperazin-1- | Aminopropyl- | 1.03-1.19 (m, 2H), |
| | yl]-6-chloro-4-pyridyl]- | bis[2-(tert- | 1.24-1.43 (m, 2H), |
| | difluoro- | butoxycarbonyl- | 1.63-1.90 (m, 6H), |
| | methyl]cyclohexanecarbonyl] | amino)ethyl]- | 1.99-2.26 (m, 2H), |
| | amino]propyl]-methyl- | methyl- | 2.58-2.70 (m, 1H), |
| | ammonium | ammonium | 2.97 (br s, 4H), 3.04 |
| | | | (br d, J = 8.44 Hz, 1H), |
| | 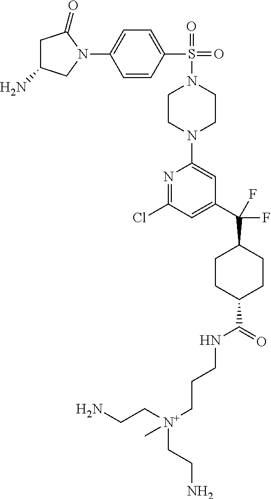 [SMILES: C[N+](CCN)(CCN)CCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 3.07-3.15 (m, 3H), 3.16-3.26 (m, 4H), 3.40-3.52 (m, 3H), 3.65 (br s, 8H), 3.89 (br d, J = 10.88 Hz, 1H), 3.97-4.11 (m, 1H), 4.21-4.31 (m, 1H), 6.71 (s, 1H), 6.79 (s, 1H), 7.79 (d, J = 8.80 Hz, 2H), 7.91 (d, J = 8.80 Hz, 2H), 8.09 (br s, 1H), 8.40- 8.69 (m, 9H). ¹⁹F NMR (376 MHz, DMSO-d₆) δ ppm −105.2-−103.8 (br, s, 2 F). MS obsd. (ESI+) [(M + H)⁺]: 768.4. |
| 066 | Trans-N-[(1R,3S)-3- | LINKER: Int- | ¹H NMR (400 MHz, |
| | aminocyclopentyl]-4-[2-[4-[4- | 5 | DMSO-d₆) δ ppm |
| | [(4R)-4-amino-2-oxo- | TAIL: Tert- | 8.70-8.61 (m, 3H), |
| | pyrrolidin-1- | butyl N- | 8.50-8.40 (m, 3H), |
| | yl]phenyl]sulfonylpiperazin-1- | [(1S,3R)-3- | 7.93-7.88 (m, 2H), |
| | yl]-6-chloro-4-pyridyl]- | aminocyclopentyl] | 7.81-7.76 (m, 2H), |
| | difluoro- | carbamate | 6.80-6.76 (m, 1H), |
| | methyl]cyclohexanecarboxamide | | 6.71-6.68 (m, 1H), |
| | | | 4.27-4.23 (m, 1H), |
| | 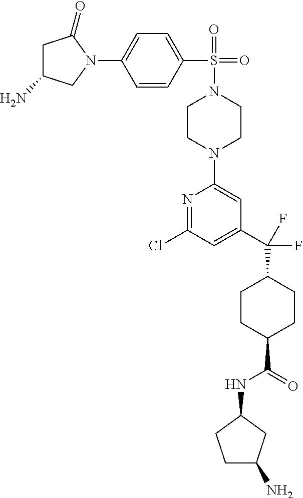 [SMILES: N[C@@H]1CC(=O)N(c2ccc(S(=O)(=O)N3CCN(c4cc(C(F)(F)[C@H]5CC[C@H](C(=O)N[C@@H]6CC[C@H](N)C6)CC5)cc(Cl)n4)CC3)cc2)C1] | | 4.13-4.01 (m, 2H), 3.95-3.89 (m, 1H), 3.83-3.80 (m, 1H), 3.67-3.63 (m, 4H), 3.53-3.44 (m, 1H), 3.10-3.02 (m, 1H), 2.96 (br s, 4H), 2.70- 2.64 (m, 1H), 2.28- 2.16 (m, 2H), 2.14- 1.93 (m, 1H), 1.89- 1.73 (m, 2H), 1.71- 1.59 (m, 3H), 1.21 (br s, 3H), 1.20-1.05 (m, 5H). ¹⁹F NMR (376 MHz, DMSO-d₆) δ ppm −103.9-−103.6 (m, 2 F). MS obsd. (ESI+) [(M + H)⁺]: 643.1. |
| 067 | Trans-(4R)-4-amino-1-[4-[4- | LINKER: Int- | ¹H NMR (376 MHz, |
| | [4-[[4-[cis-3,4- | 5 | DMSO-d₆) δ ppm 8.59 |
| | bis(aminomethyl)pyrrolidine- | TAIL: Cis- | (br d, J = 3.06 Hz, 3H), |
| | 1-carbonyl]cyclohexyl]- | tert-butyl N- | 8.09-8.34 (m, 6H), |
| | difluoro-methyl]-6-chloro-2- | [[4-[(tert- | 7.92 (d, J = 8.93 Hz, |
| | pyridyl]piperazin-1- | butoxycarbony- | 2H), 7.80 (d, |
| | yl]sulfonylphenyl]pyrrolidin-2-one | amino)methyl] | J = 8.80 Hz, 2H), 6.79 |
| | | pyrrolidin-3- | (s, 1H), 6.71 (s, 1H), |
| | 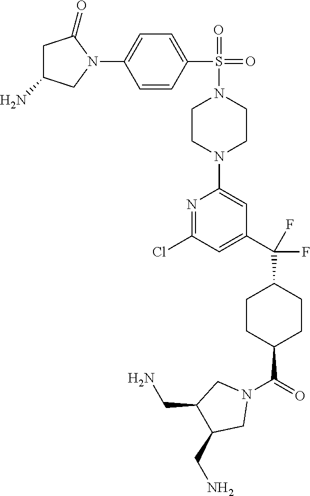 [SMILES: NC[C@@H]1CN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)C[C@@H]1CN] | yl]methyl] carbamate | 4.27 (br dd, J = 10.94, 6.79Hz, 1H), 4.07 (br s, 1H), 3.91 (br d, J = 10.15Hz, 1H), 3.78- 3.85 (m, 1H), 3.66 (br s, 5H), 3.39 (br s, 1H), 2.94-3.16 (m, 8H), 2.83 (br s, 2H), 2.63- 2.69 (m, 1H), 2.20- 2.45 (m, 4H), 1.66- 1.82 (m, 4H), 1.27- 1.41 (m, 2H), 1.08- 1.22 (m, 2H). ¹⁹F NMR (376 MHz, DMSO-d₆) δ ppm −104.00-−103.50 (m, 2 F). MS obsd. (ESI+) [(M + H)⁺]: 723.3. |
| 068 | Trans-4-[[2-[4-[4-[(4R)-4- | LINKER: Int-5 | ¹H NMR (500 MHz, |
| | amino-2-oxo-pyrrolidin-1- | TAIL: | DMSO-d₆) δ ppm 8.03 |
| | yl]phenyl]sulfonylpiperazin-1- | | (d, J = 7.2 Hz, 1H), 7.96- |
| | y1]-6-chloro-4-pyridy1]- | | 7.87 (m, J = 9.0 Hz, |
| | difluoro-methyl]-N-[(3R,4S)-4- | | 2H), 7.84-7.75 (m, J = |
| | methylpyrrolidin-3- | | 8.9 Hz, 2H), 6.79 (s, |
| | yl]cyclohexanecarboxamide | | 1H), 6.72 (s, 1H), 4.26 |
| | | | (dd, J = 6.9, 11.2 Hz, |
| | 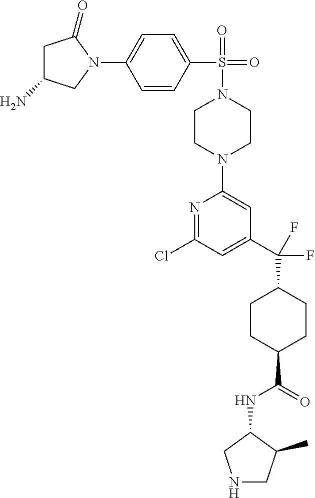 [SMILES: C[C@H]1CNC[C@@H]1NC(=O)[C@H]1CC[C@H](C(F)(F)c2cc(Cl)nc(N3CCN(S(=O)(=O)c4ccc(N5C[C@H](N)CC5=O)cc4)CC3)c2)CC1] | | 1H), 4.07 (br s, 1H), 3.93- 3.73 (m, 2H), 3.72- 3.59 (m, 4H), 3.13-3.01 (m, 1H), 3.00-2.94 (m, 4H), 2.85-2.73 (m, 2H), 2.57 (br dd, J = 2.6, 17.9 Hz, 2H), 2.26-2.16 (m, 1H), 2.12-2.02 (m, 2H), 1.75 (br t, J = 12.2 Hz, 2H), 1.66 (br d, J = 12.4 Hz, 2H), 1.43-1.24 (m, 2H), 1.17-1.09 (m, 2H), 0.97 (d, J = 6.7 Hz, 3H). ¹⁹F NMR (500 MHz, DMSO-d₆) δ ppm −108.94 (t, J = 20 Hz, 2 F). |
| 069 | Trans-N-(3-aminopropyl)-4- | LINKER: Int- | ¹H NMR (376 MHz, |
| | [[2-chloro-6-[4-[4-[(4R)-4- | 25 | DMSO-d₆) δ ppm |
| | (methylamino)-2-oxo- | TAIL: tert- | 7.65-7.85 (m, 4H), |
| | pyrrolidin-1- | butyl N-(3- | 6.68-6.70 (m, 2H), |
| | yl]phenyl]sulfonylpiperazin-1- | aminopropyl) | 4.41-4.46 (m, 1H), |
| | y1]-4-pyridyl]-difluoro- | carbamate | 4.18-4.23 (m, 1H), |
| | methyl]cyclohexanecarboxamide | | 4.10-4.15 (m, 1H), |
| | | | 3.45-3.65 (m, 4H), |
| | 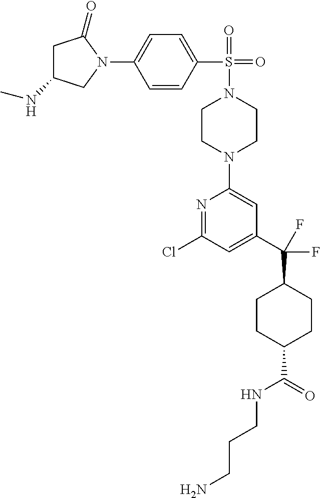 [SMILES: CN[C@@H]1CC(=O)N(c2ccc(S(=O)(=O)N3CCN(c4cc(C(F)(F)[C@H]5CC[C@H](C(=O)NCCCN)CC5)cc(Cl)n4)CC3)cc2)C1] | | 3.15-3.25 (m, 3H), 3.05-3.10 (m, 1H), 2.85-3.04 (m, 6H), 2.79 (s, 3H), 1.92-2.20 (m, 2H), 1.82 (q, J = 7.2 Hz, 3H), 1.60- 1.76 (m, 3H), 1.00- 1.36 (m, 4H). ¹⁹F NMR (376 MHz, DMSO-d₆) δ ppm −105.64-−102.83 (m, 2F). MS obsd. (ESI+) [(M + H)⁺]: 682.3. |

### Example 071: Trans-(4R)-4-amino-1-[4-[4-[4-[[4-[4-(2-aminoethyl)-4-methyl-piperazin-4-ium-1-carbonyl]cyclohexyl]-difluoro-methyl]-6-chloro-2-pyridyl]piperazin-1-yl]sulfonylphenyl]pyrrolidin-2-one

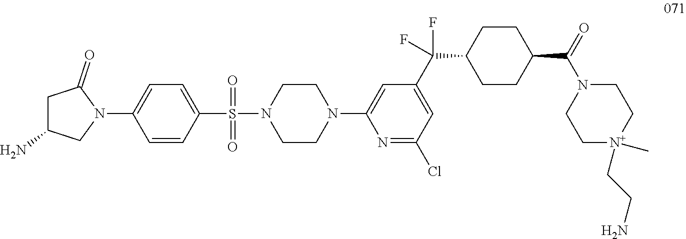 [SMILES: C[N+]1(CCN)CCN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)CC1]

### Step 1: Trans-tert-butyl N-[(3R)-1-[4-[4-[4-[[4-[4-[2-(tert-butoxycarbonylamino)ethyl]piperazine-1-carbonyl]cyclohexyl]-difluoro-methyl]-6-chloro-2-pyridyl]piperazin-1-yl]sulfonylphenyl]-5-oxo-pyrrolidin-3-yl]carbamate

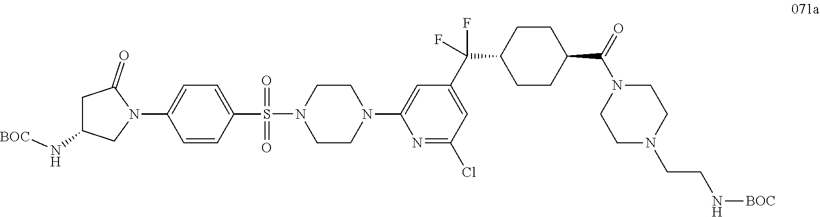 [SMILES: CC(C)(C)OC(=O)NCCN1CCN(C(=O)[C@H]2CC[C@H](C(F)(F)c3cc(Cl)nc(N4CCN(S(=O)(=O)c5ccc(N6C[C@H](NC(=O)OC(C)(C)C)CC6=O)cc5)CC4)c3)CC2)CC1]

A mixture of N-(2-piperazinoethyl)carbamic acid tert-butyl ester (72.45 mg, 0.316 mmol), compound Int-5 (150 mg, 0.211 mmol), HATU (160.07 mg, 0.421 mmol) and DIPEA (110.23 μL, 0.632 mmol) in DCM (15 mL) was stirred at room temperature for 2 hours. After completion, the mixture was diluted with EtOAc (50 mL), and washed with brine (50 mL). Then, the combined organic layer was dried over anhydrous sodium sulfate, filtered, concentrated in vacuo to give crude compound 071a (190 mg, 97.68%) as light yellow viscous oil. MS obsd. (ESI⁺) [(M+H)⁺]: 923.6.

### Step 2: Trans-tert-butyl N-[2-[4-[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]-1-methyl-piperazin-1-ium-1-yl]ethyl]carbamate

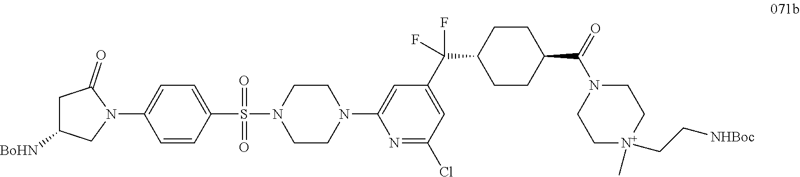

Compound 071a (100 mg, 0.108 mmol) and DIPEA (28.37 μL, 0.162 mmol) were dissolved in the DCM (5.15 mL). Into the stirring solution was added iodomethane (6.77 μL, 0.108 mmol). The reaction was stirred at room temperature overnight. Then, the reaction was concentrated in vacuo to give the desired product compound 071b (90 mg, 96 μmol, 88.56% yield) as light yellow oil, which was used directly in the next step. MS obsd. (ESI⁺) [(M+H)⁺]: 939.7.

### Step 3: Trans-(4R)-4-amino-1-[4-[4-[4-[[4-[4-(2-aminoethyl)-4-methyl-piperazin-4-ium-1-carbonyl]cyclohexyl]-difluoro-methyl]-6-chloro-2-pyridyl]piperazin-1-yl]sulfonylphenyl]pyrrolidin-2-one

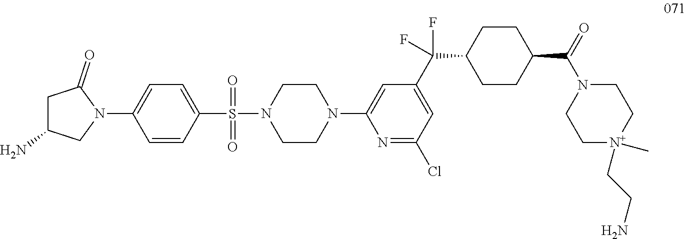 [SMILES: C[N+]1(CCN)CCN(C(=O)[C@H]2CC[C@H](C(F)(F)c3cc(Cl)nc(N4CCN(S(=O)(=O)c5ccc(N6C[C@H](N)CC6=O)cc5)CC4)c3)CC2)CC1]

Compound 071b (90 mg, 0.096 mmol) and TFA (0.5 mL, 6.5 mmol) were mixed in the DCM (5 mL). The reaction mixture was stirred at room temperature for 1 hour and went completed. After completion, the residue was concentrated in vacuo to give crude product, which was purified by prep-HPLC to give the desired product Example 071 (35 mg, 46.96%) as a white solid. MS obsd. (ESI⁺) [(M+H)⁺]: 737.4.

Example 071: ¹H NMR (400 MHz, METHANOL-d₄) δ ppm 8.43 (br s, 1H), 7.92 (m, J=9.05 Hz, 2H), 7.82 (m, J=9.05 Hz, 2H), 6.67 (d, J=7.34 Hz, 2H), 4.91-4.95 (m, 1H), 4.34 (dd, J=11.13, 6.85 Hz, 1H), 3.94-4.17 (m, 3H), 3.89 (br dd, J=11.07, 2.63 Hz, 2H), 3.79 (br d, J=15.16 Hz, 1H), 3.65-3.73 (m, 4H), 3.41-3.62 (m, 6H), 3.22-3.29 (m, 5H), 3.01-3.16 (m, 5H), 2.57-2.69 (m, 2H), 1.99-2.15 (m, 1H), 1.81 (br t, J=11.62 Hz, 4H), 1.45 (dt, J=6.69, 1.60 Hz, 2H), 1.25-1.39 (m, 2H). ¹⁹F NMR (376 MHz, METHANOL-d₄) δ ppm −106.86 (s, 1F), −106.89 (s, 1F).

### Example 072: Trans-N-[2-(2-aminoethylamino)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

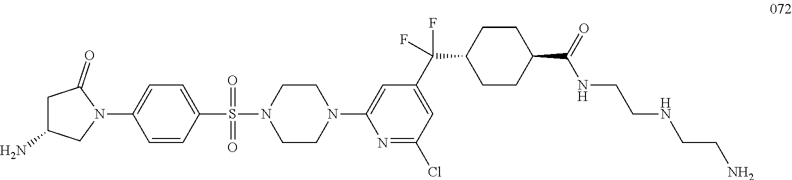 [SMILES: NCCNCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-tert-butyl N-[(3R)-1-[4-[4-[6-chloro-4-[difluoro-[4-(2-hydroxyethylcarbamoyl)cyclohexyl]methyl]-2-pyridyl]piperazin-1-yl]sulfonylphenyl]-5-oxo-pyrrolidin-3-yl]carbamate

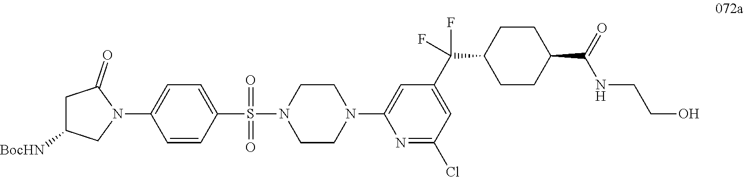 [SMILES: CC(C)(C)OC(=O)N[C@@H]1CC(=O)N(c2ccc(S(=O)(=O)N3CCN(c4cc(C(F)(F)[C@H]5CC[C@H](C(=O)NCCO)CC5)cc(Cl)n4)CC3)cc2)C1]

To a solution of compound Int-5 (300.0 mg, 0.420 mmol) in DMF (3 mL) were added HATU (208.21 mg, 0.550 mmol), DIPEA (0.22 mL, 1.26 mmol), 2-hydroxyethylamine (0.03 mL, 0.460 mmol) in one portion under nitrogen. The reaction was stirred at 25° C. for 16 hours. After completion, the reaction was diluted with water (20 mL), extracted with EtOAc (20 mL×3). The combined organic layer was washed with brine (50 mL), dried over anhydrous sodium sulfate and then filtered. The filtrate was concentrated in vacuo to give a crude residue, which was purified by prep-HPLC to afford compound 072a (220 mg, 0.290 mmol, 69.15% yield) as a white solid. MS obsd. (ESI⁺) [(M+H)]⁺: 755.6.

### Step 2: Trans-tert-butyl N-[(3R)-1-[4-[4-[6-chloro-4-[difluoro-[4-(2-oxoethylcarbamoyl)cyclohexyl]methyl]-2-pyridyl]piperazin-1-yl]sulfonylphenyl]-5-oxo-pyrrolidin-3-yl]carbamate

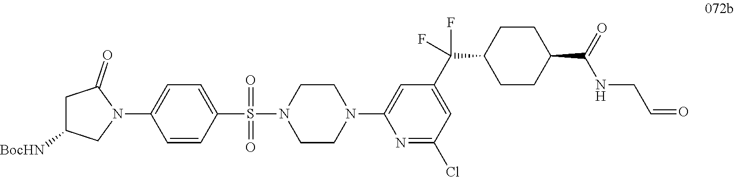 [SMILES: CC(C)(C)OC(=O)N[C@@H]1CC(=O)N(c2ccc(S(=O)(=O)N3CCN(c4cc(C(F)(F)[C@H]5CC[C@H](C(=O)NCC=O)CC5)cc(Cl)n4)CC3)cc2)C1]

To a solution of compound 072a (180.0 mg, 0.240 mmol) in DCM (3 mL) was added DMP (111.19 mg, 0.260 mmol) in one portion under nitrogen. The reaction was stirred at 25° C. for 1 hour. After completion, the reaction was quenched with sat. aq. Na₂CO₃ solution (100 mL) and extracted with EtOAc (200 mL). The organic layer was separated and dried over anhydrous sodium sulfate, filtered, and concentrated in vacuo to afford compound 072b (150 mg, 0.200 mmol, 83.56% yield) as a yellow solid, which was used directly for the next step. MS obsd. (ESI⁺) [(M+H)]⁺: 755.6.

### Step 3: Trans-tert-butyl N-[2-[2-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]ethylamino]ethyl] carbamate

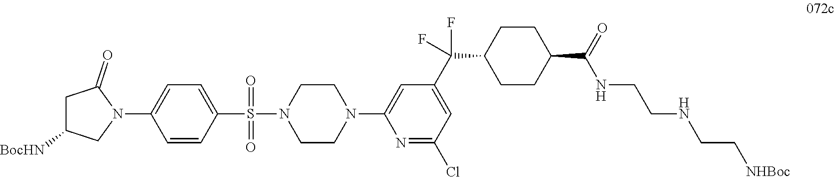 [SMILES: CC(C)(C)OC(=O)NCCNCCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound 072b (130.0 mg, 0.170 mmol), N-Boc-ethylenediamine (55.3 mg, 0.350 mmol) in methanol (1 mL) was stirred at 25° C. for 1 hour. Then sodium cynaoborohydride (54.23 mg, 0.860 mmol) was added and the reaction was stirred at 25° C. for additional 1 hour. After completion, the reaction was diluted with water (10 mL) and extracted with EtOAc (20 mL×2). The combined organic layer was washed with brine (30 mL), dried over anhydrous sodium sulfate, filtered, and concentrated in vacuo to give a residue, which was purified by prep-HPLC to afford compound 072c (85 mg, 0.090 mmol, 54.88% yield) as colorless oil. MS obsd. (ESI⁺) [(M+H)]⁺: 897.0.

### Step 4: Trans-N-[2-(2-aminoethylamino)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

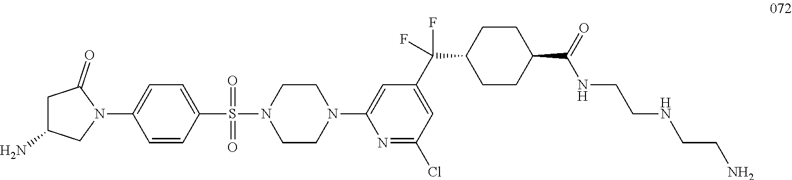 [SMILES: NCCNCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of compound 072c (35.0 mg, 0.040 mmol) in DCM (1 mL) was added TFA (0.5 mL, 6.5 mmol) in one portion. The reaction was stirred at 25° C. for 1 hour. After completion, the reaction was concentrated in vacuo to give a residue, which was purified by prep-HPLC to afford Example 072 (20 mg, 0.029 mmol, 71% yield) as a white solid. MS obsd. (ESI⁺) [(M+H)]⁺: 697.0.

Example 072: ¹H NMR (400 MHz, DMSO-d₆) δ ppm 8.34-8.26 (m, 3H), 8.21-8.12 (m, 3H), 8.10-8.03 (m, 1H), 7.94-7.90 (m, 2H), 7.82-7.78 (m, 2H), 6.81-6.77 (m, 1H), 6.73-6.69 (m, 1H), 4.28-4.22 (m, 1H), 4.10-4.04 (m, 1H), 3.87-3.82 (m, 1H), 3.68-3.63 (m, 4H), 3.21-3.14 (m, 4H), 3.14-3.05 (m, 2H), 3.05-3.00 (m, 2H), 2.97 (br d, J=2.8 Hz, 4H), 2.61 (br s, 1H), 2.21-1.98 (m, 2H), 1.87-1.78 (m, 2H), 1.70-1.63 (m, 2H), 1.39-1.25 (m, 2H), 1.19-1.08 (m, 2H). ¹⁹F NMR (376 MHz, DMSO-d₆) δ ppm −104.086 (br s, 2F).

### Example 073: Trans-2-aminoethyl-[2-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]amino]ethyl]-dimethyl-ammonium

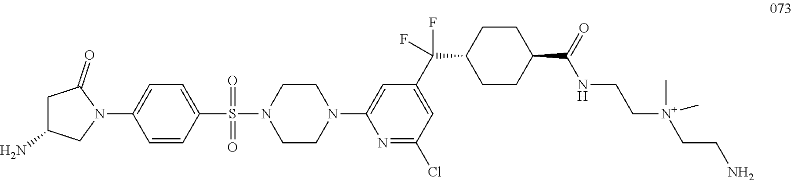 [SMILES: C[N+](C)(CCN)CCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-2-(tert-butoxycarbonylamino)ethyl-[2-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]ethyl]-dimethyl-ammonium

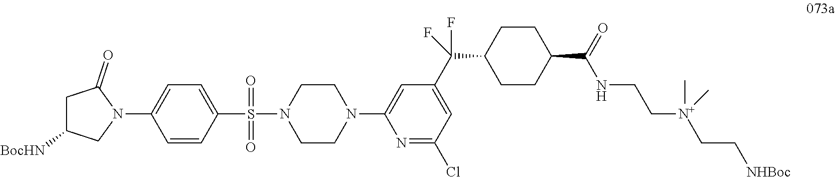 [SMILES: CC(C)(C)OC(=O)NCC[N+](C)(C)CCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound 072c (35.0 mg, 0.040 mmol) in DCM (1 mL) were added sodium hydrogen carbonate (32.76 mg, 0.390 mmol) and methyl trifluoromethane-sulfonate (19.2 mg, 0.120 mmol) in one portion. The reaction was stirred at 25° C. for 1 hour. After completion, the reaction was filtered and the filtrate was added with NH₃·H₂O. The reaction was concentrated in vacuo to give compound 073a (28 mg, 0.030 mmol, 77.49% yield) as a white solid. MS obsd. (ESI⁺) [(M+H)]⁺: 925.4.

### Step 2: Trans-2-aminoethyl-[2-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]amino]ethyl]-dimethyl-ammonium

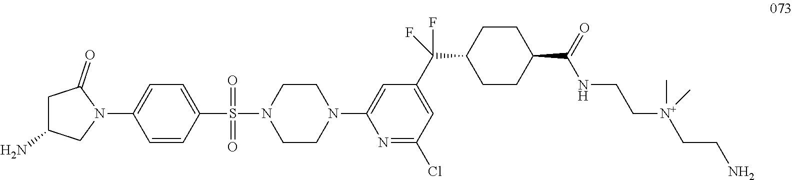 [SMILES: C[N+](C)(CCN)CCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of compound 073a (41.0 mg, 0.040 mmol) in DCM (1 mL) was added TFA (0.17 mL, 2.21 mmol) in one portion. The reaction was stirred at 25° C. for 1 hour. After completion, the reaction was concentrated in vacuo to give a residue, which was purified by prep-HPLC to give Example 073 (20 mg, 0.030 mmol, 58.33% yield) as a white solid. MS obsd. (ESI⁺) [(M+H)]⁺: 725.1.

Example 073: ¹H NMR (400 MHz, DMSO-d₆) δ ppm 8.52-8.41 (m, 5H), 8.33-8.24 (m, 1H), 7.96-7.88 (m, 2H), 7.79 (d, J=9.0 Hz, 2H), 6.78 (s, 1H), 6.71 (s, 1H), 4.31-4.22 (m, 1H), 4.11-4.02 (m, 1H), 3.90-3.85 (m, 1H), 3.69-3.57 (m, 6H), 3.48-3.41 (m, 2H), 3.38 (br s, 2H), 3.15 (s, 6H), 3.10-3.04 (m, 1H), 2.97 (br s, 4H), 2.66-2.59 (m, 1H), 2.24-2.05 (m, 2H), 1.82-1.73 (m, 2H), 1.67 (br d, J=10.1 Hz, 2H), 1.39-1.26 (m, 2H), 1.14 (br s, 2H). ¹⁹F NMR (376 MHz, DMSO-d₆) δ ppm −104.004 (br s, 2F).

### Example 074: Trans-N-[2-[1-(2-aminoethyl)pyrrolidin-1-ium-1-yl]ethyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide

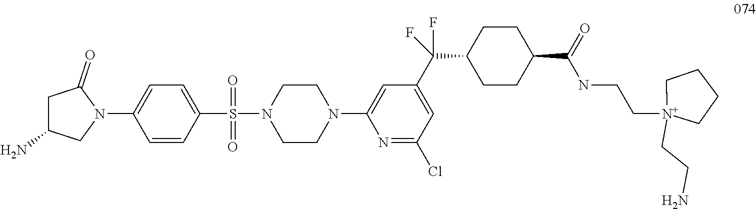 [SMILES: NCC[N+]1(CCNC(=O)[C@H]2CC[C@H](C(F)(F)c3cc(Cl)nc(N4CCN(S(=O)(=O)c5ccc(N6C[C@H](N)CC6=O)cc5)CC4)c3)CC2)CCCC1]

### Step 1: Trans-tert-butyl N-[(3R)-1-[4-[4-[6-chloro-4-[difluoro-[4-(2-pyrrolidin-1-ylethylcarbamoyl)cyclohexyl]methyl]-2-pyridyl]piperazin-1-yl]sulfonylphenyl]-5-oxo-pyrrolidin-3-yl]carbamate

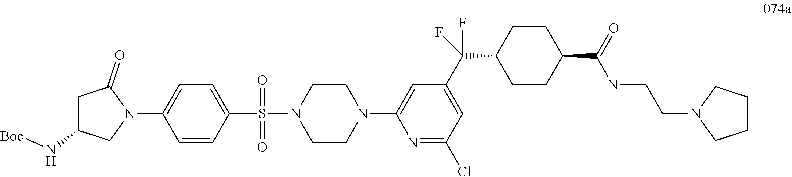 [SMILES: CC(C)(C)OC(=O)N[C@@H]1CC(=O)N(c2ccc(S(=O)(=O)N3CCN(c4cc(C(F)(F)[C@H]5CC[C@H](C(=O)NCCN6CCCC6)CC5)cc(Cl)n4)CC3)cc2)C1]

A mixture of 2-pyrrolidinoethylamine (24.05 mg, 0.211 mmol), compound Int-5 (100 mg, 0.140 mmol), HATU (106.71 mg, 0.281 mmol) and DIPEA (73.49 μL, 0.421 mmol) in DCM (10 mL) was stirred at room temperature for 2 hours. After completion, the mixture was diluted with EtOAc (10 mL), washed with water (10 mL) and brine (10 mL). Then, the combined organic layer was dried over anhydrous sodium sulfate, filtered, concentrated in vacuo to give crude 074a (100 mg, 88.1% yield) as yellow viscous oil. MS obsd. (ESI⁺) [(M+H)⁺]: 808.7.

### Step 2: Trans-tert-butyl N-[2-[1-[2-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]ethyl]pyrrolidin-1-ium-1-yl]ethyl]carbamate

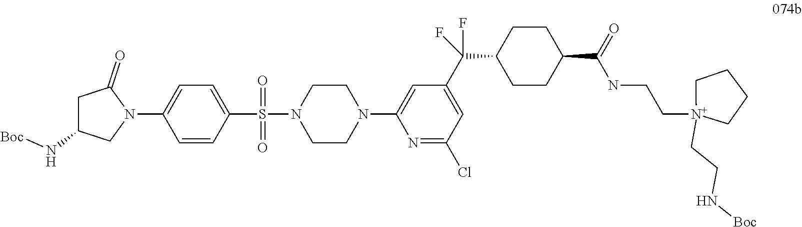 [SMILES: CC(C)(C)OC(=O)NCC[N+]1(CCNC(=O)[C@H]2CC[C@H](C(F)(F)c3cc(Cl)nc(N4CCN(S(=O)(=O)c5ccc(N6C[C@H](NC(=O)OC(C)(C)C)CC6=O)cc5)CC4)c3)CC2)CCCC1]

Compound 074a (100 mg, 0.124 mmol) and N-(2-bromoethyl)carbamic acid tert-butyl ester (138.61 mg, 0.619 mmol) were dissolved in the anhydrous MeCN (8 mL). Into the stirring solution was added DIPEA (64.81 μL, 0.371 mmol) and 0.1 mg DMAP. The reaction mixture was stirred at 90° C. overnight. LCMS showed half of the staring material was converted. Then, the reaction was cooled to room temperature and concentrated in vacuo to give crude compound 074b (57 mg, 49%) as yellow oil, which was used directly in the next step. MS obsd. (ESI⁺) [(M+H)⁺]: 951.5.

### Step 3: Trans-N-[2-[1-(2-aminoethyl)pyrrolidin-1-ium-1-yl]ethyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide

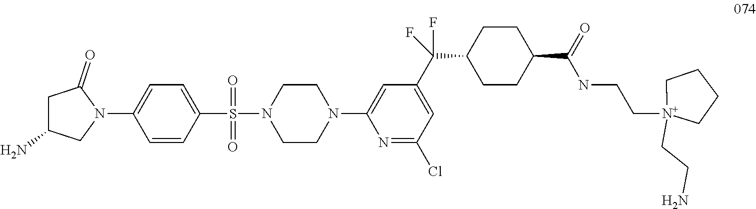 [SMILES: NCC[N+]1(CCNC(=O)[C@H]2CC[C@H](C(F)(F)c3cc(Cl)nc(N4CCN(S(=O)(=O)c5ccc(N6C[C@H](N)CC6=O)cc5)CC4)c3)CC2)CCCC1]

Compound 074b (100 mg, 0.105 mmol) and TFA (1 mL, 13 mmol) were dissolved in the DCM (10 mL). The reaction was stirred at room temperature for 2 hours. After completion, the reaction was concentrated in vacuo and purified by prep-HPLC to give the desired product Example 074 (18.1 mg, 20.63%) as a white solid. MS obsd. (ESI⁺) [(M+H)⁺]: 751.8.

Example 074: ¹H NMR (400 MHz, METHANOL-d₄) δ ppm 7.83 (m, J=8.93 Hz, 2H), 7.73 (m, J=8.93 Hz, 2H), 6.57 (d, J=7.70 Hz, 2H), 4.30 (dd, J=11.43, 7.03 Hz, 1H), 4.06-4.13 (m, 1H), 3.86 (dd, J=11.43, 2.51 Hz, 1H), 3.37-3.64 (m, 14H), 3.29-3.35 (m, 2H), 3.05-3.17 (m, 1H), 2.98 (br t, J=4.89 Hz, 4H), 2.60 (dd, J=18.10, 3.06 Hz, 1H), 1.89-2.17 (m, 7H), 1.78 (br d, J=11.13 Hz, 2H), 1.70 (br d, J=11.00 Hz, 2H), 1.09-1.37 (m, 8H). ¹⁹F NMR (376 MHz, METHANOL-d₄) δ ppm −106.97 (s, 1F), −107.01 (s, 1F).

### Example 075: Trans-N-[2-[(2-aminoacetyl)amino]ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

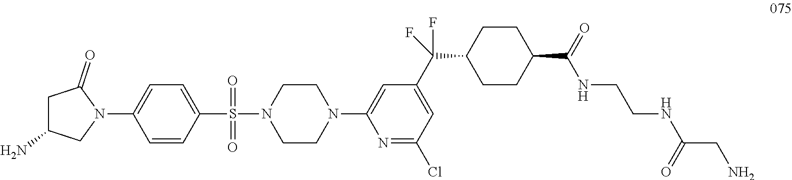 [SMILES: NCC(=O)NCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-tert-butyl N-[(3R)-1-[4-[4-[4-[[4-(2-aminoethylcarbamoyl)cyclohexyl]-difluoro-methyl]-6-chloro-2-pyridyl]piperazin-1-yl]sulfonylphenyl]-5-oxo-pyrrolidin-3-yl]carbamate

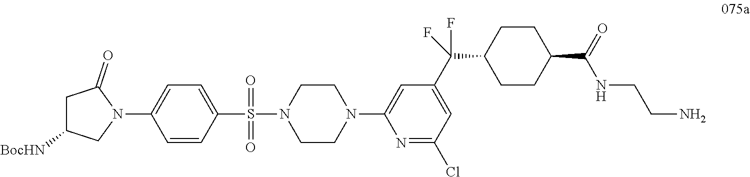 [SMILES: CC(C)(C)OC(=O)N[C@@H]1CC(=O)N(c2ccc(S(=O)(=O)N3CCN(c4cc(C(F)(F)[C@H]5CC[C@H](C(=O)NCCN)CC5)cc(Cl)n4)CC3)cc2)C1]

To a solution of compound Int-5 (100.0 mg, 0.140 mmol) in DMF was added DIPEA (52.23 mg, 0.420 mmol) and HATU (80.09 mg, 0.210 mmol). The mixture was stirred at 25° C. for 1 hour. Then the mixture was added ethylenediamine (337 mg, 5.6 mmol) in DMF (3 mL). After stirring at 25° C. for 1 hour, the mixture was diluted with EtOAc (50 mL) and water (30 mL). The organic phase was washed with brine (30 mL), dried over anhydrous sodium sulfate, filtered and concentrated in vacuo to give crude compound 075a (100 mg, 0.130 mmol, 94.42% yield) as a white solid. The residue was used directly in the next step. MS obsd. (ESI⁺) [(M+H)⁺]: 754.3.

### Step 2: Trans-tert-butyl N-[2-[2-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]ethylamino]-2-oxo-ethyl]carbamate

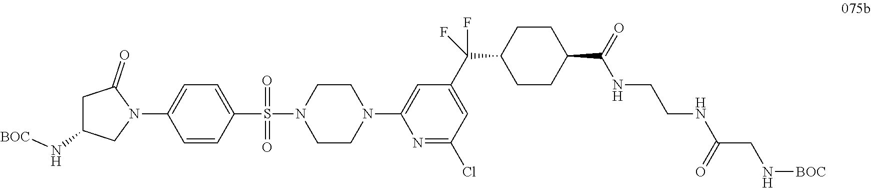 [SMILES: CC(C)(C)OC(=O)NCC(=O)NCCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound 075a (100.0 mg, 0.130 mmol) and BOC-glycine (23.22 mg, 0.130 mmol) in DMF (1 mL) was added DIPEA (49.32 mg, 0.400 mmol,) and HATU (46.79 mg, 0.200 mmol). After stirring at 25° C. for 1 hour, the mixture was diluted with EtOAc (50 mL) and water (30 mL). The organic phase was washed with brine (50 mL), dried over sodium sulfate, filtered and concentrated in vacuo to give a crude residue, which was purified by prep-TLC (PE:EA=1:1) to give compound 075b (95 mg, 0.100 mmol, 78.62% yield) as a white solid. MS obsd. (ESI⁺) [(M+H)⁺]: 911.5.

### Step 3: Trans-N-[2-[(2-aminoacetyl)amino]ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

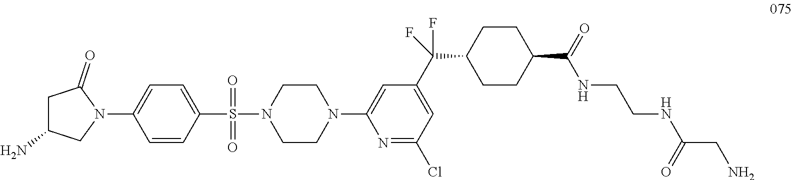 [SMILES: NCC(=O)NCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of compound 075b (90.0 mg, 0.100 mmol) in DCM (5 mL) was added TFA (56.28 mg, 0.490 mmol). The mixture was stirred at 25° C. for 2 hours. After completion, the mixture was concentrated in vacuo to give a crude residue, which was purified by prep-HPLC to afford Example 075 (22.7 mg, 0.030 mmol, 30.8% yield) as a white solid. MS obsd. (ESI⁺) [(M+H)]⁺: 711.2.

Example 075: ¹H NMR (400 MHz, DMSO-d₆) δ ppm 8.50 (d, J=12.47 Hz, 4H), 8.11 (s, 3H), 7.91 (d, J=8.93 Hz, 3H), 7.80 (d, J=8.93 Hz, 2H), 6.78 (s, 1H), 6.71 (s, 1H), 4.21-4.32 (m, 1H), 4.07 (s, 1H), 3.89 (d, J=9.29 Hz, 1H), 3.66 (s, 5H), 2.91-3.31 (m, 8H), 2.64 (dd, J=17.91, 2.75 Hz, 1H), 2.17 (t, J=11.98 Hz, 1H), 1.97-2.08 (m, 1H), 1.59-1.83 (m, 4H), 1.22-1.39 (m, 2H), 1.02-1.19 (m, 2H). ¹⁹F NMR (376 MHz, DMSO-d₆) δ ppm −105.48-−102.95 (m, 2F).

### Example 076: Trans-N-(3-amino-2-methoxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

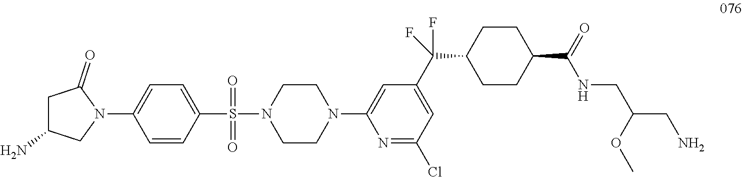 [SMILES: COC(CN)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-tert-butyl N-[3-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]-2-hydroxy-propyl]carbamate

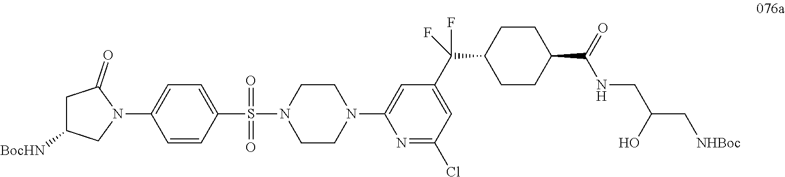 [SMILES: CC(C)(C)OC(=O)NCC(O)CNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound Int-5 (250.0 mg, 0.350 mmol) in DMF (4 mL) was added tert-butyl N-(3-amino-2-hydroxy-propyl)carbamate (133.56 mg, 0.700 mmol), HATU (200.21 mg, 0.530 mmol) and DIPEA (136.1 mg, 1.05 mmol). The reaction mixture was stirred at 20° C. for 1 hour. After completion, the reaction mixture was diluted with EtOAc (200 mL) and the organic layer was washed with brine (30 mL×3), dried over anhydrous sodium sulfate, filtered and concentrated in vacuo to give a crude residue, which was purified by reverse-phase column to give compound 076a (260 mg, 0.290 mmol, 83.75% yield) as a light yellow solid. MS obsd. (ESI⁺) [(M+H)⁺]: 886.5.

### Step 2: Trans-tert-butyl N-[3-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]-2-methoxy-propyl]carbamate

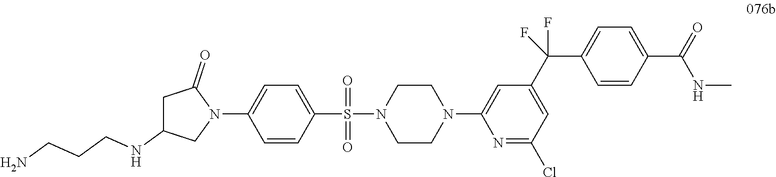 [SMILES: CNC(=O)c1ccc(C(F)(F)c2cc(Cl)nc(N3CCN(S(=O)(=O)c4ccc(N5CC(NCCCN)CC5=O)cc4)CC3)c2)cc1]

To a solution of compound 076a (150.0 mg, 0.170 mmol) in MeCN (2 mL) was added Ag₂O (589.52 mg, 2.54 mmol) and MeI (240.73 mg, 1.7 mmol) at 20° C. The reaction was stirred at 20° C. for 16 hours. Then, the reaction mixture was concentrated in vacuo and purified by prep-TLC (PE:EA=1:3) to give compound 076b (40 mg, 0.040 mmol, 26.25% yield) as a light yellow solid. MS obsd. (ESI⁺) [(M+H)⁺]: 900.2.

### Step 3: Trans-N-(3-amino-2-methoxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide

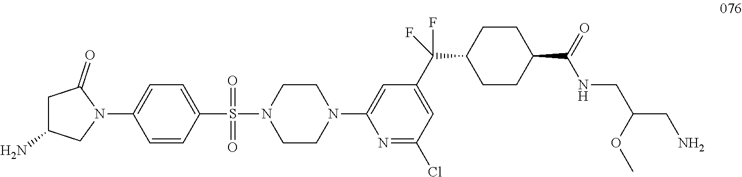 [SMILES: COC(CN)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of compound 076 (60.0 mg, 0.070 mmol) in DCM (2 mL) was added TFA (761.44 mg, 6.68 mmol) at 30° C. The reaction mixture was stirred at 30° C. for 1 hour. After completion, the reaction mixture was concentrated in vacuo to give a crude residue, which was purified by prep-HPLC to give Example 076 (23.9 mg, 0.030 mmol, 49.77% yield) as a white solid. MS obsd. (ESI⁺) [(M+H)⁺]: 700.2.

Example 076: ¹H NMR (400 MHz, DEUTERIUM OXIDE) δ ppm 7.69 (br d, J=8.6 Hz, 2H), 7.56 (br d, J=8.6 Hz, 2H), 6.56 (s, 2H), 4.39-4.11 (m, 2H), 4.10-3.95 (m, 1H), 3.57 (br s, 1H), 3.47 (br s, 4H), 3.35 (s, 3H), 3.29 (br d, J=4.2 Hz, 2H), 3.22-3.01 (m, 2H), 2.88-2.68 (m, 6H), 2.07-1.74 (m, 2H), 1.69-1.41 (m, 4H), 1.19-0.86 (m, 4H). ¹⁹F NMR (400 MHz, DEUTERIUM OXIDE) δ ppm: −105.02 (br s, 2F).

### Example 077: Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2,3-diaminopropyl)cyclohexanecarboxamide

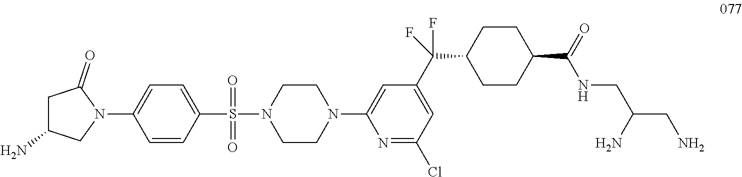 [SMILES: NCC(N)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-tert-butyl N-[3-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]-2-oxo-propyl]carbamate

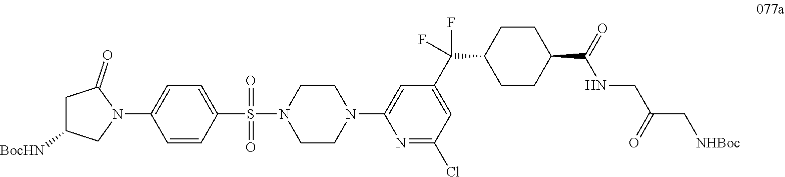 [SMILES: CC(C)(C)OC(=O)NCC(=O)CNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound 076a (100.0 mg, 0.110 mmol) in THF (2 mL) was added DMP (143.87 mg, 0.340 mmol) at 20° C. The reaction was stirred at 20° C. for 2 hours. After completion, the reaction mixture was purified by prep-TLC (PE:EtOAc=1:3) to give 077a (50 mg, 0.060 mmol, 50.11% yield) as a white solid. MS obsd. (ESI⁺) [(M+H)⁺]: 882.4.

### Step 2: Trans-tert-butyl N-[(3R)-1-[4-[4-[6-chloro-4-[difluoro-[4-[[(2E)-3-(tert-butoxycarbonylamino)-2-hydroxyimino-propyl]carbamoyl]cyclohexyl]methyl]-2-pyridyl]piperazin-1-yl]sulfonylphenyl]-5-oxo-pyrrolidin-3-yl]carbamate

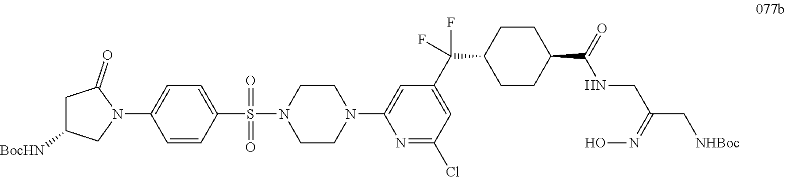 [SMILES: CC(C)(C)OC(=O)NC/C(CNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1)=N/O]

To a solution of compound 077a (120.0 mg, 0.140 mmol) in ethanol (2 mL) was added hydroxylamine hydrochloride (18.9 mg, 0.270 mmol) at 30° C. The reaction was stirred at 30° C. for 2 hours. After completion, the mixture was diluted with EtOAc (200 mL) and washed with brine (50 mL). The organic layer was dried over anhydrous sodium sulfate, filtered and concentrated in vacuo to give compound 077b (122 mg, 0.140 mmol, 99.97% yield) as a white solid. MS obsd. (ESI⁺) [(M+H)⁺]: 897.0.

### Step 3: Trans-tert-butyl N-[2-amino-3-[[4-[[2-chloro-6-[4-[4-[(4R)-4-(tert-butoxycarbonylamino)-2-oxo-pyrrolidin-1-yl]phenyl]sulfonylpiperazin-1-yl]-4-pyridyl]-difluoro-methyl]cyclohexanecarbonyl]amino]propyl]carbamate

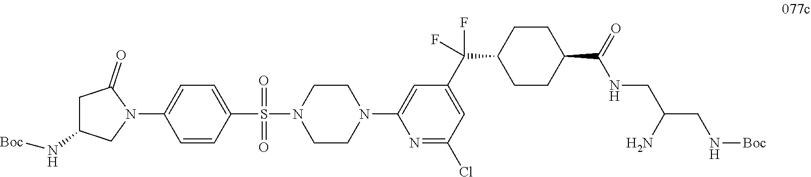 [SMILES: CC(C)(C)OC(=O)NCC(N)CNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of compound 077b (120.0 mg, 0.130 mmol) in ethanol (10 mL) was added Pt/C (500.0 mg, 7.69 mmol) at 30° C. The reaction was stirred at 30° C. for 24 hours under H₂. After completion, the reaction mixture was filtered and the filtrate was concentrated in vacuo to give a crude residue, which was purified by prep-TLC to afford compound 077c (30 mg, 0.030 mmol, 25.4% yield) as a white solid. MS obsd. (ESI⁺) [(M+H)⁺]: 883.0.

### Step 4: Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2,3-diaminopropyl)cyclohexanecarboxamide

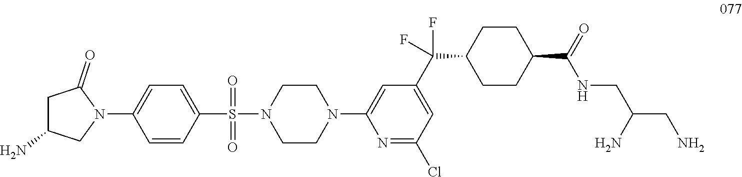 [SMILES: NCC(N)CNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of compound 077c (30.0 mg, 0.030 mmol) in DCM (1 mL) was added TFA (1.0 mL, 12.98 mmol) in one portion. After addition, the mixture was stirred at 25° C. for 30 minutes. After completion, the reaction was concentrated in vacuo and then purified by prep-HPLC to afford Example 077 (10.0 mg, 0.010 mmol, 37.67% yield) as a white powder. MS obsd. (ESI⁺) [(M+H)⁺]: 683.2.

Example 077: ¹H NMR (400 MHz, DMSO-d₆) δ ppm 8.45-8.03 (m, 7H), 7.95-7.85 (d, 2H), 7.80-7.60 (d, 2H), 6.78 (s, 1H), 6.71 (s, 1H), 4.30-4.20 (m, 2H), 3.80-3.73 (m, 1H), 3.70-3.62 (m, 4H), 3.20-2.95 (m, 9H), 2.65-2.52 (m, 2H), 2.25-2.05 (m, 2H), 1.90-1.75 (m, 2H), 1.74-1.60 (m, 2H), 1.40-1.26 (m, 2H), 1.35-1.08 (m, 2H). ¹⁹F NMR (376 MHz, DMSO-d₆) δ ppm −104.025 (br s, 2F).

### Example 078: 4-[[2-[4-[4-[4-(3-Aminopropylamino)-2-oxo-pyrrolidin-1-yl]phenyl]sulfonylpiperazin-1-yl]-6-chloro-4-pyridyl]-difluoro-methyl]-N-methyl-benzamide

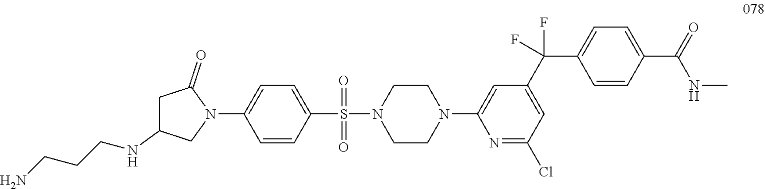 [SMILES: CNC(=O)c1ccc(C(F)(F)c2cc(Cl)nc(N3CCN(S(=O)(=O)c4ccc(N5CC(NCCCN)CC5=O)cc4)CC3)c2)cc1]

### Step 1: 4-[[2-[4-[4-[4-[Tert-butoxycarbonyl-[3-(tert-butoxycarbonylamino)propyl]-amino]-2-oxo-pyrrolidin-1-yl]phenyl]sulfonylpiperazin-1-yl]-6-chloro-4-pyridyl]-difluoro-methyl]benzoic acid

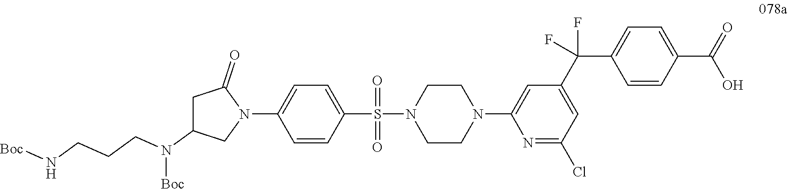 [SMILES: CC(C)(C)OC(=O)NCCCN(C(=O)OC(C)(C)C)C1CC(=O)N(c2ccc(S(=O)(=O)N3CCN(c4cc(C(F)(F)c5ccc(C(=O)O)cc5)cc(Cl)n4)CC3)cc2)C1]

Compound 078a was prepared in analogy to the preparation of compound Int-12 by using compound Int-22 instead of compound Int-2 in Step 4. Compound Int-078a (235.3 mg) was obtained as a white solid. MS obsd. (ESI⁺) [(M+H)⁺]: 863.3.

### Step 2: Tert-butyl N-[3-(tert-butoxycarbonylamino)propyl]-N-[1-[4-[4-[6-chloro-4-[difluoro-[4-(methylcarbamoyl)phenyl]methyl]-2-pyridyl]piperazin-1-yl]sulfonylphenyl]-5-oxo-pyrrolidin-3-yl]carbamate

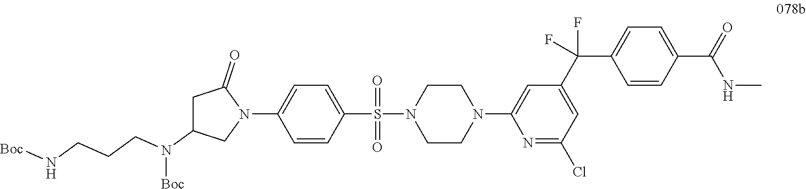 [SMILES: CNC(=O)c1ccc(C(F)(F)c2cc(Cl)nc(N3CCN(S(=O)(=O)c4ccc(N5CC(N(CCCNC(=O)OC(C)(C)C)C(=O)OC(C)(C)C)CC5=O)cc4)CC3)c2)cc1]

A mixture of compound 078a (61 mg, 70.8 μmol), methanamine hydrochloride (14.2 mg, 212 μmol), HATU (40.4 mg, 106 μmol), and TEA (21.5 mg, 212 μmol) in DCM (3 mL) was stirred at room temperature for 12 hours. After completion, the residue was washed with water (50 mL). The organic layer was dried over anhydrous sodium sulfate, filtered, and concentrated in vacuo to give a crude product compound 078b (50 mg, 0.571 mmol, 80% yield) as yellow oil. MS obsd. (ESI⁺) [(M+H)⁺]: 876.3.

### Step 4: 4-[[2-[4-[4-[4-(3-aminopropylamino)-2-oxo-pyrrolidin-1-yl]phenyl]sulfonylpiperazin-1-yl]-6-chloro-4-pyridyl]-difluoro-methyl]-N-methyl-benzamide

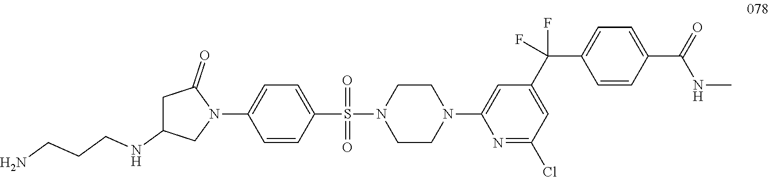 [SMILES: CNC(=O)c1ccc(C(F)(F)c2cc(Cl)nc(N3CCN(S(=O)(=O)c4ccc(N5CC(NCCCN)CC5=O)cc4)CC3)c2)cc1]

A mixture of compound 078b (34.5 mg, 39.4 μmol) and TFA (89.8 mg, 787 μmol) in DCM (3 mL) was stirred at room temperature for 1 hour. After completion, the reaction mixture was concentrated in vacuo to give a crude residue, which was purified by prep-HPLC to give Example 078 (6.8 mg, 0.01 mmol, 11% yield) as a white solid. MS obsd. (ESI⁺) [(M+H)⁺]: 676.1.

Example 078: ¹H NMR (400 MHz, METHANOL-d₄) δ ppm 1.64-1.85 (m, 2H), 2.54-2.60 (m, 1H), 2.67 (br d, J=1.83 Hz, 1H), 2.72-2.89 (m, 7H), 2.96 (m, 4H), 3.67 (m, 4H), 4.09 (m, 1H), 6.79 (s, 1H), 6.96 (s, 1H), 7.68 (d, J=8.44 Hz, 2H), 7.77 (d, J=8.93 Hz, 2H), 7.91 (t, J=8.50 Hz, 4H), 8.53-8.58 (m, 1H). ¹⁹F NMR (376 MHz, DMSO-d₆) δ ppm −92.70 (s, 2F).

### Example 079: Trans-N-[(1R,3S)-3-[(2-aminoacetyl)amino]cyclopentyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide

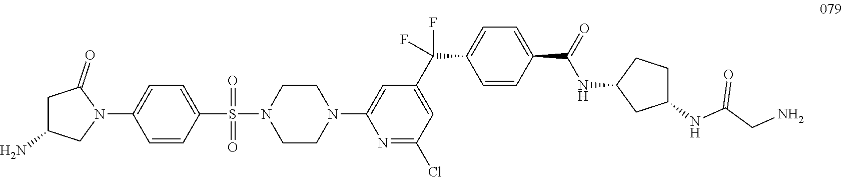

The title compound was prepared in analogy to the preparation of Example 075 by using (1S,3R)-cyclopentane-1,3-diamine instead of ethylenediamine in Step 1. Example 079 (24 mg) was obtained as a white solid. MS obsd. (ESI⁺) [(M+H)⁺]: 751.4. ¹H NMR (500 MHz, DMSO-d₆) δ ppm 8.35-8.45 (m, 1H), 8.16-8.28 (m, 2H), 7.87-8.00 (m, 2H), 7.73-7.85 (m, 2H), 6.77-6.86 (m, 1H), 6.61-6.76 (m, 1H), 4.20-4.33 (m, 1H), 4.05-4.15 (m, 1H), 3.87-4.05 (m, 3H), 3.74-3.87 (m, 2H), 3.60-3.74 (m, 5H), 3.02-3.16 (m, 2H), 2.92-3.02 (m, 4H), 2.13-2.27 (m, 2H), 1.93-2.03 (m, 1H), 1.78-1.93 (m, 3H), 1.59-1.78 (m, 5H), 1.40-1.59 (m, 3H), 1.17-1.40 (m, 4H), 0.94-1.17 (m, 3H). ¹⁹F NMR (471 MHz, DMSO-d₆) δ ppm −104.30 (br s, 1F).

### Example 080: Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[cis-3,4-bis(aminomethyl)pyrrolidin-1-yl]cyclopentyl]cyclohexanecarboxamide

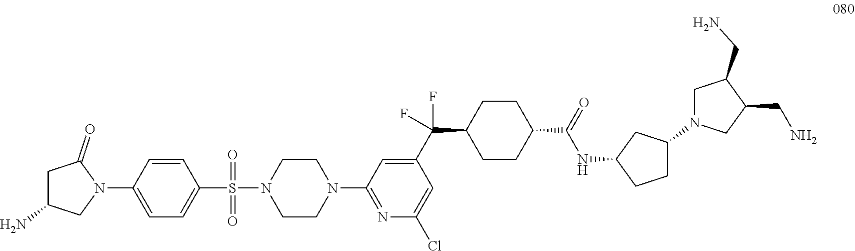 [SMILES: NC[C@@H]1CN([C@@H]2CC[C@H](NC(=O)[C@H]3CC[C@H](C(F)(F)c4cc(Cl)nc(N5CCN(S(=O)(=O)c6ccc(N7C[C@H](N)CC7=O)cc6)CC5)c4)CC3)C2)C[C@@H]1CN]

### Step 1: Benzyl N-[(1S)-3-oxocyclopentyl]carbamate

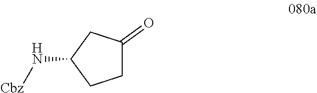 [SMILES: O=C1CC[C@H](NC(=O)OCc2ccccc2)C1]

To a solution of oxalyl chloride (0.81 g, 6.38 mmol) in DCM (10 mL) was added a solution of DMSO (0.73 g, 9.35 mmol) in DCM (10 mL) at −78° C. under N₂. The reaction was stirred at −78° C.° C. for 1 hour, and then the solution of benzyl N-[(1S)-3-hydroxycyclopentyl]-carbamate (1.0 g, 4.25 mmol) in DCM (10 mL) was added. After stirring at −78° C. for 1 hour, DIEA (1.72 g, 17 mmol) was added at −78° C. and then the reaction was allowed to warm to 20° C. After the starting material was consumed, the reaction mixture was quenched with water (100 mL) and extracted with DCM (100 mL×3).The combined organic layer was washed with brine (200 mL), dried over anhydrous sodium sulfate, and concentrated in vacuo to give the crude product, which was purified by silica gel column (elute with EtOAc:PE=30%) to afford 080a (820 mg, 3.52 mmol, 82.71% yield) as a white solid. MS obsd. (ESI⁺) [(M+H)⁺]: 234.0.

### Step 2: Tert-butyl N-[[cis-1-[(1R,3S)-3-(benzyloxycarbonylamino)cyclopentyl]-4-[(tert-butoxycarbonylamino)methyl]pyrrolidin-3-yl]methyl]carbamate

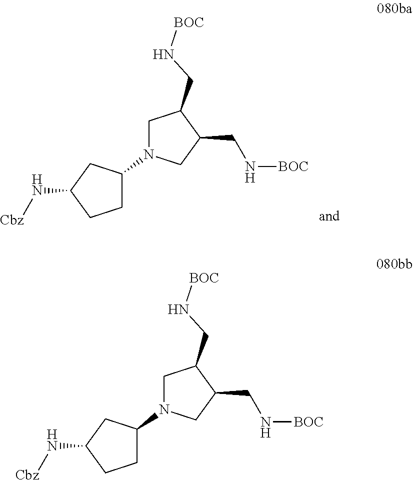 [SMILES: CC(C)(C)OC(=O)NC[C@@H]1CN([C@@H]2CC[C@H](NC(=O)OCc3ccccc3)C2)C[C@@H]1CNC(=O)OC(C)(C)C ; CC(C)(C)OC(=O)NC[C@@H]1CN([C@H]2CC[C@H](NC(=O)OCc3ccccc3)C2)C[C@@H]1CNC(=O)OC(C)(C)C]

To a solution of 080a (30.0 mg, 0.130 mmol) and tert-butyl N-[[cis-4-[(tert-butoxycarbonylamino)methyl]pyrrolidin-3-yl]methyl]carbamate (42.37 mg, 0.130 mmol) in 1,2-dichloroethane (0.5 mL) was added AcOH (46.3 mg, 0.770 mmol) and sodium triacetoxyborohydride (41 mg, 0.2 mmol). The mixture was stirred at 25° C. for 4 hours. After completion, the reaction mixture was diluted with water (150 mL) and extracted with EtOAc (200 mL×3). The combined organic layer was washed with brine (100 mL×2), dried over anhydrous sodium sulfate, filtered and concentrated in vacuo to give crude residue, which was purified by prep-HPLC and then prep-TLC to give 080ba (80 mg, 0.150 mmol, 7.11% yield) as a white solid and 080bb (100 mg, 0.180 mmol, 9% yield). 080ba: MS obsd. (ESI⁺) [(M+H)⁺]: 547.4. The cis and trans conformations were determined by 2D NMR.

### Step 3: Tert-butyl N-[[cis-1-[(1R,3S)-3-aminocyclopentyl]-4-[(tert-butoxycarbonylamino)methyl]pyrrolidin-3-yl]methyl]carbamate

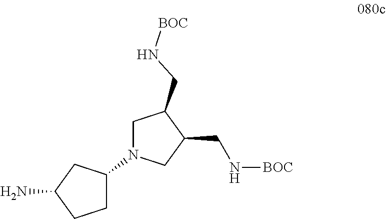 [SMILES: CC(C)(C)OC(=O)NC[C@@H]1CN([C@@H]2CC[C@H](N)C2)C[C@@H]1CNC(=O)OC(C)(C)C]

To a solution of Pd/C (110.0 mg) powder in the mixture of THF (1 mL) and IPA (1 mL) was added 080ba (100.0 mg, 0.180 mmol). The mixture was stirred at 45° C. for 3 hours under H₂ atmosphere. After the starting material was consumed, the mixture was filtrated through celite and concentrated in vacuo to give 080c (100.0 mg, 0.180 m mol) as a white solid. MS obsd. (ESI⁺) [(M+H)⁺]: 412.4.

### Step 4: Tert-butyl N-[(3R)-1-[4-[4-[4-[[4-[[(1S,3R)-3-[cis-3,4-bis[(tert-butoxycarbonylamino)methyl]pyrrolidin-1-yl]cyclopentyl]carbamoyl]cyclohexyl]-difluoro-methyl]-6-chloro-2-pyridyl]piperazin-1-yl]sulfonylphenyl]-5-oxo-pyrrolidin-3-yl]carbamate

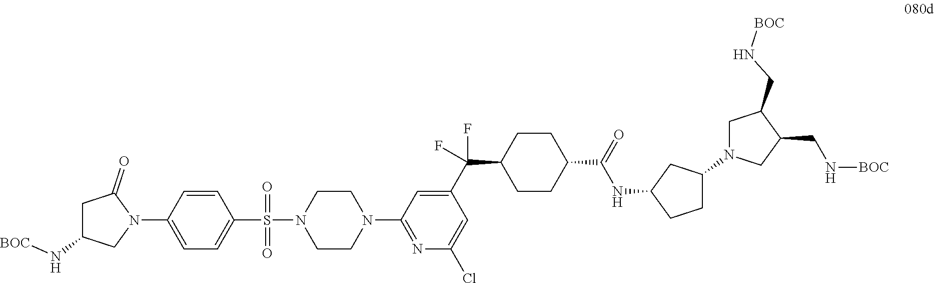 [SMILES: CC(C)(C)OC(=O)NC[C@@H]1CN([C@@H]2CC[C@H](NC(=O)[C@H]3CC[C@H](C(F)(F)c4cc(Cl)nc(N5CCN(S(=O)(=O)c6ccc(N7C[C@H](NC(=O)OC(C)(C)C)CC7=O)cc6)CC5)c4)CC3)C2)C[C@@H]1CNC(=O)OC(C)(C)C]

To the solution of Int-5 (120 mg, 0.170 mmol) in DMF (1 mL) was added HATU (96.04 mg, 0.250 mmol) and DIEA (108.68 mg, 0.840 mmol).The reaction mixture was stirred at 25° C. for 15 minutes. Then 080c (70 mg, 0.170 mmol) was added and the mixture was stirred at 25° C. for 2 hours. After the starting material was consumed, the reaction mixture was quenched by adding water (100 mL) and diluted with EtOAc (100 mL). The aqueous layer was extracted with EtOAc (100 mL×2). The combined organic layer was washed with brine (100 mL), dried over drying anhydrous sodium sulfate, filtered and concentrated in vacuo to give the crude product, which was purified by prep-HPLC and prep-TLC to give 080d (40 mg, 0.040 mmol, 21.5% yield) as a white solid. MS obsd. (ESI⁺) [(M+H)⁺]: 1106.6.

### Step 5: Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[cis-3,4-bis(aminomethyl)pyrrolidin-1-yl]cyclopentyl]cyclohexanecarboxamide

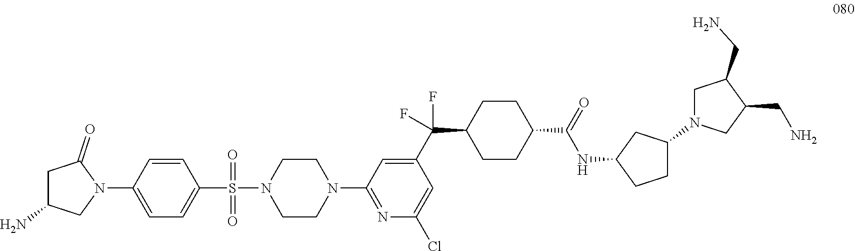 [SMILES: NC[C@@H]1CN([C@@H]2CC[C@H](NC(=O)[C@H]3CC[C@H](C(F)(F)c4cc(Cl)nc(N5CCN(S(=O)(=O)c6ccc(N7C[C@H](N)CC7=O)cc6)CC5)c4)CC3)C2)C[C@@H]1CN]

To a solution of 080d (40.0 mg, 0.040 mmol) in DCM (1 mL) was added TFA (1 mL) in one portion. The mixture was stirred at 25° C. for 1 hour. After completion, the reaction mixture was concentrated in vacuo. The crude product was purified by prep-HPLC to give Example 080 (25.5 mg, 0.030 mmol, 83.37% yield) as a yellow solid. MS obsd. (ESI⁺) [(M+H)⁺]: 806.3.

Example 080: ¹H NMR (400 MHz, DMSO-d₆) δ ppm 8.30-8.49 (m, 3H), 8.04-8.27 (m, 6H), 7.92 (d, J=9.01 Hz, 2H), 7.79 (d, J=8.88 Hz, 2H), 6.78 (s, 1H), 6.71 (s, 1H), 4.26 (dd, J=11.26, 7.00 Hz, 1H), 3.98-4.10 (m, 2H), 3.82-3.89 (m, 1H), 3.57-3.76 (m, 7H), 3.02-3.16 (m, 5H), 2.90-3.01 (m, 6H), 2.63 (br s, 2H), 2.12-2.30 (m, 2H), 2.00-2.09 (m, 1H), 1.89-1.98 (m, 2H), 1.69-1.86 (m, 4H), 1.55-1.69 (m, 4H), 1.27-1.40 (m, 2H), 1.01-1.18 (m, 2H). ¹⁹F NMR (376 MHz, DMSO-d₆) δ ppm −105.40-−102.96 (m, 2F).

### Example 081: Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[(1S)-3-[cis-3,5-diamino-1-piperidyl]cyclopentyl]cyclohexanecarboxamide

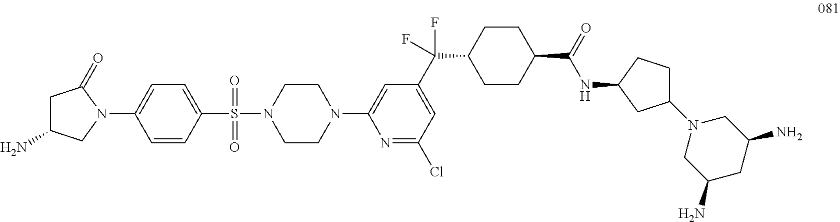 [SMILES: N[C@@H]1CC(=O)N(c2ccc(S(=O)(=O)N3CCN(c4cc(C(F)(F)[C@H]5CC[C@H](C(=O)N[C@H]6CCC(N7C[C@H](N)C[C@H](N)C7)C6)CC5)cc(Cl)n4)CC3)cc2)C1]

The title compound was prepared in analogy to the preparation of Example 080 by using tert-butyl N-[cis-5-(tert-butoxycarbonylamino)-3-piperidyl]carbamate instead of tert-butyl N-[[cis-4-[(tert-butoxycarbonylamino)methyl]pyrrolidin-3-yl]methyl]carbamate in Step 4.

Example 081 (50 mg) was obtained as a white solid. MS obsd. (ESI⁺) [(M+H)⁺]: 792.5.

Example 081: ¹H NMR (400 MHz, DMSO-d₆) δ ppm 7.84-7.92 (m, 2H), 7.75-7.83 (m, 2H), 6.70 (br d, J=18.51 Hz, 2H), 4.23-4.34 (m, 1H), 4.05 (br s, 1H), 3.82 (br d, J=10.63 Hz, 1H), 3.50-3.70 (m, 9H), 3.08 (br dd, J=18.07, 8.32 Hz, 1H), 2.94 (br s, 4H), 2.75-2.84 (m, 2H), 2.61 (br d, J=2.13 Hz, 1H), 2.30-2.47 (m, 2H), 1.96-2.14 (m, 4H), 1.52-1.92 (m, 9H), 1.29 (br d, J=11.38 Hz, 2H), 1.10 (br d, J=12.76 Hz, 2H). ¹⁹F NMR (376 MHz, DMSO-d₆) δ ppm −106.09-−102.01 (m, 2F).

### Example 082: Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[(3R)-3-aminopyrrolidin-1-yl]cyclopentyl]cyclohexanecarboxamide

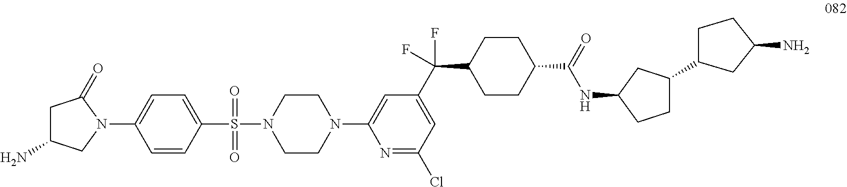

The title compound was prepared in analogy to the preparation of Example 080 by using tert-butyl N-[(3R)-pyrrolidin-3-yl]carbamate instead of tert-butyl N-[[cis-4-[(tert-butoxycarbonylamino)methyl]pyrrolidin-3-yl]methyl]carbamate in Step 4. Example 082 (60 mg) was obtained as a white solid. MS obsd. (ESI⁺) [(M+H)⁺]: 763.3.

Example 082: ¹H NMR (400 MHz, DMSO-d₆) δ ppm 7.94 (d, J=9.2 Hz, 2H), 7.83 (d, J=8.8 Hz, 2H), 6.67 (s, 1H), 4.65 (s, 1H), 4.38-4.43 (m, 1H), 4.15-4.20 (m, 2H), 4.07-4.11 (m, 1H), 3.91-3.98 (m, 2H), 3.76-3.81 (m, 1H), 3.69-3.71 (m, 4H), 3.57-3.63 (m, 1H), 3.17-3.23 (m, 2H), 3.07 (t, J=4.8 Hz, 2H), 2.71 (dd, J=3.2 Hz, J=18.0 Hz, 2H), 2.51-2.58 (m, 1H), 2.14-2.25 (m, 3H), 1.95-2.10 (m, 3H), 1.76-1.86 (m, 6H), 1.42-1.45 (m, 2H), 1.21-1.28 (m, 2H). ¹⁹F NMR (376 MHz, DMSO-d₆) δ ppm −104.55-−102.79 (m, 2F).

### Example 084: Trans-3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl-(3-aminopropyl)-dimethyl-ammonium

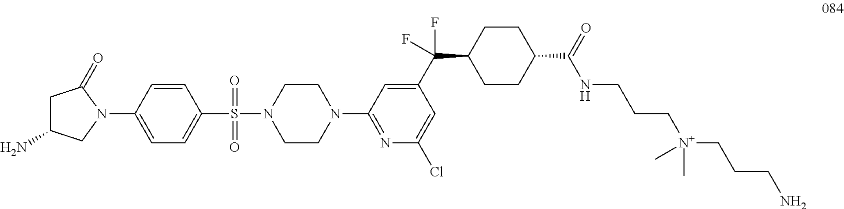 [SMILES: C[N+](C)(CCCN)CCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

### Step 1: Trans-tert-butyl N-[3-[3-[[4-[[2-[4-[4-[(4R)-4-(tert-butoxycarbonylamino)-2-oxo-pyrrolidin-1-yl]phenyl]sulfonylpiperazin-1-yl]-6-chloro-4-pyridyl]-difluoro-methyl]cyclohexanecarbonyl]amino]propyl-methyl-amino]propyl]carbamate

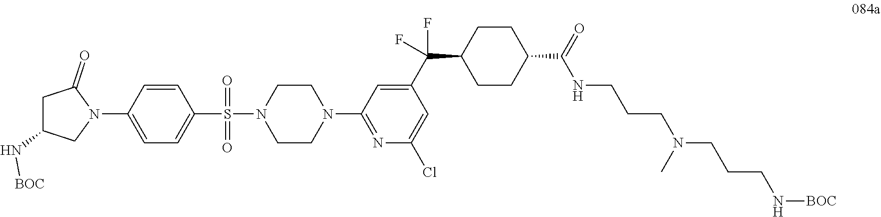 [SMILES: CN(CCCNC(=O)OC(C)(C)C)CCCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a mixture of Int-5 (110.0 mg, 0.150 mmol) and N,N-diisopropylethylamine (70.0 mg, 0.540 mmol) in anhydrous DMF (4 mL) was added HATU (75.0 mg, 0.2 mmol) in one portion. The reaction mixture was stirred for 1 hour at 20° C. Then N′-(3-aminopropyl)-N′-methylpropane-1,3-diamine (60.0 mg, 0.410 mmol) was added into the reaction mixture and stirred for 11 hours. After the starting material was consumed, the reaction mixture was filtered and purified by prep-HPLC. The resulting residue was then dissolved in DCM (3 mL) and into the solution was added with Boc₂O (100 mg). The reaction was stirred at 25° C. for 12 hours. After completion, the mixture was concentrated in vacuo to give the crude product, which was purified by prep-HPLC to give 084a (101 mg, 0.110 mmol, 79.14% yield) as a white solid. MS obsd. (ESI⁺) [(M+H)⁺]: 938.3.

### Step 2: Trans-3-[[4-[[2-[4-[4-[(4R)-4-(tert-butoxycarbonylamino)-2-oxo-pyrrolidin-1-yl]phenyl]sulfonylpiperazin-1-yl]-6-chloro-4-pyridyl]-difluoro-methyl]cyclohexanecarbonyl]amino]propyl-[3-(tert-butoxycarbonylamino)propyl]-dimethyl-ammonium

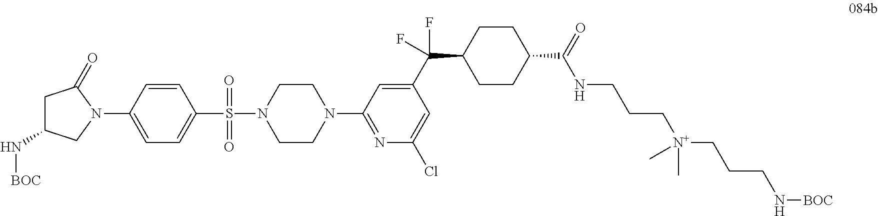 [SMILES: CC(C)(C)OC(=O)NCCC[N+](C)(C)CCCNC(=O)[C@H]1CC[C@H](C(F)(F)c2cc(Cl)nc(N3CCN(S(=O)(=O)c4ccc(N5C[C@H](NC(=O)OC(C)(C)C)CC5=O)cc4)CC3)c2)CC1]

To a solution of 084a (60.0 mg, 0.060 mmol) in DMSO (1 mL) was added sodium hydrogen carbonate (160.9 mg, 1.90 mmol) and iodomethane (0.3 mL, 0.12 mmol). The mixture was stirred at 40° C. for 12 hours. After completion, the reaction mixture was purified by prep-HPLC to give 084b (50 mg, 0.050 mmol, 82.02% yield) as a white solid. MS obsd. (ESI⁺) [(M+H)⁺]: 953.1.

### Step 3: Trans-3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl-(3-aminopropyl)-dimethyl-ammonium

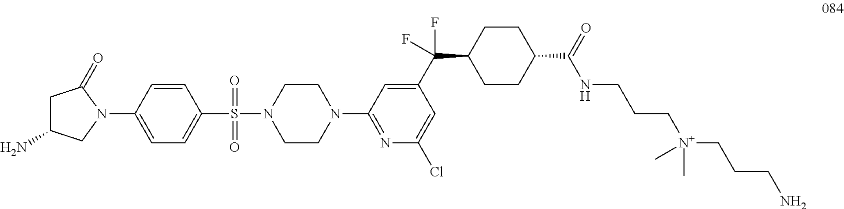 [SMILES: C[N+](C)(CCCN)CCCNC(=O)[C@H]1CC[C@H](C(F)(F)c2cc(Cl)nc(N3CCN(S(=O)(=O)c4ccc(N5C[C@H](N)CC5=O)cc4)CC3)c2)CC1]

To a solution of 084b (30.0 mg, 0.030 mmol) in DCM (0.5 mL) was added TFA (0.5 mL, 0.040 mmol). The mixture was stirred at 20° C. for 2 hours. Then, the reaction mixture was filtered and concentrated in vacuo to give a residue, which was purified by prep-HPLC to give Example 084 (10.2 mg, 0.010 mmol, 41.89% yield) as a white solid. MS obsd. (ESI⁺) [(M+H)⁺]: 753.4.

Example 084: ¹H NMR (400 MHz, METHANOL-d₄) δ ppm 7.94 (br d, J=8.56 Hz, 2H), 7.82 (br d, J=8.44 Hz, 2H), 6.66-6.70 (m, 1H), 6.65 (s, 1H), 4.42 (br d, J=4.16 Hz, 1H), 4.22 (br s, 1H), 4.00 (br d, J=10.51 Hz, 1H), 3.69 (br s, 4H), 3.48 (br dd, J=2.63, 1.41 Hz, 2H), 3.35-3.43 (m, 2H), 3.24-3.29 (m, 2H), 3.21 (br d, J=10.03 Hz, 1H), 3.14 (s, 6H), 3.06 (br s, 6H), 2.74 (dd, J=18.03, 2.26 Hz, 1H), 2.18 (br s, 3H), 1.96-2.11 (m, 3H), 1.88 (br d, J=10.15 Hz, 2H), 1.78 (br d, J=11.74 Hz, 2H), 1.43 (br d, J=12.10 Hz, 2H), 1.24 (br d, J=11.74 Hz, 2H). ¹⁹F NMR (376 MHz, METHANOL-d4) δ ppm −107.09-−106.74 (m, 2F).

Compound JH-LPH-33 disclosed in the literature (Bioorganic Chemistry (2020), 102, 104055) and reported having LPS synthesis pathway inhibition activity and displaying antibiotic activity against efflux-deficient Escherichia coli strains, was chosen as reference compound in present invention.

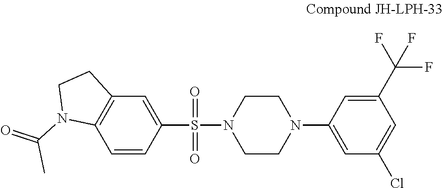 [SMILES: CC(=O)N1CCc2cc(S(=O)(=O)N3CCN(c4cc(Cl)cc(C(F)(F)F)c4)CC3)ccc21]

### BIOLOGICAL EXAMPLES

### Example 085: 50% Inhibitory Concentration (IC₅₀) of Escherichia coli UDP-2,3-diacylglucosamine hydrolase (LpxH)

The inhibitory potency of compounds of Escherichia coli UDP-2,3-diacylglucosamine hydrolase (LpxH) was determined in an enzymatic assay. UDP-2,3-diacylglucosamine (UDP-DAG) was purified from the Caulobacter crescentus LpxI D225A mutant protein that contained a tightly bound UDP-DAG molecule. The enzyme was diluted using assay buffer containing 50 mM NaCl, 20 mM Tris-HCl pH7.5, 2.5 mM MnCl₂, 0.01% Triton X-100.1 mg/mL BSA, and the final concentration is 2 nM. The compounds were diluted by Agilent liquid handler Bravo, the compound's serial dilution dose ranges from 100 μM to 1.7 nM. Then the enzyme and compounds mixture were incubated at room temperature for 10 mins. The enzymatic assay was started by adding UDP-DAG (FAC is 5 μM) and incubated for 20 mins at room temperature. The plate was then heated to 95° C. for 15 mins on a water batch to stop the reaction. The hydrolysis of UDP-DAG by LpxH yielded 2,3-diacylglucosamine 1-phosphate (lipid X) and UMP, which was converted to ATP and quantified by the luciferase reaction using the UMP/CMP-Glo™ Glycosyltransferase Assay kit from Promega. The compound's inhibitory effect of LpxH is determined by measuring the light change using a luminometer (Envision).

TABLE 3
Enzymatic IC₅₀ values of the compounds of this
invention against Escherichia coli LpxH

| Example No. | IC₅₀ (μg/mL) | Example No. | IC₅₀ (μg/mL) |
|---|---|---|---|
| JH-LPH-33 | 0.143 | 036 | 0.004 |
| 001 | 0.002 | 037 | <0.002 |
| 002 | <0.002 | 040 | 0.004 |
| 003 | <0.002 | 042 | 0.008 |
| 004 | 0.006 | 043 | 0.025 |
| 005 | 0.003 | 044 | 0.035 |
| 006 | 0.003 | 045 | 0.014 |
| 007 | 0.005 | 047 | 0.005 |
| 008 | 0.004 | 048 | 0.013 |
| 009 | <0.002 | 049 | 0.007 |
| 011 | 0.012 | 050 | 0.002 |
| 012 | 0.002 | 051 | 0.009 |
| 013 | 0.003 | 052 | <0.002 |
| 014 | <0.002 | 054 | 0.024 |
| 020 | 0.002 | 055 | 0.006 |
| 021 | 0.002 | 056 | 0.004 |
| 022 | 0.004 | 057 | 0.002 |
| 023 | <0.002 | 058 | <0.002 |
| 024 | 0.003 | 059 | <0.002 |
| 025 | <0.002 | 060 | <0.002 |
| 026 | 0.007 | 061 | 0.002 |
| 027 | 0.004 | 062 | 0.022 |
| 028 | 0.014 | 063 | 0.004 |
| 029 | 0.002 | 071 | 0.011 |
| 030 | 0.006 | 072 | 0.003 |
| 031 | 0.005 | 073 | <0.002 |
| 032 | <0.002 | 074 | 0.030 |
| 033 | 0.004 | 075 | 0.006 |
| 034 | 0.020 | 076 | <0.002 |
| 035 | 0.030 | 077 | 0.002 |
| 038 | 0.003 | 078 | 0.004 |
| 039 | 0.003 | | |

### Example 086: Minimal Inhibitory Concentration Protocol (MIC) Assay

The antibacterial activity of molecules was evaluated against the commonly used quality control strain Escherichia coli BW 25113, Escherichia coli ATCC 25922 and the rifampin-resistant mutant strain Klebsiella pneumonia ATCC 43816. Escherichia co/i BW 25113 was obtained from the Coli Genetic Stock Center (CGSC) (strain 7636). Both Escherichia coli ATCC 25922 and Klebsiella pneumonia ATCC 43816 were originally derived from human clinical samples and are available from ATCC (American Type Culture Collection). The in vitro potency of compounds to inhibit Escherichia coli (BW 25113, ATCC 25922) and Klebsiella pneumonia (ATCC 43816) growth was assessed by the MIC (Minimal Inhibitory Concentration) broth dilution method. Specifically compound dilutions were prepared from 10 mM DMSO stock solutions as follows: i) serial 2-fold dilution in 20 μL DMSO were prepared in a master plate (Greiner, Cat No: 651201), ii) 180 μL sterile distilled water was added to each aliquot and iii) 10 μL diluted compounds were transferred into a new assay plate (Costar, 3599).

Vials of each test microorganisms were maintained frozen in the vapor phase of a liquid nitrogen freezer. Single-use frozen vials of the three strains Escherichia coli BW 25113, Escherichia coli ATCC 25922 (KWTKSTTK, 0335K) and Klebsiella pneumonia ATCC 43816, with predetermined CFU/mL, were taken out from the freezer, thawed at room temperature, and diluted in Cation-Adjusted Mueller Hinton Broth (CAMVIHB) to achieve a final inoculum of 5×10⁵ CFU/mL. 90 μL bacteria containing broth was dispensed to the assay plate containing the pre-dispensed compound dilutions and mixed by pipetting 5 times.

Then the assay plates were incubated for 20 hours at 35° C. in ambient air. Following incubation, the MIC (μ/mL), the lowest concentration of drug that inhibits visible growth of the microorganism was read with the help of a magnification mirror and recorded.

TABLE 4
MIC values of the compounds of this invention
against E. coli and K. pneumonia

| | MIC (μg/mL) | | |
|---|---|---|---|
| Example | E. coli K-12 | E. coli K-12 | K. p ATCC |
| No. | BW 25113 | ATCC 25922 | 43816 |
| JH-LPH-33 | >24 | >24 | >24 |
| 001 | 0.49 | 0.98 | 0.98 |
| 002 | 1.8 | 1.8 | 1.8 |
| 003 | 1 | 4.2 | 2.1 |
| 004 | 0.83 | 1.1 | 1.1 |
| 005 | 3.3 | 2.2 | 2.2 |
| 006 | 1.1 | 1.1 | 1.1 |
| 007 | 1.1 | 1.1 | 0.53 |
| 008 | 1 | 0.52 | 0.52 |
| 009 | 1.5 | 1.5 | 1.5 |
| 011 | 1.1 | 1.1 | 1.1 |
| 012 | 0.57 | 0.57 | 0.57 |
| 013 | 2.6 | 2.6 | 1.3 |
| 014 | 1.1 | 1.1 | 1.1 |
| 015 | 0.70 | 0.70 | 0.70 |
| 016 | 0.35 | 0.35 | 0.35 |
| 017 | 0.35 | 0.30 | 0.45 |
| 018 | 0.62 | 0.62 | 0.62 |
| 019 | 0.35 | 0.71 | 0.35 |
| 020 | 0.72 | 0.72 | 0.72 |
| 021 | 2.2 | 1.3 | 1.8 |
| 022 | 1.4 | 0.71 | 0.71 |
| 023 | 1.4 | 1.4 | 2.9 |
| 024 | 0.69 | 0.69 | 0.69 |
| 026 | 0.78 | 0.78 | 0.78 |
| 027 | 1.6 | 3.2 | 3.2 |
| 028 | 1.6 | 1.6 | 1.6 |
| 029 | 2.8 | 1.4 | 1.4 |
| 030 | 1.7 | 1.1 | 1.7 |
| 031 | 1.7 | 2.2 | 2.2 |
| 032 | 3.3 | 2.2 | 2.2 |
| 033 | 1.1 | 0.57 | 0.43 |
| 034 | 1.7 | 1.1 | 1.1 |
| 036 | 1.1 | 1.1 | 1.1 |
| 037 | 0.73 | 1.5 | 1.5 |
| 038 | 1.5 | 1.5 | 1.5 |
| 039 | 0.82 | 1.6 | 1.6 |
| 041 | 0.54 | 0.54 | 0.54 |
| 042 | 2.3 | 2.3 | 2.3 |
| 043 | 1.1 | 0.57 | 1.1 |
| 044 | 1.2 | 0.62 | 1.2 |
| 045 | 1.2 | 0.62 | 0.62 |
| 046 | 2.4 | 1.2 | 1.2 |
| 047 | 0.30 | 0.35 | 0.30 |
| 048 | 1.2 | 1.2 | 1.2 |
| 049 | 0.60 | 1.2 | 1.2 |
| 050 | 2.1 | 1.1 | 1.1 |
| 051 | 2.3 | 4.6 | 4.6 |
| 052 | 0.56 | 0.56 | 0.56 |
| 053 | 0.55 | 0.57 | 0.54 |
| 055 | 0.55 | 0.85 | 0.55 |
| 056 | 1.1 | 1.2 | 1.1 |
| 057 | 1.1 | 1.1 | 1.1 |
| 058 | 0.29 | 1.1 | 1.1 |
| 059 | 1.2 | 1.2 | 1.2 |
| 060 | 2.4 | 1.8 | 1.2 |
| 061 | 1.1 | 1.1 | 1.1 |
| 064 | 0.67 | 0.57 | 0.57 |
| 065 | 0.66 | 0.66 | 0.66 |
| 066 | 1.3 | 0.63 | 0.59 |
| 067 | 0.59 | 0.30 | 0.57 |
| 068 | 0.32 | 0.32 | 0.32 |
| 069 | 0.56 | 0.56 | 0.56 |
| 072 | 0.57 | 1.1 | 1.1 |
| 073 | 1.2 | 1.2 | 2.4 |
| 074 | 1.9 | 1.9 | 1.9 |
| 075 | 2.3 | 2.3 | 1.2 |
| 076 | 1.1 | 1.1 | 1.1 |
| 077 | 2.2 | 2.2 | 2.2 |
| 078 | 1.4 | 2.8 | 2.1 |
| 079 | 3.4 | 3.4 | 0.85 |
| 080 | 0.33 | 0.33 | 0.99 |
| 081 | 0.65 | 0.65 | 0.65 |
| 083 | 1.2 | 1.2 | 0.62 |
| 084 | 0.48 | 0.48 | 0.81 |

### Example 087: Human Microsome Stability Assay

The human microsomal stability assay is used for early assessment of metabolic stability of a test compound in human liver microsomes.

Human liver microsomes (Cat.NO.: 452117, Corning, USA) were preincubated with test compound for 10 minutes at 37° C. in 100 mM potassium phosphate buffer, pH 7.4. The reactions were initiated by adding NADPH regenerating system. The final incubation mixtures contained 1 M test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 5 below. (Human microsome clearance: <7 mL/min/kg: low; 7-16.2 mL/min/kg: medium; >16.2 mL/min/kg: high)

TABLE 5
Human microsome clearance values of
the compounds of this invention

| Example | Clearance of Human |
|---|---|
| No. | microsome (mL/min/kg) |
| JH-LPH-33 | 20.94 |
| 002 | 6.15 |
| 003 | 7.41 |
| 004 | 6.15 |
| 005 | 6.15 |
| 006 | 6.15 |
| 007 | 6.90 |
| 008 | 8.07 |
| 009 | 11.13 |
| 011 | 11.93 |
| 012 | 6.19 |
| 013 | 6.77 |
| 014 | 6.15 |
| 015 | 9.58 |
| 016 | 8.63 |
| 017 | 9.36 |
| 018 | 6.15 |
| 019 | 10.63 |
| 020 | 9.48 |
| 021 | 10.85 |
| 022 | 6.15 |
| 023 | 6.15 |
| 024 | 11.22 |
| 026 | 6.15 |
| 027 | 6.15 |
| 029 | 7.22 |
| 030 | 6.90 |
| 031 | 6.15 |
| 033 | 10.77 |
| 034 | 6.15 |
| 035 | 8.90 |
| 036 | 6.15 |
| 037 | 6.66 |
| 038 | 6.80 |
| 039 | 6.15 |
| 040 | 6.15 |
| 041 | 9.73 |
| 042 | 11.12 |
| 043 | 9.94 |
| 044 | 7.30 |
| 045 | 8.79 |
| 046 | 6.15 |
| 047 | 6.15 |
| 048 | 12.06 |
| 049 | 7.38 |
| 051 | 8.70 |
| 052 | 7.41 |
| 053 | 6.15 |
| 055 | 8.58 |
| 056 | 11.37 |
| 057 | 6.15 |
| 058 | 8.04 |
| 059 | 6.15 |
| 060 | 8.12 |
| 061 | 6.15 |
| 062 | 7.74 |
| 064 | 6.15 |
| 065 | 8.21 |
| 066 | 6.15 |
| 067 | 6.15 |
| 068 | 9.49 |
| 069 | 10.85 |
| 071 | 10.73 |
| 072 | 6.15 |
| 073 | 6.15 |
| 074 | 6.15 |
| 075 | 10.47 |
| 076 | 9.06 |
| 079 | 11.16 |
| 081 | 6.71 |
| 083 | 7.71 |
| 084 | 9.7 |

1. A compound of formula (I), 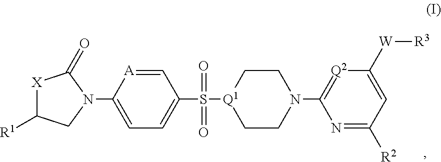
wherein
R¹ is amino, hydroxyC₁₋₆alkyl, C₁₋₆alkylamino, aminoC₁₋₆alkylamino or piperazinyl;
R² is halogen or C₁₋₆alkyl;
R³ is —Y—C(O)—R⁴; wherein
Y is C₃₋₁₀cycloalkyl, phenyl, pyrazinyl or pyridinyl;
R⁴ is ((aminoC₁₋₆alkyl)₂amino)C₁₋₆alkylamino, ((aminoC₁₋₆ alkyl)pyrrolidiniumyl)C₁₋₆alkylamino, ((aminoC₁₋₆alkylcarbonyl)amino)C₁₋₆alkylamino, ((C₁₋₆alkyl)₂amino)C₁₋₆alkylamino, ((C₁₋₆alkyl)₃ammonio)C₁₋₆alkylamino, (aminoC₁₋₆ alkoxy)C₁₋₆alkylamino, (aminoC₁₋₆alkyl(C₁₋₆alkyl)₂ammonio)C₁₋₆alkylamino, (C₁₋₆ alkyl(aminoC₁₋₆alkyl)₂ammonio)C₁₋₆alkylamino, ((aminoC₁₋₆alkylcarbonyl)amino)C₃₋₁₀cycloalkylamino, ((aminoC₁₋₆alkyl)₂pyrrolidinyl)C₃₋₁₀cycloalkylamino, (aminoC₁₋₆alkyl)₂amino, (aminoC₁₋₆alkyl)azetidinyl, (aminoC₁₋₆alkyl)C₃₋₁₀cycloalkylamino, (aminoC₁₋₆alkyl)piperazinyl, (aminoC₁₋₆alkylamino)C₁₋₆alkylamino, (aminoC₁₋₆alkylcarbonyl)aminocarbamoyl, (aminopyrrolidinyl)C₃₋₁₀cycloalkylamino, (azetidinylC₁₋₆ alkylamino)C₁₋₆alkylamino, (C₁₋₆alkylamino)C₁₋₆alkylamino, (C₁₋₆ alkylpyrrolidiniumyl)C₁₋₆alkylamino, (diaminopiperidinyl)C₁₋₆alkylamino, (diaminopiperidinyl)C₃₋₁₀cycloalkylamino, (piperidinylcarbonylamino)carbamoyl, 3-azabicyclo[3.1.0]hexanylamino, amino, amino(C₁₋₆alkoxy)C₁₋₆alkylamino, amino(C₁₋₆alkyl)piperidinyl, (aminoC₁₋₆alkyl)₂pyrrolidinyl, amino(hydroxy)C₁₋₆alkylamino, aminoC₁₋₆alkylamino, aminoC₃₋₁₀cycloalkylamino, aminocarbonylC₁₋₆alkylamino, aminopyrrolidinylamino, amino(C₁₋₆alkyl)pyrrolidinylamino, azetidinylC₁₋₆alkylamino, C₁₋₆alkoxy, C₁₋₆alkyl(aminoC₁₋₆alkyl)amino, C₁₋₆alkylpyrrolidinylamino, C₁₋₆ alkyl(aminoC₁₋₆alkyl)piperaziniumyl, C₁₋₆alkyl-2,6-diazaspiro[3.3]heptanyl, C₁₋₆alkylamino, diaminoC₁₋₆alkylamino, diaminopiperidinyl, hydroxyC₁₋₆alkylamino, hydroxypyrrolidinylamino, morpholinylC₁₋₆alkylamino or pyrrolidinylC₁₋₆alkylamino;
X is CH₂ or O;
A is CH or N;
Q¹ is CH or N;
Q² is CH or N; and
W is haloC₁₋₆alkyl;
or a pharmaceutically acceptable salt thereof.

2. A compound according to claim 1, wherein R¹ is amino, C₁₋₆alkylamino or hydroxyC₁₋₆alkyl.

3. A compound according to claim 1, wherein R¹ is amino, methylamino or hydroxymethyl.

4. A compound according to claim 1, wherein R² is halogen.

5. A compound according to claim 1, wherein R² is chloro.

6. A compound according to claim 1, wherein R³ is —Y—C(O)—R⁴; wherein Y is C₃₋₁₀cycloalkyl, phenyl or pyrazinyl.

7. A compound according to claim 1, wherein R³ is —Y—C(O)—R⁴; wherein Y is bicyclo[2.2.2]octanyl, bicyclo[3.1.0]hexanyl, cyclohexanyl, norbornanyl, phenyl or pyrazinyl.

8. A compound according to claim 1, wherein R³ is —Y—C(O)—R⁴; wherein R⁴ is amino(C₁₋₆alkyl)pyrrolidinylamino, ((aminoC₁₋₆alkyl)₂amino)C₁₋₆alkylamino, ((aminoC₁₋₆alkyl)₂pyrrolidinyl)C₃₋₁₀cycloalkylamino, ((aminoC₁₋₆ alkyl)pyrrolidiniumyl)C₁₋₆alkylamino, (aminoC₁₋₆alkyl(C₁₋₆alkyl)₂ammonio)C₁₋₆alkylamino, (aminoC₁₋₆alkyl)₂amino, (aminoC₁₋₆alkyl)₂pyrrolidinyl, (aminoC₁₋₆alkyl)C₃₋₁₀cycloalkylamino, (C₁₋₆alkyl(aminoC₁₋₆alkyl)₂ammonio)C₁₋₆alkylamino, (aminopyrrolidinyl)C₃₋₁₀cycloalkylamino, (C₁₋₆alkyl(aminoC₁₋₆alkyl)₂ammonio)C₁₋₆alkylamino, (diaminopiperidinyl)C₁₋₆alkylamino, (diaminopiperidinyl)C₃₋₁₀cycloalkylamino, amino, amino(hydroxy)C₁₋₆alkylamino, aminoC₁₋₆alkylamino, aminoC₃₋₁₀cycloalkylamino, C₁₋₆alkylpyrrolidinylamino, diaminoC₁₋₆alkylamino or diaminopiperidinyl.

9. A compound according to claim 1, wherein R³ is —Y—C(O)—R⁴; wherein R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (2-aminoethyl)₂amino, (3,5-diamino-1-piperidinyl)propylamino, (3-aminocyclopentyl)amino, (3-aminopropyl)₂amino, (4-methylpyrrolidin-3-yl)amino, [3-(3,5-diamino-1-piperidinyl)cyclopentyl]amino, [3-[3,4-bis(aminomethyl)pyrrolidin-1-yl]cyclopentyl]amino, [3-[3-aminopyrrolidin-1-yl]cyclopentyl]amino, 1-(aminomethyl)cyclopropylamino, 2-(2-aminoethylamino)ethylamino, 2,3-diaminopropylamino, 2-aminoethylamino, 3,4-bis(aminomethyl)pyrrolidin-1-yl, 3,5-diaminopiperidinyl, 3-[3-aminopropyl(dimethyl)ammonio]propylamino, 3-[bis(2-aminoethyl)-methyl-ammonio]propylamino, 3-amino-2-hydroxy-propylamino, 3-aminopropylamino, 3-hydroxypropylamino, 4-amino-2-methyl-pyrrolidin-1-yl, amino or aminopropylamino.

10. A compound according to claim 1, wherein A is CH.

11. A compound according to claim 1, wherein Q¹ is N.

12. A compound according to claim 1, wherein Q² is CH.

13. A compound according to claim 1, wherein W is CF₂.

14. A compound according to claim 1, wherein
R¹ is amino or hydroxyC₁₋₆alkyl;
R² is halogen;
R³ is —Y—C(O)—R⁴; wherein
Y is C₃₋₁₀cycloalkyl, phenyl or pyrazinyl;
R⁴ is amino(C₁₋₆alkyl)pyrrolidinylamino, ((aminoC₁₋₆alkyl)₂amino)C₁₋₆alkylamino, ((aminoC₁₋₆alkyl)₂pyrrolidinyl)C₃₋₁₀cycloalkylamino, ((aminoC₁₋₆alkyl)pyrrolidiniumyl)C₁₋₆alkylamino, (aminoC₁₋₆alkyl(C₁₋₆alkyl)₂ammonio)C₁₋₆ alkylamino, (aminoC₁₋₆alkyl)₂amino, (aminoC₁₋₆alkyl)₂pyrrolidinyl, (aminoC₁₋₆alkyl)C₃₋₁₀cycloalkylamino, (C₁₋₆alkyl(aminoC₁₋₆alkyl)₂ammonio)C₁₋₆alkylamino, (aminopyrrolidinyl)C₃₋₁₀cycloalkylamino, (C₁₋₆alkyl(aminoC₁₋₆alkyl)₂ammonio)C₁₋₆alkylamino, (diaminopiperidinyl)C₁₋₆alkylamino, (diaminopiperidinyl)C₃₋₁₀cycloalkylamino, amino, amino(hydroxy)C₁₋₆alkylamino, aminoC₁₋₆alkylamino, aminoC₃₋₁₀cycloalkylamino, C₁₋₆alkylpyrrolidinylamino, diaminoC₁₋₆alkylamino or diaminopiperidinyl;
X is CH₂ or O;
A is CH;
Q¹ is N;
Q² is CH;
W is haloC₁₋₆alkyl;
or a pharmaceutically acceptable salt thereof.

15. A compound according to claim 1, wherein
R¹ is amino or hydroxymethyl;
R² is chloro;
R³ is —Y—C(O)—R⁴; wherein
Y is bicyclo[2.2.2]octanyl, bicyclo[3.1.0]hexanyl, cyclohexanyl, phenyl or pyrazinyl;
R⁴ is ((2-aminoethyl)₂amino)propylamino, ((2-aminoethyl)pyrrolidin-1-ium-1-yl)ethylamino, (2-aminoethyl(dimethyl)ammonio)ethylamino, (2-aminoethyl)₂amino, (3,5-diamino-1-piperidinyl)propylamino, (3-aminocyclopentyl)amino, (3-aminopropyl)₂amino, (4-methylpyrrolidin-3-yl)amino, [3-(3,5-diamino-1-piperidinyl)cyclopentyl]amino, [3-[3,4-bis(aminomethyl)pyrrolidin-1-yl]cyclopentyl]amino, [3-[3-aminopyrrolidin-1-yl]cyclopentyl]amino, 1-(aminomethyl)cyclopropylamino, 2-(2-aminoethylamino)ethylamino, 2,3-diaminopropylamino, 2-aminoethylamino, 3,4-bis(aminomethyl)pyrrolidin-1-yl, 3,5-diaminopiperidinyl, 3-[3-aminopropyl(dimethyl)ammonio]propylamino, 3-[bis(2-aminoethyl)-methyl-ammonio]propylamino, 3-amino-2-hydroxy-propylamino, 3-aminopropylamino, 3-hydroxypropylamino, 4-amino-2-methyl-pyrrolidin-1-yl, amino or aminopropylamino;
X is CH₂ or O;
A is CH;
Q¹ is N;
Q² is CH;
W is CF₂;
or a pharmaceutically acceptable salt thereof.

16. A compound selected from:
Trans-methyl 4-[[2-[4-[4-(4-amino-2-oxo-pyrrolidin-1-yl)phenyl]sulfonylpiperazin-1-yl]-6-chloro-4-pyridyl]-difluoro-methyl]cyclohexanecarboxylate;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(azetidin-3-ylmethyl)cyclohexanecarboxamide;
Trans-4-[[2-[4-[4-(4-amino-2-oxo-pyrrolidin-1-yl)phenyl]sulfonylpiperazin-1-yl]-6-chloro-4-pyridyl]-difluoro-methyl]-N-(3-hydroxypropyl)cyclohexanecarboxamide;
Trans-(4R)-4-amino-1-[4-[4-[4-[[4-(4-amino-4-methyl-piperidine-1-carbonyl)cyclohexyl]-difluoro-methyl]-6-chloro-2-pyridyl]piperazin-1-yl]sulfonylphenyl]pyrrolidin-2-one;
Trans-(4R)-4-amino-1-[4-[4-[6-chloro-4-[difluoro-[4-(6-methyl-2,6-diazaspiro[3.3]heptane-2-carbonyl)cyclohexyl]methyl]-2-pyridyl]piperazin-1-yl]sulfonylphenyl]pyrrolidin-2-one;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(4-hydroxypyrrolidin-3-yl)cyclohexanecarboxamide;
Trans-N-(3-aminocyclobutyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-(2-amino-1-methyl-ethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2-pyrrolidin-1-ylethyl)cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[(1S,5R)-3-azabicyclo[3.1.0]hexan-6-yl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[cis-4-aminopyrrolidin-3-yl]cyclohexanecarboxamide;
Trans-N-[1-(aminomethyl)cyclopropyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-(4R)-4-amino-1-[4-[4-[4-[[4-[4-(2-aminoethyl)piperazine-1-carbonyl]cyclohexyl]-difluoro-methyl]-6-chloro-2-pyridyl]piperazin-1-yl]sulfonylphenyl]pyrrolidin-2-one;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
4-[[2-[4-[4-(4-amino-2-oxo-pyrrolidin-1-yl)phenyl]sulfonylpiperazin-1-yl]-6-chloro-4-pyridyl]-difluoro-methyl]-N-(3-hydroxypropyl)benzamide;
4-[[2-[4-[4-(4-amino-2-oxo-pyrrolidin-1-yl)phenyl]sulfonylpiperazin-1-yl]-6-chloro-4-pyridyl]-difluoro-methyl]-N-(3-aminopropyl)benzamide;
N-(2-aminoethyl)-4-[[2-[4-[4-(4-amino-2-oxo-pyrrolidin-1-yl)phenyl]sulfonylpiperazin-1-yl]-6-chloro-4-pyridyl]-difluoro-methyl]benzamide;
N-(3-amino-2-hydroxy-propyl)-4-[[2-[4-[4-(4-amino-2-oxo-pyrrolidin-1-yl)phenyl]sulfonylpiperazin-1-yl]-6-chloro-4-pyridyl]-difluoro-methyl]benzamide;
N-(3-amino-2-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]benzamide;
4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]benzamide;
N-(3-aminopropyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
N-(3-amino-2-hydroxy-propyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]pyrazine-2-carboxamide;
N-(2-aminoethyl)-5-[[2-chloro-6-[4-[4-[(4R)-4-amino-2-oxo-pyrrolidin-1-yl]phenyl]sulfonylpiperazin-1-yl]-4-pyridyl]-difluoro-methyl]pyrazine-2-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2-morpholinoethyl)pyrazine-2-carboxamide;
N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]pyridine-3-carboxamide;
6-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(dimethylamino)propyl]pyridine-3-carboxamide;
N-[2-(2-aminoethylamino)ethyl]-6-[[2-chloro-6-[4-[4-[(4R)-4-amino-2-oxo-pyrrolidin-1-yl]phenyl]sulfonylpiperazin-1-yl]-4-pyridyl]-difluoro-methyl]pyridine-3-carboxamide;
5-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-(methylamino)propyl]pyridine-2-carboxamide;
Trans-N-(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-methyl-cyclohexanecarboxamide;
Cis-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Cis-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(3-hydroxypropyl)cyclohexanecarboxamide;
N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[2.2.2]octane-1-carboxamide;
N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]norbornane-1-carboxamide;
N-(3-aminopropyl)-3-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclopentanecarboxamide;
Trans-N-(3-amino-2-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
3-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]benzoyl]amino]propyl-trimethyl-ammonium;
4-[[2-Chloro-6-[4-[4-[(4R)-4-amino-2-oxo-pyrrolidin-1-yl]phenyl]sulfonylpiperazin-1-yl]-4-pyridyl]-difluoro-methyl]-N-[3-(1-methylpyrrolidin-1-ium-1-yl)propyl]benzamide;
5-[[2-Chloro-6-[4-[4-[(4R)-4-amino-2-oxo-pyrrolidin-1-yl]phenyl]sulfonylpiperazin-1-yl]-4-pyridyl]-difluoro-methyl]-N-[3-(2-hydroxyethylamino)propyl]pyridine-2-carboxamide;
N-(3-aminopropyl)-3-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[1.1.1]pentane-1-carboxamide;
Trans-N-(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-[2-(2-aminoethoxy)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N,N-bis(2-aminoethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[2.2.2]octane-1-carboxamide;
N,N-bis(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]norbornane-1-carboxamide;
Trans-N-[3-[bis(2-aminoethyl)amino]propyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[6-chloro-4-[[4-cis-(3,5-diaminopiperidine-1-carbonyl)cyclohexyl]-difluoro-methyl]-2-pyridyl]piperazin-1-yl]sulfonylphenyl]pyrrolidin-2-one;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[3-[cis-3,5-diamino-1-piperidyl]propyl]cyclohexanecarboxamide;
Trans-N′-(2-aminoacetyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbohydrazide;
Trans-N′-[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]piperidine-4-carbohydrazide;
Trans-N-(2-amino-3-hydroxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
(2R,5S)-5-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-(3-aminopropyl)bicyclo[2.2.2]octane-2-carboxamide;
Trans-N-(3-aminopropyl)-4-[difluoro-[6-methyl-2-[4-[4-[(4R)-4-amino-2-oxo-pyrrolidin-1-yl]phenyl]sulfonylpiperazin-1-yl]pyrimidin-4-yl]methyl]cyclohexanecarboxamide;
N-(3-aminopropyl)-6-[[2-chloro-6-[4-[4-[(4R)-4-amino-2-oxo-pyrrolidin-1-yl]phenyl]sulfonylpiperazin-1-yl]-4-pyridyl]-difluoro-methyl]bicyclo[3.1.0]hexane-3-carboxamide;
Trans-N-(3-aminopropyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[4-[[4-[3-(aminomethyl)azetidine-1-carbonyl]cyclohexyl]-difluoro-methyl]-6-chloro-2-pyridyl]piperazin-1-yl]sulfonylphenyl]pyrrolidin-2-one;
Trans-N-(3-aminopropyl)-4-[[2-chloro-6-[4-[4-[(5S)-5-(hydroxymethyl)-2-oxo-oxazolidin-3-yl]phenyl]sulfonylpiperazin-1-yl]-4-pyridyl]-difluoro-methyl]cyclohexanecarboxamide;
Trans-N-(3-aminopropyl)-4-[[2-chloro-6-[4-[4-[(5R)-5-(hydroxymethyl)-2-oxo-oxazolidin-3-yl]phenyl]sulfonylpiperazin-1-yl]-4-pyridyl]-difluoro-methyl]cyclohexanecarboxamide;
Trans-N-(3-aminopropyl)-4-[[2-chloro-6-[4-[4-(2-oxo-4-piperazin-1-yl-pyrrolidin-1-yl)phenyl]sulfonylpiperazin-1-yl]-4-pyridyl]-difluoro-methyl]cyclohexanecarboxamide;
Trans-N-(3-aminopropyl)-4-[[2-chloro-6-[4-[[6-[(4R)-4-amino-2-oxo-pyrrolidin-1-yl]-3-pyridyl]sulfonyl]piperazin-1-yl]-4-pyridyl]-difluoro-methyl]cyclohexanecarboxamide;
Trans-N-(3-aminopropyl)-4-[[2-chloro-6-[4-[4-[(4R)-4-amino-2-oxo-pyrrolidin-1-yl]phenyl]sulfonyl-1-piperidyl]-4-pyridyl]-difluoro-methyl]cyclohexanecarboxamide;
Cis-N-(2-amino-2-oxo-ethyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
(4R)-4-amino-1-[4-[4-[4-[[4-[(2S,4S)-4-amino-2-methyl-pyrrolidine-1-carbonyl]cyclohexyl]-difluoro-methyl]-6-chloro-2-pyridyl]piperazin-1-yl]sulfonylphenyl]pyrrolidin-2-one;
Trans-bis(2-aminoethyl)-[3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl]-methyl-ammonium;
Trans-N-[(1R,3S)-3-aminocyclopentyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-(4R)-4-amino-1-[4-[4-[4-[[4-[cis-3,4-bis(aminomethyl)pyrrolidine-1-carbonyl]cyclohexyl]-difluoro-methyl]-6-chloro-2-pyridyl]piperazin-1-yl]sulfonylphenyl]pyrrolidin-2-one;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(3R,4S)-4-methylpyrrolidin-3-yl]cyclohexanecarboxamide;
Trans-N-(3-aminopropyl)-4-[[2-chloro-6-[4-[4-[(4R)-4-(methylamino)-2-oxo-pyrrolidin-1-yl]phenyl]sulfonylpiperazin-1-yl]-4-pyridyl]-difluoro-methyl]cyclohexanecarboxamide;
Trans-(4R)-4-amino-1-[4-[4-[4-[[4-[4-(2-aminoethyl)-4-methyl-piperazin-4-ium-1-carbonyl]cyclohexyl]-difluoro-methyl]-6-chloro-2-pyridyl]piperazin-1-yl]sulfonylphenyl]pyrrolidin-2-one;
Trans-N-[2-(2-aminoethylamino)ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-2-aminoethyl-[2-[[4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarbonyl]amino]ethyl]-dimethyl-ammonium;
Trans-N-[2-[1-(2-aminoethyl)pyrrolidin-1-ium-1-yl]ethyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-N-[2-[(2-aminoacetyl)amino]ethyl]-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-N-(3-amino-2-methoxy-propyl)-4-[[2-chloro-6-[4-[4-[(4R)-4-amino-2-oxo-pyrrolidin-1-yl]phenyl]sulfonylpiperazin-1-yl]-4-pyridyl]-difluoro-methyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-(2,3-diaminopropyl)cyclohexanecarboxamide;
4-[[2-[4-[4-[4-(3-Aminopropylamino)-2-oxo-pyrrolidin-1-yl]phenyl]sulfonylpiperazin-1-yl]-6-chloro-4-pyridyl]-difluoro-methyl]-N-methyl-benzamide;
Trans-N-[(1R,3S)-3-[(2-aminoacetyl)amino]cyclopentyl]-4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarboxamide;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[cis-3,4-bis(aminomethyl)pyrrolidin-1-yl]cyclopentyl]cyclohexanecarboxamide;
Trans-4-[[2-chloro-6-[4-[4-[(4R)-4-amino-2-oxo-pyrrolidin-1-yl]phenyl]sulfonylpiperazin-1-yl]-4-pyridyl]-difluoro-methyl]-N-[(1S)-3-[cis-3,5-diamino-1-piperidyl]cyclopentyl]cyclohexanecarboxamide;
Trans-4-[[2-[4-[4-[(4R)-4-amino-2-oxo-pyrrolidin-1-yl]phenyl]sulfonylpiperazin-1-yl]-6-chloro-4-pyridyl]-difluoro-methyl]-N-[(1S,3R)-3-[(3R)-3-aminopyrrolidin-1-yl]cyclopentyl]cyclohexanecarboxamide; and
Trans-3-[[4-[[2-[4-[4-[(4R)-4-amino-2-oxo-pyrrolidin-1-yl]phenyl]sulfonylpiperazin-1-yl]-6-chloro-4-pyridyl]-difluoro-methyl]cyclohexanecarbonyl]amino]propyl-(3-aminopropyl)-dimethyl-ammonium;
or a pharmaceutically acceptable salt thereof.

17. A process for the preparation of a compound according to claim 1 comprising any of the following steps:
a) formation of compound of formula (XIX), 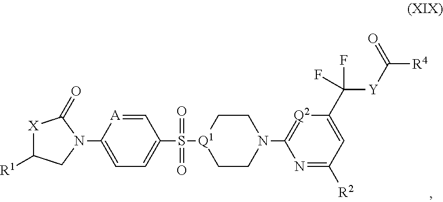
via deprotection of compound of formula (XVIII), 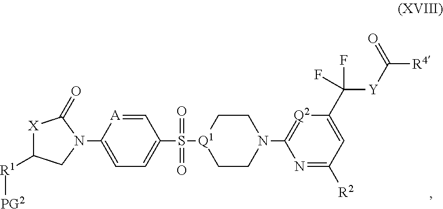
using an acid, wherein the acid is trifluoroacetic acid, or a reducing reagent, wherein the reducing agent is palladium on carbon in the presence of H₂;
b) formation of compound of formula (XXXV), 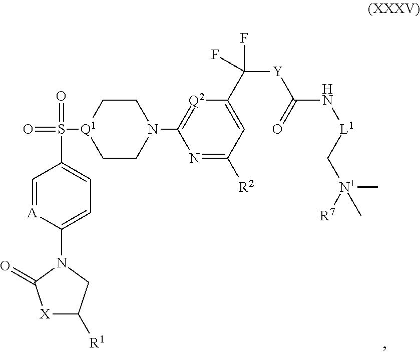
via methylation of compound of formula (XXXIII), 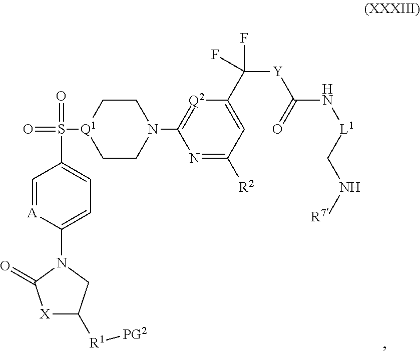
using a suitable reagent, wherein the agent is MeI, followed by deprotection with an acid, wherein the acid is trifluoroacetic acid, or a reducing reagent, wherein the reducing agent is palladium on carbon in the presence of H₂;
c) formation of compound of formula (XXXIV), 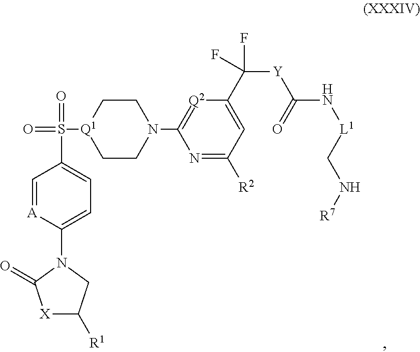
via deprotection of compound of formula (XXXIII), 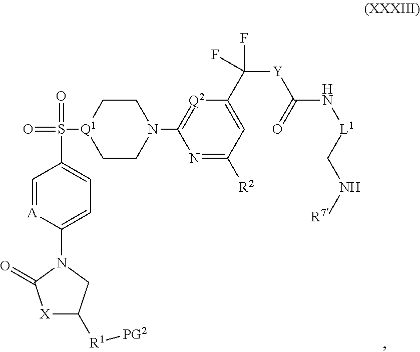
using an acid, wherein the acid is trifluoroacetic acid, or a reducing reagent, wherein the reducing agent is palladium on carbon in the presence of H₂;
d) formation of compound of formula (XXXVIII), 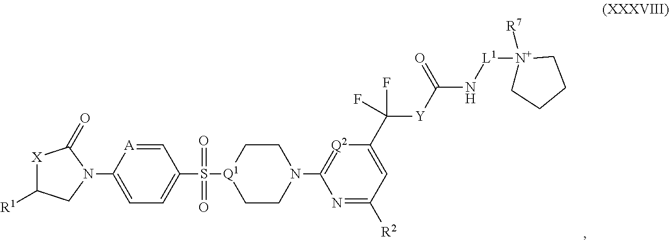
via deprotection of the compound of formula (XXXVII), 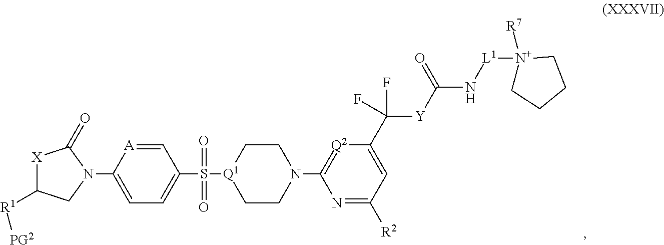
using an acid,
wherein the acid is trifluoroacetic acid, or a reducing reagent, wherein the reducing agent is palladium on carbon in the presence of H₂;
wherein R^{4′} is R⁴ or R⁴—PG³; L¹ is C₁₋₆alkyl or C₃₋₁₀cycloalkyl; R⁷ is C₁₋₆alkyl, aminoC₁₋₆ alkyl or aminoC₃₋₁₀cycloalkyl; R^{7′} is R⁷—PG⁵; PG², PG³ and PG⁵ are each independently selected from tert-butoxycarbonyl and benzyloxycarbonyl; and R¹, R², Y, A, Q¹, Q² and X are defined as in claim 1.

18. (canceled)

19. A pharmaceutical composition comprising a compound in accordance with claim 1, and a therapeutically inert carrier.

20. A method of inhibiting LpxH in gram negative bacteria comprising exposing said bacteria to a compound according to claim 1.

21-25. (canceled)

26. The method according to claim 30, wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

27. A compound according to claim 26, wherein the gram-negative bacteria is Enterobacteriaceae, wherein Enterobacteriaceae is Klebsiella pneumoniae or Escherichia coli.

28. A compound or pharmaceutically acceptable salt thereof according to claim 1 manufactured according to the process of claim 17.

29. A method for the treatment or prophylaxis of bacterial infection in a human subject which method comprises administering to said human subject a therapeutically effective amount of a compound as defined in claim 1.

30. The method of according to claim 29, wherein the bacteria infection is caused by gram-negative bacteria.

31. A compound according to claim 1, wherein R¹ is amino.

32. A compound according to claim 1, wherein X is CH₂.

33. A compound according to claim 1, wherein
R¹ is amino;
R² is chloro;
X is CH₂;
A and Q² are each CH;
Q¹ is N; and
W is —CF₂—.

34. A compound according to claim 1, wherein R³ is —Y—C(O)—R⁴; wherein Y is C₃₋₁₀cycloalkyl.

35. A compound according to claim 1, wherein R³ is —Y—C(O)—R⁴; wherein Y is cyclohexanyl.

36. A compound according to claim 1, wherein R³ is —Y—C(O)—R⁴; wherein Y is phenyl.

37. A compound according to claim 1, wherein R³ is —Y—C(O)—R⁴; wherein Y is pyrazinyl.
